SEQUENCE LISTING (301 sequences)

SEQ ID NO: 1 - protein, 105 aa, synthetic construct
  REGION 1..105: note = VL amino acid sequence of T26B373, T26B459, T26B608
DIQMTQSPSTLSASVGDRVTITCRASQSTSSWLAWYQQKPGKAPKLLIYKTSSLESGVPSRFSGSGSGTEFTLTISSLQPDDFATYYCQQYNSYSFGQGTKVEIK

SEQ ID NO: 2 - protein, 105 aa, synthetic construct
  REGION 1..105: note = VL amino acid sequence of T26B374, T26B460, T26B611
DIQMTQSPSTLSASVGDRVTITCRASQSISSWLAWYQQKPGKAPKLLIYKASSLESGVPSRFSGSRSGTEFTLTISSLQPDDFATYYCQQYNSWTFGQGTKVEIK

SEQ ID NO: 3 - protein, 105 aa, synthetic construct
  REGION 1..105: note = VL amino acid sequence of T26B375, T26B461, T26B612
DIQMTQSPSTLSASVGDRVTITCRASQSISSWLAWYQQKPGKAPKLLIYKASSLESGVPSRFSGSGSGTEFTLTISSLQPDDFATYYCQQYNSYTFGQGTKLEIK

SEQ ID NO: 4 - protein, 105 aa, synthetic construct
  REGION 1..105: note = VL amino acid sequence of T26B379, T26B462, T26B615
DIQMTQSPSTLSASVGDRVTITCRASQSISSWLAWYQQKPGKAPKLLIFKTSSLESGVPSRFSGSGSGTEFTLTISSLQPDDFATYYCQQYNSYSFGGGTKVEIK

SEQ ID NO: 5 - protein, 111 aa, synthetic construct
  REGION 1..111: note = VL amino acid sequence of T26B382, T26B463, T26B613
QSALTQPASVSGSPGQSITISCTGTSSDVGGYNYVSWYQQHPSKAPKLMIYDVSNRPSGVSNRFSGSKSGNTASLTISGLQAEDEADYYCSSYTSSSTLVVFGGGTKLTVL

SEQ ID NO: 6 - protein, 105 aa, synthetic construct
  REGION 1..105: note = VL amino acid sequence of T26B385, T26B464, T26B610
DIQMTQSPSTLSASVGDRVTITCRASQSLSSWLAWYQQKPGKAPKLLIFKTSSLESGVPSRFSGSGSGTEFTLTISSLQPDDFAIYYCQQYNSYIFGQGTRLEIK

SEQ ID NO: 7 - protein, 108 aa, synthetic construct
  REGION 1..108: note = VL amino acid sequence of T26B386, T26B465, T26B614
SSELTQDPAVSVALGQTVRITCQGDSLRNYYASWYQQKPGQAPVLVFYGKDNRPSGIPDRFSGSTSGNTASLTITGAQAQDEADYYCNSRDSSGDHLVFGGGTKLTVL

SEQ ID NO: 8 - protein, 107 aa, synthetic construct
  REGION 1..107: note = VL amino acid sequence of T26B388, T26B466, T26B609
DIQMTQSPSSLSASVGDRVTITCRASQGIRNDLGWYQQKPGKAPKRLIYAASSLQGGVPSRFSGSGSGTEFTLTISSLQPEDFATYYCLQHYTYPLTFGGGTKVEIK

SEQ ID NO: 9 - protein, 117 aa, synthetic construct
  REGION 1..117: note = VH amino acid sequence of T26B373, T26B459, T26B608
QVQLQESGPGLVKPSETLSLTCTVSGGSISSGVYYWAWIRQPPGKGLELIGTFYYSGSTYYDSSLRSRVTISVDTSKNQFSLKLSSVTAADTAVYYCARQAGDFDYWGQGTLVTVSS

SEQ ID NO: 10 - protein, 117 aa, synthetic construct
  REGION 1..117: note = VH amino acid sequence of T26B374, T26B460, T26B611
QLQLQESGPGLVKPSETLSLTCTVSGGSISSSPYYWGWIRQPPGKGLEWIGSFYYGGSNYYNPSLKSRVTISADTSKNQFSLKLSSVTAADTAVYYCARQAGDWDYWGQGTLVTVSS

SEQ ID NO: 11 - protein, 117 aa, synthetic construct
  REGION 1..117: note = VH amino acid sequence of T26B375, T26B461, T26B612
QLQLQESGPGLVKPSETLSLTCTVSGGSISSGIYNWGWIRQPPGKGLEWIGNFQYSGITYYNPSLKSRVTISVDTSKNQFSLQLSSVTAADTAVYYCARQAGDFDYWGQGTLVTVSS

SEQ ID NO: 12 - protein, 117 aa, synthetic construct
  REGION 1..117: note = VH amino acid sequence of T26B379, T26B462, T26B615
QVQLQESGPGLVKPSETLSLTCTVSGGSISSSIYYWAWIRQPPGKGLEWIGTIYYGGSPYYSPSLKSRVTISIDTSKSQFSLRLTSVTVADTAVYYCARRAGDFDYWGQGTLVTVSS

SEQ ID NO: 13 - protein, 121 aa, synthetic construct
  REGION 1..121: note = VH amino acid sequence of T26B382, T26B463, T26B613
QVQLVESGGGLVKPGGSLRLSCAASGFTFSDYYMTWIRQAPGKGLEWVSYISSSGITKYYADFVKGRFTISRDNAKNSLYLQMNSLRAEDTAVYYCARDRDRQWLLEFDYWGQGTLVTVSS

SEQ ID NO: 14 - protein, 117 aa, synthetic construct
  REGION 1..117: note = VH amino acid sequence of T26B385, T26B464, T26B610
QVQLQESGPGLVKPSETLSLTCIVSGGSVSSRNYYWGWIRQPPGKGLEWIGRIYYSGNTNYNPSLKSRVTISVDTSKNQFSLKLSSVTAADTAVYYCARWAGEIDYWGQGTLVTVSS

SEQ ID NO: 15 - protein, 122 aa, synthetic construct
  REGION 1..122: note = VH amino acid sequence of T26B386, T26B465, T26B614
QVQLQESGPGLVKPSETLSLSCTVSGGSLNSYNYYWGWVRQPPGKGLEWIGTIYSSGSAYYNPSLKSRFTISVATSKNQFSLRLSSVTAADTAVYYCARGYRNSWYALFEYWGQGTLVTVSS

SEQ ID NO: 16 - protein, 124 aa, synthetic construct
  REGION 1..124: note = VH amino acid sequence of T26B388, T26B466, T26B609
QVQLQQWGAGLLKPSETLSLTCAVYGGSFSDYYWSWIRQPPGKGLEWIGEIDHSGSTDYNPSLKSRVTISVDTSKNQFSLKLSSVTAADTAVYYCARSMYYDIWTGYHGAFDIWGQGTMVTVSS

SEQ ID NO: 17 - DNA, 315 bp, synthetic construct
  misc_feature 1..315: note = VL nucleotide sequence of T26B373, T26B459, T26B608
  source 1..315: mol_type = other DNA
gacatccagatgacccagagccctagcaccctgagcgctagcgtgggcgacagggtgaccatcacctgcagagccagccagagcaccagcagctggctggcctggtaccagcagaagcccggcaaggcccccaagctgctgatctacaagaccagcagcctggagtccggcgtgcctagcaggtttagcggcagcggcagcggcaccgagtttaccctgaccatcagcagcctccagcccgacgacttcgccacctactactgccagcagtacaacagctacagcttcggccagggcacaaaggtggagatcaag

SEQ ID NO: 18 - DNA, 315 bp, synthetic construct
  misc_feature 1..315: note = VL nucleotide sequence of T26B374, T26B460, T26B611
  source 1..315: mol_type = other DNA
gacatccagatgacccagagccctagcacactgagcgccagcgtgggcgacagagtgaccatcacctgcagggccagccagtccatcagcagctggctggcctggtaccagcagaagcccggcaaggcccccaagctgctgatctacaaggccagcagcctggagagcggcgtgcctagcaggttcagcggcagcaggagcggcaccgagttcaccctgaccatcagcagcctgcagcccgacgacttcgccacctactactgccagcagtacaacagctggaccttcggccagggcaccaaggtggagatcaag

SEQ ID NO: 19 - DNA, 315 bp, synthetic construct
  misc_feature 1..315: note = VL nucleotide sequence of T26B375, T26B461, T26B612
  source 1..315: mol_type = other DNA
gacatccagatgacccagagccctagcacactgtcagctagcgtcggcgacagggtgaccatcacatgcagggctagtcagagtatcagtagctggctggcctggtaccagcagaagcccggcaaggcccccaagctgctgatctacaaggccagcagcctggagagcggagtgcccagcagattcagtggtagcggcagtggcaccgagttcaccctgacaatcagcagcctgcagcccgacgatttcgccacctactactgccagcagtacaacagctacaccttcggccagggcaccaagctggagatcaag

SEQ ID NO: 20 - DNA, 315 bp, synthetic construct
  misc_feature 1..315: note = VL nucleotide sequence of T26B379, T26B462, T26B615
  source 1..315: mol_type = other DNA
gacatccagatgacccagagccctagcacactgagcgccagcgtgggcgacagggtgaccatcacctgcagggccagccagagcatcagcagctggctggcctggtaccagcagaagcccggcaaggcccccaagctgctgatcttcaagaccagcagcctggagagcggcgtgcctagcagatttagcggcagcggcagcggcaccgagttcaccctgaccatcagcagcctgcagcccgacgacttcgccacctactactgccagcagtacaacagctacagcttcggcggcggcaccaaggtggagatcaag

SEQ ID NO: 21 - DNA, 333 bp, synthetic construct
  misc_feature 1..333: note = VL nucleotide sequence of T26B382, T26B463, T26B613
  source 1..333: mol_type = other DNA
cagagcgccctgacccagcctgctagcgtgagcggaagccctggccagagcatcaccatcagctgcacaggcaccagcagcgatgtgggcggctacaactacgtgagctggtaccagcagcaccccagcaaggcccccaagctgatgatctacgatgtgagcaacaggcccagcggcgtgagcaataggttcagcggcagcaagagcggcaacaccgcctccctgacaatcagcggcctgcaggccgaggatgaggccgactactactgcagcagctacaccagcagctccaccctggtggtgtttggcggcggcaccaagctgaccgtgctg

SEQ ID NO: 22 - DNA, 315 bp, synthetic construct
  misc_feature 1..315: note = VL nucleotide sequence of T26B385, T26B464, T26B610
  source 1..315: mol_type = other DNA
gacatccagatgacccagagccctagcacactgagcgccagcgtgggagacagggtgaccatcacctgcagagccagccagtccctgagcagctggctggcctggtaccagcagaagcccggcaaggcccccaagctgctgatcttcaagacaagcagcctggagagcggcgtgccctccagattcagcggcagcggaagcggcaccgagttcaccctgaccatcagcagcctgcagcccgacgacttcgccatctactactgccagcagtacaactcctacatcttcggccagggcaccaggctggagatcaag

SEQ ID NO: 23 - DNA, 324 bp, synthetic construct
  misc_feature 1..324: note = VL nucleotide sequence of T26B386, T26B465, T26B614
  source 1..324: mol_type = other DNA
agcagcgagctgacccaggatcctgccgtgagcgtggccctgggacagaccgtgaggatcacctgccagggcgacagcctgaggaactactacgccagctggtaccagcagaaacccggacaggcccccgtgctggtgttctacggcaaggacaacagacccagcggcatccccgacaggttcagcggaagcaccagcggcaacaccgccagcctgaccattaccggcgctcaggcccaggacgaggccgactactactgcaacagcagggacagcagcggcgatcacctggtgtttggcggcggcaccaagctgaccgtgctg

SEQ ID NO: 24 - DNA, 321 bp, synthetic construct
  misc_feature 1..321: note = VL nucleotide sequence of T26B388, T26B466, T26B609
  source 1..321: mol_type = other DNA
gacatccagatgacccagagccctagcagcctgagcgccagcgtgggagacagggtgaccatcacctgcagggccagccagggcatcaggaacgacctgggctggtaccagcagaagcccggcaaggcccccaagaggctgatctacgctgccagcagcctgcagggcggagtgcctagcaggtttagcggctccggcagcggcacagagttcaccctgaccatctccagcctgcagcccgaggacttcgccacctactactgcctgcagcactacacctaccctctgaccttcggcggcggcaccaaggtggagatcaag

SEQ ID NO: 25 - DNA, 351 bp, synthetic construct
  misc_feature 1..351: note = VH nucleotide sequence of T26B373, T26B459, T26B608
  source 1..351: mol_type = other DNA
caggtgcagctgcaggaaagcggccccggactggtgaagcccagcgaaaccctgagcctgacctgtaccgtgagcggcggcagcatcagcagcggcgtgtactactgggcctggatcagacagccccctggcaagggcctggagctgatcggcaccttctactacagcggcagcacctactacgacagcagcctgaggagcagggtgaccatcagcgtggacaccagcaagaaccagttcagcctgaagctgagcagcgtgaccgccgccgatacagccgtgtattactgcgccaggcaggccggcgacttcgactactggggccagggaaccctggtgaccgtgagcagc

SEQ ID NO: 26 - DNA, 351 bp, synthetic construct
  misc_feature 1..351: note = VH nucleotide sequence of T26B374, T26B460, T26B611
  source 1..351: mol_type = other DNA
cagctgcagctgcaggagagcggacccggactggtgaagcccagcgagaccctgagcctgacctgcaccgtgagcggcggaagcatcagcagcagcccctactattggggctggatcaggcagcctcccggaaagggcctggagtggatcggcagcttctactacggcggcagcaactactacaaccccagcctgaagagcagggtgaccatcagcgccgacaccagcaagaaccagttcagcctgaagctgtccagcgtgaccgccgctgataccgccgtgtactactgcgccagacaggccggcgactgggattactggggccagggcacactggtgaccgtgagcagc

SEQ ID NO: 27 - DNA, 351 bp, synthetic construct
  misc_feature 1..351: note = VH nucleotide sequence of T26B375, T26B461, T26B612
  source 1..351: mol_type = other DNA
cagctgcagctgcaggagagcggacctggcctggtgaagcccagcgagaccctgagcctgacctgcaccgtgagcggcggaagcatcagcagcggcatctacaactggggctggatcaggcagccccctggcaaaggcctggagtggatcggcaacttccagtacagcggcatcacctactacaaccccagcctgaagagcagggtgaccatcagcgtggacaccagcaagaaccagttcagcctgcagctgagcagcgtgacagctgccgacaccgccgtgtactactgtgccaggcaggccggcgacttcgattactggggccagggcaccctggtgacagtgagcagc

SEQ ID NO: 28 - DNA, 351 bp, synthetic construct
  misc_feature 1..351: note = VH nucleotide sequence of T26B379, T26B462, T26B615
  source 1..351: mol_type = other DNA
caggtgcagctgcaggaatccggccctggcctggtgaaacccagcgagaccctgagcctgacctgcacagtgagtggaggtagcatcagcagcagcatctactactgggcctggatcaggcagcctcccggcaaaggactggagtggatcggtaccatctactacggcggcagcccctattacagtccctcactgaagagcagggtgaccatcagcatcgacaccagcaagtcccagttcagcctgaggctgaccagcgtgacagtggccgacaccgccgtgtattactgcgccaggagggccggcgacttcgactactggggacagggcaccctggtgaccgtgagcagc

SEQ ID NO: 29 - DNA, 363 bp, synthetic construct
  misc_feature 1..363: note = VH nucleotide sequence of T26B382, T26B463, T26B613
  source 1..363: mol_type = other DNA
caggtgcagctggtggagagcggcggaggactggtgaagcctggaggctccctgagactgagctgtgccgcctccggcttcaccttcagcgactactatatgacctggatcaggcaggcccctggcaagggactggagtgggtgagctacatcagcagcagcggcatcaccaagtactacgccgacttcgtgaagggcaggttcaccatcagcagggacaacgccaagaacagcctgtacctgcagatgaacagcctgagggccgaggacaccgccgtgtactactgcgccagggacagagacaggcagtggctgctggagtttgactactggggccagggcaccctggtgacagtgtccagc

SEQ ID NO: 30 - DNA, 351 bp, synthetic construct
  misc_feature 1..351: note = VH nucleotide sequence of T26B385, T26B464, T26B610
  source 1..351: mol_type = other DNA
caggtgcagctgcaggagagcggccctggactggtgaagcccagcgagaccctgagcctgacctgcatcgtgagcggcggcagcgtgagcagcagaaactactactggggctggattaggcagccccctggcaaaggcctggagtggatcggcaggatctactacagcggcaacaccaactacaaccccagcctgaagagcagagtgaccatctccgtggacaccagcaagaaccagttcagcctgaagctgagcagcgtgacagccgccgataccgccgtgtactactgcgccagatgggccggcgaaatcgactactggggccagggcacactggtgaccgtgagcagc

SEQ ID NO: 31 - DNA, 366 bp, synthetic construct
  misc_feature 1..366: note = VH nucleotide sequence of T26B386, T26B465, T26B614
  source 1..366: mol_type = other DNA
caggtgcagctgcaggagagcggccctggactggtgaaacccagcgagaccctgagcctgagctgcacagtgagcggcggcagcctgaacagctacaactactactggggctgggtgagacagcctcccggcaagggactggagtggatcggcaccatctacagcagcggctccgcctactacaaccccagcctgaagagcaggttcaccatcagcgtggccaccagcaagaaccagttcagcctgagactgagcagcgtgacagccgccgacaccgctgtgtactactgcgccaggggctacaggaacagctggtacgccctgttcgagtactggggacagggcaccctggtgaccgtgagcagc

SEQ ID NO: 32 - DNA, 372 bp, synthetic construct
  misc_feature 1..372: note = VH nucleotide sequence of T26B388, T26B466, T26B609
  source 1..372: mol_type = other DNA
caggtgcagctgcagcagtggggcgctggactgctgaagcccagcgagaccctgtccctgacctgtgccgtgtacggcggcagcttcagcgactactactggagctggatcagacagcctcctggcaagggcctggagtggatcggcgagatcgaccacagcggctccaccgactacaaccccagcctgaagtccagggtgaccatcagcgtggacaccagcaagaaccagttcagcctgaagctgagcagcgtgacagccgccgataccgccgtgtactactgcgccaggagcatgtactacgacatctggaccggctaccacggcgcctttgacatctggggccagggcaccatggtgaccgtgagcagc

SEQ ID NO: 33 - protein, 107 aa, synthetic construct [sequence omitted]

SEQ ID NO: 34 - protein, 125 aa, synthetic construct
  REGION 1..125: note = VH amino acid sequence of GC5B747, GC5B752
QLQLQESGPGLVKPSETLSLTCTVSGGSLSSSSYWWGWTRQPPGRGLEWIGTMYYSGNIYYNPSLQSRATISVDTSKNQFSLKLSSVTAADTAVYYCARHVGYSYGRRFWYFDLWGRGTLVTVSS

SEQ ID NO: 35 - DNA, 321 bp, synthetic construct
  misc_feature 1..321: note = VL nucleotide sequence of GC5B747, GC5B752
  source 1..321: mol_type = other DNA
gaaatcgtattgacccaatctcctgctactctcagcttgagccccggtgagagagcaaccctgtcttgtcgagcctctcaaagtgtgtcttcctacctcgcctggtatcaacagaagcctggccaagccccacgactgcttatttatgacgcttccaatcgggctactggaatccctgccaggttctccgggagtggttctggaacagacttcactctgaccatctcctcactggaacccgaggacttcgcagtctattattgccaacagaggagtaattggccccctacatttggccaaggcacaaaagtcgagattaag

SEQ ID NO: 36 - DNA, 375 bp, synthetic construct
  misc_feature 1..375: note = VH nucleotide sequence of GC5B747, GC5B752
  source 1..375: mol_type = other DNA
caacttcaacttcaggagagtggcccaggactcgttaaacctagtgagaccctgtccctgacctgtactgtgtccggtggatcactgtcatcctcctcatattggtggggctggactcgccagccaccaggcaggggattggagtggattggcactatgtattactcaggcaatatttactataatccctctctccagagtcgggctacaatatccgtcgatactagcaaaaatcaattttccctgaaattgagtagcgtaactgccgccgacacagccgtctattactgtgcacgacacgttgggtattcatacggtaggcgattttggtacttcgatttgtgggggagaggaacccttgtgactgtaagcagc

SEQ ID NO: 37 - protein, 212 aa, synthetic construct
  REGION 1..212: note = Light chain amino acid sequence of T26B373, T26B459, T26B608
DIQMTQSPSTLSASVGDRVTITCRASQSTSSWLAWYQQKPGKAPKLLIYKTSSLESGVPSRFSGSGSGTEFTLTISSLQPDDFATYYCQQYNSYSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 212 aa, synthetic construct
  REGION 1..212: note = Light chain amino acid sequence of T26B374, T26B460, T26B611
DIQMTQSPSTLSASVGDRVTITCRASQSISSWLAWYQQKPGKAPKLLIYKASSLESGVPSRFSGSRSGTEFTLTISSLQPDDFATYYCQQYNSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 212 aa, synthetic construct
  REGION 1..212: note = Light chain amino acid sequence of T26B375, T26B461, T26B612
DIQMTQSPSTLSASVGDRVTITCRASQSISSWLAWYQQKPGKAPKLLIYKASSLESGVPSRFSGSGSGTEFTLTISSLQPDDFATYYCQQYNSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 212 aa, synthetic construct
  REGION 1..212: note = Light chain amino acid sequence of T26B379, T26B462, T26B615
DIQMTQSPSTLSASVGDRVTITCRASQSISSWLAWYQQKPGKAPKLLIFKTSSLESGVPSRFSGSGSGTEFTLTISSLQPDDFATYYCQQYNSYSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 217 aa, synthetic construct
  REGION 1..217: note = Light chain amino acid sequence of T26B382, T26B463, T26B613
QSALTQPASVSGSPGQSITISCTGTSSDVGGYNYVSWYQQHPSKAPKLMIYDVSNRPSGVSNRFSGSKSGNTASLTISGLQAEDEADYYCSSYTSSSTLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - protein, 212 aa, synthetic construct
  REGION 1..212: note = Light chain amino acid sequence of T26B385, T26B464, T26B610
DIQMTQSPSTLSASVGDRVTITCRASQSLSSWLAWYQQKPGKAPKLLIFKTSSLESGVPSRFSGSGSGTEFTLTISSLQPDDFAIYYCQQYNSYIFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 214 aa, synthetic construct
  REGION 1..214: note = Light chain amino acid sequence of T26B386, T26B465, T26B614
SSELTQDPAVSVALGQTVRITCQGDSLRNYYASWYQQKPGQAPVLVFYGKDNRPSGIPDRFSGSTSGNTASLTITGAQAQDEADYYCNSRDSSGDHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 44 - protein, 214 aa, synthetic construct
  REGION 1..214: note = Light chain amino acid sequence of T26B388, T26B466, T26B609
DIQMTQSPSSLSASVGDRVTITCRASQGIRNDLGWYQQKPGKAPKRLIYAASSLQGGVPSRFSGSGSGTEFTLTISSLQPEDFATYYCLQHYTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 447 aa, synthetic construct
  REGION 1..447: note = Heavy chain amino acid sequence of T26B373
QVQLQESGPGLVKPSETLSLTCTVSGGSISSGVYYWAWIRQPPGKGLELIGTFYYSGSTYYDSSLRSRVTISVDTSKNQFSLKLSSVTAADTAVYYCARQAGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 447 aa, synthetic construct
  REGION 1..447: note = Heavy chain amino acid sequence of T26B374
QLQLQESGPGLVKPSETLSLTCTVSGGSISSSPYYWGWIRQPPGKGLEWIGSFYYGGSNYYNPSLKSRVTISADTSKNQFSLKLSSVTAADTAVYYCARQAGDWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 447 aa, synthetic construct
  REGION 1..447: note = Heavy chain amino acid sequence of T26B375
QLQLQESGPGLVKPSETLSLTCTVSGGSISSGIYNWGWIRQPPGKGLEWIGNFQYSGITYYNPSLKSRVTISVDTSKNQFSLQLSSVTAADTAVYYCARQAGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 447 aa, synthetic construct
  REGION 1..447: note = Heavy chain amino acid sequence of T26B379
QVQLQESGPGLVKPSETLSLTCTVSGGSISSSIYYWAWIRQPPGKGLEWIGTIYYGGSPYYSPSLKSRVTISIDTSKSQFSLRLTSVTVADTAVYYCARRAGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 451 aa, synthetic construct
  REGION 1..451: note = Heavy chain amino acid sequence of T26B382
QVQLVESGGGLVKPGGSLRLSCAASGFTFSDYYMTWIRQAPGKGLEWVSYISSSGITKYYADFVKGRFTISRDNAKNSLYLQMNSLRAEDTAVYYCARDRDRQWLLEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 447 aa, synthetic construct
  REGION 1..447: note = Heavy chain amino acid sequence of T26B385
QVQLQESGPGLVKPSETLSLTCIVSGGSVSSRNYYWGWIRQPPGKGLEWIGRIYYSGNTNYNPSLKSRVTISVDTSKNQFSLKLSSVTAADTAVYYCARWAGEIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 452 aa, synthetic construct
  REGION 1..452: note = Heavy chain amino acid sequence of T26B386
QVQLQESGPGLVKPSETLSLSCTVSGGSLNSYNYYWGWVRQPPGKGLEWIGTIYSSGSAYYNPSLKSRFTISVATSKNQFSLRLSSVTAADTAVYYCARGYRNSWYALFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 454 aa, synthetic construct
  REGION 1..454: note = Heavy chain amino acid sequence of T26B388
QVQLQQWGAGLLKPSETLSLTCAVYGGSFSDYYWSWIRQPPGKGLEWIGEIDHSGSTDYNPSLKSRVTISVDTSKNQFSLKLSSVTAADTAVYYCARSMYYDIWTGYHGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 447 aa, synthetic construct
  REGION 1..447: note = Heavy chain amino acid sequence of T26B459
QVQLQESGPGLVKPSETLSLTCTVSGGSISSGVYYWAWIRQPPGKGLELIGTFYYSGSTYYDSSLRSRVTISVDTSKNQFSLKLSSVTAADTAVYYCARQAGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPGK

SEQ ID NO: 54 - protein, 447 aa, synthetic construct
  REGION 1..447: note = Heavy chain amino acid sequence of T26B460
QLQLQESGPGLVKPSETLSLTCTVSGGSISSSPYYWGWIRQPPGKGLEWIGSFYYGGSNYYNPSLKSRVTISADTSKNQFSLKLSSVTAADTAVYYCARQAGDWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPGK

SEQ ID NO: 55 - protein, 447 aa, synthetic construct
  REGION 1..447: note = Heavy chain amino acid sequence of T26B461
QLQLQESGPGLVKPSETLSLTCTVSGGSISSGIYNWGWIRQPPGKGLEWIGNFQYSGITYYNPSLKSRVTISVDTSKNQFSLQLSSVTAADTAVYYCARQAGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPGK

SEQ ID NO: 56 - protein, 447 aa, synthetic construct
  REGION 1..447: note = Heavy chain amino acid sequence of T26B462
QVQLQESGPGLVKPSETLSLTCTVSGGSISSSIYYWAWIRQPPGKGLEWIGTIYYGGSPYYSPSLKSRVTISIDTSKSQFSLRLTSVTVADTAVYYCARRAGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPGK

SEQ ID NO: 57 - protein, 451 aa, synthetic construct
  REGION 1..451: note = Heavy chain amino acid sequence of T26B463
QVQLVESGGGLVKPGGSLRLSCAASGFTFSDYYMTWIRQAPGKGLEWVSYISSSGITKYYADFVKGRFTISRDNAKNSLYLQMNSLRAEDTAVYYCARDRDRQWLLEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPGK

SEQ ID NO: 58 - protein, 447 aa, synthetic construct
  REGION 1..447: note = Heavy chain amino acid sequence of T26B464
QVQLQESGPGLVKPSETLSLTCIVSGGSVSSRNYYWGWIRQPPGKGLEWIGRIYYSGNTNYNPSLKSRVTISVDTSKNQFSLKLSSVTAADTAVYYCARWAGEIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPGK

SEQ ID NO: 59 - protein, 452 aa, synthetic construct
  REGION 1..452: note = Heavy chain amino acid sequence of T26B465
QVQLQESGPGLVKPSETLSLSCTVSGGSLNSYNYYWGWVRQPPGKGLEWIGTIYSSGSAYYNPSLKSRFTISVATSKNQFSLRLSSVTAADTAVYYCARGYRNSWYALFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPGK

SEQ ID NO: 60 - protein, 454 aa, synthetic construct
  REGION 1..454: note = Heavy chain amino acid sequence of T26B466
QVQLQQWGAGLLKPSETLSLTCAVYGGSFSDYYWSWIRQPPGKGLEWIGEIDHSGSTDYNPSLKSRVTISVDTSKNQFSLKLSSVTAADTAVYYCARSMYYDIWTGYHGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPGK

SEQ ID NO: 61 - protein, 446 aa, synthetic construct
  REGION 1..446: note = Heavy chain amino acid sequence of T26B608
QVQLQESGPGLVKPSETLSLTCTVSGGSISSGVYYWAWIRQPPGKGLELIGTFYYSGSTYYDSSLRSRVTISVDTSKNQFSLKLSSVTAADTAVYYCARQAGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 453 aa, synthetic construct
  REGION 1..453: note = Heavy chain amino acid sequence of T26B609
QVQLQQWGAGLLKPSETLSLTCAVYGGSFSDYYWSWIRQPPGKGLEWIGEIDHSGSTDYNPSLKSRVTISVDTSKNQFSLKLSSVTAADTAVYYCARSMYYDIWTGYHGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 446 aa, synthetic construct
  REGION 1..446: note = Heavy chain amino acid sequence of T26B610
QVQLQESGPGLVKPSETLSLTCIVSGGSVSSRNYYWGWIRQPPGKGLEWIGRIYYSGNTNYNPSLKSRVTISVDTSKNQFSLKLSSVTAADTAVYYCARWAGEIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 446 aa, synthetic construct
  REGION 1..446: note = Heavy chain amino acid sequence of T26B611
QLQLQESGPGLVKPSETLSLTCTVSGGSISSSPYYWGWIRQPPGKGLEWIGSFYYGGSNYYNPSLKSRVTISADTSKNQFSLKLSSVTAADTAVYYCARQAGDWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 446 aa, synthetic construct
  REGION 1..446: note = Heavy chain amino acid sequence of T26B612
QLQLQESGPGLVKPSETLSLTCTVSGGSISSGIYNWGWIRQPPGKGLEWIGNFQYSGITYYNPSLKSRVTISVDTSKNQFSLQLSSVTAADTAVYYCARQAGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 450 aa, synthetic construct
  REGION 1..450: note = Heavy chain amino acid sequence of T26B613
QVQLVESGGGLVKPGGSLRLSCAASGFTFSDYYMTWIRQAPGKGLEWVSYISSSGITKYYADFVKGRFTISRDNAKNSLYLQMNSLRAEDTAVYYCARDRDRQWLLEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 451 aa, synthetic construct
  REGION 1..451: note = Heavy chain amino acid sequence of T26B614
QVQLQESGPGLVKPSETLSLSCTVSGGSLNSYNYYWGWVRQPPGKGLEWIGTIYSSGSAYYNPSLKSRFTISVATSKNQFSLRLSSVTAADTAVYYCARGYRNSWYALFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 446 aa, synthetic construct
  REGION 1..446: note = Heavy chain amino acid sequence of T26B615
QVQLQESGPGLVKPSETLSLTCTVSGGSISSSIYYWAWIRQPPGKGLEWIGTIYYGGSPYYSPSLKSRVTISIDTSKSQFSLRLTSVTVADTAVYYCARRAGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - DNA, 636 bp, synthetic construct
  misc_feature 1..636: note = Light chain nucleotide sequence of T26B373, T26B459, T26B608
  source 1..636: mol_type = other DNA
gacatccagatgacccagagccctagcaccctgagcgctagcgtgggcgacagggtgaccatcacctgcagagccagccagagcaccagcagctggctggcctggtaccagcagaagcccggcaaggcccccaagctgctgatctacaagaccagcagcctggagtccggcgtgcctagcaggtttagcggcagcggcagcggcaccgagtttaccctgaccatcagcagcctccagcccgacgacttcgccacctactactgccagcagtacaacagctacagcttcggccagggcacaaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 70 - DNA, 636 bp, synthetic construct
  misc_feature 1..636: note = Light chain nucleotide sequence of T26B374, T26B460, T26B611
  source 1..636: mol_type = other DNA
gacatccagatgacccagagccctagcacactgagcgccagcgtgggcgacagagtgaccatcacctgcagggccagccagtccatcagcagctggctggcctggtaccagcagaagcccggcaaggcccccaagctgctgatctacaaggccagcagcctggagagcggcgtgcctagcaggttcagcggcagcaggagcggcaccgagttcaccctgaccatcagcagcctgcagcccgacgacttcgccacctactactgccagcagtacaacagctggaccttcggccagggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 71 - DNA, 636 bp, synthetic construct
  misc_feature 1..636: note = Light chain nucleotide sequence of T26B375, T26B461, T26B612
  source 1..636: mol_type = other DNA
gacatccagatgacccagagccctagcacactgtcagctagcgtcggcgacagggtgaccatcacatgcagggctagtcagagtatcagtagctggctggcctggtaccagcagaagcccggcaaggcccccaagctgctgatctacaaggccagcagcctggagagcggagtgcccagcagattcagtggtagcggcagtggcaccgagttcaccctgacaatcagcagcctgcagcccgacgatttcgccacctactactgccagcagtacaacagctacaccttcggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 72 - DNA, 636 bp, synthetic construct
  misc_feature 1..636: note = Light chain nucleotide sequence of T26B379, T26B462, T26B615
  source 1..636: mol_type = other DNA
gacatccagatgacccagagccctagcacactgagcgccagcgtgggcgacagggtgaccatcacctgcagggccagccagagcatcagcagctggctggcctggtaccagcagaagcccggcaaggcccccaagctgctgatcttcaagaccagcagcctggagagcggcgtgcctagcagatttagcggcagcggcagcggcaccgagttcaccctgaccatcagcagcctgcagcccgacgacttcgccacctactactgccagcagtacaacagctacagcttcggcggcggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 73 - DNA, 651 bp, synthetic construct
  misc_feature 1..651: note = Light chain nucleotide sequence of T26B382, T26B463, T26B613
  source 1..651: mol_type = other DNA
cagagcgccctgacccagcctgctagcgtgagcggaagccctggccagagcatcaccatcagctgcacaggcaccagcagcgatgtgggcggctacaactacgtgagctggtaccagcagcaccccagcaaggcccccaagctgatgatctacgatgtgagcaacaggcccagcggcgtgagcaataggttcagcggcagcaagagcggcaacaccgcctccctgacaatcagcggcctgcaggccgaggatgaggccgactactactgcagcagctacaccagcagctccaccctggtggtgtttggcggcggcaccaagctgaccgtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 74 - DNA, 636 bp, synthetic construct
  misc_feature 1..636: note = Light chain nucleotide sequence of T26B385, T26B464, T26B610
  source 1..636: mol_type = other DNA
gacatccagatgacccagagccctagcacactgagcgccagcgtgggagacagggtgaccatcacctgcagagccagccagtccctgagcagctggctggcctggtaccagcagaagcccggcaaggcccccaagctgctgatcttcaagacaagcagcctggagagcggcgtgccctccagattcagcggcagcggaagcggcaccgagttcaccctgaccatcagcagcctgcagcccgacgacttcgccatctactactgccagcagtacaactcctacatcttcggccagggcaccaggctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 75 - DNA, 642 bp, synthetic construct
  misc_feature 1..642: note = Light chain nucleotide sequence of T26B386, T26B465, T26B614
  source 1..642: mol_type = other DNA
agcagcgagctgacccaggatcctgccgtgagcgtggccctgggacagaccgtgaggatcacctgccagggcgacagcctgaggaactactacgccagctggtaccagcagaaacccggacaggcccccgtgctggtgttctacggcaaggacaacagacccagcggcatccccgacaggttcagcggaagcaccagcggcaacaccgccagcctgaccattaccggcgctcaggcccaggacgaggccgactactactgcaacagcagggacagcagcggcgatcacctggtgtttggcggcggcaccaagctgaccgtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 76 - DNA, 642 bp, synthetic construct
  misc_feature 1..642: note = Light chain nucleotide sequence of T26B388, T26B466, T26B609
  source 1..642: mol_type = other DNA
gacatccagatgacccagagccctagcagcctgagcgccagcgtgggagacagggtgaccatcacctgcagggccagccagggcatcaggaacgacctgggctggtaccagcagaagcccggcaaggcccccaagaggctgatctacgctgccagcagcctgcagggcggagtgcctagcaggtttagcggctccggcagcggcacagagttcaccctgaccatctccagcctgcagcccgaggacttcgccacctactactgcctgcagcactacacctaccctctgaccttcggcggcggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 77 - DNA, 1341 bp, synthetic construct
  misc_feature 1..1341: note = Heavy chain nucleotide sequence of T26B373
  source 1..1341: mol_type = other DNA
caggtgcagctgcaggaaagcggccccggactggtgaagcccagcgaaaccctgagcctgacctgtaccgtgagcggcggcagcatcagcagcggcgtgtactactgggcctggatcagacagccccctggcaagggcctggagctgatcggcaccttctactacagcggcagcacctactacgacagcagcctgaggagcagggtgaccatcagcgtggacaccagcaagaaccagttcagcctgaagctgagcagcgtgaccgccgccgatacagccgtgtattactgcgccaggcaggccggcgacttcgactactggggccagggaaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagagcctctccctgtctccgggtaaa

SEQ ID NO: 78 - DNA, 1341 bp, synthetic construct
  misc_feature 1..1341: note = Heavy chain nucleotide sequence of T26B374
  source 1..1341: mol_type = other DNA
cagctgcagctgcaggagagcggacccggactggtgaagcccagcgagaccctgagcctgacctgcaccgtgagcggcggaagcatcagcagcagcccctactattggggctggatcaggcagcctcccggaaagggcctggagtggatcggcagcttctactacggcggcagcaactactacaaccccagcctgaagagcagggtgaccatcagcgccgacaccagcaagaaccagttcagcctgaagctgtccagcgtgaccgccgctgataccgccgtgtactactgcgccagacaggccggcgactgggattactggggccagggcacactggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagagcctctccctgtctccgggtaaa

SEQ ID NO: 79 - DNA, 1341 bp, synthetic construct
  misc_feature 1..1341: note = Heavy chain nucleotide sequence of T26B375
  source 1..1341: mol_type = other DNA
cagctgcagctgcaggagagcggacctggcctggtgaagcccagcgagaccctgagcctgacctgcaccgtgagcggcggaagcatcagcagcggcatctacaactggggctggatcaggcagccccctggcaaaggcctggagtggatcggcaacttccagtacagcggcatcacctactacaaccccagcctgaagagcagggtgaccatcagcgtggacaccagcaagaaccagttcagcctgcagctgagcagcgtgacagctgccgacaccgccgtgtactactgtgccaggcaggccggcgacttcgattactggggccagggcaccctggtgacagtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagagcctctccctgtctccgggtaaa

SEQ ID NO: 80 - DNA, 1341 bp, synthetic construct
  misc_feature 1..1341: note = Heavy chain nucleotide sequence of T26B379
  source 1..1341: mol_type = other DNA
caggtgcagctgcaggaatccggccctggcctggtgaaacccagcgagaccctgagcctgacctgcacagtgagtggaggtagcatcagcagcagcatctactactgggcctggatcaggcagcctcccggcaaaggactggagtggatcggtaccatctactacggcggcagcccctattacagtccctcactgaagagcagggtgaccatcagcatcgacaccagcaagtcccagttcagcctgaggctgaccagcgtgacagtggccgacaccgccgtgtattactgcgccaggagggccggcgacttcgactactggggacagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagagcctctccctgtctccgggtaaa

SEQ ID NO: 81 - DNA, 1353 bp, synthetic construct
  misc_feature 1..1353: note = Heavy chain nucleotide sequence of T26B382
  source 1..1353: mol_type = other DNA
caggtgcagctggtggagagcggcggaggactggtgaagcctggaggctccctgagactgagctgtgccgcctccggcttcaccttcagcgactactatatgacctggatcaggcaggcccctggcaagggactggagtgggtgagctacatcagcagcagcggcatcaccaagtactacgccgacttcgtgaagggcaggttcaccatcagcagggacaacgccaagaacagcctgtacctgcagatgaacagcctgagggccgaggacaccgccgtgtactactgcgccagggacagagacaggcagtggctgctggagtttgactactggggccagggcaccctggtgacagtgtccagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagagcctctccctgtctccgggtaaa

SEQ ID NO: 82 - DNA, 1341 bp, synthetic construct
  misc_feature 1..1341: note = Heavy chain nucleotide sequence of T26B385
  source 1..1341: mol_type = other DNA
caggtgcagctgcaggagagcggccctggactggtgaagcccagcgagaccctgagcctgacctgcatcgtgagcggcggcagcgtgagcagcagaaactactactggggctggattaggcagccccctggcaaaggcctggagtggatcggcaggatctactacagcggcaacaccaactacaaccccagcctgaagagcagagtgaccatctccgtggacaccagcaagaaccagttcagcctgaagctgagcagcgtgacagccgccgataccgccgtgtactactgcgccagatgggccggcgaaatcgactactggggccagggcacactggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagagcctctccctgtctccgggtaaa

SEQ ID NO: 83 - DNA, 1356 bp, synthetic construct
  misc_feature 1..1356: note = Heavy chain nucleotide sequence of T26B386
  source 1..1356: mol_type = other DNA
caggtgcagctgcaggagagcggccctggactggtgaaacccagcgagaccctgagcctgagctgcacagtgagcggcggcagcctgaacagctacaactactactggggctgggtgagacagcctcccggcaagggactggagtggatcggcaccatctacagcagcggctccgcctactacaaccccagcctgaagagcaggttcaccatcagcgtggccaccagcaagaaccagttcagcctgagactgagcagcgtgacagccgccgacaccgctgtgtactactgcgccaggggctacaggaacagctggtacgccctgttcgagtactggggacagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagagcctctccctgtctccgggtaaa

SEQ ID NO: 84 - DNA, 1362 bp, synthetic construct
  misc_feature 1..1362: note = Heavy chain nucleotide sequence of T26B388
  source 1..1362: mol_type = other DNA
caggtgcagctgcagcagtggggcgctggactgctgaagcccagcgagaccctgtccctgacctgtgccgtgtacggcggcagcttcagcgactactactggagctggatcagacagcctcctggcaagggcctggagtggatcggcgagatcgaccacagcggctccaccgactacaaccccagcctgaagtccagggtgaccatcagcgtggacaccagcaagaaccagttcagcctgaagctgagcagcgtgacagccgccgataccgccgtgtactactgcgccaggagcatgtactacgacatctggaccggctaccacggcgcctttgacatctggggccagggcaccatggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagagcctctccctgtctccgggtaaa

SEQ ID NO: 85 - DNA, 1341 bp, synthetic construct
  misc_feature 1..1341: note = Heavy chain nucleotide sequence of T26B459
  source 1..1341: mol_type = other DNA
caggtgcagctgcaggaaagcggccccggactggtgaagcccagcgaaaccctgagcctgacctgtaccgtgagcggcggcagcatcagcagcggcgtgtactactgggcctggatcagacagccccctggcaagggcctggagctgatcggcaccttctactacagcggcagcacctactacgacagcagcctgaggagcagggtgaccatcagcgtggacaccagcaagaaccagttcagcctgaagctgagcagcgtgaccgccgccgatacagccgtgtattactgcgccaggcaggccggcgacttcgactactggggccagggaaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactaccggcagaagagcctctccctgtctccgggtaaa

SEQ ID NO: 86 - DNA, 1341 bp, synthetic construct
  misc_feature 1..1341: note = Heavy chain nucleotide sequence of T26B460
  source 1..1341: mol_type = other DNA
cagctgcagctgcaggagagcggacccggactggtgaagcccagcgagaccctgagcctgacctgcaccgtgagcggcggaagcatcagcagcagcccctactattggggctggatcaggcagcctcccggaaagggcctggagtggatcggcagcttctactacggcggcagcaactactacaaccccagcctgaagagcagggtgaccatcagcgccgacaccagcaagaaccagttcagcctgaagctgtccagcgtgaccgccgctgataccgccgtgtactactgcgccagacaggccggcgactgggattactggggccagggcacactggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactaccggcagaagagcctctccctgtctccgggtaaa

SEQ ID NO: 87 - DNA, 1341 bp, synthetic construct
  misc_feature 1..1341: note = Heavy chain nucleotide sequence of T26B461
  source 1..1341: mol_type = other DNA
cagctgcagctgcaggagagcggacctggcctggtgaagcccagcgagaccctgagcctgacctgcaccgtgagcggcggaagcatcagcagcggcatctacaactggggctggatcaggcagccccctggcaaaggcctggagtggatcggcaacttccagtacagcggcatcacctactacaaccccagcctgaagagcagggtgaccatcagcgtggacaccagcaagaaccagttcagcctgcagctgagcagcgtgacagctgccgacaccgccgtgtactactgtgccaggcaggccggcgacttcgattactggggccagggcaccctggtgacagtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactaccggcagaagagcctctccctgtctccgggtaaa

SEQ ID NO: 88 - DNA, 1341 bp, synthetic construct
  misc_feature 1..1341: note = Heavy chain nucleotide sequence of T26B462
  source 1..1341: mol_type = other DNA
caggtgcagctgcaggaatccggccctggcctggtgaaacccagcgagaccctgagcctgacctgcacagtgagtggaggtagcatcagcagcagcatctactactgggcctggatcaggcagcctcccggcaaaggactggagtggatcggtaccatctactacggcggcagcccctattacagtccctcactgaagagcagggtgaccatcagcatcgacaccagcaagtcccagttcagcctgaggctgaccagcgtgacagtggccgacaccgccgtgtattactgcgccaggagggccggcgacttcgactactggggacagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactaccggcagaagagcctctccctgtctccgggtaaa

SEQ ID NO: 89 - DNA, 1353 bp, synthetic construct
  misc_feature 1..1353: note = Heavy chain nucleotide sequence of T26B463
  source 1..1353: mol_type = other DNA
caggtgcagctggtggagagcggcggaggactggtgaagcctggaggctccctgagactgagctgtgccgcctccggcttcaccttcagcgactactatatgacctggatcaggcaggcccctggcaagggactggagtgggtgagctacatcagcagcagcggcatcaccaagtactacgccgacttcgtgaagggcaggttcaccatcagcagggacaacgccaagaacagcctgtacctgcagatgaacagcctgagggccgaggacaccgccgtgtactactgcgccagggacagagacaggcagtggctgctggagtttgactactggggccagggcaccctggtgacagtgtccagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactaccggcagaagagcctctccctgtctccgggtaaa

SEQ ID NO: 90 - DNA, 1341 bp, synthetic construct
  misc_feature 1..1341: note = Heavy chain nucleotide sequence of T26B464
  source 1..1341: mol_type = other DNA
caggtgcagctgcaggagagcggccctggactggtgaagcccagcgagaccctgagcctgacctgcatcgtgagcggcggcagcgtgagcagcagaaactactactggggctggattaggcagccccctggcaaaggcctggagtggatcggcaggatctactacagcggcaacaccaactacaaccccagcctgaagagcagagtgaccatctccgtggacaccagcaagaaccagttcagcctgaagctgagcagcgtgacagccgccgataccgccgtgtactactgcgccagatgggccggcgaaatcgactactggggccagggcacactggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactaccggcagaagagcctctccctgtctccgggtaaa

SEQ ID NO: 91 - DNA, 1356 bp, synthetic construct
  misc_feature 1..1356: note = Heavy chain nucleotide sequence of T26B465
  source 1..1356: mol_type = other DNA
caggtgcagctgcaggagagcggccctggactggtgaaacccagcgagaccctgagcctgagctgcacagtgagcggcggcagcctgaacagctacaactactactggggctgggtgagacagcctcccggcaagggactggagtggatcggcaccatctacagcagcggctccgcctactacaaccccagcctgaagagcaggttcaccatcagcgtggccaccagcaagaaccagttcagcctgagactgagcagcgtgacagccgccgacaccgctgtgtactactgcgccaggggctacaggaacagctggtacgccctgttcgagtactggggacagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactaccggcagaagagcctctccctgtctccgggtaaa

SEQ ID NO: 92 - DNA, 1362 bp, synthetic construct
  misc_feature 1..1362: note = Heavy chain nucleotide sequence of T26B466
  source 1..1362: mol_type = other DNA
caggtgcagctgcagcagtggggcgctggactgctgaagcccagcgagaccctgtccctgacctgtgccgtgtacggcggcagcttcagcgactactactggagctggatcagacagcctcctggcaagggcctggagtggatcggcgagatcgaccacagcggctccaccgactacaaccccagcctgaagtccagggtgaccatcagcgtggacaccagcaagaaccagttcagcctgaagctgagcagcgtgacagccgccgataccgccgtgtactactgcgccaggagcatgtactacgacatctggaccggctaccacggcgcctttgacatctggggccagggcaccatggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactaccggcagaagagcctctccctgtctccgggtaaa

SEQ ID NO: 93 - DNA, 1338 bp, synthetic construct
  misc_feature 1..1338: note = Heavy chain nucleotide sequence of T26B608
  source 1..1338: mol_type = other DNA
caggtgcagctgcaggaaagcggccccggactggtgaagcccagcgaaaccctgagcctgacctgtaccgtgagcggcggcagcatcagcagcggcgtgtactactgggcctggatcagacagccccctggcaagggcctggagctgatcggcaccttctactacagcggcagcacctactacgacagcagcctgaggagcagggtgaccatcagcgtggacaccagcaagaaccagttcagcctgaagctgagcagcgtgaccgccgccgatacagccgtgtattactgcgccaggcaggccggcgacttcgactactggggccagggaaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 94 - DNA, 1359 bp, synthetic construct
  misc_feature 1..1359: note = Heavy chain nucleotide sequence of T26B609
  source 1..1359: mol_type = other DNA
caggtgcagctgcagcagtggggcgctggactgctgaagcccagcgagaccctgtccctgacctgtgccgtgtacggcggcagcttcagcgactactactggagctggatcagacagcctcctggcaagggcctggagtggatcggcgagatcgaccacagcggctccaccgactacaaccccagcctgaagtccagggtgaccatcagcgtggacaccagcaagaaccagttcagcctgaagctgagcagcgtgacagccgccgataccgccgtgtactactgcgccaggagcatgtactacgacatctggaccggctaccacggcgcctttgacatctggggccagggcaccatggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 95 - DNA, 1338 bp, synthetic construct
  misc_feature 1..1338: note = Heavy chain nucleotide sequence of T26B610
  source 1..1338: mol_type = other DNA
caggtgcagctgcaggagagcggccctggactggtgaagcccagcgagaccctgagcctgacctgcatcgtgagcggcggcagcgtgagcagcagaaactactactggggctggattaggcagccccctggcaaaggcctggagtggatcggcaggatctactacagcggcaacaccaactacaaccccagcctgaagagcagagtgaccatctccgtggacaccagcaagaaccagttcagcctgaagctgagcagcgtgacagccgccgataccgccgtgtactactgcgccagatgggccggcgaaatcgactactggggccagggcacactggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 96 - DNA, 1338 bp, synthetic construct
  misc_feature 1..1338: note = Heavy chain nucleotide sequence of T26B611
  source 1..1338: mol_type = other DNA
cagctgcagctgcaggagagcggacccggactggtgaagcccagcgagaccctgagcctgacctgcaccgtgagcggcggaagcatcagcagcagcccctactattggggctggatcaggcagcctcccggaaagggcctggagtggatcggcagcttctactacggcggcagcaactactacaaccccagcctgaagagcagggtgaccatcagcgccgacaccagcaagaaccagttcagcctgaagctgtccagcgtgaccgccgctgataccgccgtgtactactgcgccagacaggccggcgactgggattactggggccagggcacactggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 97 - DNA, 1338 bp, synthetic construct
  misc_feature 1..1338: note = Heavy chain nucleotide sequence of T26B612
  source 1..1338: mol_type = other DNA
cagctgcagctgcaggagagcggacctggcctggtgaagcccagcgagaccctgagcctgacctgcaccgtgagcggcggaagcatcagcagcggcatctacaactggggctggatcaggcagccccctggcaaaggcctggagtggatcggcaacttccagtacagcggcatcacctactacaaccccagcctgaagagcagggtgaccatcagcgtggacaccagcaagaaccagttcagcctgcagctgagcagcgtgacagctgccgacaccgccgtgtactactgtgccaggcaggccggcgacttcgattactggggccagggcaccctggtgacagtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 98 - DNA, 1350 bp, synthetic construct
  misc_feature 1..1350: note = Heavy chain nucleotide sequence of T26B613
  source 1..1350: mol_type = other DNA
caggtgcagctggtggagagcggcggaggactggtgaagcctggaggctccctgagactgagctgtgccgcctccggcttcaccttcagcgactactatatgacctggatcaggcaggcccctggcaagggactggagtgggtgagctacatcagcagcagcggcatcaccaagtactacgccgacttcgtgaagggcaggttcaccatcagcagggacaacgccaagaacagcctgtacctgcagatgaacagcctgagggccgaggacaccgccgtgtactactgcgccagggacagagacaggcagtggctgctggagtttgactactggggccagggcaccctggtgacagtgtccagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 99 - DNA, 1353 bp, synthetic construct
  misc_feature 1..1353: note = Heavy chain nucleotide sequence of T26B614
  source 1..1353: mol_type = other DNA
caggtgcagctgcaggagagcggccctggactggtgaaacccagcgagaccctgagcctgagctgcacagtgagcggcggcagcctgaacagctacaactactactggggctgggtgagacagcctcccggcaagggactggagtggatcggcaccatctacagcagcggctccgcctactacaaccccagcctgaagagcaggttcaccatcagcgtggccaccagcaagaaccagttcagcctgagactgagcagcgtgacagccgccgacaccgctgtgtactactgcgccaggggctacaggaacagctggtacgccctgttcgagtactggggacagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 100 - DNA, 1338 bp, synthetic construct
  misc_feature 1..1338: note = Heavy chain nucleotide sequence of T26B615
  source 1..1338: mol_type = other DNA
caggtgcagctgcaggaatccggccctggcctggtgaaacccagcgagaccctgagcctgacctgcacagtgagtggaggtagcatcagcagcagcatctactactgggcctggatcaggcagcctcccggcaaaggactggagtggatcggtaccatctactacggcggcagcccctattacagtccctcactgaagagcagggtgaccatcagcatcgacaccagcaagtcccagttcagcctgaggctgaccagcgtgacagtggccgacaccgccgtgtattactgcgccaggagggccggcgacttcgactactggggacagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccggatgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 101 - protein, 214 aa, synthetic construct
  REGION 1..214: note = Light chain amino acid sequence of GC5B747, GC5B752
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 454 aa, synthetic construct
  REGION 1..454: note = Heavy chain amino acid sequence of GC5B747
QLQLQESGPGLVKPSETLSLTCTVSGGSLSSSSYWWGWTRQPPGRGLEWIGTMYYSGNIYYNPSLQSRATISVDTSKNQFSLKLSSVTAADTAVYYCARHVGYSYGRRFWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 454 aa, synthetic construct
  REGION 1..454: note = Heavy chain amino acid sequence of GC5B752
QLQLQESGPGLVKPSETLSLTCTVSGGSLSSSSYWWGWTRQPPGRGLEWIGTMYYSGNIYYNPSLQSRATISVDTSKNQFSLKLSSVTAADTAVYYCARHVGYSYGRRFWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPG

SEQ ID NO: 104 - DNA, 642 bp, synthetic construct
  misc_feature 1..642: note = Light chain nucleotide sequence of GC5B747, GC5B752
  source 1..642: mol_type = other DNA
gaaatcgtattgacccaatctcctgctactctcagcttgagccccggtgagagagcaaccctgtcttgtcgagcctctcaaagtgtgtcttcctacctcgcctggtatcaacagaagcctggccaagccccacgactgcttatttatgacgcttccaatcgggctactggaatccctgccaggttctccgggagtggttctggaacagacttcactctgaccatctcctcactggaacccgaggacttcgcagtctattattgccaacagaggagtaattggccccctacatttggccaaggcacaaaagtcgagattaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 105 - DNA, 1362 bp, synthetic construct
  misc_feature 1..1362: note = Heavy chain nucleotide sequence of GC5B747
  source 1..1362: mol_type = other DNA
caacttcaacttcaggagagtggcccaggactcgttaaacctagtgagaccctgtccctgacctgtactgtgtccggtggatcactgtcatcctcctcatattggtggggctggactcgccagccaccaggcaggggattggagtggattggcactatgtattactcaggcaatatttactataatccctctctccagagtcgggctacaatatccgtcgatactagcaaaaatcaattttccctgaaattgagtagcgtaactgccgccgacacagccgtctattactgtgcacgacacgttgggtattcatacggtaggcgattttggtacttcgatttgtgggggagaggaacccttgtgactgtaagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 106 - DNA, 1362 bp, synthetic construct
  misc_feature 1..1362: note = Heavy chain nucleotide sequence of GC5B752
  source 1..1362: mol_type = other DNA
caacttcaacttcaggagagtggcccaggactcgttaaacctagtgagaccctgtccctgacctgtactgtgtccggtggatcactgtcatcctcctcatattggtggggctggactcgccagccaccaggcaggggattggagtggattggcactatgtattactcaggcaatatttactataatccctctctccagagtcgggctacaatatccgtcgatactagcaaaaatcaattttccctgaaattgagtagcgtaactgccgccgacacagccgtctattactgtgcacgacacgttgggtattcatacggtaggcgattttggtacttcgatttgtgggggagaggaacccttgtgactgtaagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactaccgccagaagagcctctccctgtctccgggt

SEQ ID NO: 107 - protein, 125 aa, synthetic construct
  REGION 1..125: note = VH amino acid sequence of KL2B30, KL2B870, KL2B871, KL2B872
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSS

SEQ ID NO: 108 - protein, 107 aa, synthetic construct
  REGION 1..107: note = VL amino acid sequence of KL2B30, KL2B870, KL2B871, KL2B872
DIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIK

SEQ ID NO: 109 - protein, 10 aa, synthetic construct
  REGION 1..10: note = VH CDR1 of anti-KLK2 antibody
GGSISSYYWS

SEQ ID NO: 110 - protein, 16 aa, synthetic construct
  REGION 1..16: note = VH CDR2 of anti-KLK2 antibody
YIYYSGSTNYNPSLKS

SEQ ID NO: 111 - protein, 17 aa, synthetic construct
  REGION 1..17: note = VH CDR3 of anti-KLK2 antibody
TTIFGVVTPNFYYGMDV

SEQ ID NO: 112 - protein, 11 aa, synthetic construct
  REGION 1..11: note = VL CDR1 of anti-KLK2 antibody
RASQGISSYLA

SEQ ID NO: 113 - protein, 7 aa, synthetic construct
  REGION 1..7: note = VL CDR2 of anti-KLK2 antibody
AASTLQS

SEQ ID NO: 114 - protein, 9 aa, synthetic construct
  REGION 1..9: note = VL CDR3 of anti-KLK2 antibody
QQLNSYPLT

SEQ ID NO: 115 - protein, 8 aa, synthetic construct
  REGION 1..8: note = VH CDR1 of anti-KLK2 antibody
GGSISSYY

SEQ ID NO: 116 - protein, 7 aa, synthetic construct
  REGION 1..7: note = VH CDR2 of anti-KLK2 antibody
IYYSGST

SEQ ID NO: 117 - protein, 19 aa, synthetic construct
  REGION 1..19: note = VH CDR3 of anti-KLK2 antibody
AGTTIFGVVTPNFYYGMDV

SEQ ID NO: 118 - protein, 6 aa, synthetic construct
  REGION 1..6: note = VL CDR1 of anti-KLK2 antibody
QGISSY

SEQ ID NO: 119 - skipped

SEQ ID NO: 120 - protein, 9 aa, synthetic construct
  REGION 1..9: note = VL CDR3 of anti-KLK2 antibody
QQLNSYPLT

SEQ ID NO: 121 - protein, 5 aa, synthetic construct
  REGION 1..5: note = VH CDR1 of anti-KLK2 antibody
SYYWS

SEQ ID NO: 122 - protein, 16 aa, synthetic construct
  REGION 1..16: note = VH CDR2 of anti-KLK2 antibody
YIYYSGSTNYNPSLKS

SEQ ID NO: 123 - protein, 17 aa, synthetic construct
  REGION 1..17: note = VH CDR3 of anti-KLK2 antibody
TTIFGVVTPNFYYGMDV

SEQ ID NO: 124 - protein, 11 aa, synthetic construct
  REGION 1..11: note = VL CDR1 of anti-KLK2 antibody
RASQGISSYLA

SEQ ID NO: 125 - protein, 7 aa, synthetic construct
  REGION 1..7: note = VL CDR2 of anti-KLK2 antibody
AASTLQS

SEQ ID NO: 126 - protein, 9 aa, synthetic construct
  REGION 1..9: note = VL CDR3 of anti-KLK2 antibody
QQLNSYPLT

SEQ ID NO: 127 - protein, 7 aa, synthetic construct
  REGION 1..7: note = VH CDR1 of anti-KLK2 antibody
GGSISSY

SEQ ID NO: 128 - skipped

SEQ ID NO: 129 - protein, 15 aa, synthetic construct
  REGION 1..15: note = VH CDR3 of anti-KLK2 antibody
TIFGVVTPNFYYGMD

SEQ ID NO: 130 - protein, 7 aa, synthetic construct
  REGION 1..7: note = VL CDR1 of anti-KLK2 antibody
SQGISSY

SEQ ID NO: 131 - skipped

SEQ ID NO: 132 - protein, 6 aa, synthetic construct
  REGION 1..6: note = VL CDR3 of anti-KLK2 antibody
LNSYPL

SEQ ID NO: 133 - protein, 6 aa, synthetic construct
  REGION 1..6: note = VH CDR1 of anti-KLK2 antibody
SSYYWS

SEQ ID NO: 134 - protein, 12 aa, synthetic construct
  REGION 1..12: note = VH CDR2 of anti-KLK2 antibody
WIGYIYYSGSTN

SEQ ID NO: 135 - protein, 18 aa, synthetic construct
  REGION 1..18: note = VH CDR3 of anti-KLK2 antibody
AGTTIFGVVTPNFYYGMD

SEQ ID NO: 136 - protein, 7 aa, synthetic construct
  REGION 1..7: note = VL CDR1 of anti-KLK2 antibody
SSYLAWY

SEQ ID NO: 137 - protein, 10 aa, synthetic construct
  REGION 1..10: note = VL CDR2 of anti-KLK2 antibody
FLIYAASTLQ

SEQ ID NO: 138 - protein, 8 aa, synthetic construct
  REGION 1..8: note = VL CDR3 of anti-KLK2 antibody
QQLNSYPL

SEQ ID NO: 139 - protein, 10 aa, synthetic construct
  REGION 1..10: note = VH CDR1 of anti-KLK2 antibody
GGSISSYYWS

SEQ ID NO: 140 - protein, 9 aa, synthetic construct
  REGION 1..9: note = VH CDR2 of anti-KLK2 antibody
YIYYSGSTN

SEQ ID NO: 141 - protein, 17 aa, synthetic construct
  REGION 1..17: note = VH CDR3 of anti-KLK2 antibody
TTIFGVVTPNFYYGMDV

SEQ ID NO: 142 - protein, 11 aa, synthetic construct
  REGION 1..11: note = VL CDR1 of anti-KLK2 antibody
RASQGISSYLA

SEQ ID NO: 143 - protein, 7 aa, synthetic construct
  REGION 1..7: note = VL CDR2 of anti-KLK2 antibody
AASTLQS

SEQ ID NO: 144 - protein, 9 aa, synthetic construct
  REGION 1..9: note = VL CDR3 of anti-KLK2 antibody
QQLNSYPLT

SEQ ID NO: 145 - protein, 452 aa, synthetic construct
  REGION 1..452: note = Heavy chain amino acid sequence of KL2B30
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 146 - protein, 214 aa, synthetic construct
  REGION 1..214: note = Light chain amino acid sequence of KL2B30, KL2B870, KL2B871, KL2B872
DIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 252 aa, synthetic construct
  REGION 1..252: note = Amino acid sequence of scFv41
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSGGSEGKSSGSGSESKSTGGSDIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIK

SEQ ID NO: 148 - protein, 252 aa, synthetic construct
  REGION 1..252: note = Amino acid sequence of scFv42
DIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIKGGSEGKSSGSGSESKSTGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSS

SEQ ID NO: 149 - protein, 454 aa, synthetic construct
  REGION 1..454: note = Heavy chain amino acid sequence of KLCB80
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 150 - protein, 455 aa, synthetic construct
  REGION 1..455: note = Heavy chain amino acid sequence of KL2B30 with K477 Fab
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 151 - protein, 271 aa, synthetic construct
  REGION 1..271: note = Amino acid sequence of KL2B30_HL
MAWVWTLLFLMAAAQSIQAQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSGGSEGKSSGSGSESKSTGGSDIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIK

SEQ ID NO: 152 - protein, 271 aa, synthetic construct
  REGION 1..271: note = Amino acid sequence of KL2B30_LH
MAWVWTLLFLMAAAQSIQADIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIKGGSEGKSSGSGSESKSTGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSS

SEQ ID NO: 153 - protein, 454 aa, synthetic construct
  REGION 1..454: note = Heavy chain amino acid sequence of KL2B870
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSPG

SEQ ID NO: 154 - protein, 454 aa, synthetic construct
  REGION 1..454: note = Heavy chain amino acid sequence of KL2B871
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 155 - protein, 454 aa, synthetic construct
  REGION 1..454: note = Heavy chain amino acid sequence of KL2B872
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - DNA, 375 bp, synthetic construct
  misc_feature 1..375: note = VH nucleotide sequence of KL2B870, KL2B871, KL2B872
  source 1..375: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattggatatatctattacagtgggagcaccaactacaacccctccctcaagagtcgagtcaccatatcagtagacacgtccaagaaccagttctccctgaagctgagctctgtgaccgctgcggacacggccgtgtattactgtgcggggactacgatttttggagtggttacccccaacttctactacggtatggacgtctggggccaagggaccacggtcaccgtctcctca

SEQ ID NO: 157 - DNA, 321 bp, synthetic construct
  misc_feature 1..321: note = VL nucleotide sequence of KL2B870, KL2B871, KL2B872
  source 1..321: mol_type = other DNA
gacatccagatgacccagtctccttccttcctgtctgcatctgtaggagacagagtcaccatcacttgccgggccagtcagggcattagcagttatttagcctggtatcagcaaaaaccagggaaagcccctaagttcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctctcactttcggcggagggaccaaggtggaaatcaaa

SEQ ID NO: 158 - DNA, 1356 bp, synthetic construct
  misc_feature 1..1356: note = Heavy chain nucleotide sequence of KL2B30
  source 1..1356: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattggatatatctattacagtgggagcaccaactacaacccctccctcaagagtcgagtcaccatatcagtagacacgtccaagaaccagttctccctgaagctgagctctgtgaccgctgcggacacggccgtgtattactgtgcggggactacgatttttggagtggttacccccaacttctactacggtatggacgtctggggccaagggaccacggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaaaacctacacttgcaacgtagatcacaagcccagcaacaccaaggtggacaagagagttgagtccaaatatggtcccccatgcccaccatgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcagatggcaggaggggaatgtcttctcatgctccgtgatgcatgaggctctgcacaaccactacacacagaagagcctctccctgtctctgggtaaa

SEQ ID NO: 159 - DNA, 642 bp, synthetic construct
  misc_feature 1..642: note = Light chain nucleotide sequence of KL2B30
  source 1..642: mol_type = other DNA
gacatccagatgacccagtctccttccttcctgtctgcatctgtaggagacagagtcaccatcacttgccgggccagtcagggcattagcagttatttagcctggtatcagcaaaaaccagggaaagcccctaagttcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctctcactttcggcggagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 160 - DNA, 1362 bp, synthetic construct
  misc_feature 1..1362: note = Heavy chain nucleotide sequence of KL2B870
  source 1..1362: mol_type = other DNA
caagtacagcttcaggagtcaggccccggcttggtgaaaccaagtgagaccctctccctcacctgcacagttagcggcggttcaatatcatcatactattggtcttggattcgtcagccacctggcaaaggtctcgagtggattggctatatatattattcagggtcaacaaattacaatccttcactcaagtctcgcgtcaccattagcgtagacaccagcaaaaatcagttttccctcaagctctcatcagtgaccgccgcagataccgctgtatattactgcgctggtaccacaatatttggagtggttacccctaatttctattatggaatggacgtatggggccaggggaccaccgtgacagtttcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactaccgccagaagagcctctccctgtctccgggt

SEQ ID NO: 161 - DNA, 642 bp, synthetic construct
  misc_feature 1..642: note = Light chain nucleotide sequence of KL2B870, KL2B871, KL2B872
  source 1..642: mol_type = other DNA
gatatacaaatgacccaatctccttcctttttgtcagcaagcgttggtgaccgcgtcaccataacttgtagggctagccagggtatttctagttatctggcttggtatcagcaaaaacctggcaaagcccctaaatttcttatctacgccgcaagcactctgcaatcaggcgtgcccagcagattctcaggctctggttcaggcacagagtttactctgactatatccagcctgcaacctgaagactttgctacatactattgtcagcaactcaacagttacccccttactttcgggggcggcactaaggtggaaataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 162 - DNA, 1362 bp, synthetic construct
  misc_feature 1..1362: note = Heavy chain nucleotide sequence of KL2B871
  source 1..1362: mol_type = other DNA
caagtacagcttcaggagtcaggccccggcttggtgaaaccaagtgagaccctctccctcacctgcacagttagcggcggttcaatatcatcatactattggtcttggattcgtcagccacctggcaaaggtctcgagtggattggctatatatattattcagggtcaacaaattacaatccttcactcaagtctcgcgtcaccattagcgtagacaccagcaaaaatcagttttccctcaagctctcatcagtgaccgccgcagataccgctgtatattactgcgctggtaccacaatatttggagtggttacccctaatttctattatggaatggacgtatggggccaggggaccaccgtgacagtttcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 163 - DNA, 1362 bp, synthetic construct
  misc_feature 1..1362: note = Heavy chain nucleotide sequence of KL2B872
  source 1..1362: mol_type = other DNA
caagtacagcttcaggagtcaggccccggcttggtgaaaccaagtgagaccctctccctcacctgcacagttagcggcggttcaatatcatcatactattggtcttggattcgtcagccacctggcaaaggtctcgagtggattggctatatatattattcagggtcaacaaattacaatccttcactcaagtctcgcgtcaccattagcgtagacaccagcaaaaatcagttttccctcaagctctcatcagtgaccgccgcagataccgctgtatattactgcgctggtaccacaatatttggagtggttacccctaatttctattatggaatggacgtatggggccaggggaccaccgtgacagtttcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 164 - protein, 5 aa, synthetic construct
  REGION 1..5: note = Amino acid sequence of hKLK2 bound to KL2B30
KVTEF

SEQ ID NO: 165 - protein, 5 aa, synthetic construct
  REGION 1..5: note = Amino acid sequence of hKLK2 bound to KL2B30
HYRKW

SEQ ID NO: 166 - protein, 20 aa, synthetic construct
  REGION 1..20: note = Amino acid sequence of linker
GGSEGKSSGSGSESKSTGGS

SEQ ID NO: 167 - protein, 240 aa, synthetic construct
  REGION 1..240: note = Amino acid sequence hKLK2 with 6-His
VPLIEGRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHYSEKVTEFMLCAGLWTGGKDTCGGDSGGPLVCNGVLQGITSWGPEPCALPEKPAVYTKVVHYRKWIKDTIAANPHHHHHH

SEQ ID NO: 168 - protein, 5 aa, synthetic construct
  REGION 1..5: note = VH CDR2 of anti-KLK2 antibody
YYSGS

SEQ ID NO: 169 - protein, 9 aa, synthetic construct
  REGION 1..9: note = VL CDR1 of anti-KLK2 antibody
RASQGISSY

SEQ ID NO: 170 - protein, 7 aa, synthetic construct
  REGION 1..7: note = VL CDR2 of anti-KLK2 antibody
AASTLQS

SEQ ID NO: 171 - protein, 9 aa, synthetic construct
  REGION 1..9: note = VL CDR3 of anti-KLK2 antibody
QQLNSYPLT

SEQ ID NO: 172 - protein, 113 aa, synthetic construct
  REGION 1..113: note = VL amino acid sequence of MHGB732
DIVMTQSPDSLAVSLGERATINCKSSQSVLHSSNNKNYLTWFQQKPGQPPKLLIYWASTRESGVPDRFSGSGSGTDFTLTISSLQAEDVAVYYCHQYYSTPPTFGQGTKVEIK

SEQ ID NO: 173 - protein, 113 aa, synthetic construct
  REGION 1..113: note = VL amino acid sequence of MHGB738
DIVMTQSPDSLAVSLGERATINCKSSQSVLFSSNNKNYLAWYQQKPGQPPKLLIYWASTRESGVPDRFSGSVSGTDFTLTISSLQAEDVAVYYCQQYHSTPWTFGQGTKVEIK

SEQ ID NO: 174 - protein, 125 aa, synthetic construct
  REGION 1..125: note = VH amino acid sequence of MHGB732
QVQLQQSGPGLVKPSQTLSLTCAISGDSVSSNSAAWNWIRQSPSRGLEWLGRTYYRSKWYNDYAVSVKSRITINPDTSKNQISLQLNSVTPEDTAVYYCAGDRRYGIVGLPFAYWGQGTLVTVSS

SEQ ID NO: 175 - protein, 121 aa, synthetic construct
  REGION 1..121: note = VH amino acid sequence of MHGB738
QVQLQQSGPGLVKPSQTLSLTCAISGDSVSSNRAAWNWIRQSPSRGLEWLGRTYYRSKWYNDYAVSVKSRITINPDTSKNQISLQLNSVTPEDTAVYYCARVRPGIPFDYWGQGTPVTVSS

SEQ ID NO: 176 - protein, 220 aa, synthetic construct
  REGION 1..220: note = Light chain amino acid sequence of MHGB732
DIVMTQSPDSLAVSLGERATINCKSSQSVLHSSNNKNYLTWFQQKPGQPPKLLIYWASTRESGVPDRFSGSGSGTDFTLTISSLQAEDVAVYYCHQYYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 220 aa, synthetic construct
  REGION 1..220: note = Light chain amino acid sequence of MHGB738
DIVMTQSPDSLAVSLGERATINCKSSQSVLFSSNNKNYLAWYQQKPGQPPKLLIYWASTRESGVPDRFSGSVSGTDFTLTISSLQAEDVAVYYCQQYHSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 454 aa, synthetic construct
  REGION 1..454: note = Heavy chain amino acid sequence of MHGB732
QVQLQQSGPGLVKPSQTLSLTCAISGDSVSSNSAAWNWIRQSPSRGLEWLGRTYYRSKWYNDYAVSVKSRITINPDTSKNQISLQLNSVTPEDTAVYYCAGDRRYGIVGLPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - protein, 450 aa, synthetic construct
  REGION 1..450: note = Heavy chain amino acid sequence of MHGB738
QVQLQQSGPGLVKPSQTLSLTCAISGDSVSSNRAAWNWIRQSPSRGLEWLGRTYYRSKWYNDYAVSVKSRITINPDTSKNQISLQLNSVTPEDTAVYYCARVRPGIPFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 180 - DNA, 660 bp, synthetic construct
  misc_feature 1..660: note = Light chain nucleotide sequence of MHGB732
  source 1..660: mol_type = other DNA
gacatcgtaatgacacagtcaccagattcattggcagttagtctgggtgaaagggcaacaatcaactgcaagtcttctcagagtgtactgcatagttctaacaataagaactaccttacctggtttcaacagaaaccaggtcagccccccaagttgctgatttactgggcaagcacccgcgaatccggcgttcccgatcgattttcaggttccgggagtgggaccgactttaccttgaccatctcttccttgcaggccgaagatgtagccgtctattactgccatcagtattactctactccccccacattcggtcaaggtacaaaagttgagataaaacggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 181 - DNA, 660 bp, synthetic construct
  misc_feature 1..660: note = Light chain nucleotide sequence of MHGB738
  source 1..660: mol_type = other DNA
gatattgttatgacacagtccccagattcattggcagtaagcctcggtgaacgggctactattaactgtaagtcttcccagagtgtattgttctcttcaaataacaaaaactacctggcatggtatcagcaaaagcctggtcaaccccctaaacttctcatatactgggcatccactcgggagagcggtgtgccagaccgtttctcagggagtgtgtcaggtacagattttacactcacaatttccagcctccaagccgaagacgttgcagtatattattgccaacaatatcactctacaccttggacatttggtcaaggtactaaagtcgaaatcaaacggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 182 - DNA, 1362 bp, synthetic construct
  misc_feature 1..1362: note = Heavy chain nucleotide sequence of MHGB732
  source 1..1362: mol_type = other DNA
caagtacaactgcaacaaagtggtcctgggctcgtgaagccttcccagactctcagcctcacatgcgctataagtggggattctgtttcctcaaattcagcagcctggaattggatacgacagtctccatcccgtggccttgagtggcttggtagaacttattaccgatccaagtggtacaatgattacgccgtttcagtgaagtcccgcattactattaatcccgacacatctaagaatcaaatttcattgcaactgaatagcgtaacacccgaagatacagcagtttattattgtgcaggtgatcgacgctacggcatagtgggacttcctttcgcctattggggccaagggacactggtcactgtgtcatccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 183 - DNA, 1350 bp, synthetic construct
  misc_feature 1..1350: note = Heavy chain nucleotide sequence of MHGB738
  source 1..1350: mol_type = other DNA
caggtgcagcttcaacagagcggacctggtctggttaagccttcccaaaccctgagcctgacttgtgctatttccggggatagtgttagctccaatagggcagcatggaactggatcagacagtccccaagccgtggacttgagtggcttggacgtacttattacaggagtaaatggtacaatgattatgccgtttctgtgaagagccgtattactataaacccagatacttctaaaaatcaaatttcccttcagctcaactcagttacaccagaggatactgcagtctattattgcgcaagagttcgacctggcattcccttcgattattgggggcaggggacacccgttactgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggt

SEQ ID NO: 184 - protein, 7 aa, synthetic construct
  REGION 1..7: note = VH CDR1 of anti-HLA-G antibody
SNSAAWN

SEQ ID NO: 185 - protein, 18 aa, synthetic construct
  REGION 1..18: note = VH CDR2 of anti-HLA-G antibody
RTYYRSKWYNDYAVSVKS

SEQ ID NO: 186 - protein, 13 aa, synthetic construct
  REGION 1..13: note = VH CDR3 of anti-HLA-G antibody
DRRYGIVGLPFAY

SEQ ID NO: 187 - protein, 17 aa, synthetic construct
  REGION 1..17: note = VL CDR1 of anti-HLA-G antibody
KSSQSVLHSSNNKNYLT

SEQ ID NO: 188 - protein, 7 aa, synthetic construct
  REGION 1..7: note = VL CDR2 of anti-HLA-G antibody
WASTRES

SEQ ID NO: 189 - protein, 9 aa, synthetic construct
  REGION 1..9: note = VL CDR3 of anti-HLA-G antibody
HQYYSTPPT

SEQ ID NO: 190 - protein, 9 aa, synthetic construct
  REGION 1..9: note = VH CDR1 of anti-HLA-G antibody
GDSVSSNSA

SEQ ID NO: 191 - protein, 7 aa, synthetic construct
  REGION 1..7: note = VH CDR2 of anti-HLA-G antibody
YYRSKWY

SEQ ID NO: 192 - protein, 12 aa, synthetic construct
  REGION 1..12: note = VH CDR3 of anti-HLA-G antibody
DRRYGIVGLPFA

SEQ ID NO: 193 - protein, 13 aa, synthetic construct
  REGION 1..13: note = VL CDR1 of anti-HLA-G antibody
SQSVLHSSNNKNY

SEQ ID NO: 194 - skipped

SEQ ID NO: 195 - protein, 6 aa, synthetic construct
  REGION 1..6: note = VL CDR3 of anti-HLA-G antibody
YYSTPP

SEQ ID NO: 196 - protein, 10 aa, synthetic construct
  REGION 1..10: note = VH CDR1 of anti-HLA-G antibody
GDSVSSNSAA

SEQ ID NO: 197 - protein, 9 aa, synthetic construct
  REGION 1..9: note = VH CDR2 of anti-HLA-G antibody
TYYRSKWYN

SEQ ID NO: 198 - protein, 15 aa, synthetic construct
  REGION 1..15: note = VH CDR3 of anti-HLA-G antibody
AGDRRYGIVGLPFAY

SEQ ID NO: 199 - protein, 12 aa, synthetic construct
  REGION 1..12: note = VL CDR1 of anti-HLA-G antibody
QSVLHSSNNKNY

SEQ ID NO: 200 - skipped

SEQ ID NO: 201 - protein, 9 aa, synthetic construct
  REGION 1..9: note = VL CDR3 of anti-HLA-G antibody
HQYYSTPPT

SEQ ID NO: 202 - protein, 12 aa, synthetic construct
  REGION 1..12: note = VH CDR1 of anti-HLA-G antibody
GDSVSSNSAAWN

SEQ ID NO: 203 - protein, 11 aa, synthetic construct
  REGION 1..11: note = VH CDR2 of anti-HLA-G antibody
RTYYRSKWYND

SEQ ID NO: 204 - protein, 13 aa, synthetic construct
  REGION 1..13: note = VH CDR3 of anti-HLA-G antibody
DRRYGIVGLPFAY

SEQ ID NO: 205 - protein, 17 aa, synthetic construct
  REGION 1..17: note = VL CDR1 of anti-HLA-G antibody
KSSQSVLHSSNNKNYLT

SEQ ID NO: 206 - protein, 7 aa, synthetic construct
  REGION 1..7: note = VL CDR2 of anti-HLA-G antibody
WASTRES

SEQ ID NO: 207 - protein, 9 aa, synthetic construct
  REGION 1..9: note = VL CDR3 of anti-HLA-G antibody
HQYYSTPPT

SEQ ID NO: 208 - protein, 7 aa, synthetic construct
  REGION 1..7: note = VH CDR1 of anti-HLA-G antibody
SNRAAWN

SEQ ID NO: 209 - protein, 18 aa, synthetic construct
  REGION 1..18: note = VH CDR2 of anti-HLA-G antibody
RTYYRSKWYNDYAVSVKS

SEQ ID NO: 210 - protein, 9 aa, synthetic construct
  REGION 1..9: note = VH CDR3 of anti-HLA-G antibody
VRPGIPFDY

SEQ ID NO: 211 - protein, 17 aa, synthetic construct
  REGION 1..17: note = VL CDR1 of anti-HLA-G antibody
KSSQSVLFSSNNKNYLA

SEQ ID NO: 212 - protein, 7 aa, synthetic construct
  REGION 1..7: note = VL CDR2 of anti-HLA-G antibody
WASTRES

SEQ ID NO: 213 - protein, 9 aa, synthetic construct
  REGION 1..9: note = VL CDR3 of anti-HLA-G antibody
QQYHSTPWT

SEQ ID NO: 214 - protein, 9 aa, synthetic construct
  REGION 1..9: note = VH CDR1 of anti-HLA-G antibody
GDSVSSNRA

SEQ ID NO: 215 - protein, 7 aa, synthetic construct
  REGION 1..7: note = VH CDR2 of anti-HLA-G antibody
YYRSKWY

SEQ ID NO: 216 - protein, 8 aa, synthetic construct
  REGION 1..8: note = VH CDR3 of anti-HLA-G antibody
VRPGIPFD

SEQ ID NO: 217 - protein, 13 aa, synthetic construct
  REGION 1..13: note = VL CDR1 of anti-HLA-G antibody
SQSVLFSSNNKNY

SEQ ID NO: 218 - skipped

SEQ ID NO: 219 - protein, 6 aa, synthetic construct
  REGION 1..6: note = VL CDR3 of anti-HLA-G antibody
YHSTPW

SEQ ID NO: 220 - protein, 10 aa, synthetic construct
  REGION 1..10: note = VH CDR1 of anti-HLA-G antibody
GDSVSSNRAA

SEQ ID NO: 221 - protein, 9 aa, synthetic construct
  REGION 1..9: note = VH CDR2 of anti-HLA-G antibody
TYYRSKWYN

SEQ ID NO: 222 - protein, 11 aa, synthetic construct
  REGION 1..11: note = VH CDR3 of anti-HLA-G antibody
ARVRPGIPFDY

SEQ ID NO: 223 - protein, 12 aa, synthetic construct
  REGION 1..12: note = VL CDR1 of anti-HLA-G antibody
QSVLFSSNNKNY

SEQ ID NO: 224 - skipped

SEQ ID NO: 225 - protein, 9 aa, synthetic construct
  REGION 1..9: note = VL CDR3 of anti-HLA-G antibody
QQYHSTPWT

SEQ ID NO: 226 - protein, 12 aa, synthetic construct
  REGION 1..12: note = VH CDR1 of anti-HLA-G antibody
GDSVSSNRAAWN

SEQ ID NO: 227 - protein, 11 aa, synthetic construct
  REGION 1..11: note = VH CDR2 of anti-HLA-G antibody
RTYYRSKWYND

SEQ ID NO: 228 - protein, 9 aa, synthetic construct
  REGION 1..9: note = VH CDR3 of anti-HLA-G antibody
VRPGIPFDY

SEQ ID NO: 229 - protein, 17 aa, synthetic construct
  REGION 1..17: note = VL CDR1 of anti-HLA-G antibody
KSSQSVLFSSNNKNYLA

SEQ ID NO: 230 - protein, 7 aa, synthetic construct
  REGION 1..7: note = VL CDR2 of anti-HLA-G antibody
WASTRES

SEQ ID NO: 231 - protein, 9 aa, synthetic construct
  REGION 1..9: note = VL CDR3 of anti-HLA-G antibody
QQYHSTPWT

SEQ ID NO: 232 - protein, 110 aa, synthetic construct
  REGION 1..110: note = VL amino acid sequence of PSMB896, PSMB898
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVL

SEQ ID NO: 233 - protein, 121 aa, synthetic construct
  REGION 1..121: note = VH amino acid sequence of PSMB896
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDGVGATPYYFDYWGQGTLVTVSS

SEQ ID NO: 234 - protein, 121 aa, synthetic construct
  REGION 1..121: note = VH amino acid sequence of PSMB898
EVQLVESGGGLVQPGGSLRLSCAASGFTFSSYAMSWVRQAPGKGLEWVSAISGGSGSTYYADSVKGRFTISRDNSKNTLYLQMNSLRAEDSAVYYCAKDGVGATPYYFDYWGQGTLVTVSS

SEQ ID NO: 235 - DNA, 330 bp, synthetic construct
  misc_feature 1..330: note = VL nucleotide sequence of PSMB896, PSMB898
  source 1..330: mol_type = other DNA
cagagtgtccttactcagcctcctagcgttagcgccgcccctggacagaaggttactatctcctgctcagggagttccagtaatattggaatcaattatgtgagttggtatcagcagttgcccggcaccgctcctaaattgcttatctatgacaacaataaacgacctagtggtatccctgatcgtttttctggatcaaaatctggtactagcgcaaccctcggtatcaccggactgcaaacaggtgatgaagcagactattattgcggcacctgggactcatcactctccgccgtcgttttcgggggcggaaccaaacttacagtattg

SEQ ID NO: 236 - DNA, 363 bp, synthetic construct
  misc_feature 1..363: note = VH nucleotide sequence of PSMB896
  source 1..363: mol_type = other DNA
gaggtacaacttgtggaaagtggaggcggtcttgtccaacctggaggatctctccgattgagttgcgcagccagcgggtttactttttcttcatacgccatgtcctgggtgcggcaagcaccaggtaaaggacttgagtgggtatctgctatttcaggggggataggctcaacatactatgctgatagcgtgaaaggtaggttcaccatttcccgtgacaatagtaaaaacacattgtggctccaaatgaacagccttagggctgaagacaccgctgtttactactgcgctaaagacggtgtaggggcaactccctattacttcgattattggggacaaggaaccttggtaacagtttcaagc

SEQ ID NO: 237 - DNA, 363 bp, synthetic construct
  misc_feature 1..363: note = VH nucleotide sequence of PSMB898
  source 1..363: mol_type = other DNA
gaagtccagttggtagaatctggaggcgggctggtacagcctggcggttccttgcgcctctcatgtgccgcaagcgggtttaccttcagctcttacgcaatgtcatgggtgcgtcaggcccctggaaaaggtctcgagtgggtcagtgccatttctgggggctccggctccacctactacgcagattcagttaaagggagatttacaatctcaagagataacagtaaaaacaccctctacctccagatgaactcacttcgagctgaggattcagcagtatattactgtgctaaagacggtgtaggtgcaactccctactatttcgactattggggccaagggactttggtgacagtaagtagt

SEQ ID NO: 238 - protein, 216 aa, synthetic construct
  REGION 1..216: note = Light chain amino acid sequence of PSMB896, PSMB898
QSVLTQPPSVSAAPGQKVTISCSGSSSNIGINYVSWYQQLPGTAPKLLIYDNNKRPSGIPDRFSGSKSGTSATLGITGLQTGDEADYYCGTWDSSLSAVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 239 - protein, 450 aa, synthetic construct
  REGION 1..450: note = Heavy chain amino acid sequence of PSMB896
EVQLVESGGGLVQPGGSLRLSCAASGFTFSSYAMSWVRQAPGKGLEWVSAISGGIGSTYYADSVKGRFTISRDNSKNTLWLQMNSLRAEDTAVYYCAKDGVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 240 - protein, 450 aa, synthetic construct
  REGION 1..450: note = Heavy chain amino acid sequence of PSMB898
EVQLVESGGGLVQPGGSLRLSCAASGFTFSSYAMSWVRQAPGKGLEWVSAISGGSGSTYYADSVKGRFTISRDNSKNTLYLQMNSLRAEDSAVYYCAKDGVGATPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 241 - DNA, 648 bp, synthetic construct
  misc_feature 1..648: note = Light chain nucleotide sequence of PSMB896, PSMB898
  source 1..648: mol_type = other DNA
cagagtgtccttactcagcctcctagcgttagcgccgcccctggacagaaggttactatctcctgctcagggagttccagtaatattggaatcaattatgtgagttggtatcagcagttgcccggcaccgctcctaaattgcttatctatgacaacaataaacgacctagtggtatccctgatcgtttttctggatcaaaatctggtactagcgcaaccctcggtatcaccggactgcaaacaggtgatgaagcagactattattgcggcacctgggactcatcactctccgccgtcgttttcgggggcggaaccaaacttacagtattgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 242 - DNA, 1350 bp, synthetic construct
  misc_feature 1..1350: note = Heavy chain nucleotide sequence of PSMB896
  source 1..1350: mol_type = other DNA
gaggtacaacttgtggaaagtggaggcggtcttgtccaacctggaggatctctccgattgagttgcgcagccagcgggtttactttttcttcatacgccatgtcctgggtgcggcaagcaccaggtaaaggacttgagtgggtatctgctatttcaggggggataggctcaacatactatgctgatagcgtgaaaggtaggttcaccatttcccgtgacaatagtaaaaacacattgtggctccaaatgaacagccttagggctgaagacaccgctgtttactactgcgctaaagacggtgtaggggcaactccctattacttcgattattggggacaaggaaccttggtaacagtttcaagcgcttccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagccggctcaccgtggacaagtctagatggcagcaggggaacgtcttctcatgctccgtgatgcatgaggctctgcacaaccactacacgcagaagagcctctccctgtctccgggt

SEQ ID NO: 243 - DNA, 1350 bp, synthetic construct
  misc_feature 1..1350: note = Heavy chain nucleotide sequence of PSMB898
  source 1..1350: mol_type = other DNA
gaagtccagttggtagaatctggaggcgggctggtacagcctggcggttccttgcgcctctcatgtgccgcaagcgggtttaccttcagctcttacgcaatgtcatgggtgcgtcaggcccctggaaaaggtctcgagtgggtcagtgccatttctgggggctccggctccacctactacgcagattcagttaaagggagatttacaatctcaagagataacagtaaaaacaccctctacctccagatgaactcacttcgagctgaggattcagcagtatattactgtgctaaagacggtgtaggtgcaactccctactatttcgactattggggccaagggactttggtgacagtaagtagtgcttccaccaagggcccatccgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagccggctcaccgtggacaagtctagatggcagcaggggaacgtcttctcatgctccgtgatgcatgaggctctgcacaaccactacacgcagaagagcctctccctgtctccgggt

SEQ ID NO: 244 - protein, 5 aa, synthetic construct
  REGION 1..5: note = VH CDR1 of anti-PSMA antibody
SYAMS

SEQ ID NO: 245 - protein, 17 aa, synthetic construct
  REGION 1..17: note = VH CDR2 of anti-PSMA antibody
AISGGIGSTYYADSVKG

SEQ ID NO: 246 - protein, 12 aa, synthetic construct
  REGION 1..12: note = VH CDR3 of anti-PSMA antibody
DGVGATPYYFDY

SEQ ID NO: 247 - protein, 14 aa, synthetic construct
  REGION 1..14: note = VL CDR1 of anti-PSMA antibody
SGSSSNIGINYVST

SEQ ID NO: 248 - protein, 7 aa, synthetic construct
  REGION 1..7: note = VL CDR2 of anti-PSMA antibody
DNNKRPS

SEQ ID NO: 249 - protein, 11 aa, synthetic construct
  REGION 1..11: note = VL CDR3 of anti-PSMA antibody
GTWDSSLSAVV

SEQ ID NO: 250 - protein, 5 aa, synthetic construct
  REGION 1..5: note = VH CDR1 of anti-PSMA antibody
SYAMS

SEQ ID NO: 251 - protein, 17 aa, synthetic construct
  REGION 1..17: note = VH CDR2 of anti-PSMA antibody
AISGGSGSTYYADSVKG

SEQ ID NO: 252 - protein, 12 aa, synthetic construct
  REGION 1..12: note = VH CDR3 of anti-PSMA antibody
DGVGATPYYFDY

SEQ ID NO: 253 - protein, 13 aa, synthetic construct
  REGION 1..13: note = VL CDR1 of anti-PSMA antibody
SGSSSNIGINYVS

SEQ ID NO: 254 - protein, 7 aa, synthetic construct
  REGION 1..7: note = VL CDR2 of anti-PSMA antibody
DNNKRPS

SEQ ID NO: 255 - protein, 11 aa, synthetic construct
  REGION 1..11: note = VL CDR3 of anti-PSMA antibody
GTWDSSLSAVV

SEQ ID NO: 256 - protein, 10 aa, synthetic construct
  REGION 1..10: note = VH CDR1 of anti-PSMA antibody
GFTFSSYAMS

SEQ ID NO: 257 - protein, 10 aa, synthetic construct
  REGION 1..10: note = VH CDR2 of anti-PSMA antibody
AISGGIGSTY

SEQ ID NO: 258 - protein, 12 aa, synthetic construct
  REGION 1..12: note = VH CDR3 of anti-PSMA antibody
DGVGATPYYFDY

SEQ ID NO: 259 - protein, 13 aa, synthetic construct
  REGION 1..13: note = VL CDR1 of anti-PSMA antibody
SGSSSNIGINYVS

SEQ ID NO: 260 - protein, 7 aa, synthetic construct
  REGION 1..7: note = VL CDR2 of anti-PSMA antibody
DNNKRPS

SEQ ID NO: 261 - protein, 11 aa, synthetic construct
  REGION 1..11: note = VL CDR3 of anti-PSMA antibody
GTWDSSLSAVV

SEQ ID NO: 262 - protein, 10 aa, synthetic construct
  REGION 1..10: note = VH CDR1 of anti-PSMA antibody
GFTFSSYAMS

SEQ ID NO: 263 - protein, 10 aa, synthetic construct
  REGION 1..10: note = VH CDR2 of anti-PSMA antibody
AISGGSGSTY

SEQ ID NO: 264 - protein, 12 aa, synthetic construct
  REGION 1..12: note = VH CDR3 of anti-PSMA antibody
DGVGATPYYFDY

SEQ ID NO: 265 - protein, 13 aa, synthetic construct
  REGION 1..13: note = VL CDR1 of anti-PSMA antibody
SGSSSNIGINYVS

SEQ ID NO: 266 - protein, 7 aa, synthetic construct
  REGION 1..7: note = VL CDR2 of anti-PSMA antibody
DNNKRPS

SEQ ID NO: 267 - protein, 11 aa, synthetic construct
  REGION 1..11: note = VL CDR3 of anti-PSMA antibody
GTWDSSLSAVV

SEQ ID NO: 268 - protein, 7 aa, synthetic construct
  REGION 1..7: note = VH CDR1 of anti-PSMA antibody
GFTFSSY

SEQ ID NO: 269 - protein, 6 aa, synthetic construct
  REGION 1..6: note = VH CDR2 of anti-PSMA antibody
SGGIGS

SEQ ID NO: 270 - protein, 10 aa, synthetic construct
  REGION 1..10: note = VH CDR3 of anti-PSMA antibody
GVGATPYYFD

SEQ ID NO: 271 - protein, 9 aa, synthetic construct
  REGION 1..9: note = VL CDR1 of anti-PSMA antibody
SSSNIGINY

SEQ ID NO: 272 - skipped

SEQ ID NO: 273 - protein, 8 aa, synthetic construct
  REGION 1..8: note = VL CDR3 of anti-PSMA antibody
WDSSLSAV

SEQ ID NO: 274 - protein, 7 aa, synthetic construct
  REGION 1..7: note = VH CDR1 of anti-PSMA antibody
GFTFSSY

SEQ ID NO: 275 - protein, 6 aa, synthetic construct
  REGION 1..6: note = VH CDR2 of anti-PSMA antibody
SGGSGS

SEQ ID NO: 276 - protein, 10 aa, synthetic construct
  REGION 1..10: note = VH CDR3 of anti-PSMA antibody
GVGATPYYFD

SEQ ID NO: 277 - protein, 10 aa, synthetic construct
  REGION 1..10: note = VL CDR1 of anti-PSMA antibody
SSSSNIGINY

SEQ ID NO: 278 - skipped

SEQ ID NO: 279 - protein, 8 aa, synthetic construct
  REGION 1..8: note = VL CDR3 of anti-PSMA antibody
WDSSLSAV

SEQ ID NO: 280 - protein, 5 aa, synthetic construct
  REGION 1..5: note = VH CDR1 of anti-PSMA antibody
SYAMS

SEQ ID NO: 281 - protein, 17 aa, synthetic construct
  REGION 1..17: note = VH CDR2 of anti-PSMA antibody
AISGGIGSTYYADSVKG

SEQ ID NO: 282 - protein, 12 aa, synthetic construct
  REGION 1..12: note = VH CDR3 of anti-PSMA antibody
DGVGATPYYFDY

SEQ ID NO: 283 - protein, 13 aa, synthetic construct
  REGION 1..13: note = VL CDR1 of anti-PSMA antibody
SGSSSNIGINYVS

SEQ ID NO: 284 - protein, 7 aa, synthetic construct
  REGION 1..7: note = VL CDR2 of anti-PSMA antibody
DNNKRPS

SEQ ID NO: 285 - protein, 11 aa, synthetic construct
  REGION 1..11: note = VL CDR3 of anti-PSMA antibody
GTWDSSLSAVV

SEQ ID NO: 286 - protein, 5 aa, synthetic construct
  REGION 1..5: note = VH CDR1 of anti-PSMA antibody
SYAMS

SEQ ID NO: 287 - protein, 17 aa, synthetic construct
  REGION 1..17: note = VH CDR2 of anti-PSMA antibody
AISGGSGSTYYADSVKG

SEQ ID NO: 288 - protein, 12 aa, synthetic construct
  REGION 1..12: note = VH CDR3 of anti-PSMA antibody
DGVGATPYYFDY

SEQ ID NO: 289 - protein, 13 aa, synthetic construct
  REGION 1..13: note = VL CDR1 of anti-PSMA antibody
SGSSSNIGINYVS

SEQ ID NO: 290 - protein, 7 aa, synthetic construct
  REGION 1..7: note = VL CDR2 of anti-PSMA antibody
DNNKRPS

SEQ ID NO: 291 - protein, 11 aa, synthetic construct
  REGION 1..11: note = VL CDR3 of anti-PSMA antibody
GTWDSSLSAVV

SEQ ID NO: 292 - protein, 8 aa, synthetic construct
  REGION 1..8: note = VH CDR1 of anti-BCMA antibody
GFTFSSYA

SEQ ID NO: 293 - protein, 8 aa, synthetic construct
  REGION 1..8: note = VH CDR2 of anti-BCMA antibody
ISGSGGST

SEQ ID NO: 294 - protein, 16 aa, synthetic construct
  REGION 1..16: note = VH CDR3 of anti-BCMA antibody
AKDEGYSSGHYYGMDV

SEQ ID NO: 295 - protein, 7 aa, synthetic construct
  REGION 1..7: note = VL CDR1 of anti-BCMA antibody
QSISSSF

SEQ ID NO: 296 - skipped

SEQ ID NO: 297 - protein, 10 aa, synthetic construct
  REGION 1..10: note = VL CDR3 of anti-BCMA antibody
QHYGSSPMYT

SEQ ID NO: 298 - protein, 123 aa, synthetic construct
  REGION 1..123: note = VH amino acid sequence of BCMB519
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSS

SEQ ID NO: 299 - protein, 109 aa, synthetic construct
  REGION 1..109: note = VL amino acid sequence of BCMB519
EIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIK

SEQ ID NO: 300 - protein, 502 aa, synthetic construct
  REGION 1..502: note = Heavy chain amino acid sequence of BCMB519
MAWVWTLLFLMAAAQSIQAEIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKGGSEGKSSGSGSESKSTGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 301 - protein, 6 aa, synthetic construct
HHHHHH

(19) United States
(12) Patent Application Publication
(10) Pub. No.: US 2026/0317441 A1
(43) Pub. Date: Oct. 15, 2026

(54) BIOSYNTHETIC GLYCOPROTEIN POPULATIONS

(71) Applicant: Janssen Biotech, Inc., Horsham, PA (US)

(72) Inventors: Rajkumar Ganesan, Blue Bell, PA (US); Adam ZWOLAK, Bala Cynwyd, PA (US); Jason HO, Collegeville, PA (US); Natasa OBERMAJER, Antwerp (BE); Michael DIEM, Havertown, PA (US); Sanjaya SINGH, Blue Bell, PA (US); Sathyadevi VENKATARAMANI, Blue Bell, PA (US); Theresa MCDEVITT, Warminster, PA (US); Fei SHEN, Collegeville, PA (US)

(73) Assignee: Janssen Biotech, Inc., Horsham, PA (US)

(21) Appl. No.: 19/350,694

(22) Filed: Oct. 6, 2025

Related U.S. Application Data
(63) Continuation of application No. 17/233,021, filed on Apr. 16, 2021, now abandoned.
(60) Provisional application No. 63/011,991, filed on Apr. 17, 2020.
(60) Provisional application No. 63/011,993, filed on Apr. 17, 2020.
(60) Provisional application No. 63/011,985, filed on Apr. 17, 2020.
(60) Provisional application No. 63/011,974, filed on Apr. 17, 2020.
(60) Provisional application No. 63/011,959, filed on Apr. 17, 2020.
(60) Provisional application No. 63/030,829, filed on May 27, 2020.
(60) Provisional application No. 63/030,823, filed on May 27, 2020.
(60) Provisional application No. 63/030,808, filed on May 27, 2020.
(60) Provisional application No. 63/030,787, filed on May 27, 2020.
(60) Provisional application No. 63/030,765, filed on May 27, 2020.
(60) Provisional application No. 63/058,351, filed on Jul. 29, 2020.
(60) Provisional application No. 63/058,345, filed on Jul. 29, 2020.
(60) Provisional application No. 63/058,369, filed on Jul. 29, 2020.
(60) Provisional application No. 63/058,332, filed on Jul. 29, 2020.
(60) Provisional application No. 63/058,354, filed on Jul. 29, 2020.
(60) Provisional application No. 63/142,987, filed on Jan. 28, 2021.
(60) Provisional application No. 63/142,985, filed on Jan. 28, 2021.
(60) Provisional application No. 63/142,983, filed on Jan. 28, 2021.
(60) Provisional application No. 63/142,982, filed on Jan. 28, 2021.
(60) Provisional application No. 63/142,981, filed on Jan. 28, 2021.

(51) Int. Cl. C07K 16/30; C07K 16/28

(52) U.S. Cl. CPC C07K 16/3069; C07K 16/2833; C07K 16/2878; C07K 16/2896; C07K 2317/31; C07K 2317/71; C07K 2317/732; C07K 2317/734

(57) ABSTRACT

A population of antibodies, wherein less than 80% of the oligosaccharides covalently attached to the population of the antibodies via N297 residues thereof comprise a core fucose residue; and wherein the population of the antibodies comprises an antibody which Fc region comprises K338A and T437R mutations, or K248E and T437R mutations.

### CROSS-REFERENCE TO RELATED APPLICATIONS

This application is a continuation of U.S. Ser. No. 17/233,021 filed on Apr. 16, 2021, which claims priority to U.S. Ser. No. 63/011,959, filed Apr. 17, 2020; U.S. Ser. No. 63/011,974, filed Apr. 17, 2020; U.S. Ser. No. 63/011,985, filed Apr. 17, 2020; U.S. Ser. No. 63/011,993, filed Apr. 17, 2020; U.S. Ser. No. 63/011,991, filed Apr. 17, 2020; U.S. Ser. No. 63/030,765, filed May 27, 2020; U.S. Ser. No. 63/030,787, filed May 27, 2020; U.S. Ser. No. 63/030,808, filed May 27, 2020; U.S. Ser. No. 63/030,823, filed May 27, 2020; U.S. Ser. No. 63/030,829, filed May 27, 2020; U.S. Ser. No. 63/058,354, filed Jul. 29, 2020; U.S. Ser. No. 63/058,332, filed Jul. 29, 2020; U.S. Ser. No. 63/058,369, filed Jul. 29, 2020; U.S. Ser. No. 63/058,345, filed Jul. 29, 2020; U.S. Ser. No. 63/058,351, filed Jul. 29, 2020; U.S. Ser. No. 63/142,981, filed Jan. 28, 2021; U.S. Ser. No. 63/142,982, filed Jan. 28, 2021; U.S. Ser. No. 63/142,983, filed Jan. 28, 2021; U.S. Ser. No. 63/142,985, filed Jan. 28, 2021; and U.S. Ser. No. 63/142,987, filed Jan. 28, 2021, all of which are hereby incorporated herein by reference in their entireties.

### REFERENCE TO SEQUENCE LISTING SUBMITTED ELECTRONICALLY

This application contains a sequence listing, which is submitted electronically via Patent Center as an XML formatted sequence listing with a file “JBI6274USCON1_SL.xml” and a creation date of Dec. 19, 2025 and having a size of 457,210 bytes. The sequence listing submitted via Patent Center is part of the specification and is herein incorporated by reference in its entirety.

### 1. FIELD

Provided herein are antibodies with enhanced antibody-dependent cellular cytotoxicity (ADCC) and enhanced complement-dependent cytotoxicity (CDC).

### 2. BACKGROUND

Therapeutic antibodies can bind Fc receptors expressed on immune effector cells, such as natural killer (NK) cells and macrophages, resulting in anti-tumor activity via antibody-dependent cellular cytotoxicity (ADCC). Therapeutic antibodies can also activate complement-dependent cytotoxicity (CDC) and achieve anti-tumor efficacy. There is a need in the art of an antibody with both enhanced ADCC and enhanced CDC effector functions.

### 3. SUMMARY

In one aspect, provided herein is a population of antibodies, wherein less than 80% of the oligosaccharides covalently attached to the population of the antibodies via N297 residues thereof comprise a core fucose residue; and wherein the population of the antibodies comprises an antibody which Fc region comprises K338A and T437R mutations, or K248E and T437R mutations (RE mutations), wherein amino acid residue numbering is according to the EU numbering system. In some embodiments, provided herein is a population of antibodies comprising K338A and T437R mutations, or K248E and T437R mutations (RE mutations), wherein less than 80% of the oligosaccharides covalently attached to the population of the antibodies via N297 residues thereof comprise a core fucose residue.

In some embodiments, less than 70% of the oligosaccharides covalently attached to the population of the antibodies via N297 residues thereof comprise a core fucose residue. In some embodiments, less than 60% of the oligosaccharides covalently attached to the population of the antibodies via N297 residues thereof comprise a core fucose residue. In some embodiments, less than 50% of the oligosaccharides covalently attached to the population of the antibodies via N297 residues thereof comprise a core fucose residue. In some embodiments, less than 40% of the oligosaccharides covalently attached to the population of the antibodies via N297 residues thereof comprise a core fucose residue. In some embodiments, less than 30% of the oligosaccharides covalently attached to the population of the antibodies via N297 residues thereof comprise a core fucose residue. In some embodiments, less than 20% of the oligosaccharides covalently attached to the population of the antibodies via N297 residues thereof comprise a core fucose residue. In some embodiments, less than 10% of the oligosaccharides covalently attached to the population of the antibodies via N297 residues thereof comprise a core fucose residue.

In some embodiments, the antibodies are produced by expressing a polynucleotide encoding the antibodies or a fragment thereof in a host cell that is deficient in adding a fucose to an oligosaccharide attached to an antibody.

In some embodiments, the host cell has reduced GDP-mannose 4,6-dehydratase (GMD) activity or reduced α-1,6 fucosyltransferase activity.

In some embodiments, the population of the antibodies have both enhanced antibody-dependent cellular cytotoxicity (ADCC) and enhanced complement-dependent cytotoxicity (CDC). In some embodiments, the antibodies are IgG1.

In some embodiments, the antibodies bind to HLA-G. In some embodiments, the antibodies bind to CD37. In some embodiments, the antibodies bind to GPRC5D. In some embodiments, the antibodies bind to KLK2. In some embodiments, the antibodies bind to PSMA. In some embodiments, the antibodies bind to CD3. In some embodiments, the antibodies bind to BCMA.

In some embodiments, the antibodies are monospecific antibodies. In other embodiments, the antibodies are multispecific antibodies (such as PSMA×CD3 bispecific antibodies).

In another aspect, provided herein is a population of antibodies, wherein less than 80% of the oligosaccharides covalently attached to the population of the antibodies via N297 residues thereof comprise a core fucose residue; and wherein the population of the antibodies comprises an antibody having one or more mutations in the Fc region thereof for increasing CDC activity of the antibody.

In another aspect, provided herein is a population of antibodies, comprising a first means for increasing ADCC activity of the antibodies, and a second means for increasing CDC activity of the antibodies.

In yet another aspect, provided herein is a pharmaceutical composition comprising a population of the antibodies provided herein and a pharmaceutically acceptable excipient. In another aspect, provided herein is a pharmaceutical composition comprising a population of the antibodies, wherein less than 80% of the oligosaccharides covalently attached to the population of the antibodies via N297 residues thereof comprise a core fucose residue; and wherein the population of the antibodies comprises an antibody having one or more mutations in the Fc region thereof for increasing CDC activity of the antibody, and a pharmaceutically acceptable excipient. In another aspect, provided herein is a pharmaceutical composition comprising: (a) a population of the antibodies, comprising a first means for increasing ADCC activity of the antibodies, and a second means for increasing CDC activity of the antibodies; and (b) a pharmaceutically acceptable excipient.

In yet another aspect, provided herein is a method of making a population of antibodies, comprising expressing a polynucleotide encoding the antibodies or a fragment thereof in a host cell that is deficient in adding a fucose residue to an oligosaccharide attached to an antibody via N297 residue, wherein the population of the antibodies comprises an antibody which Fc region comprises K338A and T437R mutations, or K248E and T437R mutations (RE mutations).

In some embodiments, the host cell has reduced α-1,6 fucosyltransferase activity. In some embodiments, the host cell has reduced GDP-mannose 4,6-dehydratase activity. In some embodiments, the gene encoding α-1,6 fucosyltransferase is mutated, expressed at a lower than normal level, or knocked out in the host cell. In some embodiments, the gene encoding GDP-mannose 4,6-dehydratase is mutated, expressed at a lower than normal level, or knocked out in the host cell.

In another aspect, provided herein is a method of making a population of antibodies, comprising a step for introducing K338A and T437R mutations, or K248E and T437R mutations (RE mutations) in the Fc regions of the population of the antibodies; and a step for producing the population of antibodies with reduced amount of core fucoses in the oligosaccharides attached to the antibodies via N297 residues.

In another aspect, provided herein is a method of making a population of antibodies, comprising a step of performing a function of expressing a polynucleotide encoding the antibodies or a fragment thereof in a host cell that is deficient in adding a fucose residue to an oligosaccharide attached to an antibody via N297 residue, wherein the population of the antibodies comprises an antibody which Fc region comprises K338A and T437R mutations, or K248E and T437R mutations (RE mutations).

In another aspect, provided herein is a method of making a population of antibodies, comprising a step for performing the function of introducing K338A and T437R mutations, or K248E and T437R mutations (RE mutations) in the Fc regions of the population of the antibodies; and a step for performing the function of producing the population of antibodies with reduced amount of core fucoses in the oligosaccharides attached to the antibodies via N297 residues.

In yet another aspect, provided herein is a method of treating a disease or disorder in a subject, comprising administering to the subject a population of antibodies provided herein. In some embodiments, the antibodies bind to an antigen, and wherein the disease or disorder is associated with the antigen. In some embodiments, the antigen is HLA-G. In some embodiments, the antigen is CD37. In some embodiments, the antigen is GPRC5D. In some embodiments, the antigen is KLK2. In some embodiments, the antigen is PSMA. In some embodiments, the antigen is CD3. In some embodiments, the antigen is BCMA. In some embodiments, the disease or disorder is solid tumor cancer. In some embodiments, the disease or disorder is selected from a group consisting of renal, pancreatic or lung adenocarcinoma, non-small cell lung cancer, and ovarian cancer.

In another aspect, provided herein is a method of treating a disease or disorder in a subject, comprising administering to the subject a population of antibodies, wherein less than 80% of the oligosaccharides covalently attached to the population of the antibodies via N297 residues thereof comprise a core fucose residue; and wherein the population of the antibodies comprises an antibody having one or more mutations in the Fc region thereof for increasing CDC activity of the antibody.

In another aspect, provided herein is a method of treating a disease or disorder in a subject, comprising administering to the subject (a) a population of the antibodies, comprising a first means for increasing ADCC activity of the antibodies, and a second means for increasing CDC activity of the antibodies; and (b) a pharmaceutically acceptable excipient.

In yet another aspect, provided herein is a method of modulating an immunity in a host, comprising administering to the host a population of antibodies provided herein. In another aspect, provided herein is a method of modulating an immunity in a host, comprising administering to the host a population of antibodies, wherein less than 80% of the oligosaccharides covalently attached to the population of the antibodies via N297 residues thereof comprise a core fucose residue; and wherein the population of the antibodies comprises an antibody having one or more mutations in the Fc region thereof for increasing CDC activity of the antibody. In another aspect, provided herein is a method of modulating an immunity in a host, comprising administering a population of the antibodies, comprising a first means for increasing ADCC activity of the antibodies, and a second means for increasing CDC activity of the antibodies.

### 4. BRIEF DESCRIPTION OF THE FIGURES

FIGS. 1A, 1B, 1C, 1D, 1E, 1F, 1G and 1H depict CDC activity against target cells expressing high levels of CD37 (CARNAVAL, FIGS. 1A, 1C, 1E and 1G) and low levels of CD37 (JEKO-1, FIGS. 1B, 1D, 1F and 1H) of different types of anti-CD37 antibodies with low fucosylation (FIGS. 1A and 1B), with Xencor mutations (FIGS. 1C and 1D), with RE mutations (FIGS. 1E and 1F), and with low fucosylation and RE mutations (FIGS. 1G and 1H). Target cells were incubated with titrating concentrations of antibodies as indicated for 30 minutes. Baby rabbit serum was then added to the mixture to a final concentration of 10% to provide a source of complement components. After 4 hours of incubation, cell viability was measured by addition of Cell Titer-Glo reagent (Promega) and measurement of the resulting luminescence and reported in Relative Luminescence Units (RLU).

FIG. 2 depicts CDC activity against H929 target cells of anti-GPRC5D antibodies with low fucosylation in wildtype and with RE mutations. Target cells were incubated with titrating concentrations of antibodies as indicated for 30 minutes. Baby rabbit serum was then added to the mixture to a final concentration of 10% to provide a source of complement components. After 4 hours of incubation, cell viability was measured by addition of Cell Titer-Glo reagent (Promega) and measurement of the resulting luminescence and reported in Relative Luminescence Units (RLU).

FIGS. 3A, 3B, 3C, 3D and 3E show the in vitro ADCC kinetic killing mediated by KLK2 antibodies with PBMC on VCap cells. Briefly, VcaP cells stably transfected with Nuclight Red (Incucyte®, Essen Bioscience) were plated at 10,000 cells per well in a 384-well plate (Perkin Elmer ViewPlate) in clear media (RPMI 1641+10% FBS, Thermo Fisher Scientific) to allow for cell adherence overnight. ADCC assay was performed with freshly thawed PBMC (Hemcare, PB009C-3). The ratio of effector to target cell per well was 34:1 for PBMCs as effector cells. KLK2 antibodies were tested with final concentrations ranging from 100 nM to 0.01 nM. After effector cells and antibodies were added to target cells, real time imaging was performed under Incucyte® S3 instrument (Essen BioScience). Total red intergraded signal per well was quantified with Incucyte® software. Data analysis were performed by Incucyte® software and Prism (GraphPad Software) based on values of quadruplicates. The percentage of cell killing was calculated as: (1−KLK2 mAb/no mAb control)×100%.

FIG. 4 shows the in vitro ADCC dose-response killing by PBMC on VcaP at 48 hour. The dose-response curve was generated 48 hours after effector cells and antibodies were added to target cells.

FIGS. 5A and 5B depict the ADCC activity against JEG-3 and RERF-LC-Ad-1 cells of anti-HLA-G antibodies MHGB732 and MHGB738, and their respective counterparts with low fucosylation, with RE mutations, and with low fucosylation and RE mutations. The percentage of lysis was compared to the maximum lysis of JEG-3 or RERF-LC-Ad-1 cells by Triton-X 100 detergent and calculated as (sample value−target alone value)/(maximum value−target alone value)×100%.

FIGS. 5C and 5D depict the CDC activity of anti-HLA-G antibodies MHGB732 and MHGB738, and their respective counterparts with low fucosylation, with RE mutations, and with low fucosylation and RE mutations. Target cells were incubated with antibodies as indicated for 30 minutes at 37° C. 15-20% (stock concentration) of rabbit complement and heat inactivated complement was added to the wells respectively to a volume of 25 μl/well. The mixture was incubated for 4-12 hours at 37° C. Target cell lysis was measured by addition of Cell Titer-Glo reagent (Promega) and measurement of the resulting luminescence and reported in Relative Luminescence Units (RLU).

FIGS. 6A, 6B, 6C, and 6D show the in vitro ADCC dose-response killing mediated by anti-PSMA antibodies PSMB896 and PSMB898 with low fucosylation and RE mutations on C42B and LNCap cells. Specifically, FIG. 6A shows the in vitro ADCC dose-response killing mediated by the antibodies with PBMC on C42B cells at 6 hour; FIG. 6B shows the in vitro ADCC dose-response killing mediated by the antibodies with PBMC on LNCap cells at 6 hour; FIG. 6C shows the in vitro ADCC dose-response killing mediated by the antibodies with NK cells on C42B cells at 24 hour; FIG. 6D shows the in vitro ADCC dose-response killing mediated by the antibodies with NK cells on LNCap cells at 24 hour. The dose-response curves were generated 6 or 24 hours after effector cells and antibodies were added to target cells.

### 5. DETAILED DESCRIPTION

The present disclosure is based in part on the surprising finding that a population of antibodies possess both enhanced ADCC and enhanced CDC activities when less than 80% of the oligosaccharides covalently attached to the population of the antibodies comprise a fucose residue and the Fc region of the antibodies comprises K248E and T437R mutations (RE mutations). In addition, the low fucosylation does not interfere the effect of the RE mutations on enhancing CDC, and RE mutations do not interfere the enhanced ADCC activity conferred by low fucosylation, as demonstrated in Section 7 below. In one aspect, provided herein is an antibody that comprises an RE mutation in the Fc region and does not comprise a core fucose residue in the oligosaccharides covalently attached to the Fc region of the antibody. In another aspect, provided herein is a population of antibodies that comprise an antibody that comprises an RE mutation in the Fc region and does not comprise a core fucose residue in the oligosaccharides covalently attached to the Fc region of the antibody. In some embodiments, provided herein is a population of antibodies comprising K248E and T437R mutations (RE mutations), wherein less than 80% of the oligosaccharides covalently attached to the population of the antibodies via N297 residues thereof comprise a core fucose residue. In some embodiments, less than 70% of the oligosaccharides covalently attached to the population of the antibodies via N297 residues thereof comprise a core fucose residue. In some embodiments, less than 60% of the oligosaccharides covalently attached to the population of the antibodies via N297 residues thereof comprise a core fucose residue. In some embodiments, less than 50% of the oligosaccharides covalently attached to the population of the antibodies via N297 residues thereof comprise a core fucose residue. In some embodiments, less than 40% of the oligosaccharides covalently attached to the population of the antibodies via N297 residues thereof comprise a core fucose residue. In some embodiments, less than 30% of the oligosaccharides covalently attached to the population of the antibodies via N297 residues thereof comprise a core fucose residue. In some embodiments, less than 20% of the oligosaccharides covalently attached to the population of the antibodies via N297 residues thereof comprise a core fucose residue. In some embodiments, less than 10% of the oligosaccharides covalently attached to the population of the antibodies via N297 residues thereof comprise a core fucose residue. In some embodiments, less than 5% of the oligosaccharides covalently attached to the population of the antibodies via N297 residues thereof comprise a core fucose residue.

In certain embodiments, any antibodies with any antigen binding structure or targeting any antigen are included in the present disclosure, as long as the antibodies have a Fc region. Pharmaceutical compositions comprising the present antibodies, method of making, and uses thereof are also included in the present disclosure.

### 5.1 Definitions

Techniques and procedures described or referenced herein include those that are generally well understood and/or commonly employed using conventional methodology by those skilled in the art, such as, for example, the widely utilized methodologies described in Molecular Cloning: A Laboratory Manual (Sambrook, et al., 3d ed. 2001); Current Protocols in Molecular Biology (Ausubel, et al. eds., 2003); Therapeutic Monoclonal Antibodies: From Bench to Clinic (An, ed. 2009); Monoclonal Antibodies: Methods and Protocols (Albitar, ed. 2010); and Antibody Engineering Vols 1 and 2 (Kontermann and Dübel, eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, and multispecific antibodies (e.g., bispecific antibodies so long as they exhibit the desired biological activity), formed from at least two intact antibodies, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck, ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies or their humanized variants, intrabodies, and anti-idiotypic (anti-Id) antibodies. The term “antibody” as used herein also comprises any binding molecule having a Fc region and a functional fragment (e.g., an antigen-binding fragment) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers, ed., 1995); Huston, et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen binding site as well as a constant domain (CL) and at least heavy chain constant regions, CH1, CH2 and CH3. The constant regions may include human constant regions or amino acid sequence variants thereof. In certain embodiments, an intact antibody has one or more effector functions.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody described herein, the terms such as “bind to,” “that specifically bind to,” and analogous terms are also used interchangeably herein and refer to antibodies of antigen binding domains that specifically bind to an antigen, such as a polypeptide. An antibody or antigen binding domain that binds to or specifically binds to an antigen may be cross-reactive with related antigens. In certain embodiments, an antibody or antigen binding domain that binds to or specifically binds to an antigen does not cross-react with other antigens. An antibody or antigen binding domain that binds to or specifically binds to an antigen can be identified, for example, by immunoassays, Octet®, Biacore®, or other techniques known to those of skill in the art. In some embodiments, an antibody or antigen binding domain binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically a specific or selective reaction will be at least twice background signal or noise and may be more than 10 times background. See, e.g., Fundamental Immunology 332-36 (Paul, ed., 2d ed. 1989) for a discussion regarding binding specificity. In certain embodiments, the extent of binding of an antibody or antigen binding domain to a “non-target” protein is less than about 10% of the binding of the antibody or antigen binding domain to its particular target antigen, for example, as determined by fluorescence activated cell sorting (FACS) analysis or RIA. With regard to terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding domain that binds to an antigen includes one that is capable of binding the antigen with sufficient affinity such that the antibody is useful, for example, as a diagnostic or therapeutic agent in targeting the antigen. In certain embodiments, an antibody or antigen binding domain that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding domain binds to an epitope of an antigen that is conserved among the antigen from different species (e.g., between human and cynomolgus macaque species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen, et al., J. Mol Biol, 1999, 293:865-81). The K_{D} or K_{D} value may also be measured by using biolayer interferometry (BLI) or surface plasmon resonance (SPR) assays by Octet®, using, for example, an Octet® Red96 system, or by Biacore®, using, for example, a Biacore® 2000 or a Biacore® 3000. An “on-rate” or “rate of association” or “association rate” or “kₒₙ” may also be determined with the same biolayer interferometry (BLI) or surface plasmon resonance (SPR) techniques described above using, for example, the Octet® Red96, the Biacore® 2000, or the Biacore® 3000 system.

In certain embodiments, the antibodies can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, so long as they exhibit the desired biological activity (see U.S. Pat. No. 4,816,567; and Morrison, et al., Proc. Natl. Acad. Sci. USA, 1984, 81:6851-55).

In certain embodiments, the antibodies can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones, et al., Nature, 1986, 321:522-25; Riechmann, et al., Nature, 1988, 332:323-29; Presta, Curr. Op. Struct. Biol., 1992, 2:593-96; Carter, et al., Proc. Natl. Acad. Sci. USA, 1992, 89:4285-89; U.S. Pat. Nos. 6,800,738; 6,719,971; 6,639,055; 6,407,213; and 6,054,297.

In certain embodiments, the antibodies can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat, et al. (see Kabat, et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol., 1991, 227:381; Marks, et al., 1991, J. Mol. Biol., 1991, 222:581) and yeast display libraries (Chao, et al., Nature Protocols, 2006, 1: 755-68). Also available for the preparation of human monoclonal antibodies are methods described in Cole, et al., Monoclonal Antibodies and Cancer Therapy 77 (1985); Boerner, et al., J. Immunol., 1991, 147(1):86-95; and van Dijk and van de Winkel, Curr. Opin. Pharmacol., 2001, 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol., 1995, 6(5):561-66; Brüggemann and Taussing, Curr. Opin. Biotechnol., 1997, 8(4):455-58; and U.S. Pat. Nos. 6,075,181 and 6,150,584 regarding XENOMOUSE™ technology). See also, for example, Li, et al., Proc. Natl. Acad. Sci. USA, 2006, 103:3557-62, regarding human antibodies generated via a human B-cell hybridoma technology.

In certain embodiments, the antibodies can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D., et al., Nucl. Acids Res., 1992 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson, et al., Nature, 1991, 352:624-28 and Marks, et al., J. Mol. Biol., 1991, 222:581-97, for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites, et al. eds., 8th ed. 1994); and Immunobiology (Janeway, et al. eds., 5^{th} ed. 2001).

The term “core fucose,” “core fucose residue,” “fucose,” or “fucose residue” as used herein refers to a fucose residue in an α1,6-linkage to the first GlcNAc of the Asn-297-linked N-oligosaccharide. Ferrara et al., Proc Natl Acad Sci USA, 2011, 108:12669-74. “Core fucose,” “core fucose residue,” “fucose,” and “fucose residue” are used interchangeably in the present disclosure.

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail in the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat, et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat, et al., supra). The “EU numbering system” or “EU index” is generally used when referring to a residue in an immunoglobulin heavy chain constant region (e.g., the EU index reported in Kabat, et al., supra). The “EU index as in Kabat” refers to the residue numbering of the human IgG1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat, et al., supra). Chothia refers instead to the location of the structural loops (see, e.g., Chothia and Lesk, J. Mol. Biol., 1987, 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel, eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc, et al., Dev. Comp. Immunol., 2003, 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol., 2001, 309: 657-70. Correspondence between the numbering system, including, for example, the Kabat numbering and the IMGT unique numbering system, is well known to one skilled in the art (see, e.g., Kabat, supra; Chothia and Lesk, supra; Martin, supra; Lefranc, et al., supra). The residues from each of these hypervariable regions or CDRs are noted below.

TABLE 1

| Loop | Kabat | AbM | Chothia | Contact | IMGT |
|---|---|---|---|---|---|
| CDR L1 | L24- | L24- | L24- | L30- | L27- |
| | L34 | L34 | L34 | L36 | L38 |
| CDR L2 | L50- | L50- | L50- | L46- | L56- |
| | L56 | L56 | L56 | L55 | L65 |
| CDR L3 | L89- | L89- | L89- | L89- | L105- |
| | L97 | L97 | L97 | L96 | L117 |
| CDR H1 | H31-H35B | H26- | H26- | H30- | H27- |
| | (Kabat | H35B | H32 . . . 34 | H35B | H38 |
| | Numbering) | | | | |
| CDR H1 | H31-H35 | H26- | H26- | H30- | |
| | (Chothia | H35 | H32 | H35 | |
| | Numbering) | | | | |
| CDR H2 | H50- | H50- | H52- | H47- | H56- |
| | H65 | H58 | H56 | H58 | H65 |
| CDR H3 | H95- | H95- | H95- | H93- | H105- |
| | H102 | H102 | H102 | H101 | H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues. There are typically four FR regions in each of VH and VL regions. The FR regions in VH are VH FR1, VH FR2, VH FR3, and VH FR4 (or FR H1, FR H2, FR H3 and FR H4). The FR regions in VL are VL FR1, VL FR2, VL FR3 and VL FR4 (or FR L1, FR L2, FR L3 and FR L4).

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “variant” when used in relation to an antigen or an antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a CD37 variant may result from one or more (such as, for example, about 1 to about 25, about 1 to about 20, about 1 to about 15, about 1 to about 10, or about 1 to about 5) changes to an amino acid sequence of a native CD37. Also by way of example, a variant of an anti-CD37 antibody may result from one or more (such as, for example, about 1 to about 25, about 1 to about 20, about 1 to about 15, about 1 to about 10, or about 1 to about 5) changes to an amino acid sequence of a native or previously unmodified anti-CD37 antibody. Variants may be naturally occurring, such as allelic or splice variants, or may be artificially constructed. Polypeptide variants may be prepared from the corresponding nucleic acid molecules encoding the variants. In specific embodiments, the CD37 variant or anti-CD37 antibody variant at least retains CD37 or anti-CD37 antibody functional activity, respectively. In specific embodiments, an anti-CD37 antibody variant binds CD37 and/or is antagonistic to CD37 activity. In certain embodiments, the variant is encoded by a single nucleotide polymorphism (SNP) variant of a nucleic acid molecule that encodes CD37 or anti-CD37 antibody VH or VL regions or subregions, such as one or more CDRs.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

As used herein, an “epitope” is a term in the art and refers to a localized region of an antigen to which an antibody can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three dimensional protein structure. In other embodiments, an antibody requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “vector” refers to a substance that is used to carry or include a nucleic acid sequence, including for example, a nucleic acid sequence encoding an antibody as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide,” “nucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces an antibody of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete)), or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain an antibody, for example, in isolated or purified form, together with a suitable amount of excipients.

The term “effective amount” or “therapeutically effective amount” as used herein refers to the amount of an antibody or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular, subcutaneous delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s).

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2 Low Fucosylation

Antibody glycosylation is a type of posttranslational modification that may occur via the addition of oligosaccharides to antibodies through two types of covalent linkages: linkages on asparagine residues (N-oligosaccharides)s or on serine/threonine residues (O-oligosaccharides) (Alter, G., et al., Semin Immunol., 2018, 39:102-10), and profoundly affect therapeutic functions of antibodies (Walsh, G. and Jefferis, R., Nat. Biotechnol., 2006, 24:1241-52; Jefferis, R., Nat. Rev. Drug Discov., 2009, 8(3):226-34; Dalziel, M., et al., Science, 2014, 343(6166):1235681). Notably, all IgG antibodies are glycosylated in the Fc region thereof on a conserved Asn-297 residue (Alter G., et al., supra).

An Asn-297-linked N-oligosaccharide is comprised of a conserved biantennary core structure (Liu, L., J Pharm Sci., 2015, 104(6):1866-84) consisting of two covalently-linked N-acetylglucosamine (GlcNAc) residues, further linked to a mannose, which links in a 1,3- and 1,6-branching manner to two other mannose residues (Alter, G., et al., supra). Additional monosaccharides, including two galactoses, a fucose, a bisecting GlcNAc, and two sialic acids (Alter, G., et al., supra), may extend the core structure, giving rise to considerable structural and functional heterogeneity (Jefferis, R., Biochem J., 1990, 268(3):529-37; Rudd, P. M., Science, 2001, 291(5512):2370-6; Liu, L., supra). At least 30 structures (glycoforms) for IgG Asn-297-linked N-oligosaccharides have been reported (Alter, G., et al., supra).

Antibodies expressed in mammalian cells are usually more than 80% fucosylated (Kamoda, S., et al., J Chromatogr A., 2004, 1050(2):211-6; Shinkawa, T., et al., J Biol Chem., 2003, 278(5):3466-73). For example, normal Chinese Hamster Ovary (CHO) cells and HEK293 cells add fucose to 80-98% of Asn-297-linked N-oligosaccharides to IgG antibodies (Shields, R. L. et al., J Biol Chem., 2002, 277(30):26733-40).

In one aspect, provided herein is an antibody having no fucose in the oligosaccharide attached to its Fc region and having RE mutations in the Fc region. In another aspect, provided herein is a population of antibodies comprising an antibody having no fucose in the oligosaccharide attached to its Fc region and having RE mutations in the Fc region. In yet another aspect, provided herein is a population of antibodies wherein less than 80% of the oligosaccharides covalently attached to the population of the antibodies comprise a fucose residue and the Fc region of these antibodies comprises K248E and T437R mutations (RE mutations).

In some embodiments, less than 70% of the oligosaccharides covalently attached to the population of the antibodies comprise a fucose residue. In some embodiment, less than 60% of the oligosaccharides covalently attached to the population of the antibodies comprise a fucose residue. In some embodiments, less than 50% of the oligosaccharides covalently attached to the population of the antibodies comprise a fucose residue. In other embodiments, less than 40% of the oligosaccharides covalently attached to the population of the antibodies comprise a fucose residue. In yet other embodiments, less than 30% of the oligosaccharides covalently attached to the population of the antibodies comprise a fucose residue. In yet other embodiments, less than 20% of the oligosaccharides covalently attached to the population of the antibodies comprise a fucose residue. In yet other embodiments, less than 10% of the oligosaccharides covalently attached to the population of the antibodies comprise a fucose residue.

Standard techniques known to those of skill in the art, e.g., mass spectrometry, can be used to characterize the Asn297-linked N-oligosaccharides on the antibodies (Pereira, N. A., et al., supra; Shields, R. L. et al., supra). For example, in a matrix-assisted laser desorption/ionization time-of-flight mass spectral (MALDI-TOF-MS) analysis, 50 mg of IgG antibodies were immobilized in MultiScreen 96-well IP plates (Millipore) to polyvinylidene difluoride membranes. Proteins were then reduced using 50 mL of a 0.1 M solution of DTT in RCM buffer (pH 8.6, 3.2 mM EDTA, 360 mM Tris, and 8 M urea). Next, they were incubated in the dark for 30 minutes at 25° C. in RCM buffer containing 0.1 M iodoacetic acid, in order to carboxymethylate the free sulfhydryl groups resulting from the reduction step. Membrane-bound proteins were then incubated for 1 hour at 25° C. in a 1% solution of polyvinylpyrrolidone 360 (Sigma) in water, and their oligosaccharides were cleaved from the proteins by a three-step process: incubation for 3 hours at 37° C. in pH 8.4 Tris acetate buffer (25 mL) containing 32 units of peptide:N-glycosidase F (New England Biolabs, Beverly, MA), addition of 1.5 M acetic acid (2.5 mL) to lower the pH, and incubation for 3 hours at 25° C. (Shields, R. L. et al., J Biol Chem., 2001, 276(9):6591-604).

In some embodiments, the antibodies provided herein are produced by expressing a polynucleotide encoding the antibodies or a fragment thereof in a host cell that is deficient in additing a fucose to an oligosaccharide attached to an antibody.

In mammalian cells, FUT8 encodes the only enzyme, α-1,6 fucosyltransferase, that catalyzes core fucosylation, the transfer of a GDP-fucose residue to the innermost GlcNAc via α-1,6-linkage (Imai-Nishiya, H., et al., BMC Biotechnol., 2007, 7:84). Oligosaccharide fucosylation requires intracellular GDP-fucose as substrate, which is synthesized via the de novo pathway or the salvage pathway in the cytoplasm. In the de novo pathway, GDP-mannose 4,6-dehydratase (GMD) mediates the synthesis of GDP-4-keto-6-deoxy-mannose (GKDM) from GDP-mannose, followed by the synthesis of GDP-fucose mediated by GDP-keto-6-deoxymannose 3,5-epimerase, 4-reductase (FX) (Imai-Nishiya, H., et al., supra). As such, cell lines with deficient GMD enzymes, e.g., CHO Lec13 cells, or reduced α-1,6 fucosyltransferase activity resulting from mutated FUT8 genes, have been shown to generate afucosylated antibodies (Pereira, N. A., et al., Mabs, 2018, 10(5):693-711). For example, antibodies with approximately 10% fucosylation (Shields, R. L. et al., supra) or less can be consistently produced in Lec13 cells (Shields, R. L. et al., supra; Kanda Y., Biotechnol Bioeng., 2006, 94(4):680-8), while increased fucosylation may occur when cells are cultured in a static flask to confluence (Pereira, N. A., et al., supra).

The addition of a bisecting GlcNAc to the oligosaccharide core structure creates steric hindrance for fucosylation (Alter, G., et al., supra). As such, overexpression of β-1,4-mannosyl-glycoprotein 4-β-N-acetylglucosaminyltransferase (GnT-III), which catalyzes the addition of a bisecting GlcNAc to the innermost mannose, was shown to dramatically reduce Fc fucosylation (Pereira, N. A., et al., supra).

Moreover, inactivated Golgi GDP-fucose transporter (GFT) gene (Slc35c1) has been shown to produce afucosylated antibodies, e.g., in CHO-gmt3 cells (Pereira, N. A., et al., supra). Use of biochemical inhibitors of fucosylation, e.g., fucose analogs such as 2-fluorofucose and 5-alkynylfucose, can also generate afucosylated antibodies (Pereira, N. A., et al., supra). The intermediate GKDM in the de novo fucose synthesis pathway in mammalian cells can be reduced by bacteria GDP-4-keto-6-deoxy mannose reductase (RMD) to GDP-rhamnose, thus bypassing the fucose biosynthesis pathway. Afucosylated antibodies can also be generated in cells in which bacterial RMD is heterologously expressed in the cytosol (Pereira, N. A., et al., supra).

In some embodiments, the antibodies provided herein are produced by expressing the antibodies in a host cell having a deficiency in any of the above mentioned enzymes. In some embodiments, the host cell has reduced GDP-mannose 4,6-dehydratase (GMD) activity. In some embodiments, the host cell has reduced α-1,6 fucosyltransferase activity.

### 5.3 Fc Mutations with Enhanced Effector Functions

The antibodies provided herein have mutations at the lysine at position 248 (K248) (EU numbering) and the threonine at position 437 (T437) (EU numbering) in the Fc region. Lysine at position 248 (K248) (EU numbering) and threonine at position 437 (T437) (EU numbering) are both conserved residues in the Fc regions among different IgG subtypes (Zhang, D., et al., supra). Fc mutations, T437R and K248E (EU numbering), were shown to facilitate oligomerization of antibodies upon binding antigens at the cell surface, and possess enhanced effector functions (Zhang, D., et al., supra). T437R and K248E double mutations (“RE mutations”) were shown to confer CDC activity on wildtype IgG1 antibodies that did not possess CDC activity in a dose-dependent manner (Zhang, D., et al., supra).

The “EU numbering” or “EU index” is generally used when referring to a residue in an immunoglobulin heavy chain constant region. It refers to the residue numbering of the human IgG1 EU antibody. It is computed by alignment of an antibody sequence with the Eu antibody sequence (Edelman, G. M., et al., Proc Natl Acad Sci USA, 1969, 63(1):78-85; Kabat, et al., supra), so that each residue that is homologous to a residue in the Eu antibody will have the same residue number as that Eu residue.

Antibodies comprising other Fc region mutations, e.g., S239D/I332E and S239D/I332E/A330L (EU numbering) have also been shown to exhibit significantly enhanced ADCC via enhanced binding to FcγRs (Lazar, G. A. et al., Proc Natl Acad Sci USA, 2006, 103(11):4005-10).

### 5.4 Antibody-Dependent Cell-Mediated Cytotoxicity (ADCC) and Complement-Dependent Cytotoxicity (CDC) Effector Functions

The antibody or population of antibodies provided herein has both enhanced ADCC activity and enhanced CDC activity.

Therapeutic antibodies bind Fc receptors on the cell surface of effector cells, such as natural killer (NK) cells, macrophages, mononuclear phagocytes, neutrophils and eosinophils (Saunder, K. O., Front Immunol., 2019, 10:1296), giving rise to important antibody-dependent effector functions, such as antibody-dependent cell-mediated cytotoxicity (ADCC) and antibody-dependent cell-mediated phagocytosis (ADCP). A family of receptors for IgG Fc regions was referred to as the Fcγ receptors (FcγRs) (Cohen-Solal, J. F., Immunol Lett., 2004, 92(3):199-205), and is comprised of FcγRI; FcγRII, including isoforms FcγRIIa, FcγRIIb, and FcγRIIc; and FcγRIII, including isoforms FcγRIIIa and FcγRIIIb (Jefferis, R. and Lund, J., Immunol Lett., 2002, 82(1-2):57-65).

Among various effector functions, ADCC and ADCP have been shown to possess clinically significant anti-tumor efficacy. For example, ADCC was shown to be an important mechanism for the anti-tumor efficacy of trastuzumab in vitro, as evidenced by NK cells' capability to kill trastuzumab-coated tumor cells via a FcγRIII receptor (CD16)-mediated ADCC mechanism (Cooley, S., et al., Exp Hematol., 1999; 27(10):1533-41; Carson, W. E., et al., Eur J Immunol., 2001, 31(10):3016-3025; Kubo, M., et al., Anticancer Res., 2003, 23(6a):4443-9) and in vivo, as evidenced by increased numbers of NK cells in tumor infiltrates after trastuzumab treatment (Clynes, R. A., et al., Nat Med., 2000, 6(4):443-6; Arnould, L., et al., Br J Cancer, 2006, 94(2):259-67). Additionally, macrophage-mediated ADCP has been shown to be important in the anti-tumor efficacy of trastuzumab (Shi, Y., et al., J Immunol., 2015, 194(9):4379-86).

Antibodies with no or low fucosylation have shown dramatically enhanced ADCC activity due to the enhancement in their binding capacity to FcγRIIIa binding without any detectable change in CDC or antigen binding capability (Okazaki, A., et al., J Mol Biol., 2004, 336(5):1239-49; Kanda, Y., et al., Glycobiology, 2007, 17(1):104-18). N-oligosaccharides of antibody Fc regions are essential for binding to FcγR, which engages antibody effector functions (Yamane-Ohnuki, N. and Satoh, M., Mabs, 2009, 1(3):230-6).

The absence of fucose on N-oligosaccharides of antibody Fc regions have been shown to dramatically enhance antibodies' binding capacity to FcγRIIIa receptors present on immune effector cells such as natural killer (NK) cells and macrophages, giving rise to anti-tumor therapeutic effect (Pereira, N. A., et al., supra). The FcγRIIIa receptors bind Fc regions via interactions with the hinge region and the CH2 domain of the Fc (Radaev, S., et al., J Biol Chem., 2001, 276:16469-77; Sondermann, P., et al., Nature, 2000, 406:267-73). The absence of fucose thus eliminates the steric hindrance and enhances the Fc-FcγRIIIa interaction, leading to enhanced effector functions (Pereira, N. A., et al., supra).

Complement-dependent cytotoxicity (CDC) is another important antibody effector function. In the antibody-dependent classical complement activation pathway, binding between the complement C1q heterohexameric headpiece and an oligomeric antibody complex initiates the proteolytic complement cascade (Wang, G. et al., Mol Cell, 2016, 63:135-45; Diebolder, C. A. et al., Science, 2014, 343:1260-3), which leads to the opsonization of target cells by C3-derived opsonins (e.g., C3b) and generation of potent inflammation mediators (C3a and C5a), ultimately resulting in the formation of membrane attack complex (MAC), C5b-C9, on the target cell membrane (Reis, E. S., et al., Nat Rev Immunol., 2018, 18:5-18). CDC has also been shown to possess clinically significant anti-tumor efficacy, e.g., in the anti-CD20 mAb rituximab and anti-CD38 mAb daratumumab (de Weers, M., et al., J Immunol., 2011, 186:1840-8; Lokhorst, H. M., et al., N Engl J Med., 2015, 373:1207-19; Taylor, R. P. and Lindorfer, M. A., Semin Immunol., 2016, 28:309-16).

Mutations in the Fc region that facilitate antibody oligomerization, such as the RE mutations and E345R (EU numbering), have been demonstrated to significantly enhance antibody CDC activity (Diebolder, C. A. et al., supra; Zhang, D., et al., supra).

In some embodiments, the ADCC activity of the present antibodies is 10% higher than antibodies with normal fucosylation. In some embodiments, the ADCC activity of the present antibodies is 20% higher than antibodies with normal fucosylation. In some embodiments, the ADCC activity of the present antibodies is 30% higher than antibodies with normal fucosylation. In some embodiments, the ADCC activity of the present antibodies is 40% higher than antibodies with normal fucosylation. In some embodiments, the ADCC activity of the present antibodies is 50% higher than antibodies with normal fucosylation. In some embodiments, the ADCC activity of the present antibodies is 60% higher than antibodies with normal fucosylation. In some embodiments, the ADCC activity of the present antibodies is 70% higher than antibodies with normal fucosylation. In some embodiments, the ADCC activity of the present antibodies is 80% higher than antibodies with normal fucosylation. In some embodiments, the ADCC activity of the present antibodies is 90% higher than antibodies with normal fucosylation. In some embodiments, the ADCC activity of the present antibodies is more than 2 fold of that of antibodies with normal fucosylation. In some embodiments, the ADCC activity of the present antibodies is more than 3 fold of that of antibodies with normal fucosylation. In some embodiments, the ADCC activity of the present antibodies is more than 4 fold of that of antibodies with normal fucosylation. In some embodiments, the ADCC activity of the present antibodies is more than 5 fold of that of antibodies with normal fucosylation. In some embodiments, the ADCC activity of the present antibodies is more than 6 fold of that of antibodies with normal fucosylation. In some embodiments, the ADCC activity of the present antibodies is more than 7 fold of that of antibodies with normal fucosylation. In some embodiments, the ADCC activity of the present antibodies is more than 8 fold of that of antibodies with normal fucosylation. In some embodiments, the ADCC activity of the present antibodies is more than 9 fold of that of antibodies with normal fucosylation. In some embodiments, the ADCC activity of the present antibodies is more than 10 fold of that of antibodies with normal fucosylation.

In some embodiments, the antibodies described above also have higher CDC activities. In some embodiments, the CDC activity of the present antibodies is 10% higher than antibodies without RE mutations. In some embodiments, the CDC activity of the present antibodies is 20% higher than antibodies without RE mutations. In some embodiments, the CDC activity of the present antibodies is 30% higher than antibodies without RE mutations. In some embodiments, the CDC activity of the present antibodies is 40% higher than antibodies without RE mutations. In some embodiments, the CDC activity of the present antibodies is 50% higher than antibodies without RE mutations. In some embodiments, the CDC activity of the present antibodies is 60% higher than antibodies without RE mutations. In some embodiments, the CDC activity of the present antibodies is 70% higher than antibodies without RE mutations. In some embodiments, the CDC activity of the present antibodies is 80% higher than antibodies without RE mutations. In some embodiments, the CDC activity of the present antibodies is 90% higher than antibodies without RE mutations. In some embodiments, the CDC activity of the present antibodies is more than 2 fold of that of antibodies without RE mutations. In some embodiments, the CDC activity of the present antibodies is more than 3 fold of that of antibodies without RE mutations. In some embodiments, the CDC activity of the present antibodies is more than 4 fold of that of antibodies without RE mutations. In some embodiments, the CDC activity of the present antibodies is more than 5 fold of that of antibodies without RE mutations. In some embodiments, the CDC activity of the present antibodies is more than 6 fold of that of antibodies without RE mutations. In some embodiments, the CDC activity of the present antibodies is more than 7 fold of that of antibodies without RE mutations. In some embodiments, the CDC activity of the present antibodies is more than 8 fold of that of antibodies without RE mutations. In some embodiments, the CDC activity of the present antibodies is more than 9 fold of that of antibodies without RE mutations. In some embodiments, the CDC activity of the present antibodies is more than 10 fold of that of antibodies without RE mutations.

### 5.5 Anti-HLA-G Antibodies and Related Molecules

In one aspect, provided herein are anti-HLA-G antibodies.

In some embodiments, the antibody is MHGB732 comprising a VL comprising an amino acid sequence of SEQ ID NO: 172 and a VH comprising an amino acid sequence of SEQ ID NO: 174. In some embodiments, the antibody is MHGB738 comprising a VL comprising an amino acid sequence of SEQ ID NO: 173 and a VH comprising an amino acid sequence of SEQ ID NO: 175.

In some embodiments, provided herein is an antibody that binds to HLA-G, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO: 174; and (ii) a VL comprising a VL CDR1, a VL CDR2, and a VL CDR3 having amino acid sequences of the VL CDR1, VL CDR2, and VL CDR3, respectively, of SEQ ID NO: 172. In some embodiments, provided herein is an antibody that binds to HLA-G, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO: 175; and (ii) a VL comprising a VL CDR1, a VL CDR2, and a VL CDR3 having amino acid sequences of the VL CDR1, VL CDR2, and VL CDR3, respectively, of SEQ ID NO: 173. In some embodiments, provided herein is an antibody that binds to HLA-G, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO: 174; and (ii) a VL comprising a VL CDR1, a VL CDR2, and a VL CDR3 having amino acid sequences of the VL CDR1, VL CDR2, and VL CDR3, respectively, of SEQ ID NO: 173. In some embodiments, provided herein is an antibody that binds to HLA-G, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO: 175; and (ii) a VL comprising a VL CDR1, a VL CDR2, and a VL CDR3 having amino acid sequences of the VL CDR1, VL CDR2, and VL CDR3, respectively, of SEQ ID NO: 172.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system.

In another aspect, provided herein is an antibody that binds to HLA-G, wherein the antibody comprises: (i) a VH comprising a VH CDR1 having an amino acid sequence of SNSAAWN (SEQ ID NO: 184), a VH CDR2 having an amino acid sequence of RTYYRSKWYNDYAVSVKS (SEQ ID NO: 185), and a VH CDR3 having an amino acid sequence of DRRYGIVGLPFAY (SEQ ID NO: 186); and (ii) a VL comprising a VL CDR1 having an amino acid sequence of KSSQSVLHSSNNKNYLT (SEQ ID NO: 187), a VL CDR2 having an amino acid sequence of WASTRES (SEQ ID NO: 188), and a VL CDR3 having an amino acid sequence of HQYYSTPPT (SEQ ID NO: 189).

In another aspect, provided herein is an antibody that binds to HLA-G, wherein the antibody comprises: (i) a VH comprising a VH CDR1 having an amino acid sequence of GDSVSSNSA (SEQ ID NO: 190), a VH CDR2 having an amino acid sequence of YYRSKWY (SEQ ID NO: 191), and a VH CDR3 having an amino acid sequence of DRRYGIVGLPFA (SEQ ID NO: 192); and (ii) a VL comprising a VL CDR1 having an amino acid sequence of SQSVLHSSNNKNY (SEQ ID NO: 193), a VL CDR2 having an amino acid sequence of WAS (SEQ ID NO: 194), and a VL CDR3 having an amino acid sequence of YYSTPP (SEQ ID NO: 195).

In another aspect, provided herein is an antibody that binds to HLA-G, wherein the antibody comprises: (i) a VH comprising a VH CDR1 having an amino acid sequence of GDSVSSNSAA (SEQ ID NO: 196), a VH CDR2 having an amino acid sequence of TYYRSKWYN (SEQ ID NO: 197), and a VH CDR3 having an amino acid sequence of AGDRRYGIVGLPFAY (SEQ ID NO: 198); and (ii) a VL comprising a VL CDR1 having an amino acid sequence of QSVLHSSNNKNY (SEQ ID NO: 199), a VL CDR2 having an amino acid sequence of WAS (SEQ ID NO: 200), and a VL CDR3 having an amino acid sequence of HQYYSTPPT (SEQ ID NO: 201).

In another aspect, provided herein is an antibody that binds to HLA-G, wherein the antibody comprises: (i) a VH comprising a VH CDR1 having an amino acid sequence of GDSVSSNSAAWN (SEQ ID NO: 202), a VH CDR2 having an amino acid sequence of RTYYRSKWYND (SEQ ID NO: 203), and a VH CDR3 having an amino acid sequence of DRRYGIVGLPFAY (SEQ ID NO: 204); and (ii) a VL comprising a VL CDR1 having an amino acid sequence of KSSQSVLHSSNNKNYLT (SEQ ID NO: 205), a VL CDR2 having an amino acid sequence of WASTRES (SEQ ID NO: 206), and a VL CDR3 having an amino acid sequence of HQYYSTPPT (SEQ ID NO: 207).

In another aspect, provided herein is an antibody that binds to HLA-G, wherein the antibody comprises: (i) a VH comprising a VH CDR1 having an amino acid sequence of SNRAAWN (SEQ ID NO: 208), a VH CDR2 having an amino acid sequence of RTYYRSKWYNDYAVSVKS (SEQ ID NO: 209), and a VH CDR3 having an amino acid sequence of VRPGIPFDY (SEQ ID NO: 210); and (ii) a VL comprising a VL CDR1 having an amino acid sequence of KSSQSVLFSSNNKNYLA (SEQ ID NO: 211), a VL CDR2 having an amino acid sequence of WASTRES (SEQ ID NO: 212), and a VL CDR3 having an amino acid sequence of QQYHSTPWT (SEQ ID NO: 213).

In another aspect, provided herein is an antibody that binds to HLA-G, wherein the antibody comprises: (i) a VH comprising a VH CDR1 having an amino acid sequence of GDSVSSNRA (SEQ ID NO: 214), a VH CDR2 having an amino acid sequence of YYRSKWY (SEQ ID NO: 215), and a VH CDR3 having an amino acid sequence of VRPGIPFD (SEQ ID NO: 216); and (ii) a VL comprising a VL CDR1 having an amino acid sequence of SQSVLFSSNNKNY (SEQ ID NO: 217), a VL CDR2 having an amino acid sequence of WAS (SEQ ID NO: 218), and a VL CDR3 having an amino acid sequence of YHSTPW (SEQ ID NO: 219).

In another aspect, provided herein is an antibody that binds to HLA-G, wherein the antibody comprises: (i) a VH comprising a VH CDR1 having an amino acid sequence of GDSVSSNRAA (SEQ ID NO: 220), a VH CDR2 having an amino acid sequence of TYYRSKWYN (SEQ ID NO: 221), and a VH CDR3 having an amino acid sequence of ARVRPGIPFDY (SEQ ID NO: 222); and (ii) a VL comprising a VL CDR1 having an amino acid sequence of QSVLFSSNNKNY (SEQ ID NO: 223), a VL CDR2 having an amino acid sequence of WAS (SEQ ID NO: 224), and a VL CDR3 having an amino acid sequence of QQYHSTPWT (SEQ ID NO: 225).

In another aspect, provided herein is an antibody that binds to HLA-G, wherein the antibody comprises: (i) a VH comprising a VH CDR1 having an amino acid sequence of GDSVSSNRAAWN (SEQ ID NO: 226), a VH CDR2 having an amino acid sequence of RTYYRSKWYND (SEQ ID NO: 227), and a VH CDR3 having an amino acid sequence of VRPGIPFDY (SEQ ID NO: 228); and (ii) a VL comprising a VL CDR1 having an amino acid sequence of KSSQSVLFSSNNKNYLA (SEQ ID NO: 229), a VL CDR2 having an amino acid sequence of WASTRES (SEQ ID NO: 230), and a VL CDR3 having an amino acid sequence of QQYHSTPWT (SEQ ID NO: 231).

In some embodiments, the antibody comprises a VH having an amino acid sequence of SEQ ID NO: 174. In some embodiments, the antibody comprises a VL having an amino acid sequence of SEQ ID NO: 172. In some embodiments, the antibody comprises a VH comprising an amino acid sequence having at least 95% identity to the amino acid sequence of SEQ ID NO: 174. In some embodiments, the antibody comprises a VL comprising an amino acid sequence having at least 95% identity to the amino acid sequence of SEQ ID NO: 172.

In some embodiments, the antibody comprises a VH having an amino acid sequence of SEQ ID NO: 175. In some embodiments, the antibody comprises a VL having an amino acid sequence of SEQ ID NO: 173. In some embodiments, the antibody comprises a VH comprising an amino acid sequence having at least 95% identity to the amino acid sequence of SEQ ID NO: 175. In some embodiments, the antibody comprises a VL comprising an amino acid sequence having at least 95% identity to the amino acid sequence of SEQ ID NO: 173.

In yet another aspect, provided herein are antibodies that compete with one of the antibodies thereof described above. Such antibodies may also bind to the same epitope as one of the above mentioned antibodies, or an overlapping epitope. Antibodies that compete with or bind to the same epitope as the above-mentioned antibodies are expected to show similar functional properties.

In certain embodiments, an antibody described herein comprises amino acid sequences with certain percent identity relative to the antibodies described above including the exemplary antibodies described in Section 7 below.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A., 1990, 87:2264-8, modified as in Karlin and Altschul, Proc. Natl. Acad. Sci. U.S.A. 1993, 90:5873-7. Such an algorithm is incorporated into the NBLAST and XBLAST programs of Altschul, et al., J. Mol. Biol., 1990, 215:403. BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res., 1997, 25:3389-402. Alternatively, PSI BLAST can be used to perform an iterated search which detects distant relationships between molecules (Id.). When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In certain embodiments, an antibody provided herein comprises a VH region having at least 80%, at least 85%, at least 90%, at least 95%, or at least 98% sequence identity to the amino acid sequence of SEQ ID NO: 174, and/or a VL region having at least 80%, at least 85%, at least 90%, at least 95%, or at least 98% sequence identity to the amino acid sequence of SEQ ID NO: 172, wherein the antibody binds to HLA-G.

In certain embodiments, an antibody provided herein comprises a VH region having at least 80%, at least 85%, at least 90%, at least 95%, or at least 98% sequence identity to the amino acid sequence of SEQ ID NO: 175, and/or a VL region having at least 80%, at least 85%, at least 90%, at least 95%, or at least 98% sequence identity to the amino acid sequence of SEQ ID NO: 173, wherein the antibody binds to HLA-G.

In certain embodiments, an antibody provided herein comprises a VH region having at least 80%, at least 85%, at least 90%, at least 95%, or at least 98% sequence identity to the amino acid sequence of SEQ ID NO: 174, and/or a VL region having at least 80%, at least 85%, at least 90%, at least 95%, or at least 98% sequence identity to the amino acid sequence of SEQ ID NO: 173, wherein the antibody binds to HLA-G.

In certain embodiments, an antibody provided herein comprises a VH region having at least 80%, at least 85%, at least 90%, at least 95%, or at least 98% sequence identity to the amino acid sequence of SEQ ID NO: 175, and/or a VL region having at least 80%, at least 85%, at least 90%, at least 95%, or at least 98% sequence identity to the amino acid sequence of SEQ ID NO: 172, wherein the antibody binds to HLA-G.

In some embodiments, provided herein is an antibody comprising VH, VL, or CDR sequences of the above described anti-HLA-G antibodies, and further comprising an Fc region that has RE mutations but no fucose residue.

In some embodiments, the anti-HLA-G antibody provided herein comprises a Fc region having RE mutations and no fucose residue.

Standard techniques known to those of skill in the art can be used to introduce mutations in the nucleotide sequence encoding a molecule provided herein, including, for example, site-directed mutagenesis and PCR-mediated mutagenesis which results in amino acid mutations.

In some embodiments, glutamic acid is substituted for lysine at position 248 (K248E) (EU numbering) of the Fc region of the antibody provided herein. In some embodiments, arginine is substituted for threonine at position 437 (T437R) (EU numbering) of the Fc region of the antibody provided herein. In some embodiments, glutamic acid is substituted for lysine at position 248, and arginine is substituted for threonine at position 437 (K248E/T437R) (EU numbering), of the Fc region of the antibody provided herein.

In some embodiments, the antibody provided herein comprises a heavy chain comprising the T437R mutation. In some embodiments, the antibody provided herein comprises a heavy chain comprising the K248E mutation. In some embodiments, the antibody provided herein comprises a heavy chain comprising the K338A mutation. In some embodiments, the antibody provided herein comprises a heavy chain comprising the K248E/T437R mutation. In some embodiments, the antibody provided herein comprises a heavy chain comprising the K338A/T437R mutation. In some embodiments, the antibody provided herein comprises a heavy chain comprising an amino acid sequence of SEQ ID NO: 182 but with T437R mutation. In some embodiments, the antibody provided herein comprises a heavy chain comprising an amino acid sequence of SEQ ID NO: 182 but with K248E mutation. In some embodiments, the antibody provided herein comprises a heavy chain comprising an amino acid sequence of SEQ ID NO: 182 but with K338A mutation. In some embodiments, the antibody provided herein comprises a heavy chain comprising an amino acid sequence of SEQ ID NO: 182 but with K248E/T437R mutation. In some embodiments, the antibody provided herein comprises a heavy chain comprising an amino acid sequence of SEQ ID NO: 182 but with K338A/T437R mutation. In some embodiments, the antibody provided herein comprises a heavy chain comprising an amino acid sequence of SEQ ID NO: 183 but with T437R mutation. In some embodiments, the antibody provided herein comprises a heavy chain comprising an amino acid sequence of SEQ ID NO: 183 but with K248E mutation. In some embodiments, the antibody provided herein comprises a heavy chain comprising an amino acid sequence of SEQ ID NO: 183 but with K338A mutation. In some embodiments, the antibody provided herein comprises a heavy chain comprising an amino acid sequence of SEQ ID NO: 183 but with K248E/T437R mutation. In some embodiments, the antibody provided herein comprises a heavy chain comprising an amino acid sequence of SEQ ID NO: 183 but with K338A/T437R mutation. In some embodiments, the antibody is MHGB752 comprising a heavy chain comprising an amino acid sequence of SEQ ID NO: 182 but with the K248E and T437R mutations (RE mutations). In some embodiments, the antibody is MHGB758 comprising a heavy chain comprising an amino acid sequence of SEQ ID NO: 183 but with the K248E and T437R mutations (RE mutations). In some embodiments, the antibody provided herein (including the above described anti-HLA-G antibodies) is not fucosylated.

Standard techniques known to those of skill in the art can be used to generate an antibody provided herein with a heavy chain comprising no fucose residue on the Asn-297-linked N-oligosaccharide thereof. For example, mammalian cell lines with deficient GMD enzymes (e.g., CHO Lec13 cells), with reduced α-1,6 fucosyltransferase activity resulting from mutated FUT8 genes, with overexpression of β-1,4-mannosyl-glycoprotein 4-β-N-acetylglucosaminyltransferase (GnT-III), with inactivated Golgi GDP-fucose transporter (GFT) gene Slc35c1 (e.g., CHO-gmt3 cells), with heterologous expression of bacterial RMD, or with use of biochemical inhibitors of fucosylation (e.g., fucose analogs such as 2-fluorofucose and 5-alkynylfucose) have been shown to produce antibodies with no fucose residue on the Asn-297-linked N-oligosaccharide on the heavy chain (Pereira, N. A., et al., supra; Shields, R. L. et al., supra; Kanda Y., supra).

In some embodiments, the antibody comprises a heavy chain comprising no fucose residue on the Asn-297-linked N-oligosaccharide thereof. In some embodiments, the antibody is MHGB732.CLF comprising a light chain comprising an amino acid sequence of SEQ ID NO: 180, and a heavy chain comprising an amino acid sequence of SEQ ID NO: 182, which comprises no fucose residue on the Asn-297-linked N-oligosaccharide thereof. In some embodiments, the antibody is MHGB738.CLF comprising a light chain comprising an amino acid sequence of SEQ ID NO:181, and a heavy chain comprising an amino acid sequence of SEQ ID NO: 183, which comprises no fucose residue on the Asn-297-linked N-oligosaccharide thereof. In some embodiments, the antibody comprises a heavy chain comprising an amino acid sequence of SEQ ID NO: 182, which comprises no fucose residue on the Asn-297-linked N-oligosaccharide thereof. In some embodiments, the antibody comprises a heavy chain comprising an amino acid sequence of SEQ ID NO: 183, which comprises no fucose residue on the Asn-297-linked N-oligosaccharide thereof. In some embodiments, the antibody comprises a heavy chain comprising, which comprises the K248E, K338A, T437R, K248E/T437R or K338A/T437R mutation and comprises no fucose residue on the Asn-297-linked N-oligosaccharide thereof. In some embodiments, the antibody comprises a heavy chain comprising an amino acid sequence of SEQ ID NO: 182, which comprises the K248E, K338A, T437R, K248E/T437R or K338A/T437R mutation and comprises no fucose residue on the Asn-297-linked N-oligosaccharide thereof. In some embodiments, the antibody comprises a heavy chain comprising an amino acid sequence of SEQ ID NO: 183, which comprises the K248E, K338A, T437R, K248E/T437R or K338A/T437R mutation and comprises no fucose residue on the Asn-297-linked N-oligosaccharide thereof.

### 5.6 Anti-CD37 Antibodies and Related Molecules

In some embodiments, provided herein are anti-CD37 antibodies. In some embodiments, the antibody provided herein comprises VL, VH or CDRs having amino acid sequences of the VL, VH or CDRs contained in the VL and VH sequences in Table 5 and Table 6 below.

In some embodiments, the antibody is T26B373, T26B459 or T26B608 comprising a VL comprising an amino acid sequence of SEQ ID NO: 1 and a VH comprising an amino acid sequence of SEQ ID NO: 9. In some embodiments, the antibody is T26B374, T26B460 or T26B611 comprising a VL comprising an amino acid sequence of SEQ ID NO: 2 and a VH comprising an amino acid sequence of SEQ ID NO: 10. In some embodiments, the antibody is T26B375, T26B461 or T26B612 comprising a VL comprising an amino acid sequence of SEQ ID NO: 3 and a VH comprising an amino acid sequence of SEQ ID NO: 11. In some embodiments, the antibody is T26B379, T26B462 or T26B615 comprising a VL comprising an amino acid sequence of SEQ ID NO: 4 and a VH comprising an amino acid sequence of SEQ ID NO: 12. In some embodiments, the antibody is T26B382, T26B463 or T26B613 comprising a VL comprising an amino acid sequence of SEQ ID NO: 5 and a VH comprising an amino acid sequence of SEQ ID NO: 13. In some embodiments, the antibody is T26B385, T26B464 or T26B610 comprising a VL comprising an amino acid sequence of SEQ ID NO: 6 and a VH comprising an amino acid sequence of SEQ ID NO: 14. In some embodiments, the antibody is T26B386, T26B465 or T26B614 comprising a VL comprising an amino acid sequence of SEQ ID NO: 7 and a VH comprising an amino acid sequence of SEQ ID NO: 15. In some embodiments, the antibody is T26B388, T26B466 or T26B609 comprising a VL comprising an amino acid sequence of SEQ ID NO: 8 and a VH comprising an amino acid sequence of SEQ ID NO: 16.

In some embodiments, the antibody comprises a VL comprising CDRs having amino acid sequences of the CDRs contained in the VL comprising an amino acid sequence of SEQ ID NO: 1 and a VH comprising CDRs having amino acid sequences of the CDRs contained in the VH comprising an amino acid sequence of SEQ ID NO: 9. In some embodiments, the antibody comprises a VL comprising CDRs having amino acid sequences of the CDRs contained in the VL comprising an amino acid sequence of SEQ ID NO: 1 and a VH comprising CDRs having amino acid sequences of the CDRs contained in the VH comprising an amino acid sequence of SEQ ID NO: 10. In some embodiments, the antibody comprises a VL comprising CDRs having amino acid sequences of the CDRs contained in the VL comprising an amino acid sequence of SEQ ID NO: 1 and a VH comprising CDRs having amino acid sequences of the CDRs contained in the VH comprising an amino acid sequence of SEQ ID NO: 11. In some embodiments, the antibody comprises a VL comprising CDRs having amino acid sequences of the CDRs contained in the VL comprising an amino acid sequence of SEQ ID NO: 1 and a VH comprising CDRs having amino acid sequences of the CDRs contained in the VH comprising an amino acid sequence of SEQ ID NO: 12. In some embodiments, the antibody comprises a VL comprising CDRs having amino acid sequences of the CDRs contained in the VL comprising an amino acid sequence of SEQ ID NO: 1 and a VH comprising CDRs having amino acid sequences of the CDRs contained in the VH comprising an amino acid sequence of SEQ ID NO: 13. In some embodiments, the antibody comprises a VL comprising CDRs having amino acid sequences of the CDRs contained in the VL comprising an amino acid sequence of SEQ ID NO: 1 and a VH comprising CDRs having amino acid sequences of the CDRs contained in the VH comprising an amino acid sequence of SEQ ID NO: 14. In some embodiments, the antibody comprises a VL comprising CDRs having amino acid sequences of the CDRs contained in the VL comprising an amino acid sequence of SEQ ID NO: 1 and a VH comprising CDRs having amino acid sequences of the CDRs contained in the VH comprising an amino acid sequence of SEQ ID NO: 15. In some embodiments, the antibody comprises a VL comprising CDRs having amino acid sequences of the CDRs contained in the VL comprising an amino acid sequence of SEQ ID NO: 1 and a VH comprising CDRs having amino acid sequences of the CDRs contained in the VH comprising an amino acid sequence of SEQ ID NO: 16. In some embodiments, the antibody comprises a VL comprising CDRs having amino acid sequences of the CDRs contained in the VL comprising an amino acid sequence of SEQ ID NO: 2 and a VH comprising CDRs having amino acid sequences of the CDRs contained in the VH comprising an amino acid sequence of SEQ ID NO: 9. In some embodiments, the antibody comprises a VL comprising CDRs having amino acid sequences of the CDRs contained in the VL comprising an amino acid sequence of SEQ ID NO: 2 and a VH comprising CDRs having amino acid sequences of the CDRs contained in the VH comprising an amino acid sequence of SEQ ID NO: 10. In some embodiments, the antibody comprises a VL comprising CDRs having amino acid sequences of the CDRs contained in the VL comprising an amino acid sequence of SEQ ID NO: 2 and a VH comprising CDRs having amino acid sequences of the CDRs contained in the VH comprising an amino acid sequence of SEQ ID NO: 11. In some embodiments, the antibody comprises a VL comprising CDRs having amino acid sequences of the CDRs contained in the VL comprising an amino acid sequence of SEQ ID NO: 2 and a VH comprising CDRs having amino acid sequences of the CDRs contained in the VH comprising an amino acid sequence of SEQ ID NO: 12. In some embodiments, the antibody comprises a VL comprising CDRs having amino acid sequences of the CDRs contained in the VL comprising an amino acid sequence of SEQ ID NO: 2 and a VH comprising CDRs having amino acid sequences of the CDRs contained in the VH comprising an amino acid sequence of SEQ ID NO: 13. In some embodiments, the antibody comprises a VL comprising CDRs having amino acid sequences of the CDRs contained in the VL comprising an amino acid sequence of SEQ ID NO: 2 and a VH comprising CDRs having amino acid sequences of the CDRs contained in the VH comprising an amino acid sequence of SEQ ID NO: 14. In some embodiments, the antibody comprises a VL comprising CDRs having amino acid sequences of the CDRs contained in the VL comprising an amino acid sequence of SEQ ID NO: 2 and a VH comprising CDRs having amino acid sequences of the CDRs contained in the VH comprising an amino acid sequence of SEQ ID NO: 15. In some embodiments, the antibody comprises a VL comprising CDRs having amino acid sequences of the CDRs contained in the VL comprising an amino acid sequence of SEQ ID NO: 2 and a VH comprising CDRs having amino acid sequences of the CDRs contained in the VH comprising an amino acid sequence of SEQ ID NO: 16. In some embodiments, the antibody comprises a VL comprising CDRs having amino acid sequences of the CDRs contained in the VL comprising an amino acid sequence of SEQ ID NO: 3 and a VH comprising CDRs having amino acid sequences of the CDRs contained in the VH comprising an amino acid sequence of SEQ ID NO: 9. In some embodiments, the antibody comprises a VL comprising CDRs having amino acid sequences of the CDRs contained in the VL comprising an amino acid sequence of SEQ ID NO: 3 and a VH comprising CDRs having amino acid sequences of the CDRs contained in the VH comprising an amino acid sequence of SEQ ID NO: 10. In some embodiments, the antibody comprises a VL comprising CDRs having amino acid sequences of the CDRs contained in the VL comprising an amino acid sequence of SEQ ID NO: 3 and a VH comprising CDRs having amino acid sequences of the CDRs contained in the VH comprising an amino acid sequence of SEQ ID NO: 11. In some embodiments, the antibody comprises a VL comprising CDRs having amino acid sequences of the CDRs contained in the VL comprising an amino acid sequence of SEQ ID NO: 3 and a VH comprising CDRs having amino acid sequences of the CDRs contained in the VH comprising an amino acid sequence of SEQ ID NO: 12. In some embodiments, the antibody comprises a VL comprising CDRs having amino acid sequences of the CDRs contained in the VL comprising an amino acid sequence of SEQ ID NO: 3 and a VH comprising CDRs having amino acid sequences of the CDRs contained in the VH comprising an amino acid sequence of SEQ ID NO: 13. In some embodiments, the antibody comprises a VL comprising CDRs having amino acid sequences of the CDRs contained in the VL comprising an amino acid sequence of SEQ ID NO: 3 and a VH comprising CDRs having amino acid sequences of the CDRs contained in the VH comprising an amino acid sequence of SEQ ID NO: 14. In some embodiments, the antibody comprises a VL comprising CDRs having amino acid sequences of the CDRs contained in the VL comprising an amino acid sequence of SEQ ID NO: 3 and a VH comprising CDRs having amino acid sequences of the CDRs contained in the VH comprising an amino acid sequence of SEQ ID NO: 15. In some embodiments, the antibody comprises a VL comprising CDRs having amino acid sequences of the CDRs contained in the VL comprising an amino acid sequence of SEQ ID NO: 3 and a VH comprising CDRs having amino acid sequences of the CDRs contained in the VH comprising an amino acid sequence of SEQ ID NO: 16. In some embodiments, the antibody comprises a VL comprising CDRs having amino acid sequences of the CDRs contained in the VL comprising an amino acid sequence of SEQ ID NO: 4 and a VH comprising CDRs having amino acid sequences of the CDRs contained in the VH comprising an amino acid sequence of SEQ ID NO: 9. In some embodiments, the antibody comprises a VL comprising CDRs having amino acid sequences of the CDRs contained in the VL comprising an amino acid sequence of SEQ ID NO: 4 and a VH comprising CDRs having amino acid sequences of the CDRs contained in the VH comprising an amino acid sequence of SEQ ID NO: 10. In some embodiments, the antibody comprises a VL comprising CDRs having amino acid sequences of the CDRs contained in the VL comprising an amino acid sequence of SEQ ID NO: 4 and a VH comprising CDRs having amino acid sequences of the CDRs contained in the VH comprising an amino acid sequence of SEQ ID NO: 11. In some embodiments, the antibody comprises a VL comprising CDRs having amino acid sequences of the CDRs contained in the VL comprising an amino acid sequence of SEQ ID NO: 4 and a VH comprising CDRs having amino acid sequences of the CDRs contained in the VH comprising an amino acid sequence of SEQ ID NO: 12. In some embodiments, the antibody comprises a VL comprising CDRs having amino acid sequences of the CDRs contained in the VL comprising an amino acid sequence of SEQ ID NO: 4 and a VH comprising CDRs having amino acid sequences of the CDRs contained in the VH comprising an amino acid sequence of SEQ ID NO: 13. In some embodiments, the antibody comprises a VL comprising CDRs having amino acid sequences of the CDRs contained in the VL comprising an amino acid sequence of SEQ ID NO: 4 and a VH comprising CDRs having amino acid sequences of the CDRs contained in the VH comprising an amino acid sequence of SEQ ID NO: 14. In some embodiments, the antibody comprises a VL comprising CDRs having amino acid sequences of the CDRs contained in the VL comprising an amino acid sequence of SEQ ID NO: 4 and a VH comprising CDRs having amino acid sequences of the CDRs contained in the VH comprising an amino acid sequence of SEQ ID NO: 15. In some embodiments, the antibody comprises a VL comprising CDRs having amino acid sequences of the CDRs contained in the VL comprising an amino acid sequence of SEQ ID NO: 4 and a VH comprising CDRs having amino acid sequences of the CDRs contained in the VH comprising an amino acid sequence of SEQ ID NO: 16. In some embodiments, the antibody comprises a VL comprising CDRs having amino acid sequences of the CDRs contained in the VL comprising an amino acid sequence of SEQ ID NO: 5 and a VH comprising CDRs having amino acid sequences of the CDRs contained in the VH comprising an amino acid sequence of SEQ ID NO: 9. In some embodiments, the antibody comprises a VL comprising CDRs having amino acid sequences of the CDRs contained in the VL comprising an amino acid sequence of SEQ ID NO: 5 and a VH comprising CDRs having amino acid sequences of the CDRs contained in the VH comprising an amino acid sequence of SEQ ID NO: 10. In some embodiments, the antibody comprises a VL comprising CDRs having amino acid sequences of the CDRs contained in the VL comprising an amino acid sequence of SEQ ID NO: 5 and a VH comprising CDRs having amino acid sequences of the CDRs contained in the VH comprising an amino acid sequence of SEQ ID NO: 11. In some embodiments, the antibody comprises a VL comprising CDRs having amino acid sequences of the CDRs contained in the VL comprising an amino acid sequence of SEQ ID NO: 5 and a VH comprising CDRs having amino acid sequences of the CDRs contained in the VH comprising an amino acid sequence of SEQ ID NO: 12. In some embodiments, the antibody comprises a VL comprising CDRs having amino acid sequences of the CDRs contained in the VL comprising an amino acid sequence of SEQ ID NO: 5 and a VH comprising CDRs having amino acid sequences of the CDRs contained in the VH comprising an amino acid sequence of SEQ ID NO: 13. In some embodiments, the antibody comprises a VL comprising CDRs having amino acid sequences of the CDRs contained in the VL comprising an amino acid sequence of SEQ ID NO: 5 and a VH comprising CDRs having amino acid sequences of the CDRs contained in the VH comprising an amino acid sequence of SEQ ID NO: 14. In some embodiments, the antibody comprises a VL comprising CDRs having amino acid sequences of the CDRs contained in the VL comprising an amino acid sequence of SEQ ID NO: 5 and a VH comprising CDRs having amino acid sequences of the CDRs contained in the VH comprising an amino acid sequence of SEQ ID NO: 15. In some embodiments, the antibody comprises a VL comprising CDRs having amino acid sequences of the CDRs contained in the VL comprising an amino acid sequence of SEQ ID NO: 5 and a VH comprising CDRs having amino acid sequences of the CDRs contained in the VH comprising an amino acid sequence of SEQ ID NO: 16. In some embodiments, the antibody comprises a VL comprising CDRs having amino acid sequences of the CDRs contained in the VL comprising an amino acid sequence of SEQ ID NO: 6 and a VH comprising CDRs having amino acid sequences of the CDRs contained in the VH comprising an amino acid sequence of SEQ ID NO: 9. In some embodiments, the antibody comprises a VL comprising CDRs having amino acid sequences of the CDRs contained in the VL comprising an amino acid sequence of SEQ ID NO: 6 and a VH comprising CDRs having amino acid sequences of the CDRs contained in the VH comprising an amino acid sequence of SEQ ID NO: 10. In some embodiments, the antibody comprises a VL comprising CDRs having amino acid sequences of the CDRs contained in the VL comprising an amino acid sequence of SEQ ID NO: 6 and a VH comprising CDRs having amino acid sequences of the CDRs contained in the VH comprising an amino acid sequence of SEQ ID NO: 11. In some embodiments, the antibody comprises a VL comprising CDRs having amino acid sequences of the CDRs contained in the VL comprising an amino acid sequence of SEQ ID NO: 6 and a VH comprising CDRs having amino acid sequences of the CDRs contained in the VH comprising an amino acid sequence of SEQ ID NO: 12. In some embodiments, the antibody comprises a VL comprising CDRs having amino acid sequences of the CDRs contained in the VL comprising an amino acid sequence of SEQ ID NO: 6 and a VH comprising CDRs having amino acid sequences of the CDRs contained in the VH comprising an amino acid sequence of SEQ ID NO: 13. In some embodiments, the antibody comprises a VL comprising CDRs having amino acid sequences of the CDRs contained in the VL comprising an amino acid sequence of SEQ ID NO: 6 and a VH comprising CDRs having amino acid sequences of the CDRs contained in the VH comprising an amino acid sequence of SEQ ID NO: 14. In some embodiments, the antibody comprises a VL comprising CDRs having amino acid sequences of the CDRs contained in the VL comprising an amino acid sequence of SEQ ID NO: 6 and a VH comprising CDRs having amino acid sequences of the CDRs contained in the VH comprising an amino acid sequence of SEQ ID NO: 15. In some embodiments, the antibody comprises a VL comprising CDRs having amino acid sequences of the CDRs contained in the VL comprising an amino acid sequence of SEQ ID NO: 6 and a VH comprising CDRs having amino acid sequences of the CDRs contained in the VH comprising an amino acid sequence of SEQ ID NO: 16. In some embodiments, the antibody comprises a VL comprising CDRs having amino acid sequences of the CDRs contained in the VL comprising an amino acid sequence of SEQ ID NO: 7 and a VH comprising CDRs having amino acid sequences of the CDRs contained in the VH comprising an amino acid sequence of SEQ ID NO: 9. In some embodiments, the antibody comprises a VL comprising CDRs having amino acid sequences of the CDRs contained in the VL comprising an amino acid sequence of SEQ ID NO: 7 and a VH comprising CDRs having amino acid sequences of the CDRs contained in the VH comprising an amino acid sequence of SEQ ID NO: 10. In some embodiments, the antibody comprises a VL comprising CDRs having amino acid sequences of the CDRs contained in the VL comprising an amino acid sequence of SEQ ID NO: 7 and a VH comprising CDRs having amino acid sequences of the CDRs contained in the VH comprising an amino acid sequence of SEQ ID NO: 11. In some embodiments, the antibody comprises a VL comprising CDRs having amino acid sequences of the CDRs contained in the VL comprising an amino acid sequence of SEQ ID NO: 7 and a VH comprising CDRs having amino acid sequences of the CDRs contained in the VH comprising an amino acid sequence of SEQ ID NO: 12. In some embodiments, the antibody comprises a VL comprising CDRs having amino acid sequences of the CDRs contained in the VL comprising an amino acid sequence of SEQ ID NO: 7 and a VH comprising CDRs having amino acid sequences of the CDRs contained in the VH comprising an amino acid sequence of SEQ ID NO: 13. In some embodiments, the antibody comprises a VL comprising CDRs having amino acid sequences of the CDRs contained in the VL comprising an amino acid sequence of SEQ ID NO: 7 and a VH comprising CDRs having amino acid sequences of the CDRs contained in the VH comprising an amino acid sequence of SEQ ID NO: 14. In some embodiments, the antibody comprises a VL comprising CDRs having amino acid sequences of the CDRs contained in the VL comprising an amino acid sequence of SEQ ID NO: 7 and a VH comprising CDRs having amino acid sequences of the CDRs contained in the VH comprising an amino acid sequence of SEQ ID NO: 15. In some embodiments, the antibody comprises a VL comprising CDRs having amino acid sequences of the CDRs contained in the VL comprising an amino acid sequence of SEQ ID NO: 7 and a VH comprising CDRs having amino acid sequences of the CDRs contained in the VH comprising an amino acid sequence of SEQ ID NO: 16. In some embodiments, the antibody comprises a VL comprising CDRs having amino acid sequences of the CDRs contained in the VL comprising an amino acid sequence of SEQ ID NO: 8 and a VH comprising CDRs having amino acid sequences of the CDRs contained in the VH comprising an amino acid sequence of SEQ ID NO: 9. In some embodiments, the antibody comprises a VL comprising CDRs having amino acid sequences of the CDRs contained in the VL comprising an amino acid sequence of SEQ ID NO: 8 and a VH comprising CDRs having amino acid sequences of the CDRs contained in the VH comprising an amino acid sequence of SEQ ID NO: 10. In some embodiments, the antibody comprises a VL comprising CDRs having amino acid sequences of the CDRs contained in the VL comprising an amino acid sequence of SEQ ID NO: 8 and a VH comprising CDRs having amino acid sequences of the CDRs contained in the VH comprising an amino acid sequence of SEQ ID NO: 11. In some embodiments, the antibody comprises a VL comprising CDRs having amino acid sequences of the CDRs contained in the VL comprising an amino acid sequence of SEQ ID NO: 8 and a VH comprising CDRs having amino acid sequences of the CDRs contained in the VH comprising an amino acid sequence of SEQ ID NO: 12. In some embodiments, the antibody comprises a VL comprising CDRs having amino acid sequences of the CDRs contained in the VL comprising an amino acid sequence of SEQ ID NO: 8 and a VH comprising CDRs having amino acid sequences of the CDRs contained in the VH comprising an amino acid sequence of SEQ ID NO: 13. In some embodiments, the antibody comprises a VL comprising CDRs having amino acid sequences of the CDRs contained in the VL comprising an amino acid sequence of SEQ ID NO: 8 and a VH comprising CDRs having amino acid sequences of the CDRs contained in the VH comprising an amino acid sequence of SEQ ID NO: 14. In some embodiments, the antibody comprises a VL comprising CDRs having amino acid sequences of the CDRs contained in the VL comprising an amino acid sequence of SEQ ID NO: 8 and a VH comprising CDRs having amino acid sequences of the CDRs contained in the VH comprising an amino acid sequence of SEQ ID NO: 15. In some embodiments, the antibody comprises a VL comprising CDRs having amino acid sequences of the CDRs contained in the VL comprising an amino acid sequence of SEQ ID NO: 8 and a VH comprising CDRs having amino acid sequences of the CDRs contained in the VH comprising an amino acid sequence of SEQ ID NO: 16.

In yet another aspect, provided herein are antibodies that compete with one of the antibodies thereof described above. Such antibodies may also bind to the same epitope as one of the above mentioned antibodies, or an overlapping epitope. Antibodies that compete with or bind to the same epitope as the above-mentioned antibodies are expected to show similar functional properties. The exemplified antigen binding proteins include those with the VL regions and VH regions provided herein, including those in Table 5 and Table 6.

In certain embodiments, an antibody described herein comprises amino acid sequences with certain percent identity relative to the antibodies described above including the exemplary antibodies described in Section 7 below.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A., 1990, 87:2264-8, modified as in Karlin and Altschul, Proc. Natl. Acad. Sci. U.S.A. 1993, 90:5873-7. Such an algorithm is incorporated into the NBLAST and XBLAST programs of Altschul, et al., J. Mol. Biol., 1990, 215:403. BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res., 1997, 25:3389-402. Alternatively, PSI BLAST can be used to perform an iterated search which detects distant relationships between molecules (Id.). When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In certain embodiments, an antibody provided herein comprises a VL region having at least 80%, at least 85%, at least 90%, at least 95%, or at least 98% sequence identity to the amino acid sequence of SEQ ID NO: 1, and/or a VH region having at least 80%, at least 85%, at least 90%, at least 95%, or at least 98% sequence identity to the amino acid sequence of SEQ ID NO: 9, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 1, and/or a VH region comprising CDRs having at least 80%, at least 85%, at least 90%, at least 95%, or at least 98% sequence identity to the amino acid sequence of the CDRs contained in SEQ ID NO: 9, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 1, and/or a VH region having at least 80%, at least 85%, at least 90%, at least 95%, or at least 98% sequence identity to the amino acid sequence of SEQ ID NO: 10, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 1, and/or a VH region comprising CDRs having at least 80%, at least 85%, at least 90%, at least 95%, or at least 98% sequence identity to the amino acid sequence of the CDRs contained in SEQ ID NO: 10, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 1, and/or a VH region having at least 80%, at least 85%, at least 90%, at least 95%, or at least 98% sequence identity to the amino acid sequence of SEQ ID NO: 11, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 1, and/or a VH region comprising CDRs having at least 80%, at least 85%, at least 90%, at least 95%, or at least 98% sequence identity to the amino acid sequence of the CDRs contained in SEQ ID NO: 11, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 1, and/or a VH region having at least 80%, at least 85%, at least 90%, at least 95%, or at least 98% sequence identity to the amino acid sequence of SEQ ID NO: 12, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 1, and/or a VH region comprising CDRs having at least 80%, at least 85%, at least 90%, at least 95%, or at least 98% sequence identity to the amino acid sequence of the CDRs contained in SEQ ID NO: 12, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 1, and/or a VH region having at least 80%, at least 85%, at least 90%, at least 95%, or at least 98% sequence identity to the amino acid sequence of SEQ ID NO: 13, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 1, and/or a VH region comprising CDRs having at least 80%, at least 85%, at least 90%, at least 95%, or at least 98% sequence identity to the amino acid sequence of the CDRs contained in SEQ ID NO: 13, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 1, and/or a VH region having at least 80%, at least 85%, at least 90%, at least 95%, or at least 98% sequence identity to the amino acid sequence of SEQ ID NO: 14, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 1, and/or a VH region comprising CDRs having at least 80%, at least 85%, at least 90%, at least 95%, or at least 98% sequence identity to the amino acid sequence of the CDRs contained in SEQ ID NO: 14, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 1, and/or a VH region having at least 80%, at least 85%, at least 90%, at least 95%, or at least 98% sequence identity to the amino acid sequence of SEQ ID NO: 15, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 1, and/or a VH region comprising CDRs having at least 80%, at least 85%, at least 90%, at least 95%, or at least 98% sequence identity to the amino acid sequence of the CDRs contained in SEQ ID NO: 15, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 1, and/or a VH region having at least 80%, at least 85%, at least 90%, at least 95%, or at least 98% sequence identity to the amino acid sequence of SEQ ID NO: 16, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 1, and/or a VH region comprising CDRs having at least 80%, at least 85%, at least 90%, at least 95%, or at least 98% sequence identity to the amino acid sequence of the CDRs contained in SEQ ID NO: 16, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 2, and/or a VH region having at least 80%, at least 85%, at least 90%, at least 95%, or at least 98% sequence identity to the amino acid sequence of SEQ ID NO: 9, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 2, and/or a VH region comprising CDRs having at least 80%, at least 85%, at least 90%, at least 95%, or at least 98% sequence identity to the amino acid sequence of the CDRs contained in SEQ ID NO: 9, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 2, and/or a VH region having at least 80%, at least 85%, at least 90%, at least 95%, or at least 98% sequence identity to the amino acid sequence of SEQ ID NO: 10, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 2, and/or a VH region comprising CDRs having at least 80%, at least 85%, at least 90%, at least 95%, or at least 98% sequence identity to the amino acid sequence of the CDRs contained in SEQ ID NO: 10, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 2, and/or a VH region having at least 80%, at least 85%, at least 90%, at least 95%, or at least 98% sequence identity to the amino acid sequence of SEQ ID NO: 11, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 2, and/or a VH region comprising CDRs having at least 80%, at least 85%, at least 90%, at least 95%, or at least 98% sequence identity to the amino acid sequence of the CDRs contained in SEQ ID NO: 11, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 2, and/or a VH region having at least 80%, at least 85%, at least 90%, at least 95%, or at least 98% sequence identity to the amino acid sequence of SEQ ID NO: 12, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 2, and/or a VH region comprising CDRs having at least 80%, at least 85%, at least 90%, at least 95%, or at least 98% sequence identity to the amino acid sequence of the CDRs contained in SEQ ID NO: 12, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 2, and/or a VH region having at least 80%, at least 85%, at least 90%, at least 95%, or at least 98% sequence identity to the amino acid sequence of SEQ ID NO: 13, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 2, and/or a VH region comprising CDRs having at least 80%, at least 85%, at least 90%, at least 95%, or at least 98% sequence identity to the amino acid sequence of the CDRs contained in SEQ ID NO: 13, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 2, and/or a VH region having at least 80%, at least 85%, at least 90%, at least 95%, or at least 98% sequence identity to the amino acid sequence of SEQ ID NO: 14, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 2, and/or a VH region comprising CDRs having at least 80%, at least 85%, at least 90%, at least 95%, or at least 98% sequence identity to the amino acid sequence of the CDRs contained in SEQ ID NO: 14, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 2, and/or a VH region having at least 80%, at least 85%, at least 90%, at least 95%, or at least 98% sequence identity to the amino acid sequence of SEQ ID NO: 15, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 2, and/or a VH region comprising CDRs having at least 80%, at least 85%, at least 90%, at least 95%, or at least 98% sequence identity to the amino acid sequence of the CDRs contained in SEQ ID NO: 15, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 2, and/or a VH region having at least 80%, at least 85%, at least 90%, at least 95%, or at least 98% sequence identity to the amino acid sequence of SEQ ID NO: 16, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 2, and/or a VH region comprising CDRs having at least 80%, at least 85%, at least 90%, at least 95%, or at least 98% sequence identity to the amino acid sequence of the CDRs contained in SEQ ID NO: 16, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 3, and/or a VH region having at least 80%, at least 85%, at least 90%, at least 95%, or at least 98% sequence identity to the amino acid sequence of SEQ ID NO: 9, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 3, and/or a VH region comprising CDRs having at least 80%, at least 85%, at least 90%, at least 95%, or at least 98% sequence identity to the amino acid sequence of the CDRs contained in SEQ ID NO: 9, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 3, and/or a VH region having at least 80%, at least 85%, at least 90%, at least 95%, or at least 98% sequence identity to the amino acid sequence of SEQ ID NO: 10, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 3, and/or a VH region comprising CDRs having at least 80%, at least 85%, at least 90%, at least 95%, or at least 98% sequence identity to the amino acid sequence of the CDRs contained in SEQ ID NO: 10, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 3, and/or a VH region having at least 80%, at least 85%, at least 90%, at least 95%, or at least 98% sequence identity to the amino acid sequence of SEQ ID NO: 11, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 3, and/or a VH region comprising CDRs having at least 80%, at least 85%, at least 90%, at least 95%, or at least 98% sequence identity to the amino acid sequence of the CDRs contained in SEQ ID NO: 11, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 3, and/or a VH region having at least 80%, at least 85%, at least 90%, at least 95%, or at least 98% sequence identity to the amino acid sequence of SEQ ID NO: 12, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 3, and/or a VH region comprising CDRs having at least 80%, at least 85%, at least 90%, at least 95%, or at least 98% sequence identity to the amino acid sequence of the CDRs contained in SEQ ID NO: 12, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 3, and/or a VH region having at least 80%, at least 85%, at least 90%, at least 95%, or at least 98% sequence identity to the amino acid sequence of SEQ ID NO: 13, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 3, and/or a VH region comprising CDRs having at least 80%, at least 85%, at least 90%, at least 95%, or at least 98% sequence identity to the amino acid sequence of the CDRs contained in SEQ ID NO: 13, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 3, and/or a VH region having at least 80%, at least 85%, at least 90%, at least 95%, or at least 98% sequence identity to the amino acid sequence of SEQ ID NO: 14, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 3, and/or a VH region comprising CDRs having at least 80%, at least 85%, at least 90%, at least 95%, or at least 98% sequence identity to the amino acid sequence of the CDRs contained in SEQ ID NO: 14, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 3, and/or a VH region having at least 80%, at least 85%, at least 90%, at least 95%, or at least 98% sequence identity to the amino acid sequence of SEQ ID NO: 15, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 3, and/or a VH region comprising CDRs having at least 80%, at least 85%, at least 90%, at least 95%, or at least 98% sequence identity to the amino acid sequence of the CDRs contained in SEQ ID NO: 15, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 3, and/or a VH region having at least 80%, at least 85%, at least 90%, at least 95%, or at least 98% sequence identity to the amino acid sequence of SEQ ID NO: 16, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 3, and/or a VH region comprising CDRs having at least 80%, at least 85%, at least 90%, at least 95%, or at least 98% sequence identity to the amino acid sequence of the CDRs contained in SEQ ID NO: 16, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 4, and/or a VH region having at least 80%, at least 85%, at least 90%, at least 95%, or at least 98% sequence identity to the amino acid sequence of SEQ ID NO: 9, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 4, and/or a VH region comprising CDRs having at least 80%, at least 85%, at least 90%, at least 95%, or at least 98% sequence identity to the amino acid sequence of the CDRs contained in SEQ ID NO: 9, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 4, and/or a VH region having at least 80%, at least 85%, at least 90%, at least 95%, or at least 98% sequence identity to the amino acid sequence of SEQ ID NO: 10, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 4, and/or a VH region comprising CDRs having at least 80%, at least 85%, at least 90%, at least 95%, or at least 98% sequence identity to the amino acid sequence of the CDRs contained in SEQ ID NO: 10, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 4, and/or a VH region having at least 80%, at least 85%, at least 90%, at least 95%, or at least 98% sequence identity to the amino acid sequence of SEQ ID NO: 11, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 4, and/or a VH region comprising CDRs having at least 80%, at least 85%, at least 90%, at least 95%, or at least 98% sequence identity to the amino acid sequence of the CDRs contained in SEQ ID NO: 11, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 4, and/or a VH region having at least 80%, at least 85%, at least 90%, at least 95%, or at least 98% sequence identity to the amino acid sequence of SEQ ID NO: 12, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 4, and/or a VH region comprising CDRs having at least 80%, at least 85%, at least 90%, at least 95%, or at least 98% sequence identity to the amino acid sequence of the CDRs contained in SEQ ID NO: 12, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 4, and/or a VH region having at least 80%, at least 85%, at least 90%, at least 95%, or at least 98% sequence identity to the amino acid sequence of SEQ ID NO: 13, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 4, and/or a VH region comprising CDRs having at least 80%, at least 85%, at least 90%, at least 95%, or at least 98% sequence identity to the amino acid sequence of the CDRs contained in SEQ ID NO: 13, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 4, and/or a VH region having at least 80%, at least 85%, at least 90%, at least 95%, or at least 98% sequence identity to the amino acid sequence of SEQ ID NO: 14, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 4, and/or a VH region comprising CDRs having at least 80%, at least 85%, at least 90%, at least 95%, or at least 98% sequence identity to the amino acid sequence of the CDRs contained in SEQ ID NO: 14, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 4, and/or a VH region having at least 80%, at least 85%, at least 90%, at least 95%, or at least 98% sequence identity to the amino acid sequence of SEQ ID NO: 15, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 4, and/or a VH region comprising CDRs having at least 80%, at least 85%, at least 90%, at least 95%, or at least 98% sequence identity to the amino acid sequence of the CDRs contained in SEQ ID NO: 15, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 4, and/or a VH region having at least 80%, at least 85%, at least 90%, at least 95%, or at least 98% sequence identity to the amino acid sequence of SEQ ID NO: 16, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 4, and/or a VH region comprising CDRs having at least 80%, at least 85%, at least 90%, at least 95%, or at least 98% sequence identity to the amino acid sequence of the CDRs contained in SEQ ID NO: 16, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 5, and/or a VH region having at least 80%, at least 85%, at least 90%, at least 95%, or at least 98% sequence identity to the amino acid sequence of SEQ ID NO: 9, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 5, and/or a VH region comprising CDRs having at least 80%, at least 85%, at least 90%, at least 95%, or at least 98% sequence identity to the amino acid sequence of the CDRs contained in SEQ ID NO: 9, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 5, and/or a VH region having at least 80%, at least 85%, at least 90%, at least 95%, or at least 98% sequence identity to the amino acid sequence of SEQ ID NO: 10, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 5, and/or a VH region comprising CDRs having at least 80%, at least 85%, at least 90%, at least 95%, or at least 98% sequence identity to the amino acid sequence of the CDRs contained in SEQ ID NO: 10, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 5, and/or a VH region having at least 80%, at least 85%, at least 90%, at least 95%, or at least 98% sequence identity to the amino acid sequence of SEQ ID NO: 11, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 5, and/or a VH region comprising CDRs having at least 80%, at least 85%, at least 90%, at least 95%, or at least 98% sequence identity to the amino acid sequence of the CDRs contained in SEQ ID NO: 11, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 5, and/or a VH region having at least 80%, at least 85%, at least 90%, at least 95%, or at least 98% sequence identity to the amino acid sequence of SEQ ID NO: 12, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 5, and/or a VH region comprising CDRs having at least 80%, at least 85%, at least 90%, at least 95%, or at least 98% sequence identity to the amino acid sequence of the CDRs contained in SEQ ID NO: 12, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 5, and/or a VH region having at least 80%, at least 85%, at least 90%, at least 95%, or at least 98% sequence identity to the amino acid sequence of SEQ ID NO: 13, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 5, and/or a VH region comprising CDRs having at least 80%, at least 85%, at least 90%, at least 95%, or at least 98% sequence identity to the amino acid sequence of the CDRs contained in SEQ ID NO: 13, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 5, and/or a VH region having at least 80%, at least 85%, at least 90%, at least 95%, or at least 98% sequence identity to the amino acid sequence of SEQ ID NO: 14, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 5, and/or a VH region comprising CDRs having at least 80%, at least 85%, at least 90%, at least 95%, or at least 98% sequence identity to the amino acid sequence of the CDRs contained in SEQ ID NO: 14, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 5, and/or a VH region having at least 80%, at least 85%, at least 90%, at least 95%, or at least 98% sequence identity to the amino acid sequence of SEQ ID NO: 15, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 5, and/or a VH region comprising CDRs having at least 80%, at least 85%, at least 90%, at least 95%, or at least 98% sequence identity to the amino acid sequence of the CDRs contained in SEQ ID NO: 15, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 5, and/or a VH region having at least 80%, at least 85%, at least 90%, at least 95%, or at least 98% sequence identity to the amino acid sequence of SEQ ID NO: 16, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 5, and/or a VH region comprising CDRs having at least 80%, at least 85%, at least 90%, at least 95%, or at least 98% sequence identity to the amino acid sequence of the CDRs contained in SEQ ID NO: 16, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 6, and/or a VH region having at least 80%, at least 85%, at least 90%, at least 95%, or at least 98% sequence identity to the amino acid sequence of SEQ ID NO: 9, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 6, and/or a VH region comprising CDRs having at least 80%, at least 85%, at least 90%, at least 95%, or at least 98% sequence identity to the amino acid sequence of the CDRs contained in SEQ ID NO: 9, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 6, and/or a VH region having at least 80%, at least 85%, at least 90%, at least 95%, or at least 98% sequence identity to the amino acid sequence of SEQ ID NO: 10, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 6, and/or a VH region comprising CDRs having at least 80%, at least 85%, at least 90%, at least 95%, or at least 98% sequence identity to the amino acid sequence of the CDRs contained in SEQ ID NO: 10, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 6, and/or a VH region having at least 80%, at least 85%, at least 90%, at least 95%, or at least 98% sequence identity to the amino acid sequence of SEQ ID NO: 11, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 6, and/or a VH region comprising CDRs having at least 80%, at least 85%, at least 90%, at least 95%, or at least 98% sequence identity to the amino acid sequence of the CDRs contained in SEQ ID NO: 11, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 6, and/or a VH region having at least 80%, at least 85%, at least 90%, at least 95%, or at least 98% sequence identity to the amino acid sequence of SEQ ID NO: 12, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 6, and/or a VH region comprising CDRs having at least 80%, at least 85%, at least 90%, at least 95%, or at least 98% sequence identity to the amino acid sequence of the CDRs contained in SEQ ID NO: 12, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 6, and/or a VH region having at least 80%, at least 85%, at least 90%, at least 95%, or at least 98% sequence identity to the amino acid sequence of SEQ ID NO: 13, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 6, and/or a VH region comprising CDRs having at least 80%, at least 85%, at least 90%, at least 95%, or at least 98% sequence identity to the amino acid sequence of the CDRs contained in SEQ ID NO: 13, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 6, and/or a VH region having at least 80%, at least 85%, at least 90%, at least 95%, or at least 98% sequence identity to the amino acid sequence of SEQ ID NO: 14, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 6, and/or a VH region comprising CDRs having at least 80%, at least 85%, at least 90%, at least 95%, or at least 98% sequence identity to the amino acid sequence of the CDRs contained in SEQ ID NO: 14, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 6, and/or a VH region having at least 80%, at least 85%, at least 90%, at least 95%, or at least 98% sequence identity to the amino acid sequence of SEQ ID NO: 15, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 6, and/or a VH region comprising CDRs having at least 80%, at least 85%, at least 90%, at least 95%, or at least 98% sequence identity to the amino acid sequence of the CDRs contained in SEQ ID NO: 15, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 6, and/or a VH region having at least 80%, at least 85%, at least 90%, at least 95%, or at least 98% sequence identity to the amino acid sequence of SEQ ID NO: 16, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 6, and/or a VH region comprising CDRs having at least 80%, at least 85%, at least 90%, at least 95%, or at least 98% sequence identity to the amino acid sequence of the CDRs contained in SEQ ID NO: 16, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 7, and/or a VH region having at least 80%, at least 85%, at least 90%, at least 95%, or at least 98% sequence identity to the amino acid sequence of SEQ ID NO: 9, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 7, and/or a VH region comprising CDRs having at least 80%, at least 85%, at least 90%, at least 95%, or at least 98% sequence identity to the amino acid sequence of the CDRs contained in SEQ ID NO: 9, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 7, and/or a VH region having at least 80%, at least 85%, at least 90%, at least 95%, or at least 98% sequence identity to the amino acid sequence of SEQ ID NO: 10, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 7, and/or a VH region comprising CDRs having at least 80%, at least 85%, at least 90%, at least 95%, or at least 98% sequence identity to the amino acid sequence of the CDRs contained in SEQ ID NO: 10, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 7, and/or a VH region having at least 80%, at least 85%, at least 90%, at least 95%, or at least 98% sequence identity to the amino acid sequence of SEQ ID NO: 11, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 7, and/or a VH region comprising CDRs having at least 80%, at least 85%, at least 90%, at least 95%, or at least 98% sequence identity to the amino acid sequence of the CDRs contained in SEQ ID NO: 11, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 7, and/or a VH region having at least 80%, at least 85%, at least 90%, at least 95%, or at least 98% sequence identity to the amino acid sequence of SEQ ID NO: 12, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 7, and/or a VH region comprising CDRs having at least 80%, at least 85%, at least 90%, at least 95%, or at least 98% sequence identity to the amino acid sequence of the CDRs contained in SEQ ID NO: 12, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 7, and/or a VH region having at least 80%, at least 85%, at least 90%, at least 95%, or at least 98% sequence identity to the amino acid sequence of SEQ ID NO: 13, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 7, and/or a VH region comprising CDRs having at least 80%, at least 85%, at least 90%, at least 95%, or at least 98% sequence identity to the amino acid sequence of the CDRs contained in SEQ ID NO: 13, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 7, and/or a VH region having at least 80%, at least 85%, at least 90%, at least 95%, or at least 98% sequence identity to the amino acid sequence of SEQ ID NO: 14, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 7, and/or a VH region comprising CDRs having at least 80%, at least 85%, at least 90%, at least 95%, or at least 98% sequence identity to the amino acid sequence of the CDRs contained in SEQ ID NO: 14, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 7, and/or a VH region having at least 80%, at least 85%, at least 90%, at least 95%, or at least 98% sequence identity to the amino acid sequence of SEQ ID NO: 15, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 7, and/or a VH region comprising CDRs having at least 80%, at least 85%, at least 90%, at least 95%, or at least 98% sequence identity to the amino acid sequence of the CDRs contained in SEQ ID NO: 15, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 7, and/or a VH region having at least 80%, at least 85%, at least 90%, at least 95%, or at least 98% sequence identity to the amino acid sequence of SEQ ID NO: 16, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 7, and/or a VH region comprising CDRs having at least 80%, at least 85%, at least 90%, at least 95%, or at least 98% sequence identity to the amino acid sequence of the CDRs contained in SEQ ID NO: 16, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 8, and/or a VH region having at least 80%, at least 85%, at least 90%, at least 95%, or at least 98% sequence identity to the amino acid sequence of SEQ ID NO: 9, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 8, and/or a VH region comprising CDRs having at least 80%, at least 85%, at least 90%, at least 95%, or at least 98% sequence identity to the amino acid sequence of the CDRs contained in SEQ ID NO: 9, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 8, and/or a VH region having at least 80%, at least 85%, at least 90%, at least 95%, or at least 98% sequence identity to the amino acid sequence of SEQ ID NO: 10, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 8, and/or a VH region comprising CDRs having at least 80%, at least 85%, at least 90%, at least 95%, or at least 98% sequence identity to the amino acid sequence of the CDRs contained in SEQ ID NO: 10, wherein the antibody binds to CD37.

In certain embodiments, an antibody provided herein comprises a VL region having at least 80%, at least 85%, at least 90%, at least 95%, or at least 98% sequence identity to the amino acid sequence of SEQ ID NO: 8, and/or a VH region having at least 80%, at least 85%, at least 90%, at least 95%, or at least 98% sequence identity to the amino acid sequence of SEQ ID NO: 11, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 8, and/or a VH region comprising CDRs having at least 80%, at least 85%, at least 90%, at least 95%, or at least 98% sequence identity to the amino acid sequence of the CDRs contained in SEQ ID NO: 11, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 8, and/or a VH region having at least 80%, at least 85%, at least 90%, at least 95%, or at least 98% sequence identity to the amino acid sequence of SEQ ID NO: 12, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 8, and/or a VH region comprising CDRs having at least 80%, at least 85%, at least 90%, at least 95%, or at least 98% sequence identity to the amino acid sequence of the CDRs contained in SEQ ID NO: 12, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 8, and/or a VH region having at least 80%, at least 85%, at least 90%, at least 95%, or at least 98% sequence identity to the amino acid sequence of SEQ ID NO: 13, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 8, and/or a VH region comprising CDRs having at least 80%, at least 85%, at least 90%, at least 95%, or at least 98% sequence identity to the amino acid sequence of the CDRs contained in SEQ ID NO: 13, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 8, and/or a VH region having at least 80%, at least 85%, at least 90%, at least 95%, or at least 98% sequence identity to the amino acid sequence of SEQ ID NO: 14, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 8, and/or a VH region comprising CDRs having at least 80%, at least 85%, at least 90%, at least 95%, or at least 98% sequence identity to the amino acid sequence of the CDRs contained in SEQ ID NO: 14, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 8, and/or a VH region having at least 80%, at least 85%, at least 90%, at least 95%, or at least 98% sequence identity to the amino acid sequence of SEQ ID NO: 15, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 8, and/or a VH region comprising CDRs having at least 80%, at least 85%, at least 90%, at least 95%, or at least 98% sequence identity to the amino acid sequence of the CDRs contained in SEQ ID NO: 15, wherein the antibody binds to CD37. In certain embodiments, an antibody provided herein comprises a VL region having at least 80%, at least 85%, at least 90%, at least 95%, or at least 98% sequence identity to the amino acid sequence of SEQ ID NO: 8, and/or a VH region having at least 80%, at least 85%, at least 90%, at least 95%, or at least 98% sequence identity to the amino acid sequence of SEQ ID NO: 16, wherein the antibody binds to CD37. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 8, and/or a VH region comprising CDRs having at least 80%, at least 85%, at least 90%, at least 95%, or at least 98% sequence identity to the amino acid sequence of the CDRs contained in SEQ ID NO: 16, wherein the antibody binds to CD37.

In some embodiments, provided herein is an antibody comprising VH, VL, or CDR sequences of the above described anti-CD37 antibodies, and further comprising an Fc region that has RE mutations but no fucose residue. In some embodiments, the antibody is T26B459 comprising a light chain comprising an amino acid sequence of SEQ ID NO: 37 and a heavy chain comprising an amino acid sequence of SEQ ID NO: 53. In some embodiments, the antibody is T26B460 comprising a light chain comprising an amino acid sequence of SEQ ID NO: 38 and a heavy chain comprising an amino acid sequence of SEQ ID NO: 54. In some embodiments, the antibody is T26B461 comprising a light chain comprising an amino acid sequence of SEQ ID NO: 39 and a heavy chain comprising an amino acid sequence of SEQ ID NO: 55. In some embodiments, the antibody is T26B462 comprising a light chain comprising an amino acid sequence of SEQ ID NO: 40 and a heavy chain comprising an amino acid sequence of SEQ ID NO: 56. In some embodiments, the antibody is T26B463 comprising a light chain comprising an amino acid sequence of SEQ ID NO: 41 and a heavy chain comprising an amino acid sequence of SEQ ID NO: 57. In some embodiments, the antibody is T26B464 comprising a light chain comprising an amino acid sequence of SEQ ID NO: 42 and a heavy chain comprising an amino acid sequence of SEQ ID NO: 58. In some embodiments, the antibody is T26B465 comprising a light chain comprising an amino acid sequence of SEQ ID NO: 43 and a heavy chain comprising an amino acid sequence of SEQ ID NO: 59. In some embodiments, the antibody is T26B466 comprising a light chain comprising an amino acid sequence of SEQ ID NO: 44 and a heavy chain comprising an amino acid sequence of SEQ ID NO: 60. In some embodiments, the antibody comprises a heavy chain comprising an amino acid sequence of SEQ ID NO: 45 but with K248E/T437R mutation. In some embodiments, the antibody comprises a heavy chain comprising an amino acid sequence of SEQ ID NO: 46 but with K248E/T437R mutation. In some embodiments, the antibody comprises a heavy chain comprising an amino acid sequence of SEQ ID NO: 47 but with K248E/T437R mutation. In some embodiments, the antibody comprises a heavy chain comprising an amino acid sequence of SEQ ID NO: 48 but with K248E/T437R mutation. In some embodiments, the antibody comprises a heavy chain comprising an amino acid sequence of SEQ ID NO: 49 but with K248E/T437R mutation. In some embodiments, the antibody comprises a heavy chain comprising an amino acid sequence of SEQ ID NO: 50 but with K248E/T437R mutation. In some embodiments, the antibody comprises a heavy chain comprising an amino acid sequence of SEQ ID NO: 51 but with K248E/T437R mutation. In some embodiments, the antibody comprises a heavy chain comprising an amino acid sequence of SEQ ID NO: 52 but with K248E/T437R mutation. In some embodiments, the antibody comprises a heavy chain comprising an amino acid sequence of SEQ ID NO: 53. In some embodiments, the antibody comprises a heavy chain comprising an amino acid sequence of SEQ ID NO: 54. In some embodiments, the antibody comprises a heavy chain comprising an amino acid sequence of SEQ ID NO: 55. In some embodiments, the antibody comprises a heavy chain comprising an amino acid sequence of SEQ ID NO: 56. In some embodiments, the antibody comprises a heavy chain comprising an amino acid sequence of SEQ ID NO: 57. In some embodiments, the antibody comprises a heavy chain comprising an amino acid sequence of SEQ ID NO: 58. In some embodiments, the antibody comprises a heavy chain comprising an amino acid sequence of SEQ ID NO: 59. In some embodiments, the antibody comprises a heavy chain comprising an amino acid sequence of SEQ ID NO: 60.

In some embodiments, the antibody comprises a heavy chain comprising an amino acid sequence of SEQ ID NO: 61 but with K248E/T437R mutation. In some embodiments, the antibody comprises a heavy chain comprising an amino acid sequence of SEQ ID NO: 62 but with K248E/T437R mutation. In some embodiments, the antibody comprises a heavy chain comprising an amino acid sequence of SEQ ID NO: 63 but with K248E/T437R mutation. In some embodiments, the antibody comprises a heavy chain comprising an amino acid sequence of SEQ ID NO: 64 but with K248E/T437R mutation. In some embodiments, the antibody comprises a heavy chain comprising an amino acid sequence of SEQ ID NO: 65 but with K248E/T437R mutation. In some embodiments, the antibody comprises a heavy chain comprising an amino acid sequence of SEQ ID NO: 66 but with K248E/T437R mutation. In some embodiments, the antibody comprises a heavy chain comprising an amino acid sequence of SEQ ID NO: 67 but with K248E/T437R mutation. In some embodiments, the antibody comprises a heavy chain comprising an amino acid sequence of SEQ ID NO: 68 but with K248E/T437R mutation.

In some embodiments, the antibody is T26B373.CLF comprising a light chain comprising an amino acid sequence of SEQ ID NO: 37, and a heavy chain comprising an amino acid sequence of SEQ ID NO: 45, which comprises no fucose residue on the Asn-297-linked N-oligosaccharide thereof. In some embodiments, the antibody is T26B374.CLF comprising a light chain comprising an amino acid sequence of SEQ ID NO: 38, and a heavy chain comprising an amino acid sequence of SEQ ID NO: 46, which comprises no fucose residue on the Asn-297-linked N-oligosaccharide thereof. In some embodiments, the antibody is T26B375.CLF comprising a light chain comprising an amino acid sequence of SEQ ID NO: 39, and a heavy chain comprising an amino acid sequence of SEQ ID NO: 47, which comprises no fucose residue on the Asn-297-linked N-oligosaccharide thereof. In some embodiments, the antibody is T26B379.CLF comprising a light chain comprising an amino acid sequence of SEQ ID NO: 40, and a heavy chain comprising an amino acid sequence of SEQ ID NO: 48, which comprises no fucose residue on the Asn-297-linked N-oligosaccharide thereof. In some embodiments, the antibody is T26B382.CLF comprising a light chain comprising an amino acid sequence of SEQ ID NO: 41, and a heavy chain comprising an amino acid sequence of SEQ ID NO: 49, which comprises no fucose residue on the Asn-297-linked N-oligosaccharide thereof. In some embodiments, the antibody is T26B385.CLF comprising a light chain comprising an amino acid sequence of SEQ ID NO: 42, and a heavy chain comprising an amino acid sequence of SEQ ID NO: 50, which comprises no fucose residue on the Asn-297-linked N-oligosaccharide thereof. In some embodiments, the antibody is T26B386.CLF comprising a light chain comprising an amino acid sequence of SEQ ID NO: 43, and a heavy chain comprising an amino acid sequence of SEQ ID NO: 51, which comprises no fucose residue on the Asn-297-linked N-oligosaccharide thereof. In some embodiments, the antibody is T26B388.CLF comprising a light chain comprising an amino acid sequence of SEQ ID NO: 44, and a heavy chain comprising an amino acid sequence of SEQ ID NO: 52, which comprises no fucose residue on the Asn-297-linked N-oligosaccharide thereof. In some embodiments, the antibody is T26B459.CLF comprising a light chain comprising an amino acid sequence of SEQ ID NO: 37, and a heavy chain comprising an amino acid sequence of SEQ ID NO: 53, which comprises no fucose residue on the Asn-297-linked N-oligosaccharide thereof. In some embodiments, the antibody is T26B460.CLF comprising a light chain comprising an amino acid sequence of SEQ ID NO: 38, and a heavy chain comprising an amino acid sequence of SEQ ID NO: 54, which comprises no fucose residue on the Asn-297-linked N-oligosaccharide thereof. In some embodiments, the antibody is T26B461.CLF comprising a light chain comprising an amino acid sequence of SEQ ID NO: 39, and a heavy chain comprising an amino acid sequence of SEQ ID NO: 55, which comprises no fucose residue on the Asn-297-linked N-oligosaccharide thereof. In some embodiments, the antibody is T26B462.CLF comprising a light chain comprising an amino acid sequence of SEQ ID NO: 40, and a heavy chain comprising an amino acid sequence of SEQ ID NO: 56, which comprises no fucose residue on the Asn-297-linked N-oligosaccharide thereof. In some embodiments, the antibody is T26B463.CLF comprising a light chain comprising an amino acid sequence of SEQ ID NO: 41, and a heavy chain comprising an amino acid sequence of SEQ ID NO: 57, which comprises no fucose residue on the Asn-297-linked N-oligosaccharide thereof. In some embodiments, the antibody is T26B464.CLF comprising a light chain comprising an amino acid sequence of SEQ ID NO: 42, and a heavy chain comprising an amino acid sequence of SEQ ID NO: 58, which comprises no fucose residue on the Asn-297-linked N-oligosaccharide thereof. In some embodiments, the antibody is T26B465.CLF comprising a light chain comprising an amino acid sequence of SEQ ID NO: 43, and a heavy chain comprising an amino acid sequence of SEQ ID NO: 59, which comprises no fucose residue on the Asn-297-linked N-oligosaccharide thereof. In some embodiments, the antibody is T26B466.CLF comprising a light chain comprising an amino acid sequence of SEQ ID NO: 44, and a heavy chain comprising an amino acid sequence of SEQ ID NO: 60, which comprises no fucose residue on the Asn-297-linked N-oligosaccharide thereof. In some embodiments, the antibody comprises a heavy chain comprising an amino acid sequence of SEQ ID NO: 45, which comprises no fucose residue on the Asn-297-linked N-oligosaccharide thereof. In some embodiments, the antibody comprises a heavy chain comprising an amino acid sequence of SEQ ID NO: 46, which comprises no fucose residue on the Asn-297-linked N-oligosaccharide thereof. In some embodiments, the antibody comprises a heavy chain comprising an amino acid sequence of SEQ ID NO: 47, which comprises no fucose residue on the Asn-297-linked N-oligosaccharide thereof. In some embodiments, the antibody comprises a heavy chain comprising an amino acid sequence of SEQ ID NO: 48, which comprises no fucose residue on the Asn-297-linked N-oligosaccharide thereof. In some embodiments, the antibody comprises a heavy chain comprising an amino acid sequence of SEQ ID NO: 49, which comprises no fucose residue on the Asn-297-linked N-oligosaccharide thereof. In some embodiments, the antibody comprises a heavy chain comprising an amino acid sequence of SEQ ID NO: 50, which comprises no fucose residue on the Asn-297-linked N-oligosaccharide thereof. In some embodiments, the antibody comprises a heavy chain comprising an amino acid sequence of SEQ ID NO: 51, which comprises no fucose residue on the Asn-297-linked N-oligosaccharide thereof. In some embodiments, the antibody comprises a heavy chain comprising an amino acid sequence of SEQ ID NO: 52, which comprises no fucose residue on the Asn-297-linked N-oligosaccharide thereof. In some embodiments, the antibody comprises a heavy chain comprising an amino acid sequence of SEQ ID NO: 53, which comprises no fucose residue on the Asn-297-linked N-oligosaccharide thereof. In some embodiments, the antibody comprises a heavy chain comprising an amino acid sequence of SEQ ID NO: 54, which comprises no fucose residue on the Asn-297-linked N-oligosaccharide thereof. In some embodiments, the antibody comprises a heavy chain comprising an amino acid sequence of SEQ ID NO: 55, which comprises no fucose residue on the Asn-297-linked N-oligosaccharide thereof. In some embodiments, the antibody comprises a heavy chain comprising an amino acid sequence of SEQ ID NO: 56, which comprises no fucose residue on the Asn-297-linked N-oligosaccharide thereof. In some embodiments, the antibody comprises a heavy chain comprising an amino acid sequence of SEQ ID NO: 57, which comprises no fucose residue on the Asn-297-linked N-oligosaccharide thereof. In some embodiments, the antibody comprises a heavy chain comprising an amino acid sequence of SEQ ID NO: 58, which comprises no fucose residue on the Asn-297-linked N-oligosaccharide thereof. In some embodiments, the antibody comprises a heavy chain comprising an amino acid sequence of SEQ ID NO: 59, which comprises no fucose residue on the Asn-297-linked N-oligosaccharide thereof. In some embodiments, the antibody comprises a heavy chain comprising an amino acid sequence of SEQ ID NO: 60, which comprises no fucose residue on the Asn-297-linked N-oligosaccharide thereof. In some embodiments, the antibody comprises a heavy chain comprising an amino acid sequence of SEQ ID NO: 61, which comprises no fucose residue on the Asn-297-linked N-oligosaccharide thereof. In some embodiments, the antibody comprises a heavy chain comprising an amino acid sequence of SEQ ID NO: 62, which comprises no fucose residue on the Asn-297-linked N-oligosaccharide thereof. In some embodiments, the antibody comprises a heavy chain comprising an amino acid sequence of SEQ ID NO: 63, which comprises no fucose residue on the Asn-297-linked N-oligosaccharide thereof. In some embodiments, the antibody comprises a heavy chain comprising an amino acid sequence of SEQ ID NO: 64, which comprises no fucose residue on the Asn-297-linked N-oligosaccharide thereof. In some embodiments, the antibody comprises a heavy chain comprising an amino acid sequence of SEQ ID NO: 65, which comprises no fucose residue on the Asn-297-linked N-oligosaccharide thereof. In some embodiments, the antibody comprises a heavy chain comprising an amino acid sequence of SEQ ID NO: 66, which comprises no fucose residue on the Asn-297-linked N-oligosaccharide thereof. In some embodiments, the antibody comprises a heavy chain comprising an amino acid sequence of SEQ ID NO: 67, which comprises no fucose residue on the Asn-297-linked N-oligosaccharide thereof. In some embodiments, the antibody comprises a heavy chain comprising an amino acid sequence of SEQ ID NO: 68, which comprises no fucose residue on the Asn-297-linked N-oligosaccharide thereof. In some embodiments, the antibody comprises a heavy chain comprising an amino acid sequence of SEQ ID NO: 45, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 46, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 47, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 48, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 49, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 50, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 51, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 52, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 53, which comprises no fucose residue on the Asn-297-linked N-oligosaccharide thereof. In some embodiments, the antibody comprises a heavy chain comprising an amino acid sequence of SEQ ID NO: 54, which comprises no fucose residue on the Asn-297-linked N-oligosaccharide thereof. In some embodiments, the antibody comprises a heavy chain comprising an amino acid sequence of SEQ ID NO: 55, which comprises no fucose residue on the Asn-297-linked N-oligosaccharide thereof. In some embodiments, the antibody comprises a heavy chain comprising an amino acid sequence of SEQ ID NO: 56, which comprises no fucose residue on the Asn-297-linked N-oligosaccharide thereof. In some embodiments, the antibody comprises a heavy chain comprising an amino acid sequence of SEQ ID NO: 57, which comprises no fucose residue on the Asn-297-linked N-oligosaccharide thereof. In some embodiments, the antibody comprises a heavy chain comprising an amino acid sequence of SEQ ID NO: 58, which comprises no fucose residue on the Asn-297-linked N-oligosaccharide thereof. In some embodiments, the antibody comprises a heavy chain comprising an amino acid sequence of SEQ ID NO: 59, which comprises no fucose residue on the Asn-297-linked N-oligosaccharide thereof. In some embodiments, the antibody comprises a heavy chain comprising an amino acid sequence of SEQ ID NO: 60, which comprises no fucose residue on the Asn-297-linked N-oligosaccharide thereof. In some embodiments, the antibody comprises a heavy chain comprising an amino acid sequence of SEQ ID NO: 61, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 62, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 63, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 64, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 65, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 66, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 67, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 68, which comprises the K248E, T437R or K248E/T437R mutation and comprises no fucose residue on the Asn-297-linked N-oligosaccharide thereof.

### 5.7 Anti-GPRC5D Antibodies and Related Molecules

In other embodiments, provided herein is an anti-GPRC5D antibody. In some embodiments, the antibody provided herein comprises VL, VH or CDRs having amino acid sequences of the VL, VH or CDRs contained in the VL and VH sequences in Table 13.

In some embodiments, the antibody is GC5B747 or GC5B752 comprising a VL comprising an amino acid sequence of SEQ ID NO: 33 and a VH comprising an amino acid sequence of SEQ ID NO: 34. In some embodiments, the antibody comprises a VL comprising CDRs having amino acid sequences of the CDRs contained in the VL comprising an amino acid sequence of SEQ ID NO: 33 and a VH comprising CDRs having amino acid sequences of the CDRs contained in the VH comprising an amino acid sequence of SEQ ID NO: 34.

In yet another aspect, provided herein are antibodies that compete with one of the antibodies thereof described above. Such antibodies may also bind to the same epitope as one of the above mentioned antibodies, or an overlapping epitope. Antibodies that compete with or bind to the same epitope as the above-mentioned antibodies are expected to show similar functional properties. The exemplified antigen binding proteins include those with the VL regions and VH regions provided herein, including those in Table 13.

In certain embodiments, an antibody described herein comprises amino acid sequences with certain percent identity relative to the antibodies described above including the exemplary antibodies described in Section 7 below.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A., 1990, 87:2264-8, modified as in Karlin and Altschul, Proc. Natl. Acad. Sci. U.S.A., 1993, 90:5873-7. Such an algorithm is incorporated into the NBLAST and XBLAST programs of Altschul, et al., J. Mol. Biol., 1990, 215:403. BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul, et al., Nucleic Acids Res., 1997, 25:3389-402. Alternatively, PSI BLAST can be used to perform an iterated search which detects distant relationships between molecules (Id.). When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In certain embodiments, an antibody provided herein comprises a VL region having at least 80%, at least 85%, at least 90%, at least 95%, or at least 98% sequence identity to the amino acid sequence of SEQ ID NO: 33, and/or a VH region having at least 80%, at least 85%, at least 90%, at least 95%, or at least 98% sequence identity to the amino acid sequence of SEQ ID NO: 34, wherein the antibody binds to GPRC5D. In certain embodiments, an antibody provided herein comprises a VL region comprising CDRs having at least 80%, at least 85%, at least 90%, at least 95%, or at least 98% sequence identity to the amino acid sequence of the CDRs contained in SEQ ID NO: 33, and/or a VH region comprising CDRs having at least 80%, at least 85%, at least 90%, at least 95%, or at least 98% sequence identity to the amino acid sequence of the CDRs contained in SEQ ID NO: 34, wherein the antibody binds to GPRC5D.

In some embodiments, glutamic acid is substituted for lysine at position 248 (K248E) (EU numbering) of the Fc region of the antibody provided herein. In some embodiments, arginine is substituted for threonine at position 437 (T437R) (EU numbering) of the Fc region of the antibody provided herein. In some embodiments, glutamic acid is substituted for lysine at position 248, and arginine is substituted for threonine at position 437 (K248E/T437R) (EU numbering), of the Fc region of the antibody provided herein.

In some embodiments, the antibody is GC5B747 comprising a light chain comprising an amino acid sequence of SEQ ID NO: 101 and a heavy chain comprising an amino acid sequence of SEQ ID NO: 102. In some embodiments, the antibody is GC5B752 comprising a light chain comprising an amino acid sequence of SEQ ID NO: 101 and a heavy chain comprising an amino acid sequence of SEQ ID NO: 103. In some embodiments, the antibody comprises a heavy chain comprising an amino acid sequence of SEQ ID NO: 102, which comprises the K248E, T437R or K248E/T437R mutation. In some embodiments, the antibody comprises a heavy chain comprising an amino acid sequence of SEQ ID NO: 103.

In some embodiments, the antibody provided herein comprises no fucose in the Fc region. In some embodiments, the antibody is GC5B747.CLF comprising a light chain comprising an amino acid sequence of SEQ ID NO: 101, and a heavy chain comprising an amino acid sequence of SEQ ID NO: 102, which comprises no fucose residue on the Asn-297-linked N-oligosaccharide thereof. In some embodiments, the antibody is GC5B752.CLF comprising a light chain comprising an amino acid sequence of SEQ ID NO: 101, and a heavy chain comprising an amino acid sequence of SEQ ID NO: 103, which comprises no fucose residue on the Asn-297-linked N-oligosaccharide thereof. In some embodiments, the antibody comprises a heavy chain comprising an amino acid sequence of SEQ ID NO: 102, which comprises no fucose residue on the Asn-297-linked N-oligosaccharide thereof. In some embodiments, the antibody comprises a heavy chain comprising an amino acid sequence of SEQ ID NO: 103, which comprises no fucose residue on the Asn-297-linked N-oligosaccharide thereof.

In some embodiments, the antibody provided herein comprises the K248E/T437R mutation and no fucosylation in the Fc region. In some embodiments, the antibody is GC5B752.CLF comprising a light chain comprising an amino acid sequence of SEQ ID NO: 101, and a heavy chain comprising an amino acid sequence of SEQ ID NO: 103, which comprises no fucose residue on the Asn-297-linked N-oligosaccharide thereof. In some embodiments, the antibody comprises a heavy chain comprising an amino acid sequence of SEQ ID NO: 102, which comprises the K248E, T437R or K248E/T437R mutation and comprises no fucose residue on the Asn-297-linked N-oligosaccharide thereof. In some embodiments, the antibody comprises a heavy chain comprising an amino acid sequence of SEQ ID NO: 103, which comprises no fucose residue on the Asn-297-linked N-oligosaccharide thereof.

### 5.8 Anti-KLK2 Antibodies and Related Molecules

In one aspect, provided herein are anti-KLK2 antibodies.

In some embodiments, the antibody is KL2B870 comprising a VL comprising an amino acid sequence of SEQ ID NO: 108 and a VH comprising an amino acid sequence of SEQ ID NO: 107. In some embodiments, provided herein is an antibody that binds to KLK2, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO: 107; and (ii) a VL comprising a VL CDR1, a VL CDR2, and a VL CDR3 having amino acid sequences of the VL CDR1, VL CDR2, and VL CDR3, respectively, of SEQ ID NO: 108. In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system.

In another aspect, provided herein is an antibody that binds to KLK2, wherein the antibody comprises: (i) a VH comprising a VH CDR1 having an amino acid sequence of GGSISSYYWS (SEQ ID NO: 109), a VH CDR2 having an amino acid sequence of YIYYSGSTNYNPSLKS (SEQ ID NO: 110), and a VH CDR3 having an amino acid sequence of TTIFGVVTPNFYYGMDV (SEQ ID NO: 111); and (ii) a VL comprising a VL CDR1 having an amino acid sequence of RASQGISSYLA (SEQ ID NO: 112), a VL CDR2 having an amino acid sequence of AASTLQS (SEQ ID NO: 113), and a VL CDR3 having an amino acid sequence of QQLNSYPLT (SEQ ID NO: 114). In another aspect, provided herein is an antibody that binds to KLK2, wherein the antibody comprises: (i) a VH comprising a VH CDR1 having an amino acid sequence of GGSISSYY (SEQ ID NO: 115), a VH CDR2 having an amino acid sequence of IYYSGST (SEQ ID NO: 116), and a VH CDR3 having an amino acid sequence of AGTTIFGVVTPNFYYGMDV (SEQ ID NO: 117); and (ii) a VL comprising a VL CDR1 having an amino acid sequence of QGISSY (SEQ ID NO: 118), a VL CDR2 having an amino acid sequence of AAS (SEQ ID NO: 119), and a VL CDR3 having an amino acid sequence of QQLNSYPLT (SEQ ID NO: 120). In another aspect, provided herein is an antibody that binds to KLK2, wherein the antibody comprises: (i) a VH comprising a VH CDR1 having an amino acid sequence of SYYWS (SEQ ID NO: 121), a VH CDR2 having an amino acid sequence of YIYYSGSTNYNPSLKS (SEQ ID NO: 122), and a VH CDR3 having an amino acid sequence of TTIFGVVTPNFYYGMDV (SEQ ID NO: 123); and (ii) a VL comprising a VL CDR1 having an amino acid sequence of RASQGISSYLA (SEQ ID NO: 124), a VL CDR2 having an amino acid sequence of AASTLQS (SEQ ID NO: 125), and a VL CDR3 having an amino acid sequence of QQLNSYPLT (SEQ ID NO: 126). In another aspect, provided herein is an antibody that binds to KLK2, wherein the antibody comprises: (i) a VH comprising a VH CDR1 having an amino acid sequence of GGSISSY (SEQ ID NO: 127), a VH CDR2 having an amino acid sequence of YSG (SEQ ID NO: 128), and a VH CDR3 having an amino acid sequence of TIFGVVTPNFYYGMD (SEQ ID NO: 129); and (ii) a VL comprising a VL CDR1 having an amino acid sequence of SQGISSY (SEQ ID NO: 130), a VL CDR2 having an amino acid sequence of AAS (SEQ ID NO: 131), and a VL CDR3 having an amino acid sequence of LNSYPL (SEQ ID NO: 132). In another aspect, provided herein is an antibody that binds to KLK2, wherein the antibody comprises: (i) a VH comprising a VH CDR1 having an amino acid sequence of GGSISSY (SEQ ID NO: 127), a VH CDR2 having an amino acid sequence of YYSGS (SEQ ID NO: 168), and a VH CDR3 having an amino acid sequence of TIFGVVTPNFYYGMD (SEQ ID NO: 129); and (ii) a VL comprising a VL CDR1 having an amino acid sequence of RASQGISSY (SEQ ID NO: 169), a VL CDR2 having an amino acid sequence of AASTLQS (SEQ ID NO: 170), and a VL CDR3 having an amino acid sequence of QQLNSYPLT (SEQ ID NO: 171). In another aspect, provided herein is an antibody that binds to KLK2, wherein the antibody comprises: (i) a VH comprising a VH CDR1 having an amino acid sequence of SSYYWS (SEQ ID NO: 133), a VH CDR2 having an amino acid sequence of WIGYIYYSGSTN (SEQ ID NO: 134), and a VH CDR3 having an amino acid sequence of AGTTIFGVVTPNFYYGMD (SEQ ID NO: 135); and (ii) a VL comprising a VL CDR1 having an amino acid sequence of SSYLAWY (SEQ ID NO: 136), a VL CDR2 having an amino acid sequence of FLIYAASTLQ (SEQ ID NO: 137), and a VL CDR3 having an amino acid sequence of QQLNSYPL (SEQ ID NO: 138). In another aspect, provided herein is an antibody that binds to KLK2, wherein the antibody comprises: (i) a VH comprising a VH CDR1 having an amino acid sequence of GGSISSYYWS (SEQ ID NO: 139), a VH CDR2 having an amino acid sequence of YIYYSGSTN (SEQ ID NO: 140), and a VH CDR3 having an amino acid sequence of TTIFGVVTPNFYYGMDV (SEQ ID NO: 141); and (ii) a VL comprising a VL CDR1 having an amino acid sequence of RASQGISSYLA (SEQ ID NO: 142), a VL CDR2 having an amino acid sequence of AASTLQS (SEQ ID NO: 143), and a VL CDR3 having an amino acid sequence of QQLNSYPLT (SEQ ID NO: 144). In some embodiments, the antibody comprises a VH having an amino acid sequence of SEQ ID NO: 107. In some embodiments, the antibody comprises a VL having an amino acid sequence of SEQ ID NO: 108. In some embodiments, the antibody comprises a VH comprising an amino acid sequence having at least 95% identity to the amino acid sequence of SEQ ID NO: 107. In some embodiments, the antibody comprises a VL comprising an amino acid sequence having at least 95% identity to the amino acid sequence of SEQ ID NO: 108.

In yet another aspect, provided herein are antibodies that compete with one of the antibodies thereof described above. Such antibodies may also bind to the same epitope as one of the above mentioned antibodies, or an overlapping epitope. Antibodies that compete with or bind to the same epitope as the above-mentioned antibodies are expected to show similar functional properties.

In certain embodiments, an antibody described herein comprises amino acid sequences with certain percent identity relative to the antibodies described above including the exemplary antibodies described in Section 7 below.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A., 1990, 87:2264-8, modified as in Karlin and Altschul, Proc. Natl. Acad. Sci. U.S.A. 1993, 90:5873-7. Such an algorithm is incorporated into the NBLAST and XBLAST programs of Altschul, et al., J. Mol. Biol., 1990, 215:403. BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res., 1997, 25:3389-402. Alternatively, PSI BLAST can be used to perform an iterated search which detects distant relationships between molecules (Id.). When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In certain embodiments, an antibody provided herein comprises a VH region having at least 80%, at least 85%, at least 90%, at least 95%, or at least 98% sequence identity to the amino acid sequence of SEQ ID NO: 107, and/or a VL region having at least 80%, at least 85%, at least 90%, at least 95%, or at least 98% sequence identity to the amino acid sequence of SEQ ID NO: 108, wherein the antibody binds to KLK2.

In some embodiments, provided herein is an antibody comprising VH, VL, or CDR sequences of the above described anti-KLK2 antibodies, and further comprising an Fc region that has RE mutations but no fucose residue. In some embodiments, the anti-KLK2 antibody provided herein comprises a Fc region having RE mutations and no fucose residue.

Standard techniques known to those of skill in the art can be used to introduce mutations in the nucleotide sequence encoding a molecule provided herein, including, for example, site-directed mutagenesis and PCR-mediated mutagenesis which results in amino acid mutations.

In some embodiments, glutamic acid is substituted for lysine at position 248 (K248E) (EU numbering) of the Fc region of the antibody provided herein. In some embodiments, arginine is substituted for threonine at position 437 (T437R) (EU numbering) of the Fc region of the antibody provided herein. In some embodiments, glutamic acid is substituted for lysine at position 248, and arginine is substituted for threonine at position 437 (K248E/T437R) (EU numbering), of the Fc region of the antibody provided herein.

In some embodiments, the antibody provided herein comprises a heavy chain comprising the T437R mutation. In some embodiments, the antibody provided herein comprises a heavy chain comprising the K248E mutation. In some embodiments, the antibody provided herein comprises a heavy chain comprising the K338A mutation. In some embodiments, the antibody provided herein comprises a heavy chain comprising the K248E/T437R mutation. In some embodiments, the antibody provided herein comprises a heavy chain comprising the K338A/T437R mutation. In some embodiments, the antibody provided herein comprises a heavy chain comprising an amino acid sequence of SEQ ID NO: 155 but with T437R mutation. In some embodiments, the antibody provided herein comprises a heavy chain comprising an amino acid sequence of SEQ ID NO: 155 but with K248E mutation. In some embodiments, the antibody provided herein comprises a heavy chain comprising an amino acid sequence of SEQ ID NO: 155 but with K338A mutation. In some embodiments, the antibody provided herein comprises a heavy chain comprising an amino acid sequence of SEQ ID NO: 155 but with K248E/T437R mutation. In some embodiments, the antibody provided herein comprises a heavy chain comprising an amino acid sequence of SEQ ID NO: 155 but with K338A/T437R mutation. In some embodiments, the antibody is KL2B870 comprising a heavy chain comprising an amino acid sequence of SEQ ID NO: 155 but with the K248E and T437R mutations (RE mutations).

In some embodiments, the antibody provided herein (including the above described anti-KLK2 antibodies) is not fucosylated. Standard techniques known to those of skill in the art can be used to generate an antibody provided herein with a heavy chain comprising no fucose residue on the Asn-297-linked N-oligosaccharide thereof. For example, mammalian cell lines with deficient GMD enzymes (e.g., CHO Lec13 cells), with reduced α-1,6 fucosyltransferase activity resulting from mutated FUT8 genes, with overexpression of β-1,4-mannosyl-glycoprotein 4-β-N-acetylglucosaminyltransferase (GnT-III), with inactivated Golgi GDP-fucose transporter (GFT) gene Slc35c1 (e.g., CHO-gmt3 cells), with heterologous expression of bacterial RMD, or with use of biochemical inhibitors of fucosylation (e.g., fucose analogs such as 2-fluorofucose and 5-alkynylfucose) have been shown to produce antibodies with no fucose residue on the Asn-297-linked N-oligosaccharide on the heavy chain (Pereira, N. A., et al., supra; Shields, R. L. et al., supra; Kanda Y., supra).

In some embodiments, the antibody comprises a heavy chain comprising no fucose residue on the Asn-297-linked N-oligosaccharide thereof. In some embodiments, the antibody is KL2B872.CLF comprising a light chain comprising an amino acid sequence of SEQ ID NO: 146, and a heavy chain comprising an amino acid sequence of SEQ ID NO: 155, which comprises no fucose residue on the Asn-297-linked N-oligosaccharide thereof. In some embodiments, the antibody is KL2B870.CLF comprising a light chain comprising an amino acid sequence of SEQ ID NO: 146, and a heavy chain comprising an amino acid sequence of SEQ ID NO: 153, which comprises no fucose residue on the Asn-297-linked N-oligosaccharide thereof. In some embodiments, the antibody comprises a heavy chain comprising an amino acid sequence of SEQ ID NO: 155, which comprises no fucose residue on the Asn-297-linked N-oligosaccharide thereof. In some embodiments, the antibody comprises a heavy chain comprising an amino acid sequence of SEQ ID NO: 155, which comprises the K248E, K338A, T437R, K248E/T437R or K338A/T437R mutation and comprises no fucose residue on the Asn-297-linked N-oligosaccharide thereof.

### 5.9 Anti-PSMA Antibodies and Related Molecules

In one aspect, provided herein are anti-PSMA antibodies.

In some embodiments, the antibody is PSMB896 comprising a VL comprising an amino acid sequence of SEQ ID NO: 232 and a VH comprising an amino acid sequence of SEQ ID NO: 233. In some embodiments, the antibody is PSMB898 comprising a VL comprising an amino acid sequence of SEQ ID NO: 232 and a VH comprising an amino acid sequence of SEQ ID NO: 234.

In some embodiments, provided herein is an antibody that binds to PSMA, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO: 233; and (ii) a VL comprising a VL CDR1, a VL CDR2, and a VL CDR3 having amino acid sequences of the VL CDR1, VL CDR2, and VL CDR3, respectively, of SEQ ID NO: 232. In some embodiments, provided herein is an antibody that binds to PSMA, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO: 234; and (ii) a VL comprising a VL CDR1, a VL CDR2, and a VL CDR3 having amino acid sequences of the VL CDR1, VL CDR2, and VL CDR3, respectively, of SEQ ID NO: 232. In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system.

In another aspect, provided herein is an antibody that binds to PSMA, wherein the antibody comprises: (i) a VH comprising a VH CDR1 having an amino acid sequence of SYAMS (SEQ ID NO: 244), a VH CDR2 having an amino acid sequence of AISGGIGSTYYADSVKG (SEQ ID NO: 245), and a VH CDR3 having an amino acid sequence of DGVGATPYYFDY (SEQ ID NO: 246); and (ii) a VL comprising a VL CDR1 having an amino acid sequence of SGSSSNIGINYVST (SEQ ID NO: 247), a VL CDR2 having an amino acid sequence of DNNKRPS (SEQ ID NO: 248), and a VL CDR3 having an amino acid sequence of GTWDSSLSAVV (SEQ ID NO: 249). In another aspect, provided herein is an antibody that binds to PSMA, wherein the antibody comprises: (i) a VH comprising a VH CDR1 having an amino acid sequence of SYAMS (SEQ ID NO: 250), a VH CDR2 having an amino acid sequence of AISGGSGSTYYADSVKG (SEQ ID NO: 251), and a VH CDR3 having an amino acid sequence of DGVGATPYYFDY (SEQ ID NO: 252); and (ii) a VL comprising a VL CDR1 having an amino acid sequence of SGSSSNIGINYVS (SEQ ID NO: 253), a VL CDR2 having an amino acid sequence of DNNKRPS (SEQ ID NO: 254), and a VL CDR3 having an amino acid sequence of GTWDSSLSAVV (SEQ ID NO: 255). In another aspect, provided herein is an antibody that binds to PSMA, wherein the antibody comprises: (i) a VH comprising a VH CDR1 having an amino acid sequence of GFTFSSYAMS (SEQ ID NO: 256), a VH CDR2 having an amino acid sequence of AISGGIGSTY (SEQ ID NO: 257), and a VH CDR3 having an amino acid sequence of DGVGATPYYFDY (SEQ ID NO: 258); and (ii) a VL comprising a VL CDR1 having an amino acid sequence of SGSSSNIGINYVS (SEQ ID NO: 259), a VL CDR2 having an amino acid sequence of DNNKRPS (SEQ ID NO: 260), and a VL CDR3 having an amino acid sequence of GTWDSSLSAVV (SEQ ID NO: 261). In another aspect, provided herein is an antibody that binds to PSMA, wherein the antibody comprises: (i) a VH comprising a VH CDR1 having an amino acid sequence of GFTFSSYAMS (SEQ ID NO: 262), a VH CDR2 having an amino acid sequence of AISGGSGSTY (SEQ ID NO: 263), and a VH CDR3 having an amino acid sequence of DGVGATPYYFDY (SEQ ID NO: 264); and (ii) a VL comprising a VL CDR1 having an amino acid sequence of SGSSSNIGINYVS (SEQ ID NO: 265), a VL CDR2 having an amino acid sequence of DNNKRPS (SEQ ID NO: 266), and a VL CDR3 having an amino acid sequence of GTWDSSLSAVV (SEQ ID NO: 267). In another aspect, provided herein is an antibody that binds to PSMA, wherein the antibody comprises: (i) a VH comprising a VH CDR1 having an amino acid sequence of GFTFSSY (SEQ ID NO: 268), a VH CDR2 having an amino acid sequence of SGGIGS (SEQ ID NO: 269), and a VH CDR3 having an amino acid sequence of GVGATPYYFD (SEQ ID NO: 270); and (ii) a VL comprising a VL CDR1 having an amino acid sequence of SSSNIGINY (SEQ ID NO: 271), a VL CDR2 having an amino acid sequence of DNN (SEQ ID NO: 272), and a VL CDR3 having an amino acid sequence of WDSSLSAV (SEQ ID NO: 273). In another aspect, provided herein is an antibody that binds to PSMA, wherein the antibody comprises: (i) a VH comprising a VH CDR1 having an amino acid sequence of GFTFSSY (SEQ ID NO: 274), a VH CDR2 having an amino acid sequence of SGGSGS (SEQ ID NO: 275), and a VH CDR3 having an amino acid sequence of GVGATPYYFD (SEQ ID NO: 276); and (ii) a VL comprising a VL CDR1 having an amino acid sequence of SSSSNIGINY (SEQ ID NO: 277), a VL CDR2 having an amino acid sequence of DNN (SEQ ID NO: 278), and a VL CDR3 having an amino acid sequence of WDSSLSAV (SEQ ID NO: 279). In another aspect, provided herein is an antibody that binds to PSMA, wherein the antibody comprises: (i) a VH comprising a VH CDR1 having an amino acid sequence of SYAMS (SEQ ID NO: 280), a VH CDR2 having an amino acid sequence of AISGGIGSTYYADSVKG (SEQ ID NO: 281), and a VH CDR3 having an amino acid sequence of DGVGATPYYFDY (SEQ ID NO: 282); and (ii) a VL comprising a VL CDR1 having an amino acid sequence of SGSSSNIGINYVS (SEQ ID NO: 283), a VL CDR2 having an amino acid sequence of DNNKRPS (SEQ ID NO: 284), and a VL CDR3 having an amino acid sequence of GTWDSSLSAVV (SEQ ID NO: 285). In another aspect, provided herein is an antibody that binds to PSMA, wherein the antibody comprises: (i) a VH comprising a VH CDR1 having an amino acid sequence of SYAMS (SEQ ID NO: 286), a VH CDR2 having an amino acid sequence of AISGGSGSTYYADSVKG (SEQ ID NO: 287), and a VH CDR3 having an amino acid sequence of DGVGATPYYFDY (SEQ ID NO: 288); and (ii) a VL comprising a VL CDR1 having an amino acid sequence of SGSSSNIGINYVS (SEQ ID NO: 289), a VL CDR2 having an amino acid sequence of DNNKRPS (SEQ ID NO: 290), and a VL CDR3 having an amino acid sequence of GTWDSSLSAVV (SEQ ID NO: 291).

In some embodiments, the antibody comprises a VH having an amino acid sequence of SEQ ID NO: 233. In some embodiments, the antibody comprises a VL having an amino acid sequence of SEQ ID NO: 232. In some embodiments, the antibody comprises a VH comprising an amino acid sequence having at least 95% identity to the amino acid sequence of SEQ ID NO: 233. In some embodiments, the antibody comprises a VL comprising an amino acid sequence having at least 95% identity to the amino acid sequence of SEQ ID NO: 232.

In some embodiments, the antibody comprises a VH having an amino acid sequence of SEQ ID NO: 234. In some embodiments, the antibody comprises a VL having an amino acid sequence of SEQ ID NO: 232. In some embodiments, the antibody comprises a VH comprising an amino acid sequence having at least 95% identity to the amino acid sequence of SEQ ID NO: 234. In some embodiments, the antibody comprises a VL comprising an amino acid sequence having at least 95% identity to the amino acid sequence of SEQ ID NO: 232.

In yet another aspect, provided herein are antibodies that compete with one of the antibodies thereof described above. Such antibodies may also bind to the same epitope as one of the above mentioned antibodies, or an overlapping epitope. Antibodies that compete with or bind to the same epitope as the above-mentioned antibodies are expected to show similar functional properties.

In certain embodiments, an antibody described herein comprises amino acid sequences with certain percent identity relative to the antibodies described above including the exemplary antibodies described in Section 7 below.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A., 1990, 87:2264-8, modified as in Karlin and Altschul, Proc. Natl. Acad. Sci. U.S.A. 1993, 90:5873-7. Such an algorithm is incorporated into the NBLAST and XBLAST programs of Altschul, et al., J. Mol. Biol., 1990, 215:403. BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res., 1997, 25:3389-402. Alternatively, PSI BLAST can be used to perform an iterated search which detects distant relationships between molecules (Id.). When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In certain embodiments, an antibody provided herein comprises a VH region having at least 80%, at least 85%, at least 90%, at least 95%, or at least 98% sequence identity to the amino acid sequence of SEQ ID NO: 233, and/or a VL region having at least 80%, at least 85%, at least 90%, at least 95%, or at least 98% sequence identity to the amino acid sequence of SEQ ID NO: 232, wherein the antibody binds to PSMA. In certain embodiments, an antibody provided herein comprises a VH region having at least 80%, at least 85%, at least 90%, at least 95%, or at least 98% sequence identity to the amino acid sequence of SEQ ID NO: 234, and/or a VL region having at least 80%, at least 85%, at least 90%, at least 95%, or at least 98% sequence identity to the amino acid sequence of SEQ ID NO: 232, wherein the antibody binds to PSMA.

In some embodiments, provided herein is an antibody comprising VH, VL, or CDR sequences of the above described anti-PSMA antibodies, and further comprising an Fc region that has RE mutations but no fucose residue. In some embodiments, the anti-PSMA antibody provided herein comprises a Fc region having RE mutations and no fucose residue.

Standard techniques known to those of skill in the art can be used to introduce mutations in the nucleotide sequence encoding a molecule provided herein, including, for example, site-directed mutagenesis and PCR-mediated mutagenesis which results in amino acid mutations.

In some embodiments, glutamic acid is substituted for lysine at position 248 (K248E) (EU numbering) of the Fc region of the antibody provided herein. In some embodiments, arginine is substituted for threonine at position 437 (T437R) (EU numbering) of the Fc region of the antibody provided herein. In some embodiments, glutamic acid is substituted for lysine at position 248, and arginine is substituted for threonine at position 437 (K248E/T437R) (EU numbering), of the Fc region of the antibody provided herein.

In some embodiments, the antibody provided herein comprises a heavy chain comprising the T437R mutation. In some embodiments, the antibody provided herein comprises a heavy chain comprising the K248E mutation. In some embodiments, the antibody provided herein comprises a heavy chain comprising the K338A mutation. In some embodiments, the antibody provided herein comprises a heavy chain comprising the K248E/T437R mutation. In some embodiments, the antibody provided herein comprises a heavy chain comprising the K338A/T437R mutation. In some embodiments, the antibody provided herein comprises a heavy chain comprising an amino acid sequence of SEQ ID NO: 242 but with T437R mutation. In some embodiments, the antibody provided herein comprises a heavy chain comprising an amino acid sequence of SEQ ID NO: 242 but with K248E mutation. In some embodiments, the antibody provided herein comprises a heavy chain comprising an amino acid sequence of SEQ ID NO: 242 but with K338A mutation. In some embodiments, the antibody provided herein comprises a heavy chain comprising an amino acid sequence of SEQ ID NO: 242 but with K248E/T437R mutation. In some embodiments, the antibody provided herein comprises a heavy chain comprising an amino acid sequence of SEQ ID NO: 242 but with K338A/T437R mutation. In some embodiments, the antibody provided herein comprises a heavy chain comprising an amino acid sequence of SEQ ID NO: 243 but with T437R mutation. In some embodiments, the antibody provided herein comprises a heavy chain comprising an amino acid sequence of SEQ ID NO: 243 but with K248E mutation. In some embodiments, the antibody provided herein comprises a heavy chain comprising an amino acid sequence of SEQ ID NO: 243 but with K338A mutation. In some embodiments, the antibody provided herein comprises a heavy chain comprising an amino acid sequence of SEQ ID NO: 243 but with K248E/T437R mutation. In some embodiments, the antibody provided herein comprises a heavy chain comprising an amino acid sequence of SEQ ID NO: 243 but with K338A/T437R mutation. In some embodiments, the antibody is PSMB896 comprising a heavy chain comprising an amino acid sequence of SEQ ID NO: 242 but with the K248E and T437R mutations (RE mutations). In some embodiments, the antibody is PSMB898 comprising a heavy chain comprising an amino acid sequence of SEQ ID NO: 243 but with the K248E and T437R mutations (RE mutations).

In some embodiments, the antibody provided herein (including the above described anti-PSMA antibodies) is not fucosylated. Standard techniques known to those of skill in the art can be used to generate an antibody provided herein with a heavy chain comprising no fucose residue on the Asn-297-linked N-oligosaccharide thereof. For example, mammalian cell lines with deficient GMD enzymes (e.g., CHO Lec13 cells), with reduced α-1,6 fucosyltransferase activity resulting from mutated FUT8 genes, with overexpression of β-1,4-mannosyl-glycoprotein 4-β-N-acetylglucosaminyltransferase (GnT-III), with inactivated Golgi GDP-fucose transporter (GFT) gene Slc35c1 (e.g., CHO-gmt3 cells), with heterologous expression of bacterial RMD, or with use of biochemical inhibitors of fucosylation (e.g., fucose analogs such as 2-fluorofucose and 5-alkynylfucose) have been shown to produce antibodies with no fucose residue on the Asn-297-linked N-oligosaccharide on the heavy chain (Pereira, N. A., et al., supra; Shields, R. L. et al., supra; Kanda Y., supra).

In some embodiments, the antibody comprises a heavy chain comprising no fucose residue on the Asn-297-linked N-oligosaccharide thereof. In some embodiments, the antibody is PSMB896.CLF comprising a light chain comprising an amino acid sequence of SEQ ID NO: 241, and a heavy chain comprising an amino acid sequence of SEQ ID NO: 242, which comprises no fucose residue on the Asn-297-linked N-oligosaccharide thereof. In some embodiments, the antibody is PSMB898.CLF comprising a light chain comprising an amino acid sequence of SEQ ID NO:241, and a heavy chain comprising an amino acid sequence of SEQ ID NO: 243, which comprises no fucose residue on the Asn-297-linked N-oligosaccharide thereof. In some embodiments, the antibody comprises a heavy chain comprising an amino acid sequence of SEQ ID NO: 242, which comprises no fucose residue on the Asn-297-linked N-oligosaccharide thereof. In some embodiments, the antibody comprises a heavy chain comprising an amino acid sequence of SEQ ID NO: 243, which comprises no fucose residue on the Asn-297-linked N-oligosaccharide thereof. In some embodiments, the antibody comprises a heavy chain comprising an amino acid sequence of SEQ ID NO: 242, which comprises the K248E, K338A, T437R, K248E/T437R or K338A/T437R mutation and comprises no fucose residue on the Asn-297-linked N-oligosaccharide thereof. In some embodiments, the antibody comprises a heavy chain comprising an amino acid sequence of SEQ ID NO: 243, which comprises the K248E, K338A, T437R, K248E/T437R or K338A/T437R mutation and comprises no fucose residue on the Asn-297-linked N-oligosaccharide thereof.

### 5.10 Anti-BCMA Antibodies and Related Molecules

In one aspect, provided herein are anti-BCMA antibodies.

In some embodiments, the antibody is BCMB519 comprising a VL comprising an amino acid sequence of SEQ ID NO: 299 and a VH comprising an amino acid sequence of SEQ ID NO: 298. In some embodiments, provided herein is an antibody that binds to BCMA, wherein the antibody comprises: (i) a VH comprising a VH CDR1, a VH CDR2, and a VH CDR3 having amino acid sequences of the VH CDR1, VH CDR2, and VH CDR3, respectively, of SEQ ID NO: 298; and (ii) a VL comprising a VL CDR1, a VL CDR2, and a VL CDR3 having amino acid sequences of the VL CDR1, VL CDR2, and VL CDR3, respectively, of SEQ ID NO: 299. In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system.

In another aspect, provided herein is an antibody that binds to BCMA, wherein the antibody comprises: (i) a VH comprising a VH CDR1 having an amino acid sequence of GFTFSSYA (SEQ ID NO: 292), a VH CDR2 having an amino acid sequence of ISGSGGST (SEQ ID NO: 293), and a VH CDR3 having an amino acid sequence of AKDEGYSSGHYYGMDV (SEQ ID NO: 294); and (ii) a VL comprising a VL CDR1 having an amino acid sequence of QSISSSF (SEQ ID NO: 295), a VL CDR2 having an amino acid sequence of GAS (SEQ ID NO: 296), and a VL CDR3 having an amino acid sequence of QHYGSSPMYT (SEQ ID NO: 297).

In some embodiments, the antibody comprises a VH having an amino acid sequence of SEQ ID NO: 298. In some embodiments, the antibody comprises a VL having an amino acid sequence of SEQ ID NO: 299. In some embodiments, the antibody comprises a VH comprising an amino acid sequence having at least 95% identity to the amino acid sequence of SEQ ID NO: 298. In some embodiments, the antibody comprises a VL comprising an amino acid sequence having at least 95% identity to the amino acid sequence of SEQ ID NO: 299.

In yet another aspect, provided herein are antibodies that compete with one of the antibodies thereof described above. Such antibodies may also bind to the same epitope as one of the above mentioned antibodies, or an overlapping epitope. Antibodies that compete with or bind to the same epitope as the above-mentioned antibodies are expected to show similar functional properties.

In certain embodiments, an antibody described herein comprises amino acid sequences with certain percent identity relative to the antibodies described above including the exemplary antibodies described in Section 7 below.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A., 1990, 87:2264-8, modified as in Karlin and Altschul, Proc. Natl. Acad. Sci. U.S.A. 1993, 90:5873-7. Such an algorithm is incorporated into the NBLAST and XBLAST programs of Altschul, et al., J. Mol. Biol., 1990, 215:403. BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res., 1997, 25:3389-402. Alternatively, PSI BLAST can be used to perform an iterated search which detects distant relationships between molecules (Id.). When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In certain embodiments, an antibody provided herein comprises a VH region having at least 80%, at least 85%, at least 90%, at least 95%, or at least 98% sequence identity to the amino acid sequence of SEQ ID NO: 298, and/or a VL region having at least 80%, at least 85%, at least 90%, at least 95%, or at least 98% sequence identity to the amino acid sequence of SEQ ID NO: 299, wherein the antibody binds to BCMA.

In some embodiments, provided herein is an antibody comprising VH, VL, or CDR sequences of the above described anti-BCMA antibodies, and further comprising an Fc region that has RE mutations but no fucose residue.

In some embodiments, the anti-BCMA antibody provided herein comprises a Fc region having RE mutations and no fucose residue.

Standard techniques known to those of skill in the art can be used to introduce mutations in the nucleotide sequence encoding a molecule provided herein, including, for example, site-directed mutagenesis and PCR-mediated mutagenesis which results in amino acid mutations.

In some embodiments, glutamic acid is substituted for lysine at position 248 (K248E) (EU numbering) of the Fc region of the antibody provided herein. In some embodiments, arginine is substituted for threonine at position 437 (T437R) (EU numbering) of the Fc region of the antibody provided herein. In some embodiments, glutamic acid is substituted for lysine at position 248, and arginine is substituted for threonine at position 437 (K248E/T437R) (EU numbering), of the Fc region of the antibody provided herein.

In some embodiments, the antibody provided herein comprises a heavy chain comprising the T437R mutation. In some embodiments, the antibody provided herein comprises a heavy chain comprising the K248E mutation. In some embodiments, the antibody provided herein comprises a heavy chain comprising the K338A mutation. In some embodiments, the antibody provided herein comprises a heavy chain comprising the K248E/T437R mutation. In some embodiments, the antibody provided herein comprises a heavy chain comprising the K338A/T437R mutation. In some embodiments, the antibody provided herein comprises a heavy chain comprising an amino acid sequence of SEQ ID NO: 300 but with T437R mutation. In some embodiments, the antibody provided herein comprises a heavy chain comprising an amino acid sequence of SEQ ID NO: 300 but with K248E mutation. In some embodiments, the antibody provided herein comprises a heavy chain comprising an amino acid sequence of SEQ ID NO: 300 but with K338A mutation. In some embodiments, the antibody provided herein comprises a heavy chain comprising an amino acid sequence of SEQ ID NO: 300 but with K248E/T437R mutation. In some embodiments, the antibody provided herein comprises a heavy chain comprising an amino acid sequence of SEQ ID NO: 300 but with K338A/T437R mutation.

In some embodiments, the antibody provided herein (including the above described anti-BCMA antibodies) is not fucosylated. Standard techniques known to those of skill in the art can be used to generate an antibody provided herein with a heavy chain comprising no fucose residue on the Asn-297-linked N-oligosaccharide thereof. For example, mammalian cell lines with deficient GMD enzymes (e.g., CHO Lec13 cells), with reduced α-1,6 fucosyltransferase activity resulting from mutated FUT8 genes, with overexpression of β-1,4-mannosyl-glycoprotein 4-β-N-acetylglucosaminyltransferase (GnT-III), with inactivated Golgi GDP-fucose transporter (GFT) gene Slc35c1 (e.g., CHO-gmt3 cells), with heterologous expression of bacterial RMD, or with use of biochemical inhibitors of fucosylation (e.g., fucose analogs such as 2-fluorofucose and 5-alkynylfucose) have been shown to produce antibodies with no fucose residue on the Asn-297-linked N-oligosaccharide on the heavy chain (Pereira, N. A., et al., supra; Shields, R. L. et al., supra; Kanda Y., supra).

In some embodiments, the antibody comprises a heavy chain comprising no fucose residue on the Asn-297-linked N-oligosaccharide thereof. In some embodiments, the antibody comprises a heavy chain comprising an amino acid sequence of SEQ ID NO: 300, which comprises no fucose residue on the Asn-297-linked N-oligosaccharide thereof. In some embodiments, the antibody comprises a heavy chain comprising an amino acid sequence of SEQ ID NO: 300, which comprises the K248E, K338A, T437R, K248E/T437R or K338A/T437R mutation and comprises no fucose residue on the Asn-297-linked N-oligosaccharide thereof.

### 5.11 Other Exemplary Antibodies

In some embodiments, the antibody provided herein comprises VL, VH or CDRs having amino acid sequences of the VL, VH or CDR contained in certain known antibodies, wherein the antibody comprises an Fc region with RE mutations and without fucosylation. Such exemplary known antibodies include but not limited to ReoPro (abciximab), Humira (adalimumab), Hyrimoz (adalimumab-adaz), Cyltezo (adalimumab-adbm), Abrilada (adalimumab-afzb), Amjevita (adalimumab-atto), Hadlima (adalimumab-bwwd), Campath, Lemtrada (alemtuzumab), Praluent (alirocumab), Tecentriq (atezolizumab), Bavencio (avelumab), Simulect (basiliximab), Benlysta (belimumab), Benlysta (belimumab), Fasenra (benralizumab), Avastin (bevacizumab), Mvasi (bevacizumab-awwb), Zirabev (bevacizumab-bvzr), Zinplava (bezlotoxumab), Blincyto (blinatumomab), Siliq (brodalumab), Beovu (brolucizumab-dbll), Crysvita (burosumab-twza), Ilaris (canakinumab), Cablivi (caplacizumab-yhdp), Libtayo (cemiplimab-rwlc), Erbitux (cetuximab), Adakveo (crizanlizumab-tmca), Zenapax (daclizumab), Zinbryta (daclizumab), Darzalex (daratumumab), Prolia, Xgeva (denosumab), Unituxin (dinutuximab), Dupixent (dupilumab), Imfinzi (durvalumab), Soliris (eculizumab), Empliciti (elotuzumab), Gamifant (emapalumab-lzsg), Hemlibra (emicizumab-kxwh), Vyepti (eptinezumab-jjmr), Aimovig (erenumab-aooe), Repatha (evolocumab), Ajovy (fremanezumab-vfrm), Emgality (galcanezumab-gnlm), Simponi (golimumab), Simponi Aria (golimumab), Tremfya (guselkumab), Trogarzo (ibalizumab-uiyk), Praxbind (idarucizumab), Remicade (infliximab), Renflexis (infliximab-abda), Avsola (infliximab-axxq), Inflectra (infliximab-dyyb), Ixifi (infliximab-qbtx), Yervoy (ipilimumab), Sarclisa (isatuximab-irfc), Taltz (ixekizumab), Takhzyro (lanadelumab-flyo), Nucala (mepolizumab), Nucala (mepolizumab), Poteligeo (mogamulizumab-kpkc), Tysabri (natalizumab), Portrazza (necitumumab), Opdivo (nivolumab), Anthim (obiltoxaximab), Gazyva (obinutuzumab), Ocrevus (ocrelizumab), Arzerra (ofatumumab), Lartruvo (olaratumab), Xolair (omalizumab), Synagis (palivizumab), Vectibix (panitumumab), Keytruda (pembrolizumab), Perjeta (pertuzumab), Cyramza (ramucirumab), Lucentis (ranibizumab), Ultomiris (ravulizumab-cwvz), raxibacumab (raxibacumab), Cinqair (reslizumab), Skyrizi (risankizumab-rzaa), Rituxan (rituximab), Truxima (rituximab-abbs), Ruxience (rituximab-pvvr), Evenity (romosozumab-aqqg), Kevzara (sarilumab), Cosentyx (secukinumab), Sylvant (siltuximab), Tepezza (teprotumumab-trbw), Ilumya (tildrakizumab-asmn), Actemra (tocilizumab), Actemra (tocilizumab), Herceptin (trastuzumab), Kanjinti (trastuzumab-anns), Ogivri (trastuzumab-dkst), Ontruzant (trastuzumab-dttb), Herzuma (trastuzumab-pkrb), Trazimera (trastuzumab-qyyp), Stelara (ustekinumab), Stelara (ustekinumab) and Entyvio (vedolizumab). The methods for introducing RE mutations and reducing fucosylation are described in more details in other sections provided herein and are well known in the art.

### 5.12 Polynucleotide

In certain embodiments, the disclosure encompasses polynucleotides that encode the antibodies described herein. The term “polynucleotides that encode a polypeptide” encompasses a polynucleotide that includes only coding sequences for the polypeptide as well as a polynucleotide which includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, a polynucleotide comprises the coding sequence for a polypeptide fused in the same reading frame to a polynucleotide which aids, for example, in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In certain embodiments, a polynucleotide comprises the coding sequence for a polypeptide fused in the same reading frame to a marker or tag sequence. For example, in some embodiments, a marker sequence is a hexa-histidine tag supplied by a vector that allows efficient purification of the polypeptide fused to the marker in the case of a bacterial host. In some embodiments, a marker is used in conjunction with other affinity tags.

In certain embodiments, the polynucleotide provided herein is selected from the polynucleotides listed in Table 17 below. In certain embodiments, the polynucleotide provided herein is selected from the polynucleotides listed in Table 20 and Table 22 below. In certain embodiments, the polynucleotide provided herein is selected from the polynucleotides listed in Table 26 below.

In certain embodiments, the polynucleotide provided herein is selected from the polynucleotides listed in Tables 9-12 below. In certain embodiments, the polynucleotide provided herein is selected from the polynucleotides listed in Table 15 and Table 16 below. In certain embodiments, the polynucleotide provided herein is selected from the polynucleotides listed in Table 2 below.

TABLE 2
Polynucleotide sequences of exemplary mouse antibodies

| Antibody Name | Light Chain | Heavy Chain |
|---|---|---|
| T26B373 | SEQ ID NO: 69 | SEQ ID NO: 77 |
| T26B374 | SEQ ID NO: 70 | SEQ ID NO: 78 |
| T26B375 | SEQ ID NO: 71 | SEQ ID NO: 79 |
| T26B379 | SEQ ID NO: 72 | SEQ ID NO: 80 |
| T26B382 | SEQ ID NO: 73 | SEQ ID NO: 81 |
| T26B385 | SEQ ID NO: 74 | SEQ ID NO: 82 |
| T26B386 | SEQ ID NO: 75 | SEQ ID NO: 83 |
| T26B388 | SEQ ID NO: 76 | SEQ ID NO: 84 |
| T26B459 | SEQ ID NO: 69 | SEQ ID NO: 85 |
| T26B460 | SEQ ID NO: 70 | SEQ ID NO: 86 |
| T26B461 | SEQ ID NO: 71 | SEQ ID NO: 87 |
| T26B462 | SEQ ID NO: 72 | SEQ ID NO: 88 |
| T26B463 | SEQ ID NO: 73 | SEQ ID NO: 89 |
| T26B464 | SEQ ID NO: 74 | SEQ ID NO: 90 |
| T26B465 | SEQ ID NO: 75 | SEQ ID NO: 91 |
| T26B466 | SEQ ID NO: 76 | SEQ ID NO: 92 |
| T26B608 | SEQ ID NO: 69 | SEQ ID NO: 93 |
| T26B609 | SEQ ID NO: 76 | SEQ ID NO: 94 |
| T26B610 | SEQ ID NO: 74 | SEQ ID NO: 95 |
| T26B611 | SEQ ID NO: 70 | SEQ ID NO: 96 |
| T26B612 | SEQ ID NO: 71 | SEQ ID NO: 97 |
| T26B613 | SEQ ID NO: 73 | SEQ ID NO: 98 |
| T26B614 | SEQ ID NO: 75 | SEQ ID NO: 99 |
| T26B615 | SEQ ID NO: 72 | SEQ ID NO: 100 |
| GC5B747 | SEQ ID NO: 104 | SEQ ID NO: 105 |
| GC5B752 | SEQ ID NO: 104 | SEQ ID NO: 106 |
| KL2B870 | SEQ ID NO: 161 | SEQ ID NO: 160 |
| KL2B871 | SEQ ID NO: 161 | SEQ ID NO: 162 |
| KL2B872 | SEQ ID NO: 161 | SEQ ID NO: 163 |
| MHGB732 | SEQ ID NO: 180 | SEQ ID NO: 182 |
| MHGB738 | SEQ ID NO: 181 | SEQ ID NO: 183 |

In some embodiments, the polynucleotide comprises a light chain nucleotide sequence of SEQ ID NO: 69 and/or a heavy chain nucleotide sequence of SEQ ID NO: 77. In some embodiments, the polynucleotide comprises a light chain nucleotide sequence of SEQ ID NO: 70 and/or a heavy chain nucleotide sequence of SEQ ID NO: 78. In some embodiments, the polynucleotide comprises a light chain nucleotide sequence of SEQ ID NO: 71 and/or a heavy chain nucleotide sequence of SEQ ID NO: 79. In some embodiments, the polynucleotide comprises a light chain nucleotide sequence of SEQ ID NO: 72 and/or a heavy chain nucleotide sequence of SEQ ID NO: 80. In some embodiments, the polynucleotide comprises a light chain nucleotide sequence of SEQ ID NO: 73 and/or a heavy chain nucleotide sequence of SEQ ID NO: 81. In some embodiments, the polynucleotide comprises a light chain nucleotide sequence of SEQ ID NO: 74 and/or a heavy chain nucleotide sequence of SEQ ID NO: 82. In some embodiments, the polynucleotide comprises a light chain nucleotide sequence of SEQ ID NO: 75 and/or a heavy chain nucleotide sequence of SEQ ID NO: 83. In some embodiments, the polynucleotide comprises a light chain nucleotide sequence of SEQ ID NO: 76 and/or a heavy chain nucleotide sequence of SEQ ID NO: 84. In some embodiments, the polynucleotide comprises a light chain nucleotide sequence of SEQ ID NO: 69 and/or a heavy chain nucleotide sequence of SEQ ID NO: 85. In some embodiments, the polynucleotide comprises a light chain nucleotide sequence of SEQ ID NO: 70 and/or a heavy chain nucleotide sequence of SEQ ID NO: 86. In some embodiments, the polynucleotide comprises a light chain nucleotide sequence of SEQ ID NO: 71 and/or a heavy chain nucleotide sequence of SEQ ID NO: 87. In some embodiments, the polynucleotide comprises a light chain nucleotide sequence of SEQ ID NO: 72 and/or a heavy chain nucleotide sequence of SEQ ID NO: 88. In some embodiments, the polynucleotide comprises a light chain nucleotide sequence of SEQ ID NO: 73 and/or a heavy chain nucleotide sequence of SEQ ID NO: 89. In some embodiments, the polynucleotide comprises a light chain nucleotide sequence of SEQ ID NO: 74 and/or a heavy chain nucleotide sequence of SEQ ID NO: 90. In some embodiments, the polynucleotide comprises a light chain nucleotide sequence of SEQ ID NO: 75 and/or a heavy chain nucleotide sequence of SEQ ID NO: 91. In some embodiments, the polynucleotide comprises a light chain nucleotide sequence of SEQ ID NO: 76 and/or a heavy chain nucleotide sequence of SEQ ID NO: 92. In some embodiments, the polynucleotide comprises a light chain nucleotide sequence of SEQ ID NO: 69 and/or a heavy chain nucleotide sequence of SEQ ID NO: 93. In some embodiments, the polynucleotide comprises a light chain nucleotide sequence of SEQ ID NO: 76 and/or a heavy chain nucleotide sequence of SEQ ID NO: 94. In some embodiments, the polynucleotide comprises a light chain nucleotide sequence of SEQ ID NO: 74 and/or a heavy chain nucleotide sequence of SEQ ID NO: 95. In some embodiments, the polynucleotide comprises a light chain nucleotide sequence of SEQ ID NO: 70 and/or a heavy chain nucleotide sequence of SEQ ID NO: 96. In some embodiments, the polynucleotide comprises a light chain nucleotide sequence of SEQ ID NO: 71 and/or a heavy chain nucleotide sequence of SEQ ID NO: 97. In some embodiments, the polynucleotide comprises a light chain nucleotide sequence of SEQ ID NO: 73 and/or a heavy chain nucleotide sequence of SEQ ID NO: 98. In some embodiments, the polynucleotide comprises a light chain nucleotide sequence of SEQ ID NO: 75 and/or a heavy chain nucleotide sequence of SEQ ID NO: 99. In some embodiments, the polynucleotide comprises a light chain nucleotide sequence of SEQ ID NO: 72 and/or a heavy chain nucleotide sequence of SEQ ID NO: 100.

In some embodiments, the polynucleotide comprises a light chain nucleotide sequence of SEQ ID NO: 104 and/or a heavy chain nucleotide sequence of SEQ ID NO: 105. In some embodiments, the polynucleotide comprises a light chain nucleotide sequence of SEQ ID NO: 104 and/or a heavy chain nucleotide sequence of SEQ ID NO: 106.

In some embodiments, the polynucleotide comprises a light chain nucleotide sequence of SEQ ID NO: 161 and/or a heavy chain nucleotide sequence of SEQ ID NO: 160. In some embodiments, the polynucleotide comprises a light chain nucleotide sequence of SEQ ID NO: 161 and/or a heavy chain nucleotide sequence of SEQ ID NO: 162. In some embodiments, the polynucleotide comprises a light chain nucleotide sequence of SEQ ID NO: 161 and/or a heavy chain nucleotide sequence of SEQ ID NO: 163.

In some embodiments, the polynucleotide comprises a light chain nucleotide sequence of SEQ ID NO: 180 and/or a heavy chain nucleotide sequence of SEQ ID NO: 182. In some embodiments, the polynucleotide comprises a light chain nucleotide sequence of SEQ ID NO: 181 and/or a heavy chain nucleotide sequence of SEQ ID NO: 183.

The present disclosure further relates to variants of the polynucleotides described herein, wherein the variant encodes, for example, fragments, analogs, and/or derivatives of a polypeptide. In certain embodiments, the present disclosure provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising an antibody described herein.

As used herein, the phrase “a polynucleotide having a nucleotide sequence at least, for example, 95% ‘identical’ to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations that produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In certain embodiments, the present disclosure provides a polynucleotide comprising a nucleotide sequence at least about 80% identical, at least about 85% identical, at least about 90% identical, at least about 95% identical, and in some embodiments, at least about 96%, 97%, 98% or 99% identical to a polynucleotide selected from the polynucleotides listed in Tables 9-12, 15, 16, 20, 22 and 26.

In certain embodiments, the polynucleotide provided herein further comprises one or more signal sequences before the VH and/or VL sequences listed in Tables 9-12, 15, 16, 20, 22 and 26.

In certain embodiments, a polynucleotide is isolated. In certain embodiments, a polynucleotide is substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises one or more expression vectors comprising polynucleotide molecules. In some embodiments, a host cell comprises a polynucleotide molecule. In some embodiments, a host cell comprises one or more polynucleotide molecules. Construction of the vectors provided herein is exemplified in Section 7 below.

### 5.13 Polyclonal and Monoclonal Antibodies

In some embodiments, a population of the antibodies provided herein comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a polypeptide (such HLA-G, CD37, GPRC5D, KLK2, PSMA, or BCMA or a fragment thereof) or a fusion protein thereof. It may be useful to conjugate the immunizing agent to a protein known to be immunogenic in the mammal being immunized or to immunize the mammal with the protein and one or more adjuvants. Examples of such immunogenic proteins include, but are not limited to, keyhole limpet hemocyanin, serum albumin, bovine thyroglobulin, and soybean trypsin inhibitor. Examples of adjuvants which may be employed include Ribi, CpG, Poly (I:C), Freund's complete adjuvant, and MPL-TDM adjuvant (monophosphoryl Lipid A, synthetic trehalose dicorynomycolate). The immunization protocol may be selected by one skilled in the art without undue experimentation. The mammal can then be bled, and the serum assayed for, e.g., anti-HLA-G, anti-CD37, anti-GPRC5D, anti-KLK2 antibody, anti-PSMA, or anti-BCMA titer. If desired, the mammal can be boosted until the antibody titer increases or plateaus. Additionally or alternatively, lymphocytes may be obtained from the immunized animal for fusion and preparation of monoclonal antibodies from hybridoma as described below.

In some embodiments, the antibodies provided herein comprise monoclonal antibodies. Monoclonal antibodies may be made using the hybridoma method first described by Kohler, et al., Nature, 1975, 256:495-7, or may be made by recombinant DNA methods (see, e.g., U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium, which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, VA), and those derived from MOPC-21 and MPC-11 mouse tumors available from the Salk Institute Cell Distribution Center (San Diego, CA). Human myeloma and mouse-human heteromyeloma cell lines also have been described for the production of human monoclonal antibodies (Kozbor, Immunol., 1984, 133:3001-05; and Brodeur, et al., Monoclonal Antibody Production Techniques and Applications, 1987, 51-63).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., Anal. Biochem., 1980, 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra, et al., Curr. Opinion in Immunol., 1993, 5:256-62 and Plückthun, Immunol. Revs., 1992, 130:151-88.

In some embodiments, an antibody that binds an epitope (e.g., on HLA-G, CD37, GPRC5D, KLK2, PSMA, or BCMA) comprises an amino acid sequence of a VH domain and/or an amino acid sequence of a VL domain encoded by a nucleotide sequence that hybridizes to (1) the complement of a nucleotide sequence encoding any one of the VH and/or VL domain described herein under stringent conditions (e.g., hybridization to filter-bound DNA in 6× sodium chloride/sodium citrate (SSC) at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art (see, e.g., Current Protocols in Molecular Biology Vol. I, 6.3.1-6.3.6 and 2.10.3 (Ausubel et al. eds., 1989)).

In a further embodiment, monoclonal antibodies can be isolated from antibody phage libraries generated using the techniques described in, for example, Antibody Phage Display: Methods and Protocols (O'Brien and Aitken, eds., 2002). In phage display methods, functional antibody domains are displayed on the surface of phage particles which carry the polynucleotide sequences encoding them. Examples of phage display methods that can be used to make the antibodies described herein include those disclosed in Brinkman, et al., J. Immunol. Methods, 1995, 182:41-50; Ames, et al., J. Immunol. Methods, 1995, 184:177-86; Kettleborough, et al., Eur. J. Immunol., 1994, 24:952-8; Persic, et al., Gene, 1997, 187:9-18; Burton et al., Advances in Immunology, 1994, 57:191-280; PCT Application No. PCT/GB91/O1134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 1993, 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, J. Mol. Biol., 1992, 227:381-88.

Screening of the libraries can be accomplished by various techniques known in the art. For example, HLA-G, CD37, GPRC5D, KLK2, PSMA, or BCMA (e.g., an HLA-G, CD37, GPRC5D, KLK2, PSMA, or BCMA polypeptide, fragment, or epitope) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass, et al., Proteins, 1990, 8:309-14 and WO 92/09690, and by use of a low coating density of antigen as described in Marks et al., Biotechnol., 1992, 10:779-83.

Antibodies can be obtained by designing a suitable antigen screening procedure to select for the phage clone of interest followed by construction of a full length antibody clone using VH and/or VL sequences (e.g., the Fv sequences), or various CDR sequences from VH and VL sequences, from the phage clone of interest and suitable constant region (e.g., Fc) sequences described in Kabat, et al., supra, including sequences comprising the K248E, T437R, or K248E/T437R mutation.

Antibodies described herein can also, for example, include chimeric antibodies. A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison, Science, 1985, 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

Antibodies produced using techniques such as those described herein can be isolated using standard, well known techniques. For example, antibodies can be suitably separated from, e.g., culture medium, ascites fluid, serum, cell lysate, synthesis reaction material or the like by conventional immunoglobulin purification procedures such as, for example, protein A-Sepharose, hydroxylapatite chromatography, gel electrophoresis, dialysis, or affinity chromatography. As used herein, an “isolated” or “purified” antibody is substantially free of cellular material or other proteins from the cell or tissue source from which the antibody is derived, or substantially free of chemical precursors or other chemicals when chemically synthesized.

In some specific embodiments, monoclonal antibodies are generated using the methods exemplified in Section 7 below.

### 5.14 Humanized Antibodies

In some embodiments, the antibodies provided herein can be humanized antibodies. A humanized antibody can comprise human framework region and human constant region sequences. For example, a humanized antibody can comprise human constant region sequences. In certain embodiments, a humanized antibody can be selected from any class of immunoglobulins, including IgM, IgG, IgD, IgA and IgE, and any isotype, including IgG1, IgG2, IgG3 and IgG4 (e.g., variants of IgG4 and IgG4 nullbody). In certain embodiments, a humanized antibody can comprise kappa or lambda light chain constant sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, Molecular Immunology, 1991, 28(4/5):489-498; Studnicka, et al., Protein Engineering, 1994, 7(6):805-814; and Roguska, et al., Proc. Natl. Acad. Sci. USA, 1994, 91:969-73), chain shuffling (U.S. Pat. No. 5,565,332), and techniques disclosed in, e.g., U.S. Pat. Nos. 6,407,213, 5,766,886, WO 93/17105; Tan, et al., J. Immunol., 2002, 169:1119-25; Caldas, et al., Protein Eng., 2000, 13(5):353-60; Morea et al., Methods, 2000, 20(3):267-79, Baca, et al., J. Biol. Chem., 1997, 272(16):10678-84; Roguska, et al., Protein Eng., 1996, 9(10):895 904; Couto, et al., Cancer Res., 1995, 55 (23 Supp):5973s-5977s; Couto, et al., Cancer Res., 1995, 55(8):1717-22; Sandhu, J. S., Gene, 1994, 150(2):409-10 and Pedersen, et al., J. Mol. Biol., 1994, 235(3):959-73. See also U.S. Patent Pub. No. US 2005/0042664 A1 (Feb. 24, 2005), each of which is incorporated by reference herein in its entirety.

Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones, et al., 1986, Nature 321:522-5; Riechmann, et al., Nature, 1988, 332:323-7; and Verhoeyen, et al., Science, 1988, 239:1534-6, by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan, et al. determined that only about one third of the residues in the CDRs actually contact the antigen, and termed these the “specificity determining residues,” or SDRs (Padlan, et al., FASEB J., 1995, 9:133-9). In the technique of SDR grafting, only the SDR residues are grafted onto the human antibody framework (see, e.g., Kashmiri, et al., Methods, 2005, 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., J. Immunol., 1993, 151:2296-308; and Chothia et al., J. Mol. Biol., 1987, 196:901-17). Another method uses a particular framework derived from the consensus sequence of all human antibodies of a particular subgroup of light or heavy chains. The same framework may be used for several different humanized antibodies (Carter et al., Proc. Natl. Acad. Sci. USA, 1992, 89:4285-89; and Presta et al., J. Immunol., 1993, 151:2623-32). In some cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., J. Immunol., 2002, 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng., 2000, 13:819-24), Modeller (Sali and Blundell, J. Mol. Biol., 1993, 234:779-815), and Swiss PDB Viewer (Guex and Peitsch, Electrophoresis, 1997, 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., Mol. Immunol., 2007, 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, Nat. Biotechnol., 2005, 23:1105-16; Dufner, et al., Trends Biotechnol., 2006, 24:523-9; Feldhaus, et al., Nat. Biotechnol., 2003, 21:163-70; and Schlapschy et al., Protein Eng. Des. Sel., 2004, 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, J. Mol. Biol., 1992, 224:487-99), or from the more limited set of target residues identified by Baca, et al., J. Biol. Chem., 1997, 272:10678-84.

In FR shuffling, whole FRs are combined with the non-human CDRs instead of creating combinatorial libraries of selected residue variants (see, e.g., Dall'Acqua et al., Methods, 2005, 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder, et al., Mol. Immunol., 2007, 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering, 1994, 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody. Such antibodies can comprise human constant region sequences, e.g., human light chain and/or heavy chain constant regions.

A deimmunized antibody is an antibody in which T-cell epitopes have been removed. Methods for making deimmunized antibodies have been described (see, e.g., Jones, et al., Methods Mol Biol., 2009, 525:405-23; and De Groot, et al., Cell. Immunol., 2006, 244:148-153). Deimmunized antibodies comprise T-cell epitope-depleted variable regions and human constant regions. Briefly, VH and VL of an antibody are cloned and T-cell epitopes are subsequently identified by testing overlapping peptides derived from the VH and VL of the antibody in a T cell proliferation assay. T cell epitopes are identified via in silico methods to identify peptide binding to human MHC class II. Mutations are introduced in the VH and VL to abrogate binding to human MHC class II. Mutated VH and VL are then utilized to generate the deimmunized antibody.

### 5.15 Antibody Variants

In some embodiments, amino acid sequence modification(s) of the antibodies provided herein are contemplated. For example, it may be desirable to improve the binding affinity and/or other biological properties of the antibody, including but not limited to specificity, thermostability, expression level, effector functions, glycosylation (e.g., fucosylation), reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art would appreciate that amino acid changes may alter post-translational processes of the antibody, such as changing the number or position of glycosylation sites or altering the membrane anchoring characteristics.

In some embodiments, antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Alternatively, conservative (e.g., within an amino acid group with similar properties and/or side chains) substitutions may be made, so as to maintain or not significantly change the properties. Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions entail exchanging a member of one of these classes for another class. Such substituted residues also may be introduced into the conservative substitution sites or, into the remaining (non-conserved) sites.

Accordingly, in one embodiment, an antibody that binds to an HLA-G, CD37, GPRC5D, KLK2, PSMA, or BCMA epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an antibody described herein, for examples, the antibodies described in Section 7 below.

In one embodiment, an antibody that binds to an HLA-G, CD37, GPRC5D, KLK2, PSMA, or BCMA epitope comprises an amino acid sequence that is at least 35% identical to the amino acid sequence of an antibody described herein. In one embodiment, an antibody that binds to an HLA-G, CD37, GPRC5D, KLK2, PSMA, or BCMA epitope comprises an amino acid sequence that is at least 40% identical to the amino acid sequence of an antibody described herein. In one embodiment, an antibody that binds to an HLA-G, CD37, GPRC5D, KLK2, PSMA, or BCMA epitope comprises an amino acid sequence that is at least 45% identical to the amino acid sequence of an antibody described herein. In one embodiment, an antibody that binds to an HLA-G, CD37, GPRC5D, KLK2, PSMA, or BCMA epitope comprises an amino acid sequence that is at least 50% identical to the amino acid sequence of an antibody described herein. In one embodiment, an antibody that binds to an HLA-G, CD37, GPRC5D, KLK2, PSMA, or BCMA epitope comprises an amino acid sequence that is at least 55% identical to the amino acid sequence of an antibody described herein. In one embodiment, an antibody that binds to an HLA-G, CD37, GPRC5D, KLK2, PSMA, or BCMA epitope comprises an amino acid sequence that is at least 60% identical to the amino acid sequence of an antibody described herein. In one embodiment, an antibody that binds to an HLA-G, CD37, GPRC5D, KLK2, PSMA, or BCMA epitope comprises an amino acid sequence that is at least 65% identical to the amino acid sequence of an antibody described herein. In one embodiment, an antibody that binds to an HLA-G, CD37, GPRC5D, KLK2, PSMA, or BCMA epitope comprises an amino acid sequence that is at least 70% identical to the amino acid sequence of an antibody described herein. In one embodiment, an antibody that binds to an HLA-G, CD37, GPRC5D, KLK2, PSMA, or BCMA epitope comprises an amino acid sequence that is at least 75% identical to the amino acid sequence of an antibody described herein. In one embodiment, an antibody that binds to an HLA-G, CD37, GPRC5D, KLK2, PSMA, or BCMA epitope comprises an amino acid sequence that is at least 80% identical to the amino acid sequence of an antibody described herein. In one embodiment, an antibody that binds to an HLA-G, CD37, GPRC5D, KLK2, PSMA, or BCMA epitope comprises an amino acid sequence that is at least 90% identical to the amino acid sequence of an antibody described herein. In one embodiment, an antibody that binds to an HLA-G, CD37, GPRC5D, KLK2, PSMA, or BCMA epitope comprises an amino acid sequence that is at least 95% identical to the amino acid sequence of an antibody described herein. In one embodiment, an antibody that binds to an HLA-G, CD37, GPRC5D, KLK2, PSMA, or BCMA comprises an amino acid sequence that is at least 99% identical to the amino acid sequence of an antibody described herein.

In some embodiments, provided herein is an antibody that binds to a CD37 epitope and comprises a VL region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 3, a VH region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 6, a light chain comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 7, and/or a heavy chain comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 8.

In some embodiments, provided herein is an antibody that binds to a CD37 epitope and comprises a VL region comprising an amino acid sequence that is at least 35% identical to an amino acid sequence provided in Table 5. In some embodiments, provided herein is an antibody that binds to a CD37 epitope and comprises a VL region comprising an amino acid sequence that is at least 40% identical to an amino acid sequence provided in Table 5. In some embodiments, provided herein is an antibody that binds to a CD37 epitope and comprises a VL region comprising an amino acid sequence that is at least 45% identical to an amino acid sequence provided in Table 5. In some embodiments, provided herein is an antibody that binds to a CD37 epitope and comprises a VL region comprising an amino acid sequence that is at least 50% identical to an amino acid sequence provided in Table 5. In some embodiments, provided herein is an antibody that binds to a CD37 epitope and comprises a VL region comprising an amino acid sequence that is at least 55% identical to an amino acid sequence provided in Table 5. In some embodiments, provided herein is an antibody that binds to a CD37 epitope and comprises a VL region comprising an amino acid sequence that is at least 60% identical to an amino acid sequence provided in Table 5. In some embodiments, provided herein is an antibody that binds to a CD37 epitope and comprises a VL region comprising an amino acid sequence that is at least 65% identical to an amino acid sequence provided in Table 5. In some embodiments, provided herein is an antibody that binds to a CD37 epitope and comprises a VL region comprising an amino acid sequence that is at least 70% identical to an amino acid sequence provided in Table 5. In some embodiments, provided herein is an antibody that binds to a CD37 epitope and comprises a VL region comprising an amino acid sequence that is at least 75% identical to an amino acid sequence provided in Table 5. In some embodiments, provided herein is an antibody that binds to a CD37 epitope and comprises a VL region comprising an amino acid sequence that is at least 80% identical to an amino acid sequence provided in Table 5. In some embodiments, provided herein is an antibody that binds to a CD37 epitope and comprises a VL region comprising an amino acid sequence that is at least 85% identical to an amino acid sequence provided in Table 5. In some embodiments, provided herein is an antibody that binds to a CD37 epitope and comprises a VL region comprising an amino acid sequence that is at least 90% identical to an amino acid sequence provided in Table 5. In some embodiments, provided herein is an antibody that binds to a CD37 epitope and comprises a VL region comprising an amino acid sequence that is at least 95% identical to an amino acid sequence provided in Table 5. In some embodiments, provided herein is an antibody that binds to a CD37 epitope and comprises a VL region comprising an amino acid sequence that is at least 99% identical to an amino acid sequence provided herein and Table 5.

In some embodiments, provided herein is an antibody that binds to a CD37 epitope and comprises a VH region comprising an amino acid sequence that is at least 35% identical to an amino acid sequence provided in Table 6. In some embodiments, provided herein is an antibody that binds to a CD37 epitope and comprises a VH region comprising an amino acid sequence that is at least 40% identical to an amino acid sequence provided in Table 6. In some embodiments, provided herein is an antibody that binds to a CD37 epitope and comprises a VH region comprising an amino acid sequence that is at least 45% identical to an amino acid sequence provided in Table 6. In some embodiments, provided herein is an antibody that binds to a CD37 epitope and comprises a VH region comprising an amino acid sequence that is at least 50% identical to an amino acid sequence provided in Table 6. In some embodiments, provided herein is an antibody that binds to a CD37 epitope and comprises a VH region comprising an amino acid sequence that is at least 55% identical to an amino acid sequence provided in Table 6. In some embodiments, provided herein is an antibody that binds to a CD37 epitope and comprises a VH region comprising an amino acid sequence that is at least 60% identical to an amino acid sequence provided in Table 6. In some embodiments, provided herein is an antibody that binds to a CD37 epitope and comprises a VH region comprising an amino acid sequence that is at least 65% identical to an amino acid sequence provided in Table 6. In some embodiments, provided herein is an antibody that binds to a CD37 epitope and comprises a VH region comprising an amino acid sequence that is at least 70% identical to an amino acid sequence provided in Table 6. In some embodiments, provided herein is an antibody that binds to a CD37 epitope and comprises a VH region comprising an amino acid sequence that is at least 75% identical to an amino acid sequence provided in Table 6. In some embodiments, provided herein is an antibody that binds to a CD37 epitope and comprises a VH region comprising an amino acid sequence that is at least 80% identical to an amino acid sequence provided in Table 6. In some embodiments, provided herein is an antibody that binds to a CD37 epitope and comprises a VH region comprising an amino acid sequence that is at least 85% identical to an amino acid sequence provided in Table 6. In some embodiments, provided herein is an antibody that binds to a CD37 epitope and comprises a VH region comprising an amino acid sequence that is at least 90% identical to an amino acid sequence provided in Table 6. In some embodiments, provided herein is an antibody that binds to a CD37 epitope and comprises a VH region comprising an amino acid sequence that is at least 95% identical to an amino acid sequence provided in Table 6. In some embodiments, provided herein is an antibody that binds to a CD37 epitope and comprises a VH region comprising an amino acid sequence that is at least 99% identical to an amino acid sequence provided herein and Table 6.

In some embodiments, provided herein is an antibody that binds to a CD37 epitope and comprises a light chain comprising an amino acid sequence that is at least 35% identical to an amino acid sequence provided in Table 7. In some embodiments, provided herein is an antibody that binds to a CD37 epitope and comprises a light chain comprising an amino acid sequence that is at least 40% identical to an amino acid sequence provided in Table 7. In some embodiments, provided herein is an antibody that binds to a CD37 epitope and comprises a light chain comprising an amino acid sequence that is at least 45% identical to an amino acid sequence provided in Table 7. In some embodiments, provided herein is an antibody that binds to a CD37 epitope and comprises a light chain comprising an amino acid sequence that is at least 50% identical to an amino acid sequence provided in Table 7. In some embodiments, provided herein is an antibody that binds to a CD37 epitope and comprises a light chain comprising an amino acid sequence that is at least 55% identical to an amino acid sequence provided in Table 7. In some embodiments, provided herein is an antibody that binds to a CD37 epitope and comprises a light chain comprising an amino acid sequence that is at least 60% identical to an amino acid sequence provided in Table 7. In some embodiments, provided herein is an antibody that binds to a CD37 epitope and comprises a light chain comprising an amino acid sequence that is at least 65% identical to an amino acid sequence provided in Table 7. In some embodiments, provided herein is an antibody that binds to a CD37 epitope and comprises a light chain comprising an amino acid sequence that is at least 70% identical to an amino acid sequence provided in Table 7. In some embodiments, provided herein is an antibody that binds to a CD37 epitope and comprises a light chain comprising an amino acid sequence that is at least 75% identical to an amino acid sequence provided in Table 7. In some embodiments, provided herein is an antibody that binds to a CD37 epitope and comprises a light chain comprising an amino acid sequence that is at least 80% identical to an amino acid sequence provided in Table 7. In some embodiments, provided herein is an antibody that binds to a CD37 epitope and comprises a light chain comprising an amino acid sequence that is at least 85% identical to an amino acid sequence provided in Table 7. In some embodiments, provided herein is an antibody that binds to a CD37 epitope and comprises a light chain comprising an amino acid sequence that is at least 90% identical to an amino acid sequence provided in Table 7. In some embodiments, provided herein is an antibody that binds to a CD37 epitope and comprises a light chain comprising an amino acid sequence that is at least 95% identical to an amino acid sequence provided in Table 7. In some embodiments, provided herein is an antibody that binds to a CD37 epitope and comprises a light chain comprising an amino acid sequence that is at least 99% identical to an amino acid sequence provided herein and Table 7.

In some embodiments, provided herein is an antibody that binds to a CD37 epitope and comprises a heavy chain comprising an amino acid sequence that is at least 35% identical to an amino acid sequence provided in Table 8. In some embodiments, provided herein is an antibody that binds to a CD37 epitope and comprises a heavy chain comprising an amino acid sequence that is at least 40% identical to an amino acid sequence provided in Table 8. In some embodiments, provided herein is an antibody that binds to a CD37 epitope and comprises a heavy chain comprising an amino acid sequence that is at least 45% identical to an amino acid sequence provided in Table 8. In some embodiments, provided herein is an antibody that binds to a CD37 epitope and comprises a heavy chain comprising an amino acid sequence that is at least 50% identical to an amino acid sequence provided in Table 8. In some embodiments, provided herein is an antibody that binds to a CD37 epitope and comprises a heavy chain comprising an amino acid sequence that is at least 55% identical to an amino acid sequence provided in Table 8. In some embodiments, provided herein is an antibody that binds to a CD37 epitope and comprises a heavy chain comprising an amino acid sequence that is at least 60% identical to an amino acid sequence provided in Table 8. In some embodiments, provided herein is an antibody that binds to a CD37 epitope and comprises a heavy chain comprising an amino acid sequence that is at least 65% identical to an amino acid sequence provided in Table 8. In some embodiments, provided herein is an antibody that binds to a CD37 epitope and comprises a heavy chain comprising an amino acid sequence that is at least 70% identical to an amino acid sequence provided in Table 8. In some embodiments, provided herein is an antibody that binds to a CD37 epitope and comprises a heavy chain comprising an amino acid sequence that is at least 75% identical to an amino acid sequence provided in Table 8. In some embodiments, provided herein is an antibody that binds to a CD37 epitope and comprises a heavy chain comprising an amino acid sequence that is at least 80% identical to an amino acid sequence provided in Table 8. In some embodiments, provided herein is an antibody that binds to a CD37 epitope and comprises a heavy chain comprising an amino acid sequence that is at least 85% identical to an amino acid sequence provided in Table 8. In some embodiments, provided herein is an antibody that binds to a CD37 epitope and comprises a heavy chain comprising an amino acid sequence that is at least 90% identical to an amino acid sequence provided in Table 8. In some embodiments, provided herein is an antibody that binds to a CD37 epitope and comprises a heavy chain comprising an amino acid sequence that is at least 95% identical to an amino acid sequence provided in Table 8. In some embodiments, provided herein is an antibody that binds to a CD37 epitope and comprises a heavy chain comprising an amino acid sequence that is at least 99% identical to an amino acid sequence provided herein and Table 8.

In yet another embodiment, an antibody that binds to a CD37 epitope comprises a VL CDR and/or a VH CDR amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a VL CDR amino acid sequence and/or a VH CDR amino acid sequence contained in the VL and VH sequences in Table 5 and Table 6 below.

In yet another embodiment, an antibody that binds to a CD37 epitope comprises a VL CDR and/or a VH CDR amino acid sequence that is at least 35% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 40% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 45% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 50% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 55% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 60% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 65% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 70% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 75% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 80% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 85% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 90% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 95% identical to a VL CDR amino acid sequence and/or a VH CDR amino acid sequence contained in the VL and VH sequences in Table 5 and Table 6. In yet another embodiment, an antibody that binds to a CD37 epitope comprises a VL CDR and/or a VH CDR amino acid sequence that is at least 99% identical to a VL CDR amino acid sequence and/or a VH CDR amino acid sequence contained in the VL and VH sequences in Table 5 and Table 6.

In some embodiments, provided herein is an antibody that binds to a GPRC5D epitope and comprises a VL region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 13, a VH region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 13, a light chain comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 14, and/or a heavy chain comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 14.

In some embodiments, provided herein is an antibody that binds to a GPRC5D epitope and comprises a VL region comprising an amino acid sequence that is at least 35% identical to an amino acid sequence provided in Table 13. In some embodiments, provided herein is an antibody that binds to a GPRC5D epitope and comprises a VL region comprising an amino acid sequence that is at least 40% identical to an amino acid sequence provided in Table 13. In some embodiments, provided herein is an antibody that binds to a GPRC5D epitope and comprises a VL region comprising an amino acid sequence that is at least 45% identical to an amino acid sequence provided in Table 13. In some embodiments, provided herein is an antibody that binds to a GPRC5D epitope and comprises a VL region comprising an amino acid sequence that is at least 50% identical to an amino acid sequence provided in Table 13. In some embodiments, provided herein is an antibody that binds to a GPRC5D epitope and comprises a VL region comprising an amino acid sequence that is at least 55% identical to an amino acid sequence provided in Table 13. In some embodiments, provided herein is an antibody that binds to a GPRC5D epitope and comprises a VL region comprising an amino acid sequence that is at least 60% identical to an amino acid sequence provided in Table 13. In some embodiments, provided herein is an antibody that binds to a GPRC5D epitope and comprises a VL region comprising an amino acid sequence that is at least 65% identical to an amino acid sequence provided in Table 13. In some embodiments, provided herein is an antibody that binds to a GPRC5D epitope and comprises a VL region comprising an amino acid sequence that is at least 70% identical to an amino acid sequence provided in Table 13. In some embodiments, provided herein is an antibody that binds to a GPRC5D epitope and comprises a VL region comprising an amino acid sequence that is at least 75% identical to an amino acid sequence provided in Table 13. In some embodiments, provided herein is an antibody that binds to a GPRC5D epitope and comprises a VL region comprising an amino acid sequence that is at least 80% identical to an amino acid sequence provided in Table 13. In some embodiments, provided herein is an antibody that binds to a GPRC5D epitope and comprises a VL region comprising an amino acid sequence that is at least 85% identical to an amino acid sequence provided in Table 13. In some embodiments, provided herein is an antibody that binds to a GPRC5D epitope and comprises a VL region comprising an amino acid sequence that is at least 90% identical to an amino acid sequence provided in Table 13. In some embodiments, provided herein is an antibody that binds to a GPRC5D epitope and comprises a VL region comprising an amino acid sequence that is at least 95% identical to an amino acid sequence provided in Table 13. In some embodiments, provided herein is an antibody that binds to a GPRC5D epitope and comprises a VL region comprising an amino acid sequence that is at least 99% identical to an amino acid sequence provided herein and Table 13.

In some embodiments, provided herein is an antibody that binds to a GPRC5D epitope and comprises a VH region comprising an amino acid sequence that is at least 35% identical to an amino acid sequence provided in Table 13. In some embodiments, provided herein is an antibody that binds to a GPRC5D epitope and comprises a VH region comprising an amino acid sequence that is at least 40% identical to an amino acid sequence provided in Table 13. In some embodiments, provided herein is an antibody that binds to a GPRC5D epitope and comprises a VH region comprising an amino acid sequence that is at least 45% identical to an amino acid sequence provided in Table 13. In some embodiments, provided herein is an antibody that binds to a GPRC5D epitope and comprises a VH region comprising an amino acid sequence that is at least 50% identical to an amino acid sequence provided in Table 13. In some embodiments, provided herein is an antibody that binds to a GPRC5D epitope and comprises a VH region comprising an amino acid sequence that is at least 55% identical to an amino acid sequence provided in Table 13. In some embodiments, provided herein is an antibody that binds to a GPRC5D epitope and comprises a VH region comprising an amino acid sequence that is at least 60% identical to an amino acid sequence provided in Table 13. In some embodiments, provided herein is an antibody that binds to a GPRC5D epitope and comprises a VH region comprising an amino acid sequence that is at least 65% identical to an amino acid sequence provided in Table 13. In some embodiments, provided herein is an antibody that binds to a GPRC5D epitope and comprises a VH region comprising an amino acid sequence that is at least 70% identical to an amino acid sequence provided in Table 13. In some embodiments, provided herein is an antibody that binds to a GPRC5D epitope and comprises a VH region comprising an amino acid sequence that is at least 75% identical to an amino acid sequence provided in Table 13. In some embodiments, provided herein is an antibody that binds to a GPRC5D epitope and comprises a VH region comprising an amino acid sequence that is at least 80% identical to an amino acid sequence provided in Table 13. In some embodiments, provided herein is an antibody that binds to a GPRC5D epitope and comprises a VH region comprising an amino acid sequence that is at least 85% identical to an amino acid sequence provided in Table 13. In some embodiments, provided herein is an antibody that binds to a GPRC5D epitope and comprises a VH region comprising an amino acid sequence that is at least 90% identical to an amino acid sequence provided in Table 13. In some embodiments, provided herein is an antibody that binds to a GPRC5D epitope and comprises a VH region comprising an amino acid sequence that is at least 95% identical to an amino acid sequence provided in Table 13. In some embodiments, provided herein is an antibody that binds to a GPRC5D epitope and comprises a VH region comprising an amino acid sequence that is at least 99% identical to an amino acid sequence provided herein and Table 13.

In some embodiments, provided herein is an antibody that binds to a GPRC5D epitope and comprises a light chain comprising an amino acid sequence that is at least 35% identical to an amino acid sequence provided in Table 14. In some embodiments, provided herein is an antibody that binds to a GPRC5D epitope and comprises a light chain comprising an amino acid sequence that is at least 40% identical to an amino acid sequence provided in Table 14. In some embodiments, provided herein is an antibody that binds to a GPRC5D epitope and comprises a light chain comprising an amino acid sequence that is at least 45% identical to an amino acid sequence provided in Table 14. In some embodiments, provided herein is an antibody that binds to a GPRC5D epitope and comprises a light chain comprising an amino acid sequence that is at least 50% identical to an amino acid sequence provided in Table 14. In some embodiments, provided herein is an antibody that binds to a GPRC5D epitope and comprises a light chain comprising an amino acid sequence that is at least 55% identical to an amino acid sequence provided in Table 14. In some embodiments, provided herein is an antibody that binds to a GPRC5D epitope and comprises a light chain comprising an amino acid sequence that is at least 60% identical to an amino acid sequence provided in Table 14. In some embodiments, provided herein is an antibody that binds to a GPRC5D epitope and comprises a light chain comprising an amino acid sequence that is at least 65% identical to an amino acid sequence provided in Table 14. In some embodiments, provided herein is an antibody that binds to a GPRC5D epitope and comprises a light chain comprising an amino acid sequence that is at least 70% identical to an amino acid sequence provided in Table 14. In some embodiments, provided herein is an antibody that binds to a GPRC5D epitope and comprises a light chain comprising an amino acid sequence that is at least 75% identical to an amino acid sequence provided in Table 14. In some embodiments, provided herein is an antibody that binds to a GPRC5D epitope and comprises a light chain comprising an amino acid sequence that is at least 80% identical to an amino acid sequence provided in Table 14. In some embodiments, provided herein is an antibody that binds to a GPRC5D epitope and comprises a light chain comprising an amino acid sequence that is at least 85% identical to an amino acid sequence provided in Table 14. In some embodiments, provided herein is an antibody that binds to a GPRC5D epitope and comprises a light chain comprising an amino acid sequence that is at least 90% identical to an amino acid sequence provided in Table 14. In some embodiments, provided herein is an antibody that binds to a GPRC5D epitope and comprises a light chain comprising an amino acid sequence that is at least 95% identical to an amino acid sequence provided in Table 14. In some embodiments, provided herein is an antibody that binds to a GPRC5D epitope and comprises a light chain comprising an amino acid sequence that is at least 99% identical to an amino acid sequence provided herein and Table 14.

In some embodiments, provided herein is an antibody that binds to a GPRC5D epitope and comprises a heavy chain comprising an amino acid sequence that is at least 35% identical to an amino acid sequence provided in Table 14. In some embodiments, provided herein is an antibody that binds to a GPRC5D epitope and comprises a heavy chain comprising an amino acid sequence that is at least 40% identical to an amino acid sequence provided in Table 14. In some embodiments, provided herein is an antibody that binds to a GPRC5D epitope and comprises a heavy chain comprising an amino acid sequence that is at least 45% identical to an amino acid sequence provided in Table 14. In some embodiments, provided herein is an antibody that binds to a GPRC5D epitope and comprises a heavy chain comprising an amino acid sequence that is at least 50% identical to an amino acid sequence provided in Table 14. In some embodiments, provided herein is an antibody that binds to a GPRC5D epitope and comprises a heavy chain comprising an amino acid sequence that is at least 55% identical to an amino acid sequence provided in Table 14. In some embodiments, provided herein is an antibody that binds to a GPRC5D epitope and comprises a heavy chain comprising an amino acid sequence that is at least 60% identical to an amino acid sequence provided in Table 14. In some embodiments, provided herein is an antibody that binds to a GPRC5D epitope and comprises a heavy chain comprising an amino acid sequence that is at least 65% identical to an amino acid sequence provided in Table 14. In some embodiments, provided herein is an antibody that binds to a GPRC5D epitope and comprises a heavy chain comprising an amino acid sequence that is at least 70% identical to an amino acid sequence provided in Table 14. In some embodiments, provided herein is an antibody that binds to a GPRC5D epitope and comprises a heavy chain comprising an amino acid sequence that is at least 75% identical to an amino acid sequence provided in Table 14. In some embodiments, provided herein is an antibody that binds to a GPRC5D epitope and comprises a heavy chain comprising an amino acid sequence that is at least 80% identical to an amino acid sequence provided in Table 14. In some embodiments, provided herein is an antibody that binds to a GPRC5D epitope and comprises a heavy chain comprising an amino acid sequence that is at least 85% identical to an amino acid sequence provided in Table 14. In some embodiments, provided herein is an antibody that binds to a GPRC5D epitope and comprises a heavy chain comprising an amino acid sequence that is at least 90% identical to an amino acid sequence provided in Table 14. In some embodiments, provided herein is an antibody that binds to a GPRC5D epitope and comprises a heavy chain comprising an amino acid sequence that is at least 95% identical to an amino acid sequence provided in Table 14. In some embodiments, provided herein is an antibody that binds to a GPRC5D epitope and comprises a heavy chain comprising an amino acid sequence that is at least 99% identical to an amino acid sequence provided herein and Table 14.

In yet another embodiment, an antibody that binds to a GPRC5D epitope comprises a VL CDR and/or a VH CDR amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a VL CDR amino acid sequence and/or a VH CDR amino acid sequence contained in the VL and VH sequences in Table 13 below.

In yet another embodiment, an antibody that binds to a GPRC5D epitope comprises a VL CDR and/or a VH CDR amino acid sequence that is at least 35% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 40% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 45% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 50% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 55% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 60% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 65% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 70% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 75% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 80% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 85% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 90% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 95% identical to a VL CDR amino acid sequence and/or a VH CDR amino acid sequence contained in the VL and VH sequences in Table 13. In yet another embodiment, an antibody that binds to a GPRC5D epitope comprises a VL CDR and/or a VH CDR amino acid sequence that is at least 99% identical to a VL CDR amino acid sequence and/or a VH CDR amino acid sequence contained in the VL and VH sequences in Table 13.

In one embodiment, an antibody that binds to a KLK2 epitope comprises an amino acid sequence that is at least 35% identical to the amino acid sequence of an antibody described herein. In one embodiment, an antibody that binds to a KLK2 epitope comprises an amino acid sequence that is at least 40% identical to the amino acid sequence of an antibody described herein. In one embodiment, an antibody that binds to a KLK2 epitope comprises an amino acid sequence that is at least 45% identical to the amino acid sequence of an antibody described herein. In one embodiment, an antibody that binds to a KLK2 epitope comprises an amino acid sequence that is at least 50% identical to the amino acid sequence of an antibody described herein. In one embodiment, an antibody that binds to a KLK2 epitope comprises an amino acid sequence that is at least 55% identical to the amino acid sequence of an antibody described herein. In one embodiment, an antibody that binds to a KLK2 epitope comprises an amino acid sequence that is at least 60% identical to the amino acid sequence of an antibody described herein. In one embodiment, an antibody that binds to a KLK2 epitope comprises an amino acid sequence that is at least 65% identical to the amino acid sequence of an antibody described herein. In one embodiment, an antibody that binds to a KLK2 epitope comprises an amino acid sequence that is at least 70% identical to the amino acid sequence of an antibody described herein. In one embodiment, an antibody that binds to a KLK2 epitope comprises an amino acid sequence that is at least 75% identical to the amino acid sequence of an antibody described herein. In one embodiment, an antibody that binds to a KLK2 epitope comprises an amino acid sequence that is at least 80% identical to the amino acid sequence of an antibody described herein. In one embodiment, an antibody that binds to a KLK2 epitope comprises an amino acid sequence that is at least 90% identical to the amino acid sequence of an antibody described herein. In one embodiment, an antibody that binds to a KLK2 epitope comprises an amino acid sequence that is at least 95% identical to the amino acid sequence of an antibody described herein. In one embodiment, an antibody that binds to a KLK2 comprises an amino acid sequence that is at least 99% identical to the amino acid sequence of an antibody described herein.

In some embodiments, provided herein is an antibody that binds to a KLK2 epitope and comprises a VL region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 19, a VH region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 19, a light chain comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 21, and/or a heavy chain comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 21.

In some embodiments, provided herein is an antibody that binds to a KLK2 epitope and comprises a VL region comprising an amino acid sequence that is at least 35% identical to an amino acid sequence provided in Table 19. In some embodiments, provided herein is an antibody that binds to a KLK2 epitope and comprises a VL region comprising an amino acid sequence that is at least 40% identical to an amino acid sequence provided in Table 19. In some embodiments, provided herein is an antibody that binds to a KLK2 epitope and comprises a VL region comprising an amino acid sequence that is at least 45% identical to an amino acid sequence provided in Table 19. In some embodiments, provided herein is an antibody that binds to a KLK2 epitope and comprises a VL region comprising an amino acid sequence that is at least 50% identical to an amino acid sequence provided in Table 19. In some embodiments, provided herein is an antibody that binds to a KLK2 epitope and comprises a VL region comprising an amino acid sequence that is at least 55% identical to an amino acid sequence provided in Table 19. In some embodiments, provided herein is an antibody that binds to a KLK2 epitope and comprises a VL region comprising an amino acid sequence that is at least 60% identical to an amino acid sequence provided in Table 19. In some embodiments, provided herein is an antibody that binds to a KLK2 epitope and comprises a VL region comprising an amino acid sequence that is at least 65% identical to an amino acid sequence provided in Table 19. In some embodiments, provided herein is an antibody that binds to a KLK2 epitope and comprises a VL region comprising an amino acid sequence that is at least 70% identical to an amino acid sequence provided in Table 19. In some embodiments, provided herein is an antibody that binds to a KLK2 epitope and comprises a VL region comprising an amino acid sequence that is at least 75% identical to an amino acid sequence provided in Table 19. In some embodiments, provided herein is an antibody that binds to a KLK2 epitope and comprises a VL region comprising an amino acid sequence that is at least 80% identical to an amino acid sequence provided in Table 19. In some embodiments, provided herein is an antibody that binds to a KLK2 epitope and comprises a VL region comprising an amino acid sequence that is at least 85% identical to an amino acid sequence provided in Table 19. In some embodiments, provided herein is an antibody that binds to a KLK2 epitope and comprises a VL region comprising an amino acid sequence that is at least 90% identical to an amino acid sequence provided in Table 19. In some embodiments, provided herein is an antibody that binds to a KLK2 epitope and comprises a VL region comprising an amino acid sequence that is at least 95% identical to an amino acid sequence provided in Table 19. In some embodiments, provided herein is an antibody that binds to a KLK2 epitope and comprises a VL region comprising an amino acid sequence that is at least 99% identical to an amino acid sequence provided herein and Table 19.

In some embodiments, provided herein is an antibody that binds to a KLK2 epitope and comprises a VH region comprising an amino acid sequence that is at least 35% identical to an amino acid sequence provided in Table 19. In some embodiments, provided herein is an antibody that binds to a KLK2 epitope and comprises a VH region comprising an amino acid sequence that is at least 40% identical to an amino acid sequence provided in Table 19. In some embodiments, provided herein is an antibody that binds to a KLK2 epitope and comprises a VH region comprising an amino acid sequence that is at least 45% identical to an amino acid sequence provided in Table 19. In some embodiments, provided herein is an antibody that binds to a KLK2 epitope and comprises a VH region comprising an amino acid sequence that is at least 50% identical to an amino acid sequence provided in Table 19. In some embodiments, provided herein is an antibody that binds to a KLK2 epitope and comprises a VH region comprising an amino acid sequence that is at least 55% identical to an amino acid sequence provided in Table 19. In some embodiments, provided herein is an antibody that binds to a KLK2 epitope and comprises a VH region comprising an amino acid sequence that is at least 60% identical to an amino acid sequence provided in Table 19. In some embodiments, provided herein is an antibody that binds to a KLK2 epitope and comprises a VH region comprising an amino acid sequence that is at least 65% identical to an amino acid sequence provided in Table 19. In some embodiments, provided herein is an antibody that binds to a KLK2 epitope and comprises a VH region comprising an amino acid sequence that is at least 70% identical to an amino acid sequence provided in Table 19. In some embodiments, provided herein is an antibody that binds to a KLK2 epitope and comprises a VH region comprising an amino acid sequence that is at least 75% identical to an amino acid sequence provided in Table 19. In some embodiments, provided herein is an antibody that binds to a KLK2 epitope and comprises a VH region comprising an amino acid sequence that is at least 80% identical to an amino acid sequence provided in Table 19. In some embodiments, provided herein is an antibody that binds to a KLK2 epitope and comprises a VH region comprising an amino acid sequence that is at least 85% identical to an amino acid sequence provided in Table 19. In some embodiments, provided herein is an antibody that binds to a KLK2 epitope and comprises a VH region comprising an amino acid sequence that is at least 90% identical to an amino acid sequence provided in Table 19. In some embodiments, provided herein is an antibody that binds to a KLK2 epitope and comprises a VH region comprising an amino acid sequence that is at least 95% identical to an amino acid sequence provided in Table 19. In some embodiments, provided herein is an antibody that binds to a KLK2 epitope and comprises a VH region comprising an amino acid sequence that is at least 99% identical to an amino acid sequence provided herein and Table 19.

In some embodiments, provided herein is an antibody that binds to a KLK2 epitope and comprises a light chain comprising an amino acid sequence that is at least 35% identical to an amino acid sequence provided in Table 21. In some embodiments, provided herein is an antibody that binds to a KLK2 epitope and comprises a light chain comprising an amino acid sequence that is at least 40% identical to an amino acid sequence provided in Table 21. In some embodiments, provided herein is an antibody that binds to a KLK2 epitope and comprises a light chain comprising an amino acid sequence that is at least 45% identical to an amino acid sequence provided in Table 21. In some embodiments, provided herein is an antibody that binds to a KLK2 epitope and comprises a light chain comprising an amino acid sequence that is at least 50% identical to an amino acid sequence provided in Table 21. In some embodiments, provided herein is an antibody that binds to a KLK2 epitope and comprises a light chain comprising an amino acid sequence that is at least 55% identical to an amino acid sequence provided in Table 21. In some embodiments, provided herein is an antibody that binds to a KLK2 epitope and comprises a light chain comprising an amino acid sequence that is at least 60% identical to an amino acid sequence provided in Table 21. In some embodiments, provided herein is an antibody that binds to a KLK2 epitope and comprises a light chain comprising an amino acid sequence that is at least 65% identical to an amino acid sequence provided in Table 21. In some embodiments, provided herein is an antibody that binds to a KLK2 epitope and comprises a light chain comprising an amino acid sequence that is at least 70% identical to an amino acid sequence provided in Table 21. In some embodiments, provided herein is an antibody that binds to a KLK2 epitope and comprises a light chain comprising an amino acid sequence that is at least 75% identical to an amino acid sequence provided in Table 21. In some embodiments, provided herein is an antibody that binds to a KLK2 epitope and comprises a light chain comprising an amino acid sequence that is at least 80% identical to an amino acid sequence provided in Table 21. In some embodiments, provided herein is an antibody that binds to a KLK2 epitope and comprises a light chain comprising an amino acid sequence that is at least 85% identical to an amino acid sequence provided in Table 21. In some embodiments, provided herein is an antibody that binds to a KLK2 epitope and comprises a light chain comprising an amino acid sequence that is at least 90% identical to an amino acid sequence provided in Table 21. In some embodiments, provided herein is an antibody that binds to a KLK2 epitope and comprises a light chain comprising an amino acid sequence that is at least 95% identical to an amino acid sequence provided in Table 21. In some embodiments, provided herein is an antibody that binds to a KLK2 epitope and comprises a light chain comprising an amino acid sequence that is at least 99% identical to an amino acid sequence provided herein and Table 21.

In some embodiments, provided herein is an antibody that binds to a KLK2 epitope and comprises a heavy chain comprising an amino acid sequence that is at least 35% identical to an amino acid sequence provided in Table 21. In some embodiments, provided herein is an antibody that binds to a KLK2 epitope and comprises a heavy chain comprising an amino acid sequence that is at least 40% identical to an amino acid sequence provided in Table 21. In some embodiments, provided herein is an antibody that binds to a KLK2 epitope and comprises a heavy chain comprising an amino acid sequence that is at least 45% identical to an amino acid sequence provided in Table 21. In some embodiments, provided herein is an antibody that binds to a KLK2 epitope and comprises a heavy chain comprising an amino acid sequence that is at least 50% identical to an amino acid sequence provided in Table 21. In some embodiments, provided herein is an antibody that binds to a KLK2 epitope and comprises a heavy chain comprising an amino acid sequence that is at least 55% identical to an amino acid sequence provided in Table 21. In some embodiments, provided herein is an antibody that binds to a KLK2 epitope and comprises a heavy chain comprising an amino acid sequence that is at least 60% identical to an amino acid sequence provided in Table 21. In some embodiments, provided herein is an antibody that binds to a KLK2 epitope and comprises a heavy chain comprising an amino acid sequence that is at least 65% identical to an amino acid sequence provided in Table 21. In some embodiments, provided herein is an antibody that binds to a KLK2 epitope and comprises a heavy chain comprising an amino acid sequence that is at least 70% identical to an amino acid sequence provided in Table 21. In some embodiments, provided herein is an antibody that binds to a KLK2 epitope and comprises a heavy chain comprising an amino acid sequence that is at least 75% identical to an amino acid sequence provided in Table 21. In some embodiments, provided herein is an antibody that binds to a KLK2 epitope and comprises a heavy chain comprising an amino acid sequence that is at least 80% identical to an amino acid sequence provided in Table 21. In some embodiments, provided herein is an antibody that binds to a KLK2 epitope and comprises a heavy chain comprising an amino acid sequence that is at least 85% identical to an amino acid sequence provided in Table 21. In some embodiments, provided herein is an antibody that binds to a KLK2 epitope and comprises a heavy chain comprising an amino acid sequence that is at least 90% identical to an amino acid sequence provided in Table 21. In some embodiments, provided herein is an antibody that binds to a KLK2 epitope and comprises a heavy chain comprising an amino acid sequence that is at least 95% identical to an amino acid sequence provided in Table 21. In some embodiments, provided herein is an antibody that binds to a KLK2 epitope and comprises a heavy chain comprising an amino acid sequence that is at least 99% identical to an amino acid sequence provided herein and Table 21.

In yet another embodiment, an antibody that binds to a KLK2 epitope comprises a VL CDR and/or a VH CDR amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a VL CDR amino acid sequence and/or a VH CDR amino acid sequence contained in the VL and VH sequences in Table 19 below.

In yet another embodiment, an antibody that binds to a KLK2 epitope comprises a VL CDR and/or a VH CDR amino acid sequence that is at least 35% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 40% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 45% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 50% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 55% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 60% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 65% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 70% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 75% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 80% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 85% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 90% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 95% identical to a VL CDR amino acid sequence and/or a VH CDR amino acid sequence contained in the VL and VH sequences in Table 19. In yet another embodiment, an antibody that binds to a KLK2 epitope comprises a VL CDR and/or a VH CDR amino acid sequence that is at least 99% identical to a VL CDR amino acid sequence and/or a VH CDR amino acid sequence contained in the VL and VH sequences in Table 19.

In some embodiments, provided herein is an antibody that binds to an HLA-G epitope and comprises a VL region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 24, a VH region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 24, a light chain comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 25, and/or a heavy chain comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 25.

In some embodiments, provided herein is an antibody that binds to an HLA-G epitope and comprises a VL region comprising an amino acid sequence that is at least 35% identical to an amino acid sequence provided in Table 24. In some embodiments, provided herein is an antibody that binds to an HLA-G epitope and comprises a VL region comprising an amino acid sequence that is at least 40% identical to an amino acid sequence provided in Table 24. In some embodiments, provided herein is an antibody that binds to an HLA-G epitope and comprises a VL region comprising an amino acid sequence that is at least 45% identical to an amino acid sequence provided in Table 24. In some embodiments, provided herein is an antibody that binds to an HLA-G epitope and comprises a VL region comprising an amino acid sequence that is at least 50% identical to an amino acid sequence provided in Table 24. In some embodiments, provided herein is an antibody that binds to an HLA-G epitope and comprises a VL region comprising an amino acid sequence that is at least 55% identical to an amino acid sequence provided in Table 24. In some embodiments, provided herein is an antibody that binds to an HLA-G epitope and comprises a VL region comprising an amino acid sequence that is at least 60% identical to an amino acid sequence provided in Table 24. In some embodiments, provided herein is an antibody that binds to an HLA-G epitope and comprises a VL region comprising an amino acid sequence that is at least 65% identical to an amino acid sequence provided in Table 24. In some embodiments, provided herein is an antibody that binds to an HLA-G epitope and comprises a VL region comprising an amino acid sequence that is at least 70% identical to an amino acid sequence provided in Table 24. In some embodiments, provided herein is an antibody that binds to an HLA-G epitope and comprises a VL region comprising an amino acid sequence that is at least 75% identical to an amino acid sequence provided in Table 24. In some embodiments, provided herein is an antibody that binds to an HLA-G epitope and comprises a VL region comprising an amino acid sequence that is at least 80% identical to an amino acid sequence provided in Table 24. In some embodiments, provided herein is an antibody that binds to an HLA-G epitope and comprises a VL region comprising an amino acid sequence that is at least 85% identical to an amino acid sequence provided in Table 24. In some embodiments, provided herein is an antibody that binds to an HLA-G epitope and comprises a VL region comprising an amino acid sequence that is at least 90% identical to an amino acid sequence provided in Table 24. In some embodiments, provided herein is an antibody that binds to an HLA-G epitope and comprises a VL region comprising an amino acid sequence that is at least 95% identical to an amino acid sequence provided in Table 24. In some embodiments, provided herein is an antibody that binds to an HLA-G epitope and comprises a VL region comprising an amino acid sequence that is at least 99% identical to an amino acid sequence provided herein and Table 24.

In some embodiments, provided herein is an antibody that binds to an HLA-G epitope and comprises a VH region comprising an amino acid sequence that is at least 35% identical to an amino acid sequence provided in Table 24. In some embodiments, provided herein is an antibody that binds to an HLA-G epitope and comprises a VH region comprising an amino acid sequence that is at least 40% identical to an amino acid sequence provided in Table 24. In some embodiments, provided herein is an antibody that binds to an HLA-G epitope and comprises a VH region comprising an amino acid sequence that is at least 45% identical to an amino acid sequence provided in Table 24. In some embodiments, provided herein is an antibody that binds to an HLA-G epitope and comprises a VH region comprising an amino acid sequence that is at least 50% identical to an amino acid sequence provided in Table 24. In some embodiments, provided herein is an antibody that binds to an HLA-G epitope and comprises a VH region comprising an amino acid sequence that is at least 55% identical to an amino acid sequence provided in Table 24. In some embodiments, provided herein is an antibody that binds to an HLA-G epitope and comprises a VH region comprising an amino acid sequence that is at least 60% identical to an amino acid sequence provided in Table 24. In some embodiments, provided herein is an antibody that binds to an HLA-G epitope and comprises a VH region comprising an amino acid sequence that is at least 65% identical to an amino acid sequence provided in Table 24. In some embodiments, provided herein is an antibody that binds to an HLA-G epitope and comprises a VH region comprising an amino acid sequence that is at least 70% identical to an amino acid sequence provided in Table 24. In some embodiments, provided herein is an antibody that binds to an HLA-G epitope and comprises a VH region comprising an amino acid sequence that is at least 75% identical to an amino acid sequence provided in Table 24. In some embodiments, provided herein is an antibody that binds to an HLA-G epitope and comprises a VH region comprising an amino acid sequence that is at least 80% identical to an amino acid sequence provided in Table 24. In some embodiments, provided herein is an antibody that binds to an HLA-G epitope and comprises a VH region comprising an amino acid sequence that is at least 85% identical to an amino acid sequence provided in Table 24. In some embodiments, provided herein is an antibody that binds to an HLA-G epitope and comprises a VH region comprising an amino acid sequence that is at least 90% identical to an amino acid sequence provided in Table 24. In some embodiments, provided herein is an antibody that binds to an HLA-G epitope and comprises a VH region comprising an amino acid sequence that is at least 95% identical to an amino acid sequence provided in Table 24. In some embodiments, provided herein is an antibody that binds to an HLA-G epitope and comprises a VH region comprising an amino acid sequence that is at least 99% identical to an amino acid sequence provided herein and Table 24.

In some embodiments, provided herein is an antibody that binds to an HLA-G epitope and comprises a light chain comprising an amino acid sequence that is at least 35% identical to an amino acid sequence provided in Table 25. In some embodiments, provided herein is an antibody that binds to an HLA-G epitope and comprises a light chain comprising an amino acid sequence that is at least 40% identical to an amino acid sequence provided in Table 25. In some embodiments, provided herein is an antibody that binds to an HLA-G epitope and comprises a light chain comprising an amino acid sequence that is at least 45% identical to an amino acid sequence provided in Table 25. In some embodiments, provided herein is an antibody that binds to an HLA-G epitope and comprises a light chain comprising an amino acid sequence that is at least 50% identical to an amino acid sequence provided in Table 25. In some embodiments, provided herein is an antibody that binds to an HLA-G epitope and comprises a light chain comprising an amino acid sequence that is at least 55% identical to an amino acid sequence provided in Table 25. In some embodiments, provided herein is an antibody that binds to an HLA-G epitope and comprises a light chain comprising an amino acid sequence that is at least 60% identical to an amino acid sequence provided in Table 25. In some embodiments, provided herein is an antibody that binds to an HLA-G epitope and comprises a light chain comprising an amino acid sequence that is at least 65% identical to an amino acid sequence provided in Table 25. In some embodiments, provided herein is an antibody that binds to an HLA-G epitope and comprises a light chain comprising an amino acid sequence that is at least 70% identical to an amino acid sequence provided in Table 25. In some embodiments, provided herein is an antibody that binds to an HLA-G epitope and comprises a light chain comprising an amino acid sequence that is at least 75% identical to an amino acid sequence provided in Table 25. In some embodiments, provided herein is an antibody that binds to an HLA-G epitope and comprises a light chain comprising an amino acid sequence that is at least 80% identical to an amino acid sequence provided in Table 25. In some embodiments, provided herein is an antibody that binds to an HLA-G epitope and comprises a light chain comprising an amino acid sequence that is at least 85% identical to an amino acid sequence provided in Table 25. In some embodiments, provided herein is an antibody that binds to an HLA-G epitope and comprises a light chain comprising an amino acid sequence that is at least 90% identical to an amino acid sequence provided in Table 25. In some embodiments, provided herein is an antibody that binds to an HLA-G epitope and comprises a light chain comprising an amino acid sequence that is at least 95% identical to an amino acid sequence provided in Table 25. In some embodiments, provided herein is an antibody that binds to an HLA-G epitope and comprises a light chain comprising an amino acid sequence that is at least 99% identical to an amino acid sequence provided herein and Table 25.

In some embodiments, provided herein is an antibody that binds to an HLA-G epitope and comprises a heavy chain comprising an amino acid sequence that is at least 35% identical to an amino acid sequence provided in Table 25. In some embodiments, provided herein is an antibody that binds to an HLA-G epitope and comprises a heavy chain comprising an amino acid sequence that is at least 40% identical to an amino acid sequence provided in Table 25. In some embodiments, provided herein is an antibody that binds to an HLA-G epitope and comprises a heavy chain comprising an amino acid sequence that is at least 45% identical to an amino acid sequence provided in Table 25. In some embodiments, provided herein is an antibody that binds to an HLA-G epitope and comprises a heavy chain comprising an amino acid sequence that is at least 50% identical to an amino acid sequence provided in Table 25. In some embodiments, provided herein is an antibody that binds to an HLA-G epitope and comprises a heavy chain comprising an amino acid sequence that is at least 55% identical to an amino acid sequence provided in Table 25. In some embodiments, provided herein is an antibody that binds to an HLA-G epitope and comprises a heavy chain comprising an amino acid sequence that is at least 60% identical to an amino acid sequence provided in Table 25. In some embodiments, provided herein is an antibody that binds to an HLA-G epitope and comprises a heavy chain comprising an amino acid sequence that is at least 65% identical to an amino acid sequence provided in Table 25. In some embodiments, provided herein is an antibody that binds to an HLA-G epitope and comprises a heavy chain comprising an amino acid sequence that is at least 70% identical to an amino acid sequence provided in Table 25. In some embodiments, provided herein is an antibody that binds to an HLA-G epitope and comprises a heavy chain comprising an amino acid sequence that is at least 75% identical to an amino acid sequence provided in Table 25. In some embodiments, provided herein is an antibody that binds to an HLA-G epitope and comprises a heavy chain comprising an amino acid sequence that is at least 80% identical to an amino acid sequence provided in Table 25. In some embodiments, provided herein is an antibody that binds to an HLA-G epitope and comprises a heavy chain comprising an amino acid sequence that is at least 85% identical to an amino acid sequence provided in Table 25. In some embodiments, provided herein is an antibody that binds to an HLA-G epitope and comprises a heavy chain comprising an amino acid sequence that is at least 90% identical to an amino acid sequence provided in Table 25. In some embodiments, provided herein is an antibody that binds to an HLA-G epitope and comprises a heavy chain comprising an amino acid sequence that is at least 95% identical to an amino acid sequence provided in Table 25. In some embodiments, provided herein is an antibody that binds to an HLA-G epitope and comprises a heavy chain comprising an amino acid sequence that is at least 99% identical to an amino acid sequence provided herein and Table 25.

In yet another embodiment, an antibody that binds to an HLA-G epitope comprises a VL CDR and/or a VH CDR amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a VL CDR amino acid sequence and/or a VH CDR amino acid sequence contained in the VL and VH sequences in Table 24 below.

In yet another embodiment, an antibody that binds to an HLA-G epitope comprises a VL CDR and/or a VH CDR amino acid sequence that is at least 35% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 40% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 45% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 50% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 55% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 60% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 65% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 70% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 75% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 80% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 85% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 90% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 95% identical to a VL CDR amino acid sequence and/or a VH CDR amino acid sequence contained in the VL and VH sequences in Table 24. In yet another embodiment, an antibody that binds to an HLA-G epitope comprises a VL CDR and/or a VH CDR amino acid sequence that is at least 99% identical to a VL CDR amino acid sequence and/or a VH CDR amino acid sequence contained in the VL and VH sequences in Table 24.

In some embodiments, provided herein is an antibody that binds to an PSMA epitope and comprises a VL region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 28, a VH region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 28, a light chain comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 30, and/or a heavy chain comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 30.

In some embodiments, provided herein is an antibody that binds to an PSMA epitope and comprises a VL region comprising an amino acid sequence that is at least 35% identical to an amino acid sequence provided in Table 28. In some embodiments, provided herein is an antibody that binds to an PSMA epitope and comprises a VL region comprising an amino acid sequence that is at least 40% identical to an amino acid sequence provided in Table 28. In some embodiments, provided herein is an antibody that binds to an PSMA epitope and comprises a VL region comprising an amino acid sequence that is at least 45% identical to an amino acid sequence provided in Table 28. In some embodiments, provided herein is an antibody that binds to an PSMA epitope and comprises a VL region comprising an amino acid sequence that is at least 50% identical to an amino acid sequence provided in Table 28. In some embodiments, provided herein is an antibody that binds to an PSMA epitope and comprises a VL region comprising an amino acid sequence that is at least 55% identical to an amino acid sequence provided in Table 28. In some embodiments, provided herein is an antibody that binds to an PSMA epitope and comprises a VL region comprising an amino acid sequence that is at least 60% identical to an amino acid sequence provided in Table 28. In some embodiments, provided herein is an antibody that binds to an PSMA epitope and comprises a VL region comprising an amino acid sequence that is at least 65% identical to an amino acid sequence provided in Table 28. In some embodiments, provided herein is an antibody that binds to an PSMA epitope and comprises a VL region comprising an amino acid sequence that is at least 70% identical to an amino acid sequence provided in Table 28. In some embodiments, provided herein is an antibody that binds to an PSMA epitope and comprises a VL region comprising an amino acid sequence that is at least 75% identical to an amino acid sequence provided in Table 28. In some embodiments, provided herein is an antibody that binds to an PSMA epitope and comprises a VL region comprising an amino acid sequence that is at least 80% identical to an amino acid sequence provided in Table 28. In some embodiments, provided herein is an antibody that binds to an PSMA epitope and comprises a VL region comprising an amino acid sequence that is at least 85% identical to an amino acid sequence provided in Table 28. In some embodiments, provided herein is an antibody that binds to an PSMA epitope and comprises a VL region comprising an amino acid sequence that is at least 90% identical to an amino acid sequence provided in Table 28. In some embodiments, provided herein is an antibody that binds to an PSMA epitope and comprises a VL region comprising an amino acid sequence that is at least 95% identical to an amino acid sequence provided in Table 28. In some embodiments, provided herein is an antibody that binds to an PSMA epitope and comprises a VL region comprising an amino acid sequence that is at least 99% identical to an amino acid sequence provided herein and Table 28.

In some embodiments, provided herein is an antibody that binds to an PSMA epitope and comprises a VH region comprising an amino acid sequence that is at least 35% identical to an amino acid sequence provided in Table 28. In some embodiments, provided herein is an antibody that binds to an PSMA epitope and comprises a VH region comprising an amino acid sequence that is at least 40% identical to an amino acid sequence provided in Table 28. In some embodiments, provided herein is an antibody that binds to an PSMA epitope and comprises a VH region comprising an amino acid sequence that is at least 45% identical to an amino acid sequence provided in Table 28. In some embodiments, provided herein is an antibody that binds to an PSMA epitope and comprises a VH region comprising an amino acid sequence that is at least 50% identical to an amino acid sequence provided in Table 28. In some embodiments, provided herein is an antibody that binds to an PSMA epitope and comprises a VH region comprising an amino acid sequence that is at least 55% identical to an amino acid sequence provided in Table 28. In some embodiments, provided herein is an antibody that binds to an PSMA epitope and comprises a VH region comprising an amino acid sequence that is at least 60% identical to an amino acid sequence provided in Table 28. In some embodiments, provided herein is an antibody that binds to an PSMA epitope and comprises a VH region comprising an amino acid sequence that is at least 65% identical to an amino acid sequence provided in Table 28. In some embodiments, provided herein is an antibody that binds to an PSMA epitope and comprises a VH region comprising an amino acid sequence that is at least 70% identical to an amino acid sequence provided in Table 28. In some embodiments, provided herein is an antibody that binds to an PSMA epitope and comprises a VH region comprising an amino acid sequence that is at least 75% identical to an amino acid sequence provided in Table 28. In some embodiments, provided herein is an antibody that binds to an PSMA epitope and comprises a VH region comprising an amino acid sequence that is at least 80% identical to an amino acid sequence provided in Table 28. In some embodiments, provided herein is an antibody that binds to an PSMA epitope and comprises a VH region comprising an amino acid sequence that is at least 85% identical to an amino acid sequence provided in Table 28. In some embodiments, provided herein is an antibody that binds to an PSMA epitope and comprises a VH region comprising an amino acid sequence that is at least 90% identical to an amino acid sequence provided in Table 28. In some embodiments, provided herein is an antibody that binds to an PSMA epitope and comprises a VH region comprising an amino acid sequence that is at least 95% identical to an amino acid sequence provided in Table 28. In some embodiments, provided herein is an antibody that binds to an PSMA epitope and comprises a VH region comprising an amino acid sequence that is at least 99% identical to an amino acid sequence provided herein and Table 28.

In some embodiments, provided herein is an antibody that binds to an PSMA epitope and comprises a light chain comprising an amino acid sequence that is at least 35% identical to an amino acid sequence provided in Table 30. In some embodiments, provided herein is an antibody that binds to an PSMA epitope and comprises a light chain comprising an amino acid sequence that is at least 40% identical to an amino acid sequence provided in Table 30. In some embodiments, provided herein is an antibody that binds to an PSMA epitope and comprises a light chain comprising an amino acid sequence that is at least 45% identical to an amino acid sequence provided in Table 30. In some embodiments, provided herein is an antibody that binds to an PSMA epitope and comprises a light chain comprising an amino acid sequence that is at least 50% identical to an amino acid sequence provided in Table 30. In some embodiments, provided herein is an antibody that binds to an PSMA epitope and comprises a light chain comprising an amino acid sequence that is at least 55% identical to an amino acid sequence provided in Table 30. In some embodiments, provided herein is an antibody that binds to an PSMA epitope and comprises a light chain comprising an amino acid sequence that is at least 60% identical to an amino acid sequence provided in Table 30. In some embodiments, provided herein is an antibody that binds to an PSMA epitope and comprises a light chain comprising an amino acid sequence that is at least 65% identical to an amino acid sequence provided in Table 30. In some embodiments, provided herein is an antibody that binds to an PSMA epitope and comprises a light chain comprising an amino acid sequence that is at least 70% identical to an amino acid sequence provided in Table 30. In some embodiments, provided herein is an antibody that binds to an PSMA epitope and comprises a light chain comprising an amino acid sequence that is at least 75% identical to an amino acid sequence provided in Table 30. In some embodiments, provided herein is an antibody that binds to an PSMA epitope and comprises a light chain comprising an amino acid sequence that is at least 80% identical to an amino acid sequence provided in Table 30. In some embodiments, provided herein is an antibody that binds to an PSMA epitope and comprises a light chain comprising an amino acid sequence that is at least 85% identical to an amino acid sequence provided in Table 30. In some embodiments, provided herein is an antibody that binds to an PSMA epitope and comprises a light chain comprising an amino acid sequence that is at least 90% identical to an amino acid sequence provided in Table 30. In some embodiments, provided herein is an antibody that binds to an PSMA epitope and comprises a light chain comprising an amino acid sequence that is at least 95% identical to an amino acid sequence provided in Table 30. In some embodiments, provided herein is an antibody that binds to an PSMA epitope and comprises a light chain comprising an amino acid sequence that is at least 99% identical to an amino acid sequence provided herein and Table 30.

In some embodiments, provided herein is an antibody that binds to an PSMA epitope and comprises a heavy chain comprising an amino acid sequence that is at least 35% identical to an amino acid sequence provided in Table 30. In some embodiments, provided herein is an antibody that binds to an PSMA epitope and comprises a heavy chain comprising an amino acid sequence that is at least 40% identical to an amino acid sequence provided in Table 30. In some embodiments, provided herein is an antibody that binds to an PSMA epitope and comprises a heavy chain comprising an amino acid sequence that is at least 45% identical to an amino acid sequence provided in Table 30. In some embodiments, provided herein is an antibody that binds to an PSMA epitope and comprises a heavy chain comprising an amino acid sequence that is at least 50% identical to an amino acid sequence provided in Table 30. In some embodiments, provided herein is an antibody that binds to an PSMA epitope and comprises a heavy chain comprising an amino acid sequence that is at least 55% identical to an amino acid sequence provided in Table 30. In some embodiments, provided herein is an antibody that binds to an PSMA epitope and comprises a heavy chain comprising an amino acid sequence that is at least 60% identical to an amino acid sequence provided in Table 30. In some embodiments, provided herein is an antibody that binds to an PSMA epitope and comprises a heavy chain comprising an amino acid sequence that is at least 65% identical to an amino acid sequence provided in Table 30. In some embodiments, provided herein is an antibody that binds to an PSMA epitope and comprises a heavy chain comprising an amino acid sequence that is at least 70% identical to an amino acid sequence provided in Table 30. In some embodiments, provided herein is an antibody that binds to an PSMA epitope and comprises a heavy chain comprising an amino acid sequence that is at least 75% identical to an amino acid sequence provided in Table 30. In some embodiments, provided herein is an antibody that binds to an PSMA epitope and comprises a heavy chain comprising an amino acid sequence that is at least 80% identical to an amino acid sequence provided in Table 30. In some embodiments, provided herein is an antibody that binds to an PSMA epitope and comprises a heavy chain comprising an amino acid sequence that is at least 85% identical to an amino acid sequence provided in Table 30. In some embodiments, provided herein is an antibody that binds to an PSMA epitope and comprises a heavy chain comprising an amino acid sequence that is at least 90% identical to an amino acid sequence provided in Table 30. In some embodiments, provided herein is an antibody that binds to an PSMA epitope and comprises a heavy chain comprising an amino acid sequence that is at least 95% identical to an amino acid sequence provided in Table 30. In some embodiments, provided herein is an antibody that binds to an PSMA epitope and comprises a heavy chain comprising an amino acid sequence that is at least 99% identical to an amino acid sequence provided herein and Table 30.

In yet another embodiment, an antibody that binds to an PSMA epitope comprises a VL CDR and/or a VH CDR amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a VL CDR amino acid sequence and/or a VH CDR amino acid sequence contained in the VL and VH sequences in Table 28 below.

In yet another embodiment, an antibody that binds to an PSMA epitope comprises a VL CDR and/or a VH CDR amino acid sequence that is at least 35% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 40% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 45% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 50% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 55% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 60% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 65% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 70% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 75% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 80% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 85% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 90% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 95% identical to a VL CDR amino acid sequence and/or a VH CDR amino acid sequence contained in the VL and VH sequences in Table 28. In yet another embodiment, an antibody that binds to an PSMA epitope comprises a VL CDR and/or a VH CDR amino acid sequence that is at least 99% identical to a VL CDR amino acid sequence and/or a VH CDR amino acid sequence contained in the VL and VH sequences in Table 28.

In some embodiments, provided herein is an antibody that binds to an BCMA epitope and comprises a VL region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 33, a VH region comprising an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence provided in Table 33.

In some embodiments, provided herein is an antibody that binds to an BCMA epitope and comprises a VL region comprising an amino acid sequence that is at least 35% identical to an amino acid sequence provided in Table 33. In some embodiments, provided herein is an antibody that binds to an BCMA epitope and comprises a VL region comprising an amino acid sequence that is at least 40% identical to an amino acid sequence provided in Table 33. In some embodiments, provided herein is an antibody that binds to an BCMA epitope and comprises a VL region comprising an amino acid sequence that is at least 45% identical to an amino acid sequence provided in Table 33. In some embodiments, provided herein is an antibody that binds to an BCMA epitope and comprises a VL region comprising an amino acid sequence that is at least 50% identical to an amino acid sequence provided in Table 33. In some embodiments, provided herein is an antibody that binds to an BCMA epitope and comprises a VL region comprising an amino acid sequence that is at least 55% identical to an amino acid sequence provided in Table 33. In some embodiments, provided herein is an antibody that binds to an BCMA epitope and comprises a VL region comprising an amino acid sequence that is at least 60% identical to an amino acid sequence provided in Table 33. In some embodiments, provided herein is an antibody that binds to an BCMA epitope and comprises a VL region comprising an amino acid sequence that is at least 65% identical to an amino acid sequence provided in Table 33. In some embodiments, provided herein is an antibody that binds to an BCMA epitope and comprises a VL region comprising an amino acid sequence that is at least 70% identical to an amino acid sequence provided in Table 33. In some embodiments, provided herein is an antibody that binds to an BCMA epitope and comprises a VL region comprising an amino acid sequence that is at least 75% identical to an amino acid sequence provided in Table 33. In some embodiments, provided herein is an antibody that binds to an BCMA epitope and comprises a VL region comprising an amino acid sequence that is at least 80% identical to an amino acid sequence provided in Table 33. In some embodiments, provided herein is an antibody that binds to an BCMA epitope and comprises a VL region comprising an amino acid sequence that is at least 85% identical to an amino acid sequence provided in Table 33. In some embodiments, provided herein is an antibody that binds to an BCMA epitope and comprises a VL region comprising an amino acid sequence that is at least 90% identical to an amino acid sequence provided in Table 33. In some embodiments, provided herein is an antibody that binds to an BCMA epitope and comprises a VL region comprising an amino acid sequence that is at least 95% identical to an amino acid sequence provided in Table 33. In some embodiments, provided herein is an antibody that binds to an BCMA epitope and comprises a VL region comprising an amino acid sequence that is at least 99% identical to an amino acid sequence provided herein and Table 33.

In some embodiments, provided herein is an antibody that binds to an BCMA epitope and comprises a VH region comprising an amino acid sequence that is at least 35% identical to an amino acid sequence provided in Table 33. In some embodiments, provided herein is an antibody that binds to an BCMA epitope and comprises a VH region comprising an amino acid sequence that is at least 40% identical to an amino acid sequence provided in Table 33. In some embodiments, provided herein is an antibody that binds to an BCMA epitope and comprises a VH region comprising an amino acid sequence that is at least 45% identical to an amino acid sequence provided in Table 33. In some embodiments, provided herein is an antibody that binds to an BCMA epitope and comprises a VH region comprising an amino acid sequence that is at least 50% identical to an amino acid sequence provided in Table 33. In some embodiments, provided herein is an antibody that binds to an BCMA epitope and comprises a VH region comprising an amino acid sequence that is at least 55% identical to an amino acid sequence provided in Table 33. In some embodiments, provided herein is an antibody that binds to an BCMA epitope and comprises a VH region comprising an amino acid sequence that is at least 60% identical to an amino acid sequence provided in Table 33. In some embodiments, provided herein is an antibody that binds to an BCMA epitope and comprises a VH region comprising an amino acid sequence that is at least 65% identical to an amino acid sequence provided in Table 33. In some embodiments, provided herein is an antibody that binds to an BCMA epitope and comprises a VH region comprising an amino acid sequence that is at least 70% identical to an amino acid sequence provided in Table 33. In some embodiments, provided herein is an antibody that binds to an BCMA epitope and comprises a VH region comprising an amino acid sequence that is at least 75% identical to an amino acid sequence provided in Table 33. In some embodiments, provided herein is an antibody that binds to an BCMA epitope and comprises a VH region comprising an amino acid sequence that is at least 80% identical to an amino acid sequence provided in Table 33. In some embodiments, provided herein is an antibody that binds to an BCMA epitope and comprises a VH region comprising an amino acid sequence that is at least 85% identical to an amino acid sequence provided in Table 33. In some embodiments, provided herein is an antibody that binds to an BCMA epitope and comprises a VH region comprising an amino acid sequence that is at least 90% identical to an amino acid sequence provided in Table 33. In some embodiments, provided herein is an antibody that binds to an BCMA epitope and comprises a VH region comprising an amino acid sequence that is at least 95% identical to an amino acid sequence provided in Table 33. In some embodiments, provided herein is an antibody that binds to an BCMA epitope and comprises a VH region comprising an amino acid sequence that is at least 99% identical to an amino acid sequence provided herein and Table 33.

In some embodiments, provided herein is an antibody that binds to an BCMA epitope and comprises a heavy chain comprising an amino acid sequence that is at least 35% identical to an amino acid sequence provided in Table 34. In some embodiments, provided herein is an antibody that binds to an BCMA epitope and comprises a heavy chain comprising an amino acid sequence that is at least 40% identical to an amino acid sequence provided in Table 34. In some embodiments, provided herein is an antibody that binds to an BCMA epitope and comprises a heavy chain comprising an amino acid sequence that is at least 45% identical to an amino acid sequence provided in Table 34. In some embodiments, provided herein is an antibody that binds to an BCMA epitope and comprises a heavy chain comprising an amino acid sequence that is at least 50% identical to an amino acid sequence provided in Table 34. In some embodiments, provided herein is an antibody that binds to an BCMA epitope and comprises a heavy chain comprising an amino acid sequence that is at least 55% identical to an amino acid sequence provided in Table 34. In some embodiments, provided herein is an antibody that binds to an BCMA epitope and comprises a heavy chain comprising an amino acid sequence that is at least 60% identical to an amino acid sequence provided in Table 34. In some embodiments, provided herein is an antibody that binds to an BCMA epitope and comprises a heavy chain comprising an amino acid sequence that is at least 65% identical to an amino acid sequence provided in Table 34. In some embodiments, provided herein is an antibody that binds to an BCMA epitope and comprises a heavy chain comprising an amino acid sequence that is at least 70% identical to an amino acid sequence provided in Table 34. In some embodiments, provided herein is an antibody that binds to an BCMA epitope and comprises a heavy chain comprising an amino acid sequence that is at least 75% identical to an amino acid sequence provided in Table 34. In some embodiments, provided herein is an antibody that binds to an BCMA epitope and comprises a heavy chain comprising an amino acid sequence that is at least 80% identical to an amino acid sequence provided in Table 34. In some embodiments, provided herein is an antibody that binds to an BCMA epitope and comprises a heavy chain comprising an amino acid sequence that is at least 85% identical to an amino acid sequence provided in Table 34. In some embodiments, provided herein is an antibody that binds to an BCMA epitope and comprises a heavy chain comprising an amino acid sequence that is at least 90% identical to an amino acid sequence provided in Table 34. In some embodiments, provided herein is an antibody that binds to an BCMA epitope and comprises a heavy chain comprising an amino acid sequence that is at least 95% identical to an amino acid sequence provided in Table 34. In some embodiments, provided herein is an antibody that binds to an BCMA epitope and comprises a heavy chain comprising an amino acid sequence that is at least 99% identical to an amino acid sequence provided herein and Table 34.

In yet another embodiment, an antibody that binds to an BCMA epitope comprises a VL CDR and/or a VH CDR amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a VL CDR amino acid sequence and/or a VH CDR amino acid sequence contained in the VL and VH sequences in Table 33 below.

In yet another embodiment, an antibody that binds to an BCMA epitope comprises a VL CDR and/or a VH CDR amino acid sequence that is at least 35% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 40% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 45% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 50% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 55% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 60% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 65% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 70% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 75% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 80% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 85% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 90% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 95% identical to a VL CDR amino acid sequence and/or a VH CDR amino acid sequence contained in the VL and VH sequences in Table 33. In yet another embodiment, an antibody that binds to an BCMA epitope comprises a VL CDR and/or a VH CDR amino acid sequence that is at least 99% identical to a VL CDR amino acid sequence and/or a VH CDR amino acid sequence contained in the VL and VH sequences in Table 33.

### 5.16 Multivalent Antibodies

A multivalent antibody may be internalized (and/or catabolized) faster than a bivalent antibody by a cell expressing an antigen to which the antibodies bind. The antibodies of the present disclosure can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. In certain embodiments, the dimerization domain comprises (or consists of) an Fc region or a hinge region. In this scenario, the antibody will comprise an Fc region and three or more antigen binding sites amino-terminal to the Fc region. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (e.g., two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

### 5.17 Multispecific Antibodies

Multispecific antibodies such as bispecific antibodies are monoclonal antibodies that have binding specificities for at least two different antigens. In certain embodiments, the multispecific antibodies can be constructed based on the sequences of the antibodies provided herein, e.g., the CDR sequences contained in the VL and VH sequences provided herein. In certain embodiments, the multispecific antibodies provided herein are bispecific antibodies. In certain embodiments, bispecific antibodies are mouse, chimeric, human or humanized antibodies. Methods for making multispecific antibodies are known in the art, such as, by co-expression of two immunoglobulin heavy chain-light chain pairs, where the two heavy chains have different specificities (see, e.g., Milstein and Cuello, Nature, 1983, 305:537-40). For further details of generating multispecific antibodies (e.g., bispecific antibodies), see, for example, Bispecific Antibodies (Kontermann, ed., 2011).

### 5.18 Methods of Making the Antibodies

In yet another aspect, provided herein are methods for making the various antibodies provided herein.

In some embodiments, recombinant expression of an antibody provided herein (e.g., a full-length antibody, the heavy and/or light chain of an antibody provided herein, or a mutant thereof) that binds to an antigen (e.g., HLA-G, CD37, GPRC5D, KLK2, PSMA, or BCMA) requires construction of an expression vector containing a polynucleotide that encodes the antibody. Once a polynucleotide encoding an antibody molecule or the heavy or light chain of an antibody provided herein has been obtained, variants of such polynucleotide that comprise the K248E, T437R or K248E/T437R mutation in the full-length antibody or the heavy chain thereof can be generated by gene synthesis (see, e.g., Zhang, D., et al., supra), and/or site-directed mutagenesis (Clynes, R. A., et al., Nature Medicine, 2000, 6(4):443-6). In some embodiments, the polynucleotide comprising the K248E/T437R mutation comprises a heavy chain nucleotide sequence of any of SEQ ID NOs: 85, 86, 87, 88, 89, 90, 91, 92, 106, 160.

The vector for the production of the antibody molecule may be produced by recombinant DNA technology using techniques well-known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. In some embodiments, these methods are, for example, in vitro recombinant DNA techniques, synthetic techniques, and in vivo genetic recombination. In some embodiments, these expression vectors are replicable vectors comprising a nucleotide sequence encoding an antibody molecule provided herein, or a heavy or light chain of an antibody, operably linked to a promoter. In some embodiments, these vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy chain, the entire light chain, or both the entire heavy and light chains.

In some embodiments, the expression vector is transferred to a host cell by conventional techniques and the transfected cells are then cultured by conventional techniques to produce an antibody provided herein. In some embodiments, host cells contain a polynucleotide encoding an antibody provided herein, or a heavy or light chain thereof, operably linked to a heterologous promoter. In certain embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules provided herein (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule provided herein in situ. In some embodiments, these host-expression systems are microorganisms such as bacteria (e.g., E. coli and B. subtilis) transformed with recombinant bacteriophage DNA, plasmid DNA or cosmid DNA expression vectors containing antibody coding sequences. In some embodiments, these host-expression systems are yeast (e.g., Saccharomyces pichia) transformed with recombinant yeast expression vectors containing antibody coding sequences. In some embodiments, these host-expression systems are insect cell systems infected with recombinant virus expression vectors (e.g., baculovirus) containing antibody coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing antibody coding sequences. In some embodiments, these host-expression systems are mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some embodiments, these host-expression systems are bacterial cells such as Escherichia coli, or eukaryotic cells, especially for the expression of whole recombinant antibody molecule, which can be used for the expression of a recombinant antibody molecule. In some embodiments, mammalian cells such as Chinese hamster ovary cells (CHO), in conjunction with a vector such as the major intermediate early gene promoter element from human cytomegalovirus, is an effective expression system for antibodies (Foecking, et al., Gene, 1986, 45:101; and Cockett, et al., Bio/Technology, 1990, 8:2). In some embodiments, antibodies provided herein are produced in CHO cells. In some embodiments, the expression of nucleotide sequences encoding antibodies provided herein which immunospecifically bind to an HLA-G, CD37, GPRC5D, KLK2, PSMA, or BCMA antigen is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther, et al., EMBO, 1983, 12:1791), in which the antibody coding sequence may be ligated individually into the vector in frame with the lac Z coding region so that a fusion protein is produced; pIN vectors (Inouye & Inouye, Nucleic Acids Res., 1985, 13:3101-3109; Van Heeke & Schuster, J. Biol. Chem., 1989, 24:5503-9); and the like. pGEX vectors may also be used to express foreign polypeptides as fusion proteins with glutathione 5-transferase (GST). In some embodiments, such fusion proteins are soluble and can easily be purified from lysed cells by adsorption and binding to matrix glutathione agarose beads followed by elution in the presence of free glutathione. In some embodiments, the pGEX vectors are designed to include thrombin or factor Xa protease cleavage sites so that the cloned target gene product can be released from the GST moiety.

In some embodiments, Autographa californica nuclear polyhedrosis virus (AcNPV) is used as a vector to express foreign genes in an insect system. In some embodiments, the virus grows in Spodoptera frugiperda cells. In some embodiments, the antibody coding sequence may be cloned individually into non-essential regions (for example the polyhedrin gene) of the virus and placed under control of an AcNPV promoter (for example the polyhedrin promoter).

In some embodiments, viral vectors, e.g., tobacco mosaic virus (TMV)-based viral vectors, are used in plant cells and are systematically delivered to multiple parts of a plant body by Agrobacterium for coexpression of multiple antibodies of interest (Giritch, A., et al., Proc. Natl. Acad. Sci. USA, 2006, 103(40):14701-6). In some embodiments, the use of plant cells as expression systems results in significant increases in production speed and yield of antibodies, and the capability to synthesize mammalian-type complex N-oligosaccharides (Loos, A. and Steinkellner, H., Arch Biochem Biophys., 2012, 526-172(2):167-73).

In mammalian host cells, a number of viral-based expression systems may be utilized. In some embodiments, an adenovirus is used as an expression vector, and the antibody coding sequence of interest may be ligated to an adenovirus transcription/translation control complex, e.g., the late promoter and tripartite leader sequence. In some embodiments, this chimeric gene may then be inserted in the adenovirus genome by in vitro or in vivo recombination. In some embodiments, insertion in a non-essential region of the viral genome (e.g., region El or E3) will result in a recombinant virus that is viable and capable of expressing the antibody molecule in infected hosts (see, e.g., Logan & Shenk, Proc. Natl. Acad. Sci. USA, 1984, 8(1):355-9). In some embodiments, specific initiation signals may also be required for efficient translation of inserted antibody coding sequences. In some embodiments, these signals include the ATG initiation codon and adjacent sequences. In some embodiments, the initiation codon must be in phase with the reading frame of the desired coding sequence to ensure translation of the entire insert. In some embodiments, these exogenous translational control signals and initiation codons can be of a variety of origins, both natural and synthetic. In some embodiments, the efficiency of expression may be enhanced by the inclusion of appropriate transcription enhancer elements, transcription terminators, etc. (see, e.g., Bittner, et al., Methods in Enzymol., 1987, 153:51-544).

In some embodiments, a host cell strain may be chosen which modulates the expression of the inserted sequences, or modifies and processes the gene product in the specific fashion desired. Such modifications (e.g., glycosylation, including fucosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells. In some embodiments, fully human monoclonal antibodies provided herein are produced in mammalian cells, such as CHO cells.

Standard techniques known to those of skill in the art can be used to generate an antibody provided herein with a heavy chain comprising no fucose residue on the Asn-297-linked N-oligosaccharide thereof. In some embodiments, cells with deficient GMD enzymes (e.g., CHO Lec13 cells) are used to generate an antibody with no fucose residue on the Asn-297-linked N-oligosaccharide on the heavy chain. In some embodiments, cells with reduced α-1,6 fucosyltransferase activity resulting from mutated or inactivated FUT8 genes (e.g., rat hybridoma YB2/0 cell lines) are used to generate an antibody with no fucose residue on the Asn-297-linked N-oligosaccharide on the heavy chain. In some embodiments, cells with overexpression of β-1,4-mannosyl-glycoprotein 4-β-N-acetylglucosaminyltransferase (GnT-III) are used to generate an antibody with no fucose residue on the Asn-297-linked N-oligosaccharide on the heavy chain. In some embodiments, cells with inactivated Golgi GDP-fucose transporter (GFT) gene Slc35c1 (e.g., CHO-gmt3 cells) are used to generate an antibody with no fucose residue on the Asn-297-linked N-oligosaccharide on the heavy chain. In some embodiments, cells with heterologous expression of bacterial RMD are used to generate an antibody with no fucose residue on the Asn-297-linked N-oligosaccharide on the heavy chain. In some embodiments, biochemical inhibitors of fucosylation (e.g., fucose analogs such as 2-fluorofucose and 5-alkynylfucose) are used in expression systems to produce antibodies with no fucose residue on the Asn-297-linked N-oligosaccharide on the heavy chain (Pereira, N. A., et al., supra; Shields, R. L. et al., supra; Kanda Y., supra). In some embodiments, plant cells with disrupted α1,3-fucosyltransferase (FucT) and β1,2-xylosyltransferase (XylT) are used to generate an antibody with no fucose residue on the Asn-297-linked N-oligosaccharide on the heavy chain (Pereira, N. A., et al., supra). In some embodiments, chemoenzymic remodeling is used to generate an antibody with no fucose residue on the Asn-297-linked N-oligosaccharide on the heavy chain (Pereira, N. A., et al., supra). For example, an exoglycosidase such as fucosidase may be used to remove the fucose on the Asn-297-linked N-oligosaccharide on the heavy chain of an antibody (Pereira, N. A., et al., supra).

For long-term, high-yield production of recombinant proteins, stable expression can be utilized. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. In some embodiments, the selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express the antibody molecule. Such engineered cell lines may be particularly useful in screening and evaluation of compositions that interact directly or indirectly with the antibody molecule.

A number of selection systems may be used, including, but not limited to, the herpes simplex virus thymidine kinase (Wigler, et al., Cell, 1977, 11:223), hypoxanthineguanine phosphoribosyltransferase (Szybalska & Szybalski, Proc. Natl. Acad. Sci. USA, 1992, 48:202), and adenine phosphoribosyltransferase (Lowy, et al., Cell, 1980, 22:8-17) genes can be employed in tk-, hgprt- or aprt-cells, respectively. Also, antimetabolite resistance may be used as the basis of selection for the following genes: dhfr, which confers resistance to methotrexate (Wigler, et al., Natl. Acad. Sci. USA, 1980, 77:357; O'Hare, et al., Proc. Natl. Acad. Sci. USA, 1981, 78:1527); gpt, which confers resistance to mycophenolic acid (Mulligan & Berg, Proc. Natl. Acad. Sci. USA, 1981, 78:2072); neo, which confers resistance to the aminoglycoside G-418 (Wu and Wu, Biotherapy, 1991, 3:87-95; Tolstoshev, Ann. Rev. Pharmacol. Toxicol., 1993, 32:573-96; Mulligan, Science, 1993, 260:926-32; and Morgan and Anderson, Ann. Rev. Biochem., 1993, 62:191-217); and hygro, which confers resistance to hygromycin (Santerre, et al., Gene, 1984, 30:147). Methods commonly known in the art of recombinant DNA technology may be routinely applied to select the desired recombinant clone, and such methods are described, for example, in Ausubel, et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli, et al. (eds.), Current Protocols in Human Genetics, John Wiley & Sons, NY (1994); Colberre-Garapin, et al., J. Mol. Biol., 1981, 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). In some embodiments, a marker in the vector system expressing antibody is amplifiable, and the increase in the level of inhibitor present in culture of the host cell will increase the number of copies of the marker gene and the production of the antibody since the amplified region is associated with the antibody gene (Crouse et al., 1983, Mol. Cell. Biol. 3:257).

The host cell may be co-transfected with two expression vectors provided herein, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature, 1986, 322:52; and Kohler, Proc. Natl. Acad. Sci. USA, 1980, 77:2197-9). The coding sequences for the heavy and light chains may comprise cDNA or genomic DNA.

Once an antibody molecule provided herein has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies provided herein can be fused to heterologous polypeptide sequences described herein or otherwise known in the art to facilitate purification. Certain methods of making the antibodies provided herein are described in Section 7 below.

### 5.19 Pharmaceutical Compositions

In one aspect, the present disclosure further provides pharmaceutical compositions comprising at least one antibody of the present disclosure. In some embodiments, a pharmaceutical composition comprises therapeutically effective amount of an antibody or antigen binding fragment thereof provided herein and a pharmaceutically acceptable excipient.

In some embodiments, pharmaceutical compositions comprising an antibody are prepared for storage by mixing the fusion protein having the desired degree of purity with optional physiologically acceptable excipients (see, e.g., Remington, Remington's Pharmaceutical Sciences (18th ed. 1980)) in the form of aqueous solutions or lyophilized or other dried forms.

The antibody of the present disclosure may be formulated in any suitable form for delivery to a target cell/tissue, e.g., as microcapsules or macroemulsions (Remington, supra; Park, et al., Molecules, 2005, 10:146-61; Malik, et al., Curr. Drug. Deliv., 2007, 4:141-51), as sustained release formulations (Putney and Burke, Nature Biotechnol., 1998, 16:153-57), or in liposomes (Maclean, et al., Int. J. Oncol., 1997, 11:325-32; Kontermann, Curr. Opin. Mol. Ther., 2006, 8:39-45).

An antibody provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody as described herein, including, but not limited to, encapsulation in liposomes, microparticles, microcapsules, recombinant cells capable of expressing the antibody, receptor-mediated endocytosis (see, e.g., Wu and Wu, J. Biol. Chem., 1987, 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, Science, 1990, 249:1527-33; Sefton, Crit. Ref. Biomed. Eng., 1987, 14:201-40; Buchwald, et al., Surgery, 1980, 88:507-16; and Saudek et al., N. Engl. J. Med., 1989, 321:569-74). In another embodiment, polymeric materials can be used to achieve controlled or sustained release of a prophylactic or therapeutic agent (e.g., an antibody as described herein) or a composition provided herein (see, e.g., Medical Applications of Controlled Release (Langer and Wise, eds., 1974); Controlled Drug Bioavailability, Drug Product Design and Performance (Smolen and Ball, eds., 1984); Ranger and Peppas, J. Macromol. Sci. Rev. Macromol. Chem., 1983, 23:61-126; Levy, et al., Science, 1985, 228:190-92; During, et al., Ann. Neurol., 1989, 25:351-6; Howard, et al., J. Neurosurg., 1989, 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxyethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, supra. Any technique known to one of skill in the art can be used to produce sustained release formulations comprising one or more antibody as described herein (see, e.g., U.S. Pat. No. 4,526,938, PCT publication Nos. WO 91/05548 and WO 96/20698, Ning, et al., Radiotherapy & Oncology, 1996, 39:179-89; Song, et al., PDA J. of Pharma. Sci. & Tech., 1995, 50:372-97; Cleek, et al., Pro. Int'l. Symp. Control. Rel. Bioact. Mater., 1997, 24:853-4; and Lam, et al., Proc. Int'l. Symp. Control Rel. Bioact. Mater., 1997, 24:759-60).

### 5.20 Methods of Using, Administrating and Dosing

In one aspect, provided herein is a method of enhancing the ADCC and/or CDC activity of an antibody.

In some embodiments, the ADCC activity of anti-HLA-G antibodies, anti-CD37 antibodies, anti-GPRC5D antibodies, anti-KLK2 antibodies, anti-PSMA antibodies, or anti-BCSMA antibodies provided herein are determined by measuring the percentage of target cell death mediated by effector cells, e.g., NK cells, macrophages, mononuclear phagocytes, neutrophils or eosinophils. For example, target cells that endogenously expresses HLA-G, CD37 may be loaded with BATDA, a cell-labeling reagent that is released upon cell death and can be detected in the cell culture supernatant after forming a fluorescent chelate. In such situations, anti-HLA-G antibodies or anti-CD37 antibodies are added to BATDA-labelled target cells. Effector cells, e.g., peripheral blood mononuclear cells (PBMC), cultured overnight can be added at an effector to target cell ratio (E:T ratio) of 50:1 to target cells at 5,000 cells/well. In some embodiments, the effector and target cell mixture is incubated for 4 hours at 37° C. Europium (III) chelate, a BATDA chelate substrate, can be added to the mixture at a ratio of 1:10. The amount of released BATDA can be determined by measuring emitted fluorescence at 615 nm. In some embodiments, the fluorescence signal for 100% target cell death is determined using a well containing BATDA-labeled target cells mixed with Triton-X 100 detergent.

As another example, VcaP cells stably transfected with Nuclight Red (Incucyte®, Essen Bioscience) are plated at 10,000 cells per well in a 384-well plate (Perkin Elmer ViewPlate) in clear media (RPMI 1641+10% FBS, Thermo Fisher Scientific) to allow for cell adherence overnight. ADCC assay can be performed with freshly thawed PBMC (Hemcare, PB009C-3). The ratio of effector to target cell per well can be 34:1 for PBMCs as effector cells. In some embodiments, KLK2 antibodies were tested with final concentrations ranging from 100 nM to 0.01 nM. After effector cells and antibodies are added to target cells, real time imaging can be performed under Incucyte® S3 instrument (Essen BioScience). Total red intergraded signal per well can be quantified with Incucyte® software. Data analysis can be performed by Incucyte® software and Prism (GraphPad Software) based on values of quadruplicates. In some embodiments, the percentage of cell killing is calculated as: (1−KLK2 mAb/no mAb control)×100%.

As another example, C42B and LNCap cells stably transfected with GFP are plated at 9,000 cells per well in a 384-well plate (Perkin Elmer ViewPlate) in clear media (RPMI 1641+10% FBS, Thermo Fisher Scientific) to allow for cell adherence overnight. ADCC assay can be performed with freshly thawed PBMC (Hemcare, PB009C-3) or NK cells isolated from the frozen PBMC by RoboSep™ Cell Separation Instruments. Isolated NK cells can be either used immediately or primed overnight with low dose IL-2 (1 ng/ml, Miltenyi Biotec). The ratio of effector to target cell per well can be 34:1 for PBMC and 5:1 for isolated NK cells. In some embodiments, anti-PSMA antibodies are tested with final concentrations ranging from 100 nM to 0.01 nM. After effector cells and antibodies are added to target cells, real time imaging can be performed under Incucyte® S3 instrument (Essen BioScience). Total GFP intergraded signal per well can be quantified with Incucyte® software. Data analysis can be performed by Incucyte® software and Prism (GraphPad Software) based on values of quadruplicates. The percentage of cell killing can be calculated as: (1−PSMA mAb/no mAb control)×100%.

As another example, C42B and LNCap cells stably transfected with GFP are plated at 9,000 cells per well in a 384-well plate (Perkin Elmer ViewPlate) in clear media (RPMI 1641+10% FBS, Thermo Fisher Scientific) to allow for cell adherence overnight. ADCC assay can be performed with freshly thawed PBMC (Hemcare, PB009C-3) or NK cells isolated from the frozen PBMC by RoboSep™ Cell Separation Instruments. Isolated NK cells can be either used immediately or primed overnight with low dose IL-2 (1 ng/ml, Miltenyi Biotec). The ratio of effector to target cell per well can be 34:1 for PBMC and 5:1 for isolated NK cells. In some embodiments, anti-BCMA antibodies are tested with final concentrations ranging from 100 nM to 0.01 nM. After effector cells and antibodies are added to target cells, real time imaging can be performed under Incucyte® S3 instrument (Essen BioScience). Total GFP intergraded signal per well can be quantified with Incucyte® software. Data analysis can be performed by Incucyte® software and Prism (GraphPad Software) based on values of quadruplicates. The percentage of cell killing can be calculated as: (1−BCMA mAb/no mAb control)×100%.

In some embodiments, the antibody provided herein causes at least 10% target cell death via ADCC. In some embodiments, the antibody provided herein causes at least 20% target cell death via ADCC. In some embodiments, the antibody provided herein causes at least 30% target cell death via ADCC. In some embodiments, the antibody provided herein causes at least 40% target cell death via ADCC. In some embodiments, the antibody provided herein causes at least 50% target cell death via ADCC. In some embodiments, the antibody provided herein causes at least 60% target cell death via ADCC. In some embodiments, the antibody provided herein causes at least 70% target cell death via ADCC. In some embodiments, the antibody provided herein causes at least 80% target cell death via ADCC. In some embodiments, the antibody provided herein causes at least 90% target cell death via ADCC. In some embodiments, the antibody provided herein causes at least 95% target cell death via ADCC.

In some embodiments, the CDC activity of anti-HLA-G antibodies, anti-CD37 antibodies, anti-GPRC5D antibodies, anti-KLK2 antibodies, anti-PSMA antibodies, or anti-BCSMA antibodies provided herein are determined by measuring the percentage of target cell death. For example, target cells that endogenously expresses HLA-G, CD37, GPRC5D, KLK2, PSMA, or BCMA can be cultured in DMEM medium with 10% Fetal Bovine Serum (FBS). In some embodiments, antibodies are added to target cells and incubated for 30 minutes at 37° C., with baby rabbit serum then added to target cells to a final concentration of 10% to provide a source of complement components for CDC. In some embodiments, such mixture is incubated for 4 hours at 37° C. 100 μl of CellTiter-Glo reagent (Promega) can be added to the mixture followed by incubation for 10 minutes at room temperature. Target cell viability can be determined by measuring luminescence with a Tecan SPARK Reader. Target cell death can thus be determined as the difference between 100% and target cell viability.

In some embodiments, the antibody provided herein causes at least 10% target cell death via CDC. In some embodiments, the antibody provided herein causes at least 20% target cell death via CDC. In some embodiments, the antibody provided herein causes at least 30% target cell death via CDC. In some embodiments, the antibody provided herein causes at least 40% target cell death via CDC. In some embodiments, the antibody provided herein causes at least 50% target cell death via CDC. In some embodiments, the antibody provided herein causes at least 60% target cell death via CDC. In some embodiments, the antibody provided herein causes at least 70% target cell death via CDC. In some embodiments, the antibody provided herein causes at least 80% target cell death via CDC. In some embodiments, the antibody provided herein causes at least 90% target cell death via CDC. In some embodiments, the antibody provided herein causes at least 95% target cell death via CDC.

In some embodiments, the antibody provided herein enhances ADCC activity by at least about 2-fold. In some embodiments, the antibody provided herein enhances ADCC activity by at least about 5-fold. In some embodiments, the antibody provided herein enhances ADCC activity by at least about 10-fold. In some embodiments, the antibody provided herein enhances ADCC activity by at least about 20-fold. In some embodiments, the antibody provided herein enhances ADCC activity by at least about 30-fold. In some embodiments, the antibody provided herein enhances ADCC activity by at least about 40-fold. In some embodiments, the antibody provided herein enhances ADCC activity by at least about 50-fold. In some embodiments, the antibody provided herein enhances ADCC activity by at least about 60-fold. In some embodiments, the antibody provided herein enhances ADCC activity by at least about 70-fold. In some embodiments, the antibody provided herein enhances ADCC activity by at least about 80-fold. In some embodiments, the antibody provided herein enhances ADCC activity by at least about 90-fold. In some embodiments, the antibody provided herein enhances ADCC activity by at least about 100-fold. In some embodiments, the antibody provided herein enhances ADCC activity by at least about 200-fold. In some embodiments, the antibody provided herein enhances ADCC activity by at least about 500-fold.

In some embodiments, the antibody provided herein enhances CDC activity by at least about 2-fold. In some embodiments, the antibody provided herein enhances CDC activity by at least about 5-fold. In some embodiments, the antibody provided herein enhances CDC activity by at least about 10-fold. In some embodiments, the antibody provided herein enhances CDC activity by at least about 20-fold. In some embodiments, the antibody provided herein enhances CDC activity by at least about 30-fold. In some embodiments, the antibody provided herein enhances CDC activity by at least about 40-fold. In some embodiments, the antibody provided herein enhances CDC activity by at least about 50-fold. In some embodiments, the antibody provided herein enhances CDC activity by at least about 60-fold. In some embodiments, the antibody provided herein enhances CDC activity by at least about 70-fold. In some embodiments, the antibody provided herein enhances CDC activity by at least about 80-fold. In some embodiments, the antibody provided herein enhances CDC activity by at least about 90-fold. In some embodiments, the antibody provided herein enhances CDC activity by at least about 100-fold. In some embodiments, the antibody provided herein enhances CDC activity by at least about 200-fold. In some embodiments, the antibody provided herein enhances CDC activity by at least about 500-fold.

In some embodiments, the antibody provided herein enhances both ADCC and CDC activity by at least about 2-fold. In some embodiments, the antibody provided herein enhances both ADCC and CDC activity by at least about 5-fold. In some embodiments, the antibody provided herein enhances both ADCC and CDC activity by at least about 10-fold. In some embodiments, the antibody provided herein enhances both ADCC and CDC activity by at least about 20-fold. In some embodiments, the antibody provided herein enhances both ADCC and CDC activity by at least about 30-fold. In some embodiments, the antibody provided herein enhances both ADCC and CDC activity by at least about 40-fold. In some embodiments, the antibody provided herein enhances both ADCC and CDC activity by at least about 50-fold. In some embodiments, the antibody provided herein enhances both ADCC and CDC activity by at least about 60-fold. In some embodiments, the antibody provided herein enhances both ADCC and CDC activity by at least about 70-fold. In some embodiments, the antibody provided herein enhances both ADCC and CDC activity by at least about 80-fold. In some embodiments, the antibody provided herein enhances both ADCC and CDC activity by at least about 90-fold. In some embodiments, the antibody provided herein enhances both ADCC and CDC activity by at least about 100-fold. In some embodiments, the antibody provided herein enhances both ADCC and CDC activity by at least about 200-fold. In some embodiments, the antibody provided herein enhances both ADCC and CDC activity by at least about 500-fold.

In another aspect, provided herein is a method of treating a disease or disorder in a subject comprising administering to the subject an effective amount of an antibody provided herein. In one embodiment, the disease or disorder is an HLA-G-mediated disease or disorder. In one embodiment, the disease or disorder is a CD37-mediated disease or disorder. In one embodiment, the disease or disorder is a GPRC5D-mediated disease or disorder. In one embodiment, the disease or disorder is a KLK2-mediated disease or disorder. In one embodiment, the disease or disorder is a PSMA-mediated disease or disorder. In one embodiment, the disease or disorder is a BCMA-mediated disease or disorder. In some embodiments, the disease or disorder is selected from a group consisting of cancers, autoimmune diseases, inflammatory diseases, cardiovascular diseases, genetic diseases, hematologic diseases and/or pulmonary diseases affecting joints, skin, kidney, liver, intestine, heart, lung, muscle, stomach, spleen, pancreas, gall bladder, bladder, appendix, thymus, brain, esophagus, eye or ear, wherein optionally the disease or disorder is selected from a group consisting of rheumatoid arthritis, bullous pemphigoid, discoid cutaneous lupus, urticarial vasculitis, Henoch-Schonlein Purpura, IgA nephropathy, atopic dermatitis (atopic eczema), psoriasis (psoriasis vulgaris), seborrheic eczema, asthma, proteinuric kidney disease, liver disease, lupus nephritis, polymyositis, dermatomyositis, calcineurin inhibitor induced nephrotoxicity, myotonic dystrophy, cardiac dysfunction and failure, Alport syndrome, ulcerative colitis, Crohn's disease, cutaneous vasculitis, cachexia, and inflammatory bowel disease, and wherein optionally the disease or disorder is related to fibrosis and optionally selected from a group consisting of tissue fibrosis, idiopathic pulmonary fibrosis, interstitial lung disease, scleroderma (systemic sclerosis), cancer, cancer-associated cachexia, muscle wasting, keloids, inclusion body myositis, and tissue remodeling (see, e.g., Heider, K. H., et al., Blood, 2011, 118(15):4159-68; Carosella, E. D., et al., Adv. Immunol., 2015, 127:33-144; Stathis, S., et al., Invest New Drugs, 2018, 36(5):869-76; Stilgenbauer, S., et al., Leukemia, 2019, 33:2531-5; Gomes, R. G., et al., Hum. Immunol., 2018, 79(6):477-84; Moroso, V., et al., Transplantation, 2015, 99(12):2514-22; Lazarte, J., et al., Hum. Immunol., 2018, 79(8):587-93; Rached, M. R., et al., Eur J Obstet Gynecol Reprod Biol., 2019, 235:36-41; Koc, A., et al., Adv Clin Exp Med., 2018, 27(9):1233-7; Ribeyre, C., et al., Front Immunol., 2018, 9:278; Smith, E. L., et al., Sci Transl Med., 2019, 11(485):eaau7746; Kodama, T., et al., Mol Cancer Ther., 2019, 18(9):1555-64; Cohen, Y., et al., Hematology, 2013, 18(6):348-51).

Also provided herein is a method of treatment of a disease or disorder, wherein the subject is administered one or more therapeutic agents in combination with the antibody provided herein.

In another aspect, provided herein is the use of the antibody provided herein in the manufacture of a medicament for treating a disease or disorder in a subject.

In another aspect, provided herein is the use of a pharmaceutical composition provided herein in the manufacture of a medicament for treating a disease or disorder in a subject.

In a specific embodiment, provided herein is a composition for use in the prevention and/or treatment of a disease or condition comprising an antibody provided herein. In one embodiment, provided herein is a composition for use in the prevention of a disease or condition, wherein the composition comprises an antibody provided herein. In one embodiment, provided herein is a composition for use in the treatment of a disease or condition, wherein the composition comprises an antibody provided herein. In some embodiments, the disease or condition is an HLA-G-mediated disease. In some embodiments, the disease or condition is a CD37-mediated disease. In some embodiments, the disease or condition is a GPRC5D-mediated disease. In some embodiments, the disease or condition is a KLK2-mediated disease. In one embodiment, the disease or condition is a PSMA-mediated disease. In one embodiment, the disease or condition is a BCMA-mediated disease. In some embodiments, the disease or disorder is selected from a group consisting of cancers, autoimmune diseases, inflammatory diseases, cardiovascular diseases, genetic diseases, hematologic diseases and/or pulmonary diseases affecting joints, skin, kidney, liver, intestine, heart, lung, muscle, stomach, spleen, pancreas, gall bladder, bladder, appendix, thymus, brain, esophagus, eye or ear, wherein optionally the disease or disorder is selected from a group consisting of rheumatoid arthritis, bullous pemphigoid, discoid cutaneous lupus, urticarial vasculitis, Henoch-Schonlein Purpura, IgA nephropathy, atopic dermatitis (atopic eczema), psoriasis (psoriasis vulgaris), seborrheic eczema, asthma, proteinuric kidney disease, liver disease, lupus nephritis, polymyositis, dermatomyositis, calcineurin inhibitor induced nephrotoxicity, myotonic dystrophy, cardiac dysfunction and failure, Alport syndrome, ulcerative colitis, Crohn's disease, cutaneous vasculitis, cachexia, and inflammatory bowel disease, and wherein optionally the disease or disorder is related to fibrosis and optionally selected from a group consisting of tissue fibrosis, idiopathic pulmonary fibrosis, interstitial lung disease, scleroderma (systemic sclerosis), cancer, cancer-associated cachexia, muscle wasting, keloids, inclusion body myositis, and tissue remodeling.

In certain embodiments, the subject is a subject in need thereof. In some embodiments, the subject has the disease or condition. In other embodiments, the subject is at risk of having the disease or condition. In some embodiments, the administration results in the prevention, management, treatment or amelioration of the disease or condition.

In one embodiment, provided herein is a composition for use in the prevention and/or treatment of a symptom of a disease or condition, wherein the composition comprises an antibody provided herein. In one embodiment, provided herein is a composition for use in the prevention of a symptom of a disease or condition, wherein the composition comprises an antibody provided herein. In one embodiment, provided herein is a composition for use in the treatment of a symptom of a disease or condition, wherein the composition comprises an antibody provided herein. In some embodiments, the disease or condition is an HLA-G-mediated, CD37-mediated, GPRC5D-mediated, KLK2-mediated disease, PSMA-mediated disease, and/or BCMA-mediated disease. In some embodiments, the disease or disorder is selected from a group consisting of cancers, autoimmune diseases, inflammatory diseases, cardiovascular diseases, genetic diseases, hematologic diseases and/or pulmonary diseases affecting joints, skin, kidney, liver, intestine, heart, lung, muscle, stomach, spleen, pancreas, gall bladder, bladder, appendix, thymus, brain, esophagus, eye or ear, wherein optionally the disease or disorder is selected from a group consisting of rheumatoid arthritis, bullous pemphigoid, discoid cutaneous lupus, urticarial vasculitis, Henoch-Schonlein Purpura, IgA nephropathy, atopic dermatitis (atopic eczema), psoriasis (psoriasis vulgaris), seborrheic eczema, asthma, proteinuric kidney disease, liver disease, lupus nephritis, polymyositis, dermatomyositis, calcineurin inhibitor induced nephrotoxicity, myotonic dystrophy, cardiac dysfunction and failure, Alport syndrome, ulcerative colitis, Crohn's disease, cutaneous vasculitis, cachexia, and inflammatory bowel disease, and wherein optionally the disease or disorder is related to fibrosis and optionally selected from a group consisting of tissue fibrosis, idiopathic pulmonary fibrosis, interstitial lung disease, scleroderma (systemic sclerosis), cancer, cancer-associated cachexia, muscle wasting, keloids, inclusion body myositis, and tissue remodeling.

In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

In another embodiment, provided herein is a method of preventing and/or treating a disease or condition in a subject, comprising administering an effective amount of an antibody provided herein. In one embodiment, provided herein is a method of preventing a disease or condition in a subject, comprising administering an effective amount of an antibody provided herein. In one embodiment, provided herein is a method of treating a disease or condition in a subject, comprising administering an effective amount of an antibody provided herein. In some embodiments, the disease or condition is an HLA-G-mediated, CD37-mediated, GPRC5D-mediated, KLK2-mediated disease, PSMA-mediated disease, and/or BCMA-mediated disease. In some embodiments, the disease or disorder is selected from a group consisting of cancers, autoimmune diseases, inflammatory diseases, cardiovascular diseases, genetic diseases, hematologic diseases and/or pulmonary diseases affecting joints, skin, kidney, liver, intestine, heart, lung, muscle, stomach, spleen, pancreas, gall bladder, bladder, appendix, thymus, brain, esophagus, eye or ear, wherein optionally the disease or disorder is selected from a group consisting of rheumatoid arthritis, bullous pemphigoid, discoid cutaneous lupus, urticarial vasculitis, Henoch-Schonlein Purpura, IgA nephropathy, atopic dermatitis (atopic eczema), psoriasis (psoriasis vulgaris), seborrheic eczema, asthma, proteinuric kidney disease, liver disease, lupus nephritis, polymyositis, dermatomyositis, calcineurin inhibitor induced nephrotoxicity, myotonic dystrophy, cardiac dysfunction and failure, Alport syndrome, ulcerative colitis, Crohn's disease, cutaneous vasculitis, cachexia, and inflammatory bowel disease, and wherein optionally the disease or disorder is related to fibrosis and optionally selected from a group consisting of tissue fibrosis, idiopathic pulmonary fibrosis, interstitial lung disease, scleroderma (systemic sclerosis), cancer, cancer-associated cachexia, muscle wasting, keloids, inclusion body myositis, and tissue remodeling.

In certain embodiments, the subject is a subject in need thereof. In some embodiments, the subject has the disease or condition. In other embodiments, the subject is at risk of having the disease or condition. In some embodiments, the administration results in the prevention or treatment of the disease or condition.

In another embodiment, provided herein is a method of preventing and/or treating a symptom of a disease or condition in a subject, comprising administering an effective amount of an antibody provided herein. In one embodiment, provided herein is a method of preventing a symptom of a disease or condition in a subject, comprising administering an effective amount of an antibody provided herein. In one embodiment, provided herein is a method of treating a symptom of a disease or condition in a subject, comprising administering an effective amount of an antibody provided herein. In some embodiments, the disease or condition is an HLA-G-mediated, CD37-mediated, GPRC5D-mediated, KLK-2 mediated, PSMA-mediated disease, and/or BCMA-mediated disease. In some embodiments, the disease or disorder is selected from a group consisting of cancers, autoimmune diseases, inflammatory diseases, cardiovascular diseases, genetic diseases, hematologic diseases and/or pulmonary diseases affecting joints, skin, kidney, liver, intestine, heart, lung, muscle, stomach, spleen, pancreas, gall bladder, bladder, appendix, thymus, brain, esophagus, eye or ear, wherein optionally the disease or disorder is selected from a group consisting of rheumatoid arthritis, bullous pemphigoid, discoid cutaneous lupus, urticarial vasculitis, Henoch-Schonlein Purpura, IgA nephropathy, atopic dermatitis (atopic eczema), psoriasis (psoriasis vulgaris), seborrheic eczema, asthma, proteinuric kidney disease, liver disease, lupus nephritis, polymyositis, dermatomyositis, calcineurin inhibitor induced nephrotoxicity, myotonic dystrophy, cardiac dysfunction and failure, Alport syndrome, ulcerative colitis, Crohn's disease, cutaneous vasculitis, cachexia, and inflammatory bowel disease, and wherein optionally the disease or disorder is related to fibrosis and optionally selected from a group consisting of tissue fibrosis, idiopathic pulmonary fibrosis, interstitial lung disease, scleroderma (systemic sclerosis), cancer, cancer-associated cachexia, muscle wasting, keloids, inclusion body myositis, and tissue remodeling.

In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

Also provided herein are methods of preventing and/or treating a disease or condition by administrating to a subject of an effective amount of an antibody provided herein, or pharmaceutical composition comprising an antibody provided herein. In one aspect, the antibody is substantially purified (i.e., substantially free from substances that limit its effect or produce undesired side-effects). The subject administered a therapy can be a mammal such as non-primate (e.g., cows, pigs, horses, cats, dogs, rats etc.) or a primate (e.g., a monkey, such as a cynomolgus macaque monkey, or a human). In a one embodiment, the subject is a human. In another embodiment, the subject is a human with a disease or condition.

Various delivery systems are known and can be used to administer a prophylactic or therapeutic agent (e.g., an antibody provided herein), including, but not limited to, encapsulation in liposomes, microparticles, microcapsules, recombinant cells capable of expressing the antibody, receptor-mediated endocytosis (see, e.g., Wu and Wu, J. Biol. Chem., 1987 262:4429-4432), construction of a nucleic acid as part of a retroviral or other vector, etc. Methods of administering a prophylactic or therapeutic agent (e.g., an antibody provided herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antibody provided herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

In a specific embodiment, it may be desirable to administer a prophylactic or therapeutic agent, or a pharmaceutical composition provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. In some embodiments, when administering an antibody provided herein, care must be taken to use materials to which the antibody does not absorb.

In another embodiment, a prophylactic or therapeutic agent, or a composition provided herein can be delivered in a vesicle, in particular a liposome (see Langer, Science, 1990, 249:1527-1533; Treat, et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, a prophylactic or therapeutic agent, or a composition provided herein can be delivered in a controlled release or sustained release system. In one embodiment, a pump may be used to achieve controlled or sustained release (see Langer, supra; Sefton, CRC Crit. Ref. Biomed. Eng., 1987, 14:20; Buchwald et al., Surgery, 1980, 88:507; Saudek et al., N. Engl. J. Med., 1989, 321:574). In another embodiment, polymeric materials can be used to achieve controlled or sustained release of a prophylactic or therapeutic agent (e.g., an antibody provided herein) or a composition provided herein (see e.g., Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, J., Macromol. Sci. Rev. Macromol. Chem., 1983, 23:61; see also Levy, et al., Science, 1985, 228:190; During, et al., Ann. Neurol., 1989, 25:351; Howard, et al., J. Neurosurg., 1989, 7(1):105; U.S. Pat. Nos. 5,679,377; 5,916,597; 5,912,015; 5,989,463; 5,128,326; PCT Publication No. WO 99/15154; and PCT Publication No. WO 99/20253). Examples of polymers used in sustained release formulations include, but are not limited to, poly(2-hydroxyethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the nasal passages or lungs, thus requiring only a fraction of the systemic dose (see, e.g., Goodson, in Medical Applications of Controlled Release, supra, vol. 2, pp. 115-138 (1984)). Controlled release systems are discussed in the review by Langer, Science, 1990, 249:1527-33). Any technique known to one of skill in the art can be used to produce sustained release formulations comprising one or more antibody provided herein. See, e.g., U.S. Pat. No. 4,526,938, PCT publication WO 91/05548, PCT publication WO 96/20698, Ning, et al., Radiotherapy & Oncology, 1996, 39:179-89; Song, et al., PDA J of Pharmaceutical Sci & Technol., 1995, 50:372-97; Cleek, et al., Pro. Int'l. Symp. Control. Rel. Bioact. Mater., 1997, 24:853-54; and Lam, et al., Proc. Int'l. Symp. Control Rel. Bioact. Mater., 1997, 24:759-60, each of which is incorporated herein by reference in their entirety.

In a specific embodiment, where the composition provided herein is a nucleic acid encoding a prophylactic or therapeutic agent (e.g., an antibody provided herein), the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (see, e.g., Joliot, et al., Proc. Natl. Acad. Sci. USA, 1991, 88:1864-8), etc. Alternatively, a nucleic acid can be introduced intracellularly and incorporated within host cell DNA for expression by homologous recombination.

In a specific embodiment, a composition provided herein comprises one, two or more antibodies provided herein. In another embodiment, a composition provided herein comprises one, two or more antibodies provided herein and a prophylactic or therapeutic agent other than an antibody provided herein. In one embodiment, the agents are known to be useful for or have been or are currently used for the prevention, management, treatment and/or amelioration of a disease or condition. In addition to prophylactic or therapeutic agents, the compositions provided herein may also comprise an excipient.

The compositions provided herein include bulk drug compositions useful in the manufacture of pharmaceutical compositions (e.g., compositions that are suitable for administration to a subject or patient) that can be used in the preparation of unit dosage forms. In an embodiment, a composition provided herein is a pharmaceutical composition. Such compositions comprise a prophylactically or therapeutically effective amount of one or more prophylactic or therapeutic agents (e.g., an antibody provided herein or other prophylactic or therapeutic agent), and a pharmaceutically acceptable excipient. The pharmaceutical compositions can be formulated to be suitable for the route of administration to a subject.

In a specific embodiment, the term “excipient” can also refer to a diluent, adjuvant (e.g., Freunds' adjuvant (complete or incomplete) or vehicle. Pharmaceutical excipients can be sterile liquids, such as water and oils, including those of petroleum, animal, vegetable or synthetic origin, such as peanut oil, soybean oil, mineral oil, sesame oil and the like. Water is an exemplary excipient when the pharmaceutical composition is administered intravenously. Saline solutions and aqueous dextrose and glycerol solutions can also be employed as liquid excipients, particularly for injectable solutions. Suitable pharmaceutical excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard excipients such as pharmaceutical grades of mannitol, lactose, starch, magnesium stearate, sodium saccharine, cellulose, magnesium carbonate, etc. Examples of suitable pharmaceutical excipients are described in Remington's Pharmaceutical Sciences (1990) Mack Publishing Co., Easton, PA. Such compositions will contain a prophylactically or therapeutically effective amount of the antibody provided herein, such as in purified form, together with a suitable amount of excipient so as to provide the form for proper administration to the patient. The formulation should suit the mode of administration.

In an embodiment, the composition is formulated in accordance with routine procedures as a pharmaceutical composition adapted for intravenous administration to human beings. In some embodiments, compositions for intravenous administration are solutions in sterile isotonic aqueous buffer. Where necessary, the composition may also include a solubilizing agent and a local anesthetic such as lignocaine to ease pain at the site of the injection. Such compositions, however, may be administered by a route other than intravenous.

The ingredients of compositions provided herein may be supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

An antibody provided herein can be packaged in a hermetically sealed container such as an ampoule or sachette indicating the quantity of antibody. In one embodiment, the antibody is supplied as a dry sterilized lyophilized powder or water free concentrate in a hermetically sealed container and can be reconstituted, e.g., with water or saline to the appropriate concentration for administration to a subject. The lyophilized antibody can be stored at between 2 and 8° C. in its original container and the antibody can be administered within 12 hours, such as within 6 hours, within 5 hours, within 3 hours, or within 1 hour after being reconstituted. In an alternative embodiment, an antibody provided herein is supplied in liquid form in a hermetically sealed container indicating the quantity and concentration of the antibody.

The compositions provided herein can be formulated as neutral or salt forms. Pharmaceutically acceptable salts include, but are not limited to, those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a prophylactic or therapeutic agent (e.g., an antibody provided herein), or a composition provided herein that will be effective in the prevention and/or treatment of a disease or condition can be determined by standard clinical techniques. In addition, in vitro assays may optionally be employed to help identify optimal dosage ranges. In some embodiments, the precise dose to be employed in the formulation will also depend on the route of administration, and the seriousness of a disease or condition, and should be decided according to the judgment of the practitioner and each patient's circumstances.

Effective doses may be extrapolated from dose-response curves derived from in vitro or animal model test systems.

In certain embodiments, the route of administration for a dose of an antibody provided herein to a patient is intranasal, intramuscular, intravenous, subcutaneous, or a combination thereof, but other routes described herein are also acceptable. Each dose may or may not be administered by an identical route of administration. In some embodiments, an antibody provided herein may be administered via multiple routes of administration simultaneously or subsequently to other doses of the same or a different antibody provided herein.

In certain embodiments, the antibody provided herein are administered prophylactically or therapeutically to a subject. The antibody provided herein can be prophylactically or therapeutically administered to a subject so as to prevent, lessen or ameliorate a disease or symptom thereof.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### 6. EMBODIMENTS

This invention provides the following non-limiting embodiments.

In one set of embodiments (embodiment set A), provided are:
A1. A population of antibodies,
   (i) wherein less than 80% of the oligosaccharides covalently attached to the population of the antibodies via N297 residues thereof comprise a core fucose residue; and
   (ii) wherein the population of the antibodies comprises an antibody which Fc region comprises K338A and T437R mutations, or K248E and T437R mutations (RE mutations),
wherein amino acid residue numbering is according to the EU numbering system, and wherein optionally each antibody in the population of the antibodies comprises the RE mutations.
A2. The population of the antibodies of embodiment A1, wherein less than 70% of the oligosaccharides covalently attached to the population of the antibodies via N297 residues thereof comprise a core fucose residue.
A3. The population of the antibodies of embodiment A1, wherein less than 60% of the oligosaccharides covalently attached to the population of the antibodies via N297 residues thereof comprise a core fucose residue.
A4. The population of the antibodies of embodiment A1, wherein less than 50% of the oligosaccharides covalently attached to the population of the antibodies via N297 residues thereof comprise a core fucose residue.
A5. The population of the antibodies of embodiment A1, wherein less than 40% of the oligosaccharides covalently attached to the population of the antibodies via N297 residues thereof comprise a core fucose residue.
A6. The population of the antibodies of embodiment A1, wherein less than 30% of the oligosaccharides covalently attached to the population of the antibodies via N297 residues thereof comprise a core fucose residue.
A7. The population of the antibodies of embodiment A1, wherein less than 20% of the oligosaccharides covalently attached to the population of the antibodies via N297 residues thereof comprise a core fucose residue.
A8. The population of the antibodies of embodiment A1, wherein less than 10% of the oligosaccharides covalently attached to the population of the antibodies via N297 residues thereof comprise a core fucose residue.
A9. The population of the antibodies of any one of embodiments A1 to A8, wherein the antibodies are produced by expressing a polynucleotide encoding the antibodies or a fragment thereof in a host cell that is deficient in adding a fucose to an oligosaccharide attached to an antibody.
A10. The population of the antibodies of embodiment A9, wherein the host cell has reduced GDP-mannose 4,6-dehydratase (GMD) activity or reduced α-1,6 fucosyltransferase activity.
A11. The population of the antibodies of any one of embodiments A1 to A10, wherein the population of the antibodies have both enhanced antibody-dependent cellular cytotoxicity (ADCC) and enhanced complement-dependent cytotoxicity (CDC).
A12. The population of the antibodies of any one of embodiments A1 to A11, wherein the antibodies are IgG1.
A13. The population of the antibodies of any one of embodiments A1 to A12, wherein the antibodies bind to HLA-G, wherein optionally the antibodies are as described in Section 5.5 above.
A14. The population of the antibodies of embodiment A13, wherein the antibodies binding to HLA-G comprise a light chain nucleotide sequence of SEQ ID NO: 180 and a heavy chain nucleotide sequence of SEQ ID NO: 182 but with K338A and T437R mutations or K248E and T437R mutations (RE mutations), or a light chain nucleotide sequence of SEQ ID NO: 181 and a heavy chain nucleotide sequence of SEQ ID NO: 183 but with K338A and T437R mutations or K248E and T437R mutations (RE mutations).
A15. The population of the antibodies of any one of embodiments A1 to A12, wherein the antibodies bind to CD37, wherein optionally the antibodies are as described in Section 5.6 above.
A16. The population of the antibodies of embodiment A15, wherein the antibodies binding to CD37 comprise a light chain nucleotide sequence of SEQ ID NO: 69 and a heavy chain nucleotide sequence of SEQ ID NO: 85, a light chain nucleotide sequence of SEQ ID NO: 70 and a heavy chain nucleotide sequence of SEQ ID NO: 86, a light chain nucleotide sequence of SEQ ID NO: 71 and a heavy chain nucleotide sequence of SEQ ID NO: 87, a light chain nucleotide sequence of SEQ ID NO: 72 and a heavy chain nucleotide sequence of SEQ ID NO: 88, a light chain nucleotide sequence of SEQ ID NO: 73 and a heavy chain nucleotide sequence of SEQ ID NO: 89, a light chain nucleotide sequence of SEQ ID NO: 74 and a heavy chain nucleotide sequence of SEQ ID NO: 90, a light chain nucleotide sequence of SEQ ID NO: 75 and a heavy chain nucleotide sequence of SEQ ID NO: 91, or a light chain nucleotide sequence of SEQ ID NO: 76 and a heavy chain nucleotide sequence of SEQ ID NO: 92.
A17. The population of the antibodies of any one of embodiments A1 to A12, wherein the antibodies bind to GPRC5D, wherein optionally the antibodies are as described in Section 5.7 above.
A18. The population of the antibodies of embodiment A17, wherein the antibodies binding to GPRC5D comprise a light chain nucleotide sequence of SEQ ID NO: 104 and a heavy chain nucleotide sequence of SEQ ID NO: 106.
A19. The population of the antibodies of any one of embodiments A1 to A12, wherein the antibodies bind to KLK2, wherein optionally the antibodies are as described in Section 5.8 above.
A20. The population of the antibodies of embodiment A19, wherein the antibodies binding to KLK2 comprise a light chain nucleotide sequence of SEQ ID NO: 161 and a heavy chain nucleotide sequence of SEQ ID NO: 160, a light chain nucleotide sequence of SEQ ID NO: 161 and a heavy chain nucleotide sequence of SEQ ID NO: 162, or a light chain nucleotide sequence of SEQ ID NO: 161 and a heavy chain nucleotide sequence of SEQ ID NO: 163.
A21. The population of the antibodies of any one of embodiments A1 to A12, wherein the antibodies bind to PSMA, wherein optionally the antibodies are as described in Section 5.9 above.
A22. The population of the antibodies of embodiment A21, wherein the antibodies binding to PSMA comprise a light chain nucleotide sequence of SEQ ID NO: 241 and a heavy chain nucleotide sequence of SEQ ID NO: 242 but with K338A and T437R mutations or K248E and T437R mutations (RE mutations), or a light chain nucleotide sequence of SEQ ID NO: 241 and a heavy chain nucleotide sequence of SEQ ID NO: 243 but with K338A and T437R mutations or K248E and T437R mutations (RE mutations).
A23. The population of the antibodies of any one of embodiments A1 to A12, wherein the antibodies bind to BCMA, wherein optionally the antibodies are as described in Section 5.10 above.
A24. A population of antibodies,
   (i) wherein less than 80% of the oligosaccharides covalently attached to the population of the antibodies via N297 residues thereof comprise a core fucose residue; and
   (ii) wherein the population of the antibodies comprises an antibody having one or more mutations in the Fc region thereof for increasing CDC activity of the antibody.
A25. A population of antibodies, comprising a first means for increasing ADCC activity of the antibodies, and a second means for increasing CDC activity of the antibodies.

In another set of embodiments (embodiment set B), provided are:
B1. A pharmaceutical composition comprising:
   (a) a population of the antibodies,
   (i) wherein less than 80% of the oligosaccharides covalently attached to the population of the antibodies via N297 residues thereof comprise a core fucose residue; and
   (ii) wherein the population of the antibodies comprises an antibody which Fc region comprises K338A and T437R mutations, or K248E and T437R mutations (RE mutations),
wherein amino acid residue numbering is according to the EU numbering system, and
   (b) a pharmaceutically acceptable excipient.
B2. The pharmaceutical composition of embodiment B1, wherein less than 70% of the oligosaccharides covalently attached to the population of the antibodies via N297 residues thereof comprise a core fucose residue.
B3. The pharmaceutical composition of embodiment B1, wherein less than 60% of the oligosaccharides covalently attached to the population of the antibodies via N297 residues thereof comprise a core fucose residue.
B4. The pharmaceutical composition of embodiment B1, wherein less than 50% of the oligosaccharides covalently attached to the population of the antibodies via N297 residues thereof comprise a core fucose residue.
B5. The pharmaceutical composition of embodiment B1, wherein less than 40% of the oligosaccharides covalently attached to the population of the antibodies via N297 residues thereof comprise a core fucose residue.
B6. The pharmaceutical composition of embodiment B1, wherein less than 30% of the oligosaccharides covalently attached to the population of the antibodies via N297 residues thereof comprise a core fucose residue.
B7. The pharmaceutical composition of embodiment B1, wherein less than 20% of the oligosaccharides covalently attached to the population of the antibodies via N297 residues thereof comprise a core fucose residue.
B8. The pharmaceutical composition of embodiment B1, wherein less than 10% of the oligosaccharides covalently attached to the population of the antibodies via N297 residues thereof comprise a core fucose residue.
B9. The pharmaceutical composition of any one of embodiments B1 to B8, wherein the antibodies are produced by expressing a polynucleotide encoding the antibodies or a fragment thereof in a host cell that is deficient in adding a fucose to an oligosaccharide attached to an antibody.
B10. The pharmaceutical composition of embodiment B9, wherein the host cell has reduced GDP-mannose 4,6-dehydratase (GMD) activity or reduced α-1,6 fucosyltransferase activity.
B11. The pharmaceutical composition of any one of embodiments B1 to B10, wherein the population of the antibodies have both enhanced antibody-dependent cellular cytotoxicity (ADCC) and enhanced complement-dependent cytotoxicity (CDC).
B12. The pharmaceutical composition of any one of embodiments B1 to B11, wherein the antibodies are IgG1.
B13. The pharmaceutical composition of any one of embodiments B1 to B12, wherein the antibodies bind to HLA-G, wherein optionally the antibodies are as described in Section 5.5 above.
B14. The pharmaceutical composition of embodiment B13, wherein the antibodies binding to HLA-G comprise a light chain nucleotide sequence of SEQ ID NO: 180 and a heavy chain nucleotide sequence of SEQ ID NO: 182 but with K338A and T437R mutations or K248E and T437R mutations (RE mutations), or a light chain nucleotide sequence of SEQ ID NO: 181 and a heavy chain nucleotide sequence of SEQ ID NO: 183 but with K338A and T437R mutations or K248E and T437R mutations (RE mutations).
B15. The pharmaceutical composition of any one of embodiments B1 to B12, wherein the antibodies bind to CD37, wherein optionally the antibodies are as described in Section 5.6 above.
B16. The pharmaceutical composition of embodiment B15, wherein the antibodies binding to CD37 comprise a light chain nucleotide sequence of SEQ ID NO: 69 and a heavy chain nucleotide sequence of SEQ ID NO: 85, a light chain nucleotide sequence of SEQ ID NO: 70 and a heavy chain nucleotide sequence of SEQ ID NO: 86, a light chain nucleotide sequence of SEQ ID NO: 71 and a heavy chain nucleotide sequence of SEQ ID NO: 87, a light chain nucleotide sequence of SEQ ID NO: 72 and a heavy chain nucleotide sequence of SEQ ID NO: 88, a light chain nucleotide sequence of SEQ ID NO: 73 and a heavy chain nucleotide sequence of SEQ ID NO: 89, a light chain nucleotide sequence of SEQ ID NO: 74 and a heavy chain nucleotide sequence of SEQ ID NO: 90, a light chain nucleotide sequence of SEQ ID NO: 75 and a heavy chain nucleotide sequence of SEQ ID NO: 91, or a light chain nucleotide sequence of SEQ ID NO: 76 and a heavy chain nucleotide sequence of SEQ ID NO: 92.
B17. The pharmaceutical composition of any one of embodiments B1 to B12, wherein the antibodies bind to GPRC5D, wherein optionally the antibodies are as described in Section 5.7 above.
B18. The pharmaceutical composition of embodiment B17, wherein the antibodies binding to GPRC5D comprise a light chain nucleotide sequence of SEQ ID NO: 104 and a heavy chain nucleotide sequence of SEQ ID NO: 106.
B19. The pharmaceutical composition of any one of embodiments B1 to B12, wherein the antibodies bind to KLK2, wherein optionally the antibodies are as described in Section 5.8 above.
B20. The pharmaceutical composition of embodiment B19, wherein the antibodies binding to KLK2 comprise a light chain nucleotide sequence of SEQ ID NO: 161 and a heavy chain nucleotide sequence of SEQ ID NO: 160, a light chain nucleotide sequence of SEQ ID NO: 161 and a heavy chain nucleotide sequence of SEQ ID NO: 162, or a light chain nucleotide sequence of SEQ ID NO: 161 and a heavy chain nucleotide sequence of SEQ ID NO: 163.
B21. The pharmaceutical composition of any one of embodiments B1 to B12, wherein the antibodies bind to PSMA, wherein optionally the antibodies are as described in Section 5.9 above.
B22. The pharmaceutical composition of embodiment B21, wherein the antibodies binding to PSMA comprise a light chain nucleotide sequence of SEQ ID NO: 241 and a heavy chain nucleotide sequence of SEQ ID NO: 242 but with K338A and T437R mutations or K248E and T437R mutations (RE mutations), or a light chain nucleotide sequence of SEQ ID NO: 241 and a heavy chain nucleotide sequence of SEQ ID NO: 243 but with K338A and T437R mutations or K248E and T437R mutations (RE mutations).
B23. The pharmaceutical composition of any one of embodiments B1 to B12, wherein the antibodies bind to BCMA, wherein optionally the antibodies are as described in Section 5.10 above.
B24. A pharmaceutical composition comprising:
   (a) a population of the antibodies,
   (i) wherein less than 80% of the oligosaccharides covalently attached to the population of the antibodies via N297 residues thereof comprise a core fucose residue; and
   (ii) wherein the population of the antibodies comprises an antibody having one or more mutations in the Fc region thereof for increasing CDC activity of the antibody, and
   (b) a pharmaceutically acceptable excipient.
B25. A pharmaceutical composition comprising:
   (a) a population of the antibodies, comprising a first means for increasing ADCC activity of the antibodies, and a second means for increasing CDC activity of the antibodies; and
   (b) a pharmaceutically acceptable excipient.

In another set of embodiments (embodiment set C), provided are:
C1. A method of making a population of antibodies, comprising expressing a polynucleotide encoding the antibodies or a fragment thereof in a host cell that is deficient in adding a fucose residue to an oligosaccharide attached to an antibody via N297 residue, wherein the population of the antibodies comprises an antibody which Fc region comprises K338A and T437R mutations, or K248E and T437R mutations (RE mutations).
C2. The method of embodiment C1, wherein the host cell has reduced α-1,6 fucosyltransferase activity.
C3. The method of embodiment C1, wherein the host cell has reduced GDP-mannose 4,6-dehydratase activity.
C4. The method of embodiment C1, wherein the gene encoding α-1,6 fucosyltransferase is mutated, expressed at a lower than normal level, or knocked out in the host cell.
C5. The method of embodiment C1, wherein the gene encoding GDP-mannose 4,6-dehydratase is mutated, expressed at a lower than normal level, or knocked out in the host cell.
C6. The method of any one of embodiments C1 to C5, wherein less than 80% of the oligosaccharides covalently attached to the population of the antibodies via N297 residues thereof comprise a core fucose residue.
C7. The method of any one of embodiments C1 to C5, wherein less than 70% of the oligosaccharides covalently attached to the population of the antibodies via N297 residues thereof comprise a core fucose residue.
C8. The method of any one of embodiments C1 to C5, wherein less than 60% of the oligosaccharides covalently attached to the population of the antibodies via N297 residues thereof comprise a core fucose residue.
C9. The method of any one of embodiments C1 to C5, wherein less than 50% of the oligosaccharides covalently attached to the population of the antibodies via N297 residues thereof comprise a core fucose residue.
C10. The method of any one of embodiments C1 to C5, wherein less than 40% of the oligosaccharides covalently attached to the population of the antibodies via N297 residues thereof comprise a core fucose residue.
C11. The method of any one of embodiments C1 to C5, wherein less than 30% of the oligosaccharides covalently attached to the population of the antibodies via N297 residues thereof comprise a core fucose residue.
C12. The method of any one of embodiments C1 to C5, wherein less than 20% of the oligosaccharides covalently attached to the population of the antibodies via N297 residues thereof comprise a core fucose residue.
C13. The method of any one of embodiments C1 to C5, wherein less than 10% of the oligosaccharides covalently attached to the population of the antibodies via N297 residues thereof comprise a core fucose residue.
C14. The method of any one of embodiments C1 to C13, wherein the population of the antibodies have both enhanced antibody-dependent cellular cytotoxicity (ADCC) and enhanced complement-dependent cytotoxicity (CDC).
C15. The method of any one of embodiments C1 to C14, wherein the antibodies are IgG1.
C16. The method of any one of embodiments C1 to C15, wherein the antibodies bind to HLA-G, and wherein optionally the antibodies are as described in Section 5.5.
C17. The method of embodiment C16, wherein the antibodies binding to HLA-G comprise a light chain nucleotide sequence of SEQ ID NO: 180 and a heavy chain nucleotide sequence of SEQ ID NO: 182 but with K338A and T437R mutations or K248E and T437R mutations (RE mutations), or a light chain nucleotide sequence of SEQ ID NO: 181 and a heavy chain nucleotide sequence of SEQ ID NO: 183 but with K338A and T437R mutations or K248E and T437R mutations (RE mutations).
C18. The method of any one of embodiments C1 to C14, wherein the antibodies bind to CD37, wherein optionally the antibodies are as described in Section 5.6.
C19. The method of embodiment C18, wherein the antibodies binding to CD37 comprise a light chain nucleotide sequence of SEQ ID NO: 69 and a heavy chain nucleotide sequence of SEQ ID NO: 85, a light chain nucleotide sequence of SEQ ID NO: 70 and a heavy chain nucleotide sequence of SEQ ID NO: 86, a light chain nucleotide sequence of SEQ ID NO: 71 and a heavy chain nucleotide sequence of SEQ ID NO: 87, a light chain nucleotide sequence of SEQ ID NO: 72 and a heavy chain nucleotide sequence of SEQ ID NO: 88, a light chain nucleotide sequence of SEQ ID NO: 73 and a heavy chain nucleotide sequence of SEQ ID NO: 89, a light chain nucleotide sequence of SEQ ID NO: 74 and a heavy chain nucleotide sequence of SEQ ID NO: 90, a light chain nucleotide sequence of SEQ ID NO: 75 and a heavy chain nucleotide sequence of SEQ ID NO: 91, or a light chain nucleotide sequence of SEQ ID NO: 76 and a heavy chain nucleotide sequence of SEQ ID NO: 92.
C20. The method of any one of embodiments C1 to C14, wherein the antibodies bind to GPRC5D, wherein optionally the antibodies are as described in Section 5.7.
C21. The method of embodiment C20, wherein the antibodies binding to GPRC5D comprise a light chain nucleotide sequence of SEQ ID NO: 104 and a heavy chain nucleotide sequence of SEQ ID NO: 106.
C22. The method of any one of embodiments C1 to C14, wherein the antibodies bind to KLK2, wherein optionally the antibodies are as described in Section 5.8.
C23. The method of embodiment C22, wherein the antibodies binding to KLK2 comprise a light chain nucleotide sequence of SEQ ID NO: 161 and a heavy chain nucleotide sequence of SEQ ID NO: 160, a light chain nucleotide sequence of SEQ ID NO: 161 and a heavy chain nucleotide sequence of SEQ ID NO: 162, or a light chain nucleotide sequence of SEQ ID NO: 161 and a heavy chain nucleotide sequence of SEQ ID NO: 163.
C24. The method of any one of embodiments C1 to C14, wherein the antibodies bind to PSMA, wherein optionally the antibodies are as described in Section 5.9.
C25. The method of embodiment C24, wherein the antibodies binding to PSMA comprise a light chain nucleotide sequence of SEQ ID NO: 241 and a heavy chain nucleotide sequence of SEQ ID NO: 242 but with K338A and T437R mutations or K248E and T437R mutations (RE mutations), or a light chain nucleotide sequence of SEQ ID NO: 241 and a heavy chain nucleotide sequence of SEQ ID NO: 243 but with K338A and T437R mutations or K248E and T437R mutations (RE mutations).
C26. The method of any one of embodiments of C1 to C14, wherein the antibodies bind to BCMA, and wherein optionally the antibodies are as described in Section 5.10.
C27. A method of making a population of antibodies, comprising
   (i) a step for introducing K338A and T437R mutations, or K248E and T437R mutations (RE mutations) in the Fc regions of the population of the antibodies; and
   (ii) a step for producing the population of antibodies with reduced amount of core fucoses in the oligosaccharides attached to the antibodies via N297 residues.
C28. A method of making a population of antibodies, comprising a step of performing a function of expressing a polynucleotide encoding the antibodies or a fragment thereof in a host cell that is deficient in adding a fucose residue to an oligosaccharide attached to an antibody via N297 residue, wherein the population of the antibodies comprises an antibody which Fc region comprises K338A and T437R mutations, or K248E and T437R mutations (RE mutations).
C29. A method of making a population of antibodies, comprising
   (i) a step for performing the function of introducing K338A and T437R mutations, or K248E and T437R mutations (RE mutations) in the Fc regions of the population of the antibodies; and
   (ii) a step for performing the function of producing the population of antibodies with reduced amount of core fucoses in the oligosaccharides attached to the antibodies via N297 residues.

In yet another set of embodiments (embodiment set D), provided are:
D1. A method of treating a disease or disorder in a subject, comprising administering to the subject a population of antibodies,
   (i) wherein less than 80% of the oligosaccharides covalently attached to the population of the antibodies via N297 residues thereof comprise a core fucose residue; and
   (ii) wherein the population of the antibodies comprises an antibody which Fc region comprises K338A and T437R mutations, or K248E and T437R mutations (RE mutations),
wherein amino acid residue numbering is according to the EU numbering system.
D2. The method of embodiment D1, wherein less than 70% of the oligosaccharides covalently attached to the population of the antibodies via N297 residues thereof comprise a core fucose residue.
D3. The method of embodiment D1, wherein less than 60% of the oligosaccharides covalently attached to the population of the antibodies via N297 residues thereof comprise a core fucose residue.
D4. The method of embodiment D1, wherein less than 50% of the oligosaccharides covalently attached to the population of the antibodies via N297 residues thereof comprise a core fucose residue.
D5. The method of embodiment D1, wherein less than 40% of the oligosaccharides covalently attached to the population of the antibodies via N297 residues thereof comprise a core fucose residue.
D6. The method of embodiment D1, wherein less than 30% of the oligosaccharides covalently attached to the population of the antibodies via N297 residues thereof comprise a core fucose residue.
D7. The method of embodiment D1, wherein less than 20% of the oligosaccharides covalently attached to the population of the antibodies via N297 residues thereof comprise a core fucose residue.
D8. The method of embodiment D1, wherein less than 10% of the oligosaccharides covalently attached to the population of the antibodies via N297 residues thereof comprise a core fucose residue.
D9. The method of any one of embodiments D1 to D8, wherein the antibodies are produced by expressing a polynucleotide encoding the antibodies or a fragment thereof in a host cell that is deficient in adding a fucose to an oligosaccharide attached to an antibody.
D10. The method of embodiment D9, wherein the host cell has reduced GDP-mannose 4,6-dehydratase (GMD) activity or reduced α-1,6 fucosyltransferase activity.
D11. The method of any one of embodiments D1 to D10, wherein the population of the antibodies have both enhanced antibody-dependent cellular cytotoxicity (ADCC) and enhanced complement-dependent cytotoxicity (CDC).
D12. The method of any one of embodiments D1 to D11, wherein the antibodies are IgG1.
D13. The method of any one of embodiments D1 to D12, wherein the antibodies bind to HLA-G, wherein optionally the antibodies are as described in Section 5.5.
D14. The method of embodiment D13, wherein the antibodies binding to HLA-G comprise a light chain nucleotide sequence of SEQ ID NO: 180 and a heavy chain nucleotide sequence of SEQ ID NO: 182 but with K338A and T437R mutations or K248E and T437R mutations (RE mutations), or a light chain nucleotide sequence of SEQ ID NO: 181 and a heavy chain nucleotide sequence of SEQ ID NO: 183 but with K338A and T437R mutations or K248E and T437R mutations (RE mutations).
D15. The method of any one of embodiments D1 to D12, wherein the antibodies bind to CD37, and wherein optionally the antibodies are as described in Section 5.6.
D16. The method of embodiment D15, wherein the antibodies binding to CD37 comprise a light chain nucleotide sequence of SEQ ID NO: 69 and a heavy chain nucleotide sequence of SEQ ID NO: 85, a light chain nucleotide sequence of SEQ ID NO: 70 and a heavy chain nucleotide sequence of SEQ ID NO: 86, a light chain nucleotide sequence of SEQ ID NO: 71 and a heavy chain nucleotide sequence of SEQ ID NO: 87, a light chain nucleotide sequence of SEQ ID NO: 72 and a heavy chain nucleotide sequence of SEQ ID NO: 88, a light chain nucleotide sequence of SEQ ID NO: 73 and a heavy chain nucleotide sequence of SEQ ID NO: 89, a light chain nucleotide sequence of SEQ ID NO: 74 and a heavy chain nucleotide sequence of SEQ ID NO: 90, a light chain nucleotide sequence of SEQ ID NO: 75 and a heavy chain nucleotide sequence of SEQ ID NO: 91, or a light chain nucleotide sequence of SEQ ID NO: 76 and a heavy chain nucleotide sequence of SEQ ID NO: 92.
D17. The method of any one of embodiments D1 to D12, wherein the antibodies bind to GPRC5D, wherein optionally the antibodies are as described in Section 5.7.
D18. The method of embodiment D17, wherein the antibodies binding to GPRC5D comprise a light chain nucleotide sequence of SEQ ID NO: 104 and a heavy chain nucleotide sequence of SEQ ID NO: 106.
D19. The method of any one of embodiments D1 to D12, wherein the antibodies bind to KLK2, wherein optionally the antibodies are as described in Section 5.8.
D20. The method of embodiment D19, wherein the antibodies binding to KLK2 comprise a light chain nucleotide sequence of SEQ ID NO: 161 and a heavy chain nucleotide sequence of SEQ ID NO: 160, a light chain nucleotide sequence of SEQ ID NO: 161 and a heavy chain nucleotide sequence of SEQ ID NO: 162, or a light chain nucleotide sequence of SEQ ID NO: 161 and a heavy chain nucleotide sequence of SEQ ID NO: 163.
D21. The method of any one of embodiments D1 to D12, wherein the antibodies bind to PSMA, and wherein optionally the antibodies are as described in Section 5.9.
D22. The method of embodiment D21, wherein the antibodies binding to PSMA comprise a light chain nucleotide sequence of SEQ ID NO: 241 and a heavy chain nucleotide sequence of SEQ ID NO: 242 but with K338A and T437R mutations or K248E and T437R mutations (RE mutations), or a light chain nucleotide sequence of SEQ ID NO: 241 and a heavy chain nucleotide sequence of SEQ ID NO: 243 but with K338A and T437R mutations or K248E and T437R mutations (RE mutations).
D23. The method of any one of embodiments D1 to D12, wherein the antibodies bind to BCMA, and wherein optionally the antibodies are as described in Section 5.10.
D24. The method of any one of embodiments D1 to D11, wherein the antibodies bind to an antigen, and wherein the disease or disorder is associated with the antigen.
D25. The method of embodiment D24, wherein the antigen is HLA-G.
D26. The method of embodiment D24, wherein the antigen is CD37.
D27. The method of embodiment D24, wherein the antigen is GPRC5D.
D28. The method of embodiment D24, wherein the antigen is KLK2.
D29. The method of embodiment D24, wherein the antigen is PSMA.
D30. The method of embodiment D24, wherein the antigen is BCMA.
D31. The method of any one of embodiments D24 to D30, wherein the disease or disorder is solid tumor cancer.
D32. The method of any one of embodiments D24 to D30, wherein the disease or disorder is selected from a group consisting of renal, pancreatic or lung adenocarcinoma, non-small cell lung cancer, and ovarian cancer.
D33. A method of treating a disease or disorder in a subject, comprising administering to the subject a population of antibodies,
   (i) wherein less than 80% of the oligosaccharides covalently attached to the population of the antibodies via N297 residues thereof comprise a core fucose residue; and
   (ii) wherein the population of the antibodies comprises an antibody having one or more mutations in the Fc region thereof for increasing CDC activity of the antibody.
D34. A method of treating a disease or disorder in a subject, comprising administering to the subject (a) a population of the antibodies, comprising a first means for increasing ADCC activity of the antibodies, and a second means for increasing CDC activity of the antibodies; and (b) a pharmaceutically acceptable excipient.

In yet another set of embodiments (embodiment set E), provided are:
E1. A method of modulating an immunity in a host, comprising administering to the host a population of antibodies,
   (i) wherein less than 80% of the oligosaccharides covalently attached to the population of the antibodies via N297 residues thereof comprise a core fucose residue; and
   (ii) wherein the population of the antibodies comprises an antibody which Fc region comprises K338A and T437R mutations, or K248E and T437R mutations (RE mutations),
wherein amino acid residue numbering is according to the EU numbering system.
E2. The method of embodiment E1, wherein less than 70% of the oligosaccharides covalently attached to the population of the antibodies via N297 residues thereof comprise a core fucose residue.
E3. The method of embodiment E1, wherein less than 60% of the oligosaccharides covalently attached to the population of the antibodies via N297 residues thereof comprise a core fucose residue.
E4. The method of embodiment E1, wherein less than 50% of the oligosaccharides covalently attached to the population of the antibodies via N297 residues thereof comprise a core fucose residue.
E5. The method of embodiment E1, wherein less than 40% of the oligosaccharides covalently attached to the population of the antibodies via N297 residues thereof comprise a core fucose residue.
E6. The method of embodiment E1, wherein less than 30% of the oligosaccharides covalently attached to the population of the antibodies via N297 residues thereof comprise a core fucose residue.
E7. The method of embodiment E1, wherein less than 20% of the oligosaccharides covalently attached to the population of the antibodies via N297 residues thereof comprise a core fucose residue.
E8. The method of embodiment E1, wherein less than 10% of the oligosaccharides covalently attached to the population of the antibodies via N297 residues thereof comprise a core fucose residue.
E9. The method of any one of embodiments E1 to E8, wherein the antibodies are produced by expressing a polynucleotide encoding the antibodies or a fragment thereof in a host cell that is deficient in adding a fucose to an oligosaccharide attached to an antibody.
E10. The method of embodiment E9, wherein the host cell has reduced GDP-mannose 4,6-dehydratase (GMD) activity or reduced α-1,6 fucosyltransferase activity.
E11. The method of any one of embodiments E1 to E10, wherein the population of the antibodies have both enhanced antibody-dependent cellular cytotoxicity (ADCC) and enhanced complement-dependent cytotoxicity (CDC).
E12. The method of any one of embodiments E1 to E11, wherein the antibodies are IgG1.
E13. The method of any one of embodiments E1 to E12, wherein the antibodies bind to HLA-G, and wherein optionally the antibodies are as described in Section 5.5.
E14. The method of embodiment E13, wherein the antibodies binding to HLA-G comprise a light chain nucleotide sequence of SEQ ID NO: 180 and a heavy chain nucleotide sequence of SEQ ID NO: 182 but with K338A and T437R mutations or K248E and T437R mutations (RE mutations), or a light chain nucleotide sequence of SEQ ID NO: 181 and a heavy chain nucleotide sequence of SEQ ID NO: 183 but with K338A and T437R mutations or K248E and T437R mutations (RE mutations).
E15. The method of any one of embodiments E1 to E12, wherein the antibodies bind to CD37, and wherein optionally the antibodies are as described in Section 5.6.
E16. The method of embodiment E15, wherein the antibodies binding to CD37 comprise a light chain nucleotide sequence of SEQ ID NO: 69 and a heavy chain nucleotide sequence of SEQ ID NO: 85, a light chain nucleotide sequence of SEQ ID NO: 70 and a heavy chain nucleotide sequence of SEQ ID NO: 86, a light chain nucleotide sequence of SEQ ID NO: 71 and a heavy chain nucleotide sequence of SEQ ID NO: 87, a light chain nucleotide sequence of SEQ ID NO: 72 and a heavy chain nucleotide sequence of SEQ ID NO: 88, a light chain nucleotide sequence of SEQ ID NO: 73 and a heavy chain nucleotide sequence of SEQ ID NO: 89, a light chain nucleotide sequence of SEQ ID NO: 74 and a heavy chain nucleotide sequence of SEQ ID NO: 90, a light chain nucleotide sequence of SEQ ID NO: 75 and a heavy chain nucleotide sequence of SEQ ID NO: 91, or a light chain nucleotide sequence of SEQ ID NO: 76 and a heavy chain nucleotide sequence of SEQ ID NO: 92.
E17. The method of any one of embodiments E1 to E12, wherein the antibodies bind to GPRC5D, and wherein optionally the antibodies are as described in Section 5.7.
E18. The method of embodiment E17, wherein the antibodies binding to GPRC5D comprise a light chain nucleotide sequence of SEQ ID NO: 104 and a heavy chain nucleotide sequence of SEQ ID NO: 106.
E19. The method of any one of embodiments E1 to E12, wherein the antibodies bind to KLK2, and wherein optionally the antibodies are as described in Section 5.8.
E20. The method of embodiment E19, wherein the antibodies binding to KLK2 comprise a light chain nucleotide sequence of SEQ ID NO: 161 and a heavy chain nucleotide sequence of SEQ ID NO: 160, a light chain nucleotide sequence of SEQ ID NO: 161 and a heavy chain nucleotide sequence of SEQ ID NO: 162, or a light chain nucleotide sequence of SEQ ID NO: 161 and a heavy chain nucleotide sequence of SEQ ID NO: 163.
E21. The method of any one of embodiments E1 to E12, wherein the antibodies bind to PSMA, and wherein optionally the antibodies are as described in Section 5.9.
E22. The method of embodiment E21, wherein the antibodies binding to PSMA comprise a light chain nucleotide sequence of SEQ ID NO: 241 and a heavy chain nucleotide sequence of SEQ ID NO: 242 but with K338A and T437R mutations or K248E and T437R mutations (RE mutations), or a light chain nucleotide sequence of SEQ ID NO: 241 and a heavy chain nucleotide sequence of SEQ ID NO: 243 but with K338A and T437R mutations or K248E and T437R mutations (RE mutations).
E23. The method of any one of embodiments E1 to E12, wherein the antibodies bind to BCMA, and wherein optionally the antibodies are as described in Section 5.10.
E24. A method of modulating an immunity in a host, comprising administering a population of antibodies,
   (i) wherein less than 80% of the oligosaccharides covalently attached to the population of the antibodies via N297 residues thereof comprise a core fucose residue; and
   (ii) wherein the population of the antibodies comprises an antibody having one or more mutations in the Fc region thereof for increasing CDC activity of the antibody.
E25. A method of modulating an immunity in a host, comprising administering a population of the antibodies, comprising a first means for increasing ADCC activity of the antibodies, and a second means for increasing CDC activity of the antibodies.

### 7. EXAMPLES

The following is a description of various methods and materials used in the studies. They are put forth so as to provide those of ordinary skill in the art with a complete disclosure and description of how to make and use the present invention, and are not intended to limit the scope of what the inventors regard as their invention, nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### Example 1—ADCC and CDC Functional Analysis of Anti-HLA-G Antibodies

Anti-HLA-G antibodies and their variants with modified constant regions (i.e., low fucosylated and/or with K248E and T437R (RE) mutations) are tested in this study, and these antibodies are summarized in the Table 3 below.

TABLE 3
Anti-HLA-G antibodies and their variants

| Antibody | Description |
|---|---|
| Wildtype antibody | IgG1 |
| Nagative control | L234A, L235A, D265S |
| Antibody with RE | IgG1, K248E, T437R (RE) |
| mutations | |
| Antibodies with low | IgG1, low fucosylation |
| fucosylation | |
| Antibodies with RE | IgG1, K248E, T437R (RE), |
| mutations and low | low fucosylation |
| fucosylation | |

ADCC Functional Analysis of Antibodies with Normal and Low Fucosylation

Anti-HLA-G antibodies with normal and low fucosylation are tested for their ability to mediate tumor cell killing via ADCC in the choriocarcinoma cell line JEG-3, which endogenously expresses HLA-G. Briefly, JEG-3 cells are loaded with BATDA, a cell-labeling reagent that is released upon cell death and can be detected in the cell culture supernatant aftering forming a fluorescent chelate. Anti-HLA-G antibodies are added to BATDA-labelled JEG-3 cells. Peripheral blood mononuclear cells (PBMC) cultured overnight are added at an effector to target cell ratio (E:T ratio) of 50:1 to JEG-3 cells at 5,000 cells/well. The mixture is incubated for 4 hours at 37° C. Europium (III) chelate, a BATDA chelate substrate, is added to the mixture at a ratio of 1:10. The amount of released BATDA is determined by measuring emitted fluorescence at 615 nm. The fluorescence signal for 100% tumor cell killing is determined using a well containing BATDA-labeled target cells mixed with Triton-X 100 detergent.

An anti-HLA-G antibody is expressed in fucosylation-deficient Chinese Hamster Ovary (CHO) cells to produce antibodies with less than 10% fucosylation. A modified version of the wildtype antibody with an Fc region that does not bind Fc receptors is generated as a negative control, which comprises the L234A, L235A and D265S mutations. Populations of the wildtype antibodies with normal and low fucosylation are tested for their abilities to induce NK cell-based ADCC against either JEG-3 cells, at an effector:target ratio of 50:1.

### CDC Functional Analysis of Antibodies Comprising RE Mutations

Anti-HLA-G antibodies with RE mutations are tested for their ability to mediate tumor cell killing via CDC in the choriocarcinoma cell line JEG-3 or adenocarcinoma cell line RERF-LC-Ad-1. Briefly, JEG-3 cells are cultured in DMEM medium with 10% Fetal Bovine Serum (FBS), and RERF-LC-Ad-1 are cultured in RPMI medium with 10% FBS. Antibodies are added to target cells and incubated for 30 minutes at 37° C. Baby rabbit serum is then added to target cells to a final concentration of 10% to provide a source of complement components for CDC. The mixture is incubated for 4 hours at 37° C. 100 μl of CellTiter-Glo reagent (Promega) is added to the mixture followed by incubation for 10 minutes at room temperature. Target cell viability is determined by measuring luminescence with a Tecan SPARK Reader and reported in Relative Luminescence Units (RLU).

ADCC and CDC Functional Analysis of Antibodies with Normal and Low Fucosylation Comprising RE Mutations

Antibodies comprising RE mutations are expressed in fucosylation-deficient CHO host cells to produce antibodies with less than 10% fucosylation. Antibodies with normal and low fucosylation are tested for their abilities to mediate CDC against cells.

Antibodies with RE mutations and low fucosylation are also tested for their ADCC activity. Briefly, antibodies are added to JEG-3 cells that have been pre-loaded with BATDA. Peripheral blood mononuclear cells (PBMC) cultured overnight are added at an effector to target cell ratio (E:T ratio) of 50:1 to JEG-3 cells at 5,000 cells/well. The mixture is incubated for 4 hours at 37° C. Europium (III) chelate is added to the mixture at a ratio of 1:10. The amount of released BATDA is determined by measuring emitted fluorescence at 615 nm. The fluorescence signal for 100% tumor cell killing is determined using a well containing BATDA-labeled target cells mixed with Triton-X 100 detergent.

### Example 2—CDC Functional Analysis of Anti-CD37 Antibodies

Eight different types of anti-CD37 antibodies (Table 2) were tested for their ability to mediate tumor cell killing via CDC in the cell lines CARNAVAL, which expresses high levels of CD37, and JEKO-1, which expresses lower levels of CD37. Briefly, for each type of anti-CD37 antibody, an antibody with low fucosylation (i.e., T26B375.CLF, T26B382.CLF, T26B386.CLF, T26B379.CLF, T26B373.CLF, T26B388.CLF, T26B385.CLF and T26B374.CLF, respectively), an antibody comprising S239D/I332E (Xencor) mutations (i.e., T26B612, T26B613, T26B614, T26B615, T26B608, T26B609, T26B610 and T26B611, respectively), an antibody comprising RE mutations (i.e., T26B461, T26B463, T26B465, T26B462, T26B459, T26B466, T26B464 and T26B460, respectively), and an antibody comprising RE mutations with low fucosylation (i.e., T26B461.CLF, T26B463.CLF, T26B465.CLF, T26B462.CLF, T26B459.CLF, T26B466.CLF, T26B464.CLF and T26B460.CLF, respectively) were generated. Anti-CD37 antibodies were added to the CARNAVAL target cells and incubated for 30 minutes at 37° C. Baby rabbit serum was then added to target cells to a final concentration of 10% to provide a source of complement components for CDC. The mixture was incubated for 4 hours at 37° C. 100 μl of CellTiter-Glo reagent (Promega) was added to the mixture followed by incubation for 10 minutes at room temperature. Target cell viability was determined by measuring luminescence with a Tecan SPARK Reader and reported in Relative Luminescence Units (RLU).

Anti-CD37 antibodies and their variants with modified constant regions are summarized in the Table 4 below.

TABLE 4
Anti-CD37 antibodies and their variants

| | | | | Antibodies |
|---|---|---|---|---|
| | | | | with RE |
| | IgG1 antibodies | Antibodies | Antibodies | mutations |
| | with low | with Xencor | with RE | and low |
| | fucosylation | mutations | mutations | fucosylation |
| 1 | T26B375.CLF | T26B612 | T26B461 | T26B461.CLF |
| 2 | T26B382.CLF | T26B613 | T26B463 | T26B463.CLF |
| 3 | T26B386.CLF | T26B614 | T26B465 | T26B465.CLF |
| 4 | T26B379.CLF | T26B615 | T26B462 | T26B462.CLF |
| 5 | T26B373.CLF | T26B608 | T26B459 | T26B459.CLF |
| 6 | T26B388.CLF | T26B609 | T26B466 | T26B466.CLF |
| 7 | T26B385.CLF | T26B610 | T26B464 | T26B464.CLF |
| 8 | T26B374.CLF | T26B611 | T26B460 | T26B460.CLF |

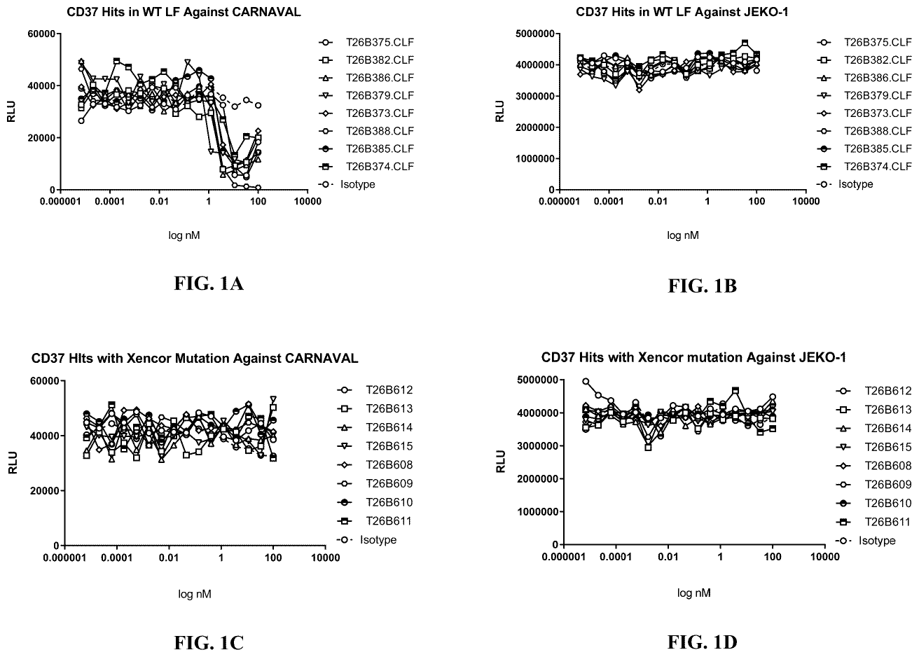

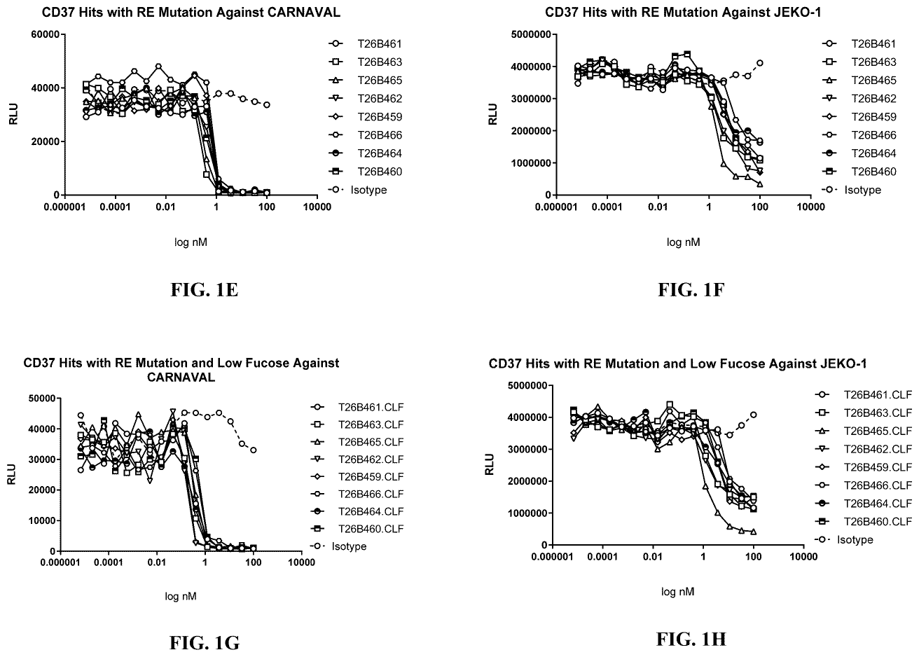

The anti-CD37 antibodies with low fucosylation showed CDC activity against CARNAVAL cells but not JEKO-1 cells (FIGS. 1A and 1B). Xencor mutations in these antibodies ablated their CDC activity against CARNAVAL cells significantly (FIG. 1C). RE mutations further increased the CDC potency of these antibodies against CARNAVAL cells, while conferring these antibodies CDC activity against JEKO-1 cells (FIGS. 1E and 1F). Importantly, the potentiation of CDC activity by RE mutations were unaffected when antibodies with low fucosylation were used (FIGS. 1G and 1H) compared to those with normal fucosylation (FIGS. 1E and 1F).

Taken together, these results indicated that the CDC potentiation characteristics of the RE mutations are not target dependent. They also showed that, while the S239D/I332E mutations were shown to improve ADCC activity of antibodies (Lazar, G. A. et al., supra), they negatively affect CDC. In contrast, the RE mutations can enhance CDC activity of antibodies. This potentiation is not affected by low fucosylation of the antibodies, which can potentiate their ADCC activity.

The VL and VH amino acid sequences of exemplary anti-CD37 antibodies are summarized in Table 5 and Table 6 below.

TABLE 5
VL amino acid sequences of exemplary anti-CD37 antibodies

| | Variable Sequence |
|---|---|
| Antibody Name | (SEQ ID NO) |
| T26B373 | DIQMTQSPSTLSASVGDRVTITCRASQSTSSWLAWYQQKPG |
| | KAPKLLIYKTSSLESGVPSRFSGSGSGTEFTLTISSLQPDDFA |
| | TYYCQQYNSYSFGQGTKVEIK (SEQ ID NO: 1) |
| T26B374 | DIQMTQSPSTLSASVGDRVTITCRASQSISSWLAWYQQKPG |
| | KAPKLLIYKASSLESGVPSRFSGSRSGTEFTLTISSLQPDDFA |
| | TYYCQQYNSWTFGQGTKVEIK (SEQ ID NO: 2) |
| T26B375 | DIQMTQSPSTLSASVGDRVTITCRASQSISSWLAWYQQKPG |
| | KAPKLLIYKASSLESGVPSRFSGSGSGTEFTLTISSLQPDDFA |
| | TYYCQQYNSYTFGQGTKLEIK (SEQ ID NO: 3) |
| T26B379 | DIQMTQSPSTLSASVGDRVTITCRASQSISSWLAWYQQKPG |
| | KAPKLLIFKTSSLESGVPSRFSGSGSGTEFTLTISSLQPDDFAT |
| | YYCQQYNSYSFGGGTKVEIK (SEQ ID NO: 4) |
| T26B382 | QSALTQPASVSGSPGQSITISCTGTSSDVGGYNYVSWYQQH |
| | PSKAPKLMIYDVSNRPSGVSNRFSGSKSGNTASLTISGLQAE |
| | DEADYYCSSYTSSSTLVVFGGGTKLTVL (SEQ ID NO: 5) |
| T26B385 | DIQMTQSPSTLSASVGDRVTITCRASQSLSSWLAWYQQKPG |
| | KAPKLLIFKTSSLESGVPSRFSGSGSGTEFTLTISSLQPDDFAI |
| | YYCQQYNSYIFGQGTRLEIK (SEQ ID NO: 6) |
| T26B386 | SSELTQDPAVSVALGQTVRITCQGDSLRNYYASWYQQKPG |
| | QAPVLVFYGKDNRPSGIPDRFSGSTSGNTASLTITGAQAQD |
| | EADYYCNSRDSSGDHLVFGGGTKLTVL (SEQ ID NO: 7) |
| T26B388 | DIQMTQSPSSLSASVGDRVTITCRASQGIRNDLGWYQQKPG |
| | KAPKRLIYAASSLQGGVPSRFSGSGSGTEFTLTISSLQPEDFA |
| | TYYCLQHYTYPLTFGGGTKVEIK (SEQ ID NO: 8) |
| T26B459 | DIQMTQSPSTLSASVGDRVTITCRASQSTSSWLAWYQQKPG |
| | KAPKLLIYKTSSLESGVPSRFSGSGSGTEFTLTISSLQPDDFA |
| | TYYCQQYNSYSFGQGTKVEIK (SEQ ID NO: 1) |
| T26B460 | DIQMTQSPSTLSASVGDRVTITCRASQSISSWLAWYQQKPG |
| | KAPKLLIYKASSLESGVPSRFSGSRSGTEFTLTISSLQPDDFA |
| | TYYCQQYNSWTFGQGTKVEIK (SEQ ID NO: 2) |
| T26B461 | DIQMTQSPSTLSASVGDRVTITCRASQSISSWLAWYQQKPG |
| | KAPKLLIYKASSLESGVPSRFSGSGSGTEFTLTISSLQPDDFA |
| | TYYCQQYNSYTFGQGTKLEIK (SEQ ID NO: 3) |
| T26B462 | DIQMTQSPSTLSASVGDRVTITCRASQSISSWLAWYQQKPG |
| | KAPKLLIFKTSSLESGVPSRFSGSGSGTEFTLTISSLQPDDFAT |
| | YYCQQYNSYSFGGGTKVEIK (SEQ ID NO: 4) |
| T26B463 | QSALTQPASVSGSPGQSITISCTGTSSDVGGYNYVSWYQQH |
| | PSKAPKLMIYDVSNRPSGVSNRFSGSKSGNTASLTISGLQAE |
| | DEADYYCSSYTSSSTLVVFGGGTKLTVL (SEQ ID NO: 5) |
| T26B464 | DIQMTQSPSTLSASVGDRVTITCRASQSLSSWLAWYQQKPG |
| | KAPKLLIFKTSSLESGVPSRFSGSGSGTEFTLTISSLQPDDFAI |
| | YYCQQYNSYIFGQGTRLEIK (SEQ ID NO: 6) |
| T26B465 | SSELTQDPAVSVALGQTVRITCQGDSLRNYYASWYQQKPG |
| | QAPVLVFYGKDNRPSGIPDRFSGSTSGNTASLTITGAQAQD |
| | EADYYCNSRDSSGDHLVFGGGTKLTVL (SEQ ID NO: 7) |
| T26B466 | DIQMTQSPSSLSASVGDRVTITCRASQGIRNDLGWYQQKPG |
| | KAPKRLIYAASSLQGGVPSRFSGSGSGTEFTLTISSLQPEDFA |
| | TYYCLQHYTYPLTFGGGTKVEIK (SEQ ID NO: 8) |
| T26B608 | DIQMTQSPSTLSASVGDRVTITCRASQSTSSWLAWYQQKPG |
| | KAPKLLIYKTSSLESGVPSRFSGSGSGTEFTLTISSLQPDDFA |
| | TYYCQQYNSYSFGQGTKVEIK (SEQ ID NO: 1) |
| T26B609 | DIQMTQSPSSLSASVGDRVTITCRASQGIRNDLGWYQQKPG |
| | KAPKRLIYAASSLQGGVPSRFSGSGSGTEFTLTISSLQPEDFA |
| | TYYCLQHYTYPLTFGGGTKVEIK (SEQ ID NO: 8) |
| T26B610 | DIQMTQSPSTLSASVGDRVTITCRASQSLSSWLAWYQQKPG |
| | KAPKLLIFKTSSLESGVPSRFSGSGSGTEFTLTISSLQPDDFAI |
| | YYCQQYNSYIFGQGTRLEIK (SEQ ID NO: 6) |
| T26B611 | DIQMTQSPSTLSASVGDRVTITCRASQSISSWLAWYQQKPG |
| | KAPKLLIYKASSLESGVPSRFSGSRSGTEFTLTISSLQPDDFA |
| | TYYCQQYNSWTFGQGTKVEIK (SEQ ID NO: 2) |
| T26B612 | DIQMTQSPSTLSASVGDRVTITCRASQSISSWLAWYQQKPG |
| | KAPKLLIYKASSLESGVPSRFSGSGSGTEFTLTISSLQPDDFA |
| | TYYCQQYNSYTFGQGTKLEIK (SEQ ID NO: 3) |
| T26B613 | QSALTQPASVSGSPGQSITISCTGTSSDVGGYNYVSWYQQH |
| | PSKAPKLMIYDVSNRPSGVSNRFSGSKSGNTASLTISGLQAE |
| | DEADYYCSSYTSSSTLVVFGGGTKLTVL (SEQ ID NO: 5) |
| T26B614 | SSELTQDPAVSVALGQTVRITCQGDSLRNYYASWYQQKPG |
| | QAPVLVFYGKDNRPSGIPDRFSGSTSGNTASLTITGAQAQD |
| | EADYYCNSRDSSGDHLVFGGGTKLTVL (SEQ ID NO: 7) |
| T26B615 | DIQMTQSPSTLSASVGDRVTITCRASQSISSWLAWYQQKPG |
| | KAPKLLIFKTSSLESGVPSRFSGSGSGTEFTLTISSLQPDDFAT |
| | YYCQQYNSYSFGGGTKVEIK (SEQ ID NO: 4) |

TABLE 6
VH amino acid sequences of exemplary anti-CD37 antibodies

| | Variable Sequence |
|---|---|
| Antibody Name | (SEQ ID NO) |
| T26B373 | QVQLQESGPGLVKPSETLSLTCTVSGGSISSGVYYWAWIRQ |
| | PPGKGLELIGTFYYSGSTYYDSSLRSRVTISVDTSKNQFSLK |
| | LSSVTAADTAVYYCARQAGDFDYWGQGTLVTVSS (SEQ ID |
| | NO: 9) |
| T26B374 | QLQLQESGPGLVKPSETLSLTCTVSGGSISSSPYYWGWIRQP |
| | PGKGLEWIGSFYYGGSNYYNPSLKSRVTISADTSKNQFSLK |
| | LSSVTAADTAVYYCARQAGDWDYWGQGTLVTVSS (SEQ |
| | ID NO: 10) |
| T26B375 | QLQLQESGPGLVKPSETLSLTCTVSGGSISSGIYNWGWIRQP |
| | PGKGLEWIGNFQYSGITYYNPSLKSRVTISVDTSKNQFSLQL |
| | SSVTAADTAVYYCARQAGDFDYWGQGTLVTVSS (SEQ ID |
| | NO: 11) |
| T26B379 | QVQLQESGPGLVKPSETLSLTCTVSGGSISSSIYYWAWIRQP |
| | PGKGLEWIGTIYYGGSPYYSPSLKSRVTISIDTSKSQFSLRLT |
| | SVTVADTAVYYCARRAGDFDYWGQGTLVTVSS (SEQ ID |
| | NO: 12) |
| T26B382 | QVQLVESGGGLVKPGGSLRLSCAASGFTFSDYYMTWIRQA |
| | PGKGLEWVSYISSSGITKYYADFVKGRFTISRDNAKNSLYL |
| | QMNSLRAEDTAVYYCARDRDRQWLLEFDYWGQGTLVTVS |
| | S (SEQ ID NO: 13) |
| T26B385 | QVQLQESGPGLVKPSETLSLTCIVSGGSVSSRNYYWGWIRQ |
| | PPGKGLEWIGRIYYSGNTNYNPSLKSRVTISVDTSKNQFSLK |
| | LSSVTAADTAVYYCARWAGEIDYWGQGTLVTVSS(SEQ ID |
| | NO: 14) |
| T26B386 | QVQLQESGPGLVKPSETLSLSCTVSGGSLNSYNYYWGWVR |
| | QPPGKGLEWIGTIYSSGSAYYNPSLKSRFTISVATSKNQFSL |
| | RLSSVTAADTAVYYCARGYRNSWYALFEYWGQGTLVTVS |
| | S (SEQ ID NO: 15) |
| T26B388 | QVQLQQWGAGLLKPSETLSLTCAVYGGSFSDYYWSWIRQP |
| | PGKGLEWIGEIDHSGSTDYNPSLKSRVTISVDTSKNQFSLKL |
| | SSVTAADTAVYYCARSMYYDIWTGYHGAFDIWGQGTMVT |
| | VSS (SEQ ID NO: 16) |
| T26B459 | QVQLQESGPGLVKPSETLSLTCTVSGGSISSGVYYWAWIRQ |
| | PPGKGLELIGTFYYSGSTYYDSSLRSRVTISVDTSKNQFSLK |
| | LSSVTAADTAVYYCARQAGDFDYWGQGTLVTVSS (SEQ ID |
| | NO: 9) |
| T26B460 | QLQLQESGPGLVKPSETLSLTCTVSGGSISSSPYYWGWIRQP |
| | PGKGLEWIGSFYYGGSNYYNPSLKSRVTISADTSKNQFSLK |
| | LSSVTAADTAVYYCARQAGDWDYWGQGTLVTVSS (SEQ |
| | ID NO: 10) |
| T26B461 | QLQLQESGPGLVKPSETLSLTCTVSGGSISSGIYNWGWIRQP |
| | PGKGLEWIGNFQYSGITYYNPSLKSRVTISVDTSKNQFSLQL |
| | SSVTAADTAVYYCARQAGDFDYWGQGTLVTVSS (SEQ ID |
| | NO: 11) |
| T26B462 | QVQLQESGPGLVKPSETLSLTCTVSGGSISSSIYYWAWIRQP |
| | PGKGLEWIGTIYYGGSPYYSPSLKSRVTISIDTSKSQFSLRLT |
| | SVTVADTAVYYCARRAGDFDYWGQGTLVTVSS (SEQ ID |
| | NO: 12) |
| T26B463 | QVQLVESGGGLVKPGGSLRLSCAASGFTFSDYYMTWIRQA |
| | PGKGLEWVSYISSSGITKYYADFVKGRFTISRDNAKNSLYL |
| | QMNSLRAEDTAVYYCARDRDRQWLLEFDYWGQGTLVTVS |
| | S (SEQ ID NO: 13) |
| T26B464 | QVQLQESGPGLVKPSETLSLTCIVSGGSVSSRNYYWGWIRQ |
| | PPGKGLEWIGRIYYSGNTNYNPSLKSRVTISVDTSKNQFSLK |
| | LSSVTAADTAVYYCARWAGEIDYWGQGTLVTVSS (SEQ ID |
| | NO: 14) |
| T26B465 | QVQLQESGPGLVKPSETLSLSCTVSGGSLNSYNYYWGWVR |
| | QPPGKGLEWIGTIYSSGSAYYNPSLKSRFTISVATSKNQFSL |
| | RLSSVTAADTAVYYCARGYRNSWYALFEYWGQGTLVTVS |
| | S (SEQ ID NO: 15) |
| T26B466 | QVQLQQWGAGLLKPSETLSLTCAVYGGSFSDYYWSWIRQP |
| | PGKGLEWIGEIDHSGSTDYNPSLKSRVTISVDTSKNQFSLKL |
| | SSVTAADTAVYYCARSMYYDIWTGYHGAFDIWGQGTMVT |
| | VSS (SEQ ID NO: 16) |
| T26B608 | QVQLQESGPGLVKPSETLSLTCTVSGGSISSGVYYWAWIRQ |
| | PPGKGLELIGTFYYSGSTYYDSSLRSRVTISVDTSKNQFSLK |
| | LSSVTAADTAVYYCARQAGDFDYWGQGTLVTVSS (SEQ ID |
| | NO: 9) |
| T26B609 | QVQLQQWGAGLLKPSETLSLTCAVYGGSFSDYYWSWIRQP |
| | PGKGLEWIGEIDHSGSTDYNPSLKSRVTISVDTSKNQFSLKL |
| | SSVTAADTAVYYCARSMYYDIWTGYHGAFDIWGQGTMVT |
| | VSS (SEQ ID NO: 16) |
| T26B610 | QVQLQESGPGLVKPSETLSLTCIVSGGSVSSRNYYWGWIRQ |
| | PPGKGLEWIGRIYYSGNTNYNPSLKSRVTISVDTSKNQFSLK |
| | LSSVTAADTAVYYCARWAGEIDYWGQGTLVTVSS (SEQ ID |
| | NO: 14) |
| T26B611 | QLQLQESGPGLVKPSETLSLTCTVSGGSISSSPYYWGWIRQP |
| | PGKGLEWIGSFYYGGSNYYNPSLKSRVTISADTSKNQFSLK |
| | LSSVTAADTAVYYCARQAGDWDYWGQGTLVTVSS (SEQ |
| | ID NO: 10) |
| T26B612 | QLQLQESGPGLVKPSETLSLTCTVSGGSISSGIYNWGWIRQP |
| | PGKGLEWIGNFQYSGITYYNPSLKSRVTISVDTSKNQFSLQL |
| | SSVTAADTAVYYCARQAGDFDYWGQGTLVTVSS (SEQ ID |
| | NO: 11) |
| T26B613 | QVQLVESGGGLVKPGGSLRLSCAASGFTFSDYYMTWIRQA |
| | PGKGLEWVSYISSSGITKYYADFVKGRFTISRDNAKNSLYL |
| | QMNSLRAEDTAVYYCARDRDRQWLLEFDYWGQGTLVTVS |
| | S (SEQ ID NO: 13) |
| T26B614 | QVQLQESGPGLVKPSETLSLSCTVSGGSLNSYNYYWGWVR |
| | QPPGKGLEWIGTIYSSGSAYYNPSLKSRFTISVATSKNQFSL |
| | RLSSVTAADTAVYYCARGYRNSWYALFEYWGQGTLVTVS |
| | S (SEQ ID NO: 15) |
| T26B615 | QVQLQESGPGLVKPSETLSLTCTVSGGSISSSIYYWAWIRQP |
| | PGKGLEWIGTIYYGGSPYYSPSLKSRVTISIDTSKSQFSLRLT |
| | SVTVADTAVYYCARRAGDFDYWGQGTLVTVSS (SEQ ID |
| | NO: 12) |

The complete amino acid sequences of the light chains and heavy chains of exemplary anti-CD37 antibodies are summarized in Table 7 and Table 8 below.

TABLE 7
Complete amino acid sequences of the light chains of
exemplary anti-CD37 antibodies

| | Complete Sequence |
|---|---|
| Antibody Name | (SEQ ID NO) |
| T26B373 | DIQMTQSPSTLSASVGDRVTITCRASQSTSSWLAWYQQKPG |
| | KAPKLLIYKTSSLESGVPSRFSGSGSGTEFTLTISSLQPDDFA |
| | TYYCQQYNSYSFGQGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGEC (SEQ ID NO: 37) |
| T26B374 | DIQMTQSPSTLSASVGDRVTITCRASQSISSWLAWYQQKPG |
| | KAPKLLIYKASSLESGVPSRFSGSRSGTEFTLTISSLQPDDFA |
| | TYYCQQYNSWTFGQGTKVEIKRTVAAPSVFIFPPSDEQLKS |
| | GTASVVCLLNNFYPREAKVQWKVDNALQSGNSQESVTEQ |
| | DSKDSTYSLSSTLTLSKADYEKHKVYACEVTHQGLSSPVTK |
| | SFNRGEC (SEQ ID NO: 38) |
| T26B375 | DIQMTQSPSTLSASVGDRVTITCRASQSISSWLAWYQQKPG |
| | KAPKLLIYKASSLESGVPSRFSGSGSGTEFTLTISSLQPDDFA |
| | TYYCQQYNSYTFGQGTKLEIKRTVAAPSVFIFPPSDEQLKSG |
| | TASVVCLLNNFYPREAKVQWKVDNALQSGNSQESVTEQDS |
| | KDSTYSLS STLTL SKADYEKHKVYACEVTHQGLSSPVTKSF |
| | NRGEC (SEQ ID NO: 39) |
| T26B379 | DIQMTQSPSTLSASVGDRVTITCRASQSISSWLAWYQQKPG |
| | KAPKLLIFKTSSLESGVPSRFSGSGSGTEFTLTISSLQPDDFAT |
| | YYCQQYNSYSFGGGTKVEIKRTVAAPSVFIFPPSDEQLKSGT |
| | ASVVCLLNNFYPREAKVQWKVDNALQSGNSQESVTEQDS |
| | KDSTYSLSSTLTLSKADYEKHKVYACEVTHQGLSSPVTKSF |
| | NRGEC (SEQ ID NO: 40) |
| T26B382 | QSALTQPASVSGSPGQSITISCTGTSSDVGGYNYVSWYQQH |
| | PSKAPKLMIYDVSNRPSGVSNRFSGSKSGNTASLTISGLQAE |
| | DEADYYCSSYTSSSTLVVFGGGTKLTVLGQPKAAPSVTLFP |
| | PSSEELQANKATLVCLISDFYPGAVTVAWKADSSPVKAGV |
| | ETTTPSKQSNNKYAASSYLSLTPEQWKSHRSYSCQVTHEGS |
| | TVEKTVAPTECS (SEQ ID NO: 41) |
| T26B385 | DIQMTQSPSTLSASVGDRVTITCRASQSLSSWLAWYQQKPG |
| | KAPKLLIFKTSSLESGVPSRFSGSGSGTEFTLTISSLQPDDFAI |
| | YYCQQYNSYIFGQGTRLEIKRTVAAPSVFIFPPSDEQLKSGT |
| | ASVVCLLNNFYPREAKVQWKVDNALQSGNSQESVTEQDS |
| | KDSTYSLSSTLTLSKADYEKHKVYACEVTHQGLSSPVTKSF |
| | NRGEC (SEQ ID NO: 42) |
| T26B386 | SSELTQDPAVSVALGQTVRITCQGDSLRNYYASWYQQKPG |
| | QAPVLVFYGKDNRPSGIPDRFSGSTSGNTASLTITGAQAQD |
| | EADYYCNSRDSSGDHLVFGGGTKLTVLGQPKAAPSVTLFPP |
| | SSEELQANKATLVCLISDFYPGAVTVAWKADSSPVKAGVE |
| | TTTPSKQSNNKYAASSYLSLTPEQWKSHRSYSCQVTHEGST |
| | VEKTVAPTECS (SEQ ID NO: 43) |
| T26B388 | DIQMTQSPSSLSASVGDRVTITCRASQGIRNDLGWYQQKPG |
| | KAPKRLIYAASSLQGGVPSRFSGSGSGTEFTLTISSLQPEDFA |
| | TYYCLQHYTYPLTFGGGTKVEIKRTVAAPSVFIFPPSDEQLK |
| | SGTASVVCLLNNFYPREAKVQWKVDNALQSGNSQESVTEQ |
| | DSKDSTYSLSSTLTLSKADYEKHKVYACEVTHQGLSSPVTK |
| | SFNRGEC (SEQ ID NO: 44) |
| T26B459 | DIQMTQSPSTLSASVGDRVTITCRASQSTSSWLAWYQQKPG |
| | KAPKLLIYKTSSLESGVPSRFSGSGSGTEFTLTISSLQPDDFA |
| | TYYCQQYNSYSFGQGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGEC (SEQ ID NO: 37) |
| T26B460 | DIQMTQSPSTLSASVGDRVTITCRASQSISSWLAWYQQKPG |
| | KAPKLLIYKASSLESGVPSRFSGSRSGTEFTLTISSLQPDDFA |
| | TYYCQQYNSWTFGQGTKVEIKRTVAAPSVFIFPPSDEQLKS |
| | GTASVVCLLNNFYPREAKVQWKVDNALQSGNSQESVTEQ |
| | DSKDSTYSLSSTLTLSKADYEKHKVYACEVTHQGLSSPVTK |
| | SFNRGEC (SEQ ID NO: 38) |
| T26B461 | DIQMTQSPSTLSASVGDRVTITCRASQSISSWLAWYQQKPG |
| | KAPKLLIYKASSLESGVPSRFSGSGSGTEFTLTISSLQPDDFA |
| | TYYCQQYNSYTFGQGTKLEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGEC (SEQ ID NO: 39) |
| T26B462 | DIQMTQSPSTLSASVGDRVTITCRASQSISSWLAWYQQKPG |
| | KAPKLLIFKTSSLESGVPSRFSGSGSGTEFTLTISSLQPDDFAT |
| | YYCQQYNSYSFGGGTKVEIKRTVAAPSVFIFPPSDEQLKSGT |
| | ASVVCLLNNFYPREAKVQWKVDNALQSGNSQESVTEQDS |
| | KDSTYSLSSTLTLSKADYEKHKVYACEVTHQGLSSPVTKSF |
| | NRGEC (SEQ ID NO: 40) |
| T26B463 | QSALTQPASVSGSPGQSITISCTGTSSDVGGYNYVSWYQQH |
| | PSKAPKLMIYDVSNRPSGVSNRFSGSKSGNTASLTISGLQAE |
| | DEADYYCSSYTSSSTLVVFGGGTKLTVLGQPKAAPSVTLFP |
| | PSSEELQANKATLVCLISDFYPGAVTVAWKADSSPVKAGV |
| | ETTTPSKQSNNKYAASSYLSLTPEQWKSHRSYSCQVTHEGS |
| | TVEKTVAPTECS (SEQ ID NO: 41) |
| T26B464 | DIQMTQSPSTLSASVGDRVTITCRASQSLSSWLAWYQQKPG |
| | KAPKLLIFKTSSLESGVPSRFSGSGSGTEFTLTISSLQPDDFAI |
| | YYCQQYNSYIFGQGTRLEIKRTVAAPSVFIFPPSDEQLKSGT |
| | ASVVCLLNNFYPREAKVQWKVDNALQSGNSQESVTEQDS |
| | KDSTYSLSSTLTLSKADYEKHKVYACEVTHQGLSSPVTKSF |
| | NRGEC (SEQ ID NO: 42) |
| T26B465 | SSELTQDPAVSVALGQTVRITCQGDSLRNYYASWYQQKPG |
| | QAPVLVFYGKDNRPSGIPDRFSGSTSGNTASLTITGAQAQD |
| | EADYYCNSRDSSGDHLVFGGGTKLTVLGQPKAAPSVTLFPP |
| | SSEELQANKATLVCLISDFYPGAVTVAWKADSSPVKAGVE |
| | TTTPSKQSNNKYAASSYLSLTPEQWKSHRSYSCQVTHEGST |
| | VEKTVAPTECS (SEQ ID NO: 43) |
| T26B466 | DIQMTQSPSSLSASVGDRVTITCRASQGIRNDLGWYQQKPG |
| | KAPKRLIYAASSLQGGVPSRFSGSGSGTEFTLTISSLQPEDFA |
| | TYYCLQHYTYPLTFGGGTKVEIKRTVAAPSVFIFPPSDEQLK |
| | SGTASVVCLLNNFYPREAKVQWKVDNALQSGNSQESVTEQ |
| | DSKDSTYSLSSTLTLSKADYEKHKVYACEVTHQGLSSPVTK |
| | SFNRGEC (SEQ ID NO: 44) |
| T26B608 | DIQMTQSPSTLSASVGDRVTITCRASQSTSSWLAWYQQKPG |
| | KAPKLLIYKTSSLESGVPSRFSGSGSGTEFTLTISSLQPDDFA |
| | TYYCQQYNSYSFGQGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGEC (SEQ ID NO: 37) |
| T26B609 | DIQMTQSPSSLSASVGDRVTITCRASQGIRNDLGWYQQKPG |
| | KAPKRLIYAASSLQGGVPSRFSGSGSGTEFTLTISSLQPEDFA |
| | TYYCLQHYTYPLTFGGGTKVEIKRTVAAPSVFIFPPSDEQLK |
| | SGTASVVCLLNNFYPREAKVQWKVDNALQSGNSQESVTEQ |
| | DSKDSTYSLSSTLTLSKADYEKHKVYACEVTHQGLSSPVTK |
| | SFNRGEC (SEQ ID NO: 44) |
| T26B610 | DIQMTQSPSTLSASVGDRVTITCRASQSLSSWLAWYQQKPG |
| | KAPKLLIFKTSSLESGVPSRFSGSGSGTEFTLTISSLQPDDFAI |
| | YYCQQYNSYIFGQGTRLEIKRTVAAPSVFIFPPSDEQLKSGT |
| | ASVVCLLNNFYPREAKVQWKVDNALQSGNSQESVTEQDS |
| | KDSTYSLSSTLTLSKADYEKHKVYACEVTHQGLSSPVTKSF |
| | NRGEC (SEQ ID NO: 42) |
| T26B611 | DIQMTQSPSTLSASVGDRVTITCRASQSISSWLAWYQQKPG |
| | KAPKLLIYKASSLESGVPSRFSGSRSGTEFTLTISSLQPDDFA |
| | TYYCQQYNSWTFGQGTKVEIKRTVAAPSVFIFPPSDEQLKS |
| | GTASVVCLLNNFYPREAKVQWKVDNALQSGNSQESVTEQ |
| | DSKDSTYSLSSTLTLSKADYEKHKVYACEVTHQGLSSPVTK |
| | SFNRGEC (SEQ ID NO: 38) |
| T26B612 | DIQMTQSPSTLSASVGDRVTITCRASQSISSWLAWYQQKPG |
| | KAPKLLIYKASSLESGVPSRFSGSGSGTEFTLTISSLQPDDFA |
| | TYYCQQYNSYTFGQGTKLEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGEC (SEQ ID NO: 39) |
| T26B613 | QSALTQPASVSGSPGQSITISCTGTSSDVGGYNYVSWYQQH |
| | PSKAPKLMIYDVSNRPSGVSNRFSGSKSGNTASLTISGLQAE |
| | DEADYYCSSYTSSSTLVVFGGGTKLTVLGQPKAAPSVTLFP |
| | PSSEELQANKATLVCLISDFYPGAVTVAWKADSSPVKAGV |
| | ETTTPSKQSNNKYAASSYLSLTPEQWKSHRSYSCQVTHEGS |
| | TVEKTVAPTECS (SEQ ID NO: 41) |
| T26B614 | SSELTQDPAVSVALGQTVRITCQGDSLRNYYASWYQQKPG |
| | QAPVLVFYGKDNRPSGIPDRFSGSTSGNTASLTITGAQAQD |
| | EADYYCNSRDSSGDHLVFGGGTKLTVLGQPKAAPSVTLFPP |
| | SSEELQANKATLVCLISDFYPGAVTVAWKADSSPVKAGVE |
| | TTTPSKQSNNKYAASSYLSLTPEQWKSHRSYSCQVTHEGST |
| | VEKTVAPTECS (SEQ ID NO: 43) |
| T26B615 | DIQMTQSPSTLSASVGDRVTITCRASQSISSWLAWYQQKPG |
| | KAPKLLIFKTSSLESGVPSRFSGSGSGTEFTLTISSLQPDDFAT |
| | YYCQQYNSYSFGGGTKVEIKRTVAAPSVFIFPPSDEQLKSGT |
| | ASVVCLLNNFYPREAKVQWKVDNALQSGNSQESVTEQDS |
| | KDSTYSLSSTLTLSKADYEKHKVYACEVTHQGLSSPVTKSF |
| | NRGEC (SEQ ID NO: 40) |

TABLE 8
Complete amino acid sequences of the heavy chains of
exemplary anti-CD37 antibodies

| | Complete Sequence |
|---|---|
| Antibody Name | (SEQ ID NO) |
| T26B373 | QVQLQESGPGLVKPSETLSLTCTVSGGSISSGVYYWAWIRQ |
| | PPGKGLELIGTFYYSGSTYYDSSLRSRVTISVDTSKNQFSLK |
| | LSSVTAADTAVYYCARQAGDFDYWGQGTLVTVSSASTKG |
| | PSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCDKTHTCPPCPAPELLGGPSVFLFPPKP |
| | KDTLMISRTPEVTCVVVDVSHEDPEVKFNWYVDGVEVHN |
| | AKTKPREEQYNSTYRVVSVLTVLHQDWLNGKEYKCKVSN |
| | KALPAPIEKTISKAKGQPREPQVYTLPPSREEMTKNQVSLTC |
| | LVKGFYPSDIAVEWESNGQPENNYKTTPPVLDSDGSFFLYS |
| | KLTVDKSRWQQGNVFSCSVMHEALHNHYTQKSLSLSPGK |
| | (SEQ ID NO: 45) |
| T26B374 | QLQLQESGPGLVKPSETLSLTCTVSGGSISSSPYYWGWIRQP |
| | PGKGLEWIGSFYYGGSNYYNPSLKSRVTISADTSKNQFSLK |
| | LSSVTAADTAVYYCARQAGDWDYWGQGTLVTVSSASTKG |
| | PSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCDKTHTCPPCPAPELLGGPSVFLFPPKP |
| | KDTLMISRTPEVTCVVVDVSHEDPEVKFNWYVDGVEVHN |
| | AKTKPREEQYNSTYRVVSVLTVLHQDWLNGKEYKCKVSN |
| | KALPAPIEKTISKAKGQPREPQVYTLPPSREEMTKNQVSLTC |
| | LVKGFYPSDIAVEWESNGQPENNYKTTPPVLDSDGSFFLYS |
| | KLTVDKSRWQQGNVFSCSVMHEALHNHYTQKSLSLSPGK |
| | (SEQ ID NO: 46) |
| T26B375 | QLQLQESGPGLVKPSETLSLTCTVSGGSISSGIYNWGWIRQP |
| | PGKGLEWIGNFQYSGITYYNPSLKSRVTISVDTSKNQFSLQL |
| | SSVTAADTAVYYCARQAGDFDYWGQGTLVTVSSASTKGP |
| | SVFPLAPSSKSTSGGTAALGCLVKDYFPEPVTVSWNSGALT |
| | SGVHTFPAVLQSSGLYSLSSVVTVPSSSLGTQTYICNVNHKP |
| | SNTKVDKKVEPKSCDKTHTCPPCPAPELLGGPSVFLFPPKPK |
| | DTLMISRTPEVTCVVVDVSHEDPEVKFNWYVDGVEVHNA |
| | KTKPREEQYNSTYRVVSVLTVLHQDWLNGKEYKCKVSNK |
| | ALPAPIEKTISKAKGQPREPQVYTLPPSREEMTKNQVSLTCL |
| | VKGFYPSDIAVEWESNGQPENNYKTTPPVLDSDGSFFLYSK |
| | LTVDKSRWQQGNVFSCSVMHEALHNHYTQKSLSLSPGK |
| | (SEQ ID NO: 47) |
| T26B379 | QVQLQESGPGLVKPSETLSLTCTVSGGSISSSIYYWAWIRQP |
| | PGKGLEWIGTIYYGGSPYYSPSLKSRVTISIDTSKSQFSLRLT |
| | SVTVADTAVYYCARRAGDFDYWGQGTLVTVSSASTKGPS |
| | VFPLAPSSKSTSGGTAALGCLVKDYFPEPVTVSWNSGALTS |
| | GVHTFPAVLQSSGLYSLSSVVTVPSSSLGTQTYICNVNHKPS |
| | NTKVDKKVEPKSCDKTHTCPPCPAPELLGGPSVFLFPPKPK |
| | DTLMISRTPEVTCVVVDVSHEDPEVKFNWYVDGVEVHNA |
| | KTKPREEQYNSTYRVVSVLTVLHQDWLNGKEYKCKVSNK |
| | ALPAPIEKTISKAKGQPREPQVYTLPPSREEMTKNQVSLTCL |
| | VKGFYPSDIAVEWESNGQPENNYKTTPPVLDSDGSFFLYSK |
| | LTVDKSRWQQGNVFSCSVMHEALHNHYTQKSLSLSPGK |
| | (SEQ ID NO: 48) |
| T26B382 | QVQLVESGGGLVKPGGSLRLSCAASGFTFSDYYMTWIRQA |
| | PGKGLEWVSYISSSGITKYYADFVKGRFTISRDNAKNSLYL |
| | QMNSLRAEDTAVYYCARDRDRQWLLEFDYWGQGTLVTVS |
| | SASTKGPSVFPLAPSSKSTSGGTAALGCLVKDYFPEPVTVS |
| | WNSGALTSGVHTFPAVLQSSGLYSLSSVVTVPSSSLGTQTYI |
| | CNVNHKPSNTKVDKKVEPKSCDKTHTCPPCPAPELLGGPSV |
| | FLFPPKPKDTLMISRTPEVTCVVVDVSHEDPEVKFNWYVDG |
| | VEVHNAKTKPREEQYNSTYRVVSVLTVLHQDWLNGKEYK |
| | CKVSNKALPAPIEKTISKAKGQPREPQVYTLPPSREEMTKN |
| | QVSLTCLVKGFYPSDIAVEWESNGQPENNYKTTPPVLDSDG |
| | SFFLYSKLTVDKSRWQQGNVFSCSVMHEALHNHYTQKSLS |
| | LSPGK (SEQ ID NO: 49) |
| T26B385 | QVQLQESGPGLVKPSETLSLTCIVSGGSVSSRNYYWGWIRQ |
| | PPGKGLEWIGRIYYSGNTNYNPSLKSRVTISVDTSKNQFSLK |
| | LSSVTAADTAVYYCARWAGEIDYWGQGTLVTVSSASTKGP |
| | SVFPLAPSSKSTSGGTAALGCLVKDYFPEPVTVSWNSGALT |
| | SGVHTFPAVLQSSGLYSLSSVVTVPSSSLGTQTYICNVNHKP |
| | SNTKVDKKVEPKSCDKTHTCPPCPAPELLGGPSVFLFPPKPK |
| | DTLMISRTPEVTCVVVDVSHEDPEVKFNWYVDGVEVHNA |
| | KTKPREEQYNSTYRVVSVLTVLHQDWLNGKEYKCKVSNK |
| | ALPAPIEKTISKAKGQPREPQVYTLPPSREEMTKNQVSLTCL |
| | VKGFYPSDIAVEWESNGQPENNYKTTPPVLDSDGSFFLYSK |
| | LTVDKSRWQQGNVFSCSVMHEALHNHYTQKSLSLSPGK |
| | (SEQ ID NO: 50) |
| T26B386 | QVQLQESGPGLVKPSETLSLSCTVSGGSLNSYNYYWGWVR |
| | QPPGKGLEWIGTIYSSGSAYYNPSLKSRFTISVATSKNQFSL |
| | RLSSVTAADTAVYYCARGYRNSWYALFEYWGQGTLVTVS |
| | SASTKGPSVFPLAPSSKSTSGGTAALGCLVKDYFPEPVTVS |
| | WNSGALTSGVHTFPAVLQSSGLYSLSSVVTVPSSSLGTQTYI |
| | CNVNHKPSNTKVDKKVEPKSCDKTHTCPPCPAPELLGGPSV |
| | FLFPPKPKDTLMISRTPEVTCVVVDVSHEDPEVKFNWYVDG |
| | VEVHNAKTKPREEQYNSTYRVVSVLTVLHQDWLNGKEYK |
| | CKVSNKALPAPIEKTISKAKGQPREPQVYTLPPSREEMTKN |
| | QVSLTCLVKGFYPSDIAVEWESNGQPENNYKTTPPVLDSDG |
| | SFFLYSKLTVDKSRWQQGNVFSCSVMHEALHNHYTQKSLS |
| | LSPGK (SEQ ID NO: 51) |
| T26B388 | QVQLQQWGAGLLKPSETLSLTCAVYGGSFSDYYWSWIRQP |
| | PGKGLEWIGEIDHSGSTDYNPSLKSRVTISVDTSKNQFSLKL |
| | SSVTAADTAVYYCARSMYYDIWTGYHGAFDIWGQGTMVT |
| | VSSASTKGPSVFPLAPSSKSTSGGTAALGCLVKDYFPEPVTV |
| | SWNSGALTSGVHTFPAVLQSSGLYSLSSVVTVPSSSLGTQT |
| | YICNVNHKPSNTKVDKKVEPKSCDKTHTCPPCPAPELLGGP |
| | SVFLFPPKPKDTLMISRTPEVTCVVVDVSHEDPEVKFNWYV |
| | DGVEVHNAKTKPREEQYNSTYRVVSVLTVLHQDWLNGKE |
| | YKCKVSNKALPAPIEKTISKAKGQPREPQVYTLPPSREEMT |
| | KNQVSLTCLVKGFYPSDIAVEWESNGQPENNYKTTPPVLDS |
| | DGSFFLYSKLTVDKSRWQQGNVFSCSVMHEALHNHYTQK |
| | SLSLSPGK (SEQ ID NO: 52) |
| T26B459 | QVQLQESGPGLVKPSETLSLTCTVSGGSISSGVYYWAWIRQ |
| | PPGKGLELIGTFYYSGSTYYDSSLRSRVTISVDTSKNQFSLK |
| | LSSVTAADTAVYYCARQAGDFDYWGQGTLVTVSSASTKG |
| | PSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCDKTHTCPPCPAPELLGGPSVFLFPPKP |
| | EDTLMISRTPEVTCVVVDVSHEDPEVKFNWYVDGVEVHNA |
| | KTKPREEQYNSTYRVVSVLTVLHQDWLNGKEYKCKVSNK |
| | ALPAPIEKTISKAKGQPREPQVYTLPPSREEMTKNQVSLTCL |
| | VKGFYPSDIAVEWESNGQPENNYKTTPPVLDSDGSFFLYSK |
| | LTVDKSRWQQGNVFSCSVMHEALHNHYRQKSLSLSPGK |
| | (SEQ ID NO: 53) |
| T26B460 | QLQLQESGPGLVKPSETLSLTCTVSGGSISSSPYYWGWIRQP |
| | PGKGLEWIGSFYYGGSNYYNPSLKSRVTISADTSKNQFSLK |
| | LSSVTAADTAVYYCARQAGDWDYWGQGTLVTVSSASTKG |
| | PSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCDKTHTCPPCPAPELLGGPSVFLFPPKP |
| | EDTLMISRTPEVTCVVVDVSHEDPEVKFNWYVDGVEVHNA |
| | KTKPREEQYNSTYRVVSVLTVLHQDWLNGKEYKCKVSNK |
| | ALPAPIEKTISKAKGQPREPQVYTLPPSREEMTKNQVSLTCL |
| | VKGFYPSDIAVEWESNGQPENNYKTTPPVLDSDGSFFLYSK |
| | LTVDKSRWQQGNVFSCSVMHEALHNHYRQKSLSLSPGK |
| | (SEQ ID NO: 54) |
| T26B461 | QLQLQESGPGLVKPSETLSLTCTVSGGSISSGIYNWGWIRQP |
| | PGKGLEWIGNFQYSGITYYNPSLKSRVTISVDTSKNQFSLQL |
| | SSVTAADTAVYYCARQAGDFDYWGQGTLVTVSSASTKGP |
| | SVFPLAPSSKSTSGGTAALGCLVKDYFPEPVTVSWNSGALT |
| | SGVHTFPAVLQSSGLYSLSSVVTVPSSSLGTQTYICNVNHKP |
| | SNTKVDKKVEPKSCDKTHTCPPCPAPELLGGPSVFLFPPKPE |
| | DTLMISRTPEVTCVVVDVSHEDPEVKFNWYVDGVEVHNA |
| | KTKPREEQYNSTYRVVSVLTVLHQDWLNGKEYKCKVSNK |
| | ALPAPIEKTISKAKGQPREPQVYTLPPSREEMTKNQVSLTCL |
| | VKGFYPSDIAVEWESNGQPENNYKTTPPVLDSDGSFFLYSK |
| | LTVDKSRWQQGNVFSCSVMHEALHNHYRQKSLSLSPGK |
| | (SEQ ID NO: 55) |
| T26B462 | QVQLQESGPGLVKPSETLSLTCTVSGGSISSSIYYWAWIRQP |
| | PGKGLEWIGTIYYGGSPYYSPSLKSRVTISIDTSKSQFSLRLT |
| | SVTVADTAVYYCARRAGDFDYWGQGTLVTVSSASTKGPS |
| | VFPLAPSSKSTSGGTAALGCLVKDYFPEPVTVSWNSGALTS |
| | GVHTFPAVLQSSGLYSLSSVVTVPSSSLGTQTYICNVNHKPS |
| | NTKVDKKVEPKSCDKTHTCPPCPAPELLGGPSVFLFPPKPE |
| | DTLMISRTPEVTCVVVDVSHEDPEVKFNWYVDGVEVHNA |
| | KTKPREEQYNSTYRVVSVLTVLHQDWLNGKEYKCKVSNK |
| | ALPAPIEKTISKAKGQPREPQVYTLPPSREEMTKNQVSLTCL |
| | VKGFYPSDIAVEWESNGQPENNYKTTPPVLDSDGSFFLYSK |
| | LTVDKSRWQQGNVFSCSVMHEALHNHYRQKSLSLSPGK |
| | (SEQ ID NO: 56) |
| T26B463 | QVQLVESGGGLVKPGGSLRLSCAASGFTFSDYYMTWIRQA |
| | PGKGLEWVSYISSSGITKYYADFVKGRFTISRDNAKNSLYL |
| | QMNSLRAEDTAVYYCARDRDRQWLLEFDYWGQGTLVTVS |
| | SASTKGPSVFPLAPSSKSTSGGTAALGCLVKDYFPEPVTVS |
| | WNSGALTSGVHTFPAVLQSSGLYSLSSVVTVPSSSLGTQTYI |
| | CNVNHKPSNTKVDKKVEPKSCDKTHTCPPCPAPELLGGPSV |
| | FLFPPKPEDTLMISRTPEVTCVVVDVSHEDPEVKFNWYVDG |
| | VEVHNAKTKPREEQYNSTYRVVSVLTVLHQDWLNGKEYK |
| | CKVSNKALPAPIEKTISKAKGQPREPQVYTLPPSREEMTKN |
| | QVSLTCLVKGFYPSDIAVEWESNGQPENNYKTTPPVLDSDG |
| | SFFLYSKLTVDKSRWQQGNVFSCSVMHEALHNHYRQKSLS |
| | LSPGK (SEQ ID NO: 57) |
| T26B464 | QVQLQESGPGLVKPSETLSLTCIVSGGSVSSRNYYWGWIRQ |
| | PPGKGLEWIGRIYYSGNTNYNPSLKSRVTISVDTSKNQFSLK |
| | LSSVTAADTAVYYCARWAGEIDYWGQGTLVTVSSASTKGP |
| | SVFPLAPSSKSTSGGTAALGCLVKDYFPEPVTVSWNSGALT |
| | SGVHTFPAVLQSSGLYSLSSVVTVPSSSLGTQTYICNVNHKP |
| | SNTKVDKKVEPKSCDKTHTCPPCPAPELLGGPSVFLFPPKPE |
| | DTLMISRTPEVTCVVVDVSHEDPEVKFNWYVDGVEVHNA |
| | KTKPREEQYNSTYRVVSVLTVLHQDWLNGKEYKCKVSNK |
| | ALPAPIEKTISKAKGQPREPQVYTLPPSREEMTKNQVSLTCL |
| | VKGFYPSDIAVEWESNGQPENNYKTTPPVLDSDGSFFLYSK |
| | LTVDKSRWQQGNVFSCSVMHEALHNHYRQKSLSLSPGK |
| | (SEQ ID NO: 58) |
| T26B465 | QVQLQESGPGLVKPSETLSLSCTVSGGSLNSYNYYWGWVR |
| | QPPGKGLEWIGTIYSSGSAYYNPSLKSRFTISVATSKNQFSL |
| | RLSSVTAADTAVYYCARGYRNSWYALFEYWGQGTLVTVS |
| | SASTKGPSVFPLAPSSKSTSGGTAALGCLVKDYFPEPVTVS |
| | WNSGALTSGVHTFPAVLQSSGLYSLSSVVTVPSSSLGTQTYI |
| | CNVNHKPSNTKVDKKVEPKSCDKTHTCPPCPAPELLGGPSV |
| | FLFPPKPEDTLMISRTPEVTCVVVDVSHEDPEVKFNWYVDG |
| | VEVHNAKTKPREEQYNSTYRVVSVLTVLHQDWLNGKEYK |
| | CKVSNKALPAPIEKTISKAKGQPREPQVYTLPPSREEMTKN |
| | QVSLTCLVKGFYPSDIAVEWESNGQPENNYKTTPPVLDSDG |
| | SFFLYSKLTVDKSRWQQGNVFSCSVMHEALHNHYRQKSLS |
| | LSPGK (SEQ ID NO: 59) |
| T26B466 | QVQLQQWGAGLLKPSETLSLTCAVYGGSFSDYYWSWIRQP |
| | PGKGLEWIGEIDHSGSTDYNPSLKSRVTISVDTSKNQFSLKL |
| | SSVTAADTAVYYCARSMYYDIWTGYHGAFDIWGQGTMVT |
| | VSSASTKGPSVFPLAPSSKSTSGGTAALGCLVKDYFPEPVTV |
| | SWNSGALTSGVHTFPAVLQSSGLYSLSSVVTVPSSSLGTQT |
| | YICNVNHKPSNTKVDKKVEPKSCDKTHTCPPCPAPELLGGP |
| | SVFLFPPKPEDTLMISRTPEVTCVVVDVSHEDPEVKFNWYV |
| | DGVEVHNAKTKPREEQYNSTYRVVSVLTVLHQDWLNGKE |
| | YKCKVSNKALPAPIEKTISKAKGQPREPQVYTLPPSREEMT |
| | KNQVSLTCLVKGFYPSDIAVEWESNGQPENNYKTTPPVLDS |
| | DGSFFLYSKLTVDKSRWQQGNVFSCSVMHEALHNHYRQK |
| | SLSLSPGK (SEQ ID NO: 60) |
| T26B608 | QVQLQESGPGLVKPSETLSLTCTVSGGSISSGVYYWAWIRQ |
| | PPGKGLELIGTFYYSGSTYYDSSLRSRVTISVDTSKNQFSLK |
| | LSSVTAADTAVYYCARQAGDFDYWGQGTLVTVSSASTKG |
| | PSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCDKTHTCPPCPAPELLGGPDVFLFPPKP |
| | KDTLMISRTPEVTCVVVDVSHEDPEVKFNWYVDGVEVHN |
| | AKTKPREEQYNSTYRVVSVLTVLHQDWLNGKEYKCKVSN |
| | KALPAPEEKTISKAKGQPREPQVYTLPPSREEMTKNQVSLT |
| | CLVKGFYPSDIAVEWESNGQPENNYKTTPPVLDSDGSFFLY |
| | SKLTVDKSRWQQGNVFSCSVMHEALHNHYTQKSLSLSPG |
| | (SEQ ID NO: 61) |
| T26B609 | QVQLQQWGAGLLKPSETLSLTCAVYGGSFSDYYWSWIRQP |
| | PGKGLEWIGEIDHSGSTDYNPSLKSRVTISVDTSKNQFSLKL |
| | SSVTAADTAVYYCARSMYYDIWTGYHGAFDIWGQGTMVT |
| | VSSASTKGPSVFPLAPSSKSTSGGTAALGCLVKDYFPEPVTV |
| | SWNSGALTSGVHTFPAVLQSSGLYSLSSVVTVPSSSLGTQT |
| | YICNVNHKPSNTKVDKKVEPKSCDKTHTCPPCPAPELLGGP |
| | DVFLFPPKPKDTLMISRTPEVTCVVVDVSHEDPEVKFNWYV |
| | DGVEVHNAKTKPREEQYNSTYRVVSVLTVLHQDWLNGKE |
| | YKCKVSNKALPAPEEKTISKAKGQPREPQVYTLPPSREEMT |
| | KNQVSLTCLVKGFYPSDIAVEWESNGQPENNYKTTPPVLDS |
| | DGSFFLYSKLTVDKSRWQQGNVFSCSVMHEALHNHYTQK |
| | SLSLSPG (SEQ ID NO: 62) |
| T26B610 | QVQLQESGPGLVKPSETLSLTCIVSGGSVSSRNYYWGWIRQ |
| | PPGKGLEWIGRIYYSGNTNYNPSLKSRVTISVDTSKNQFSLK |
| | LSSVTAADTAVYYCARWAGEIDYWGQGTLVTVSSASTKGP |
| | SVFPLAPSSKSTSGGTAALGCLVKDYFPEPVTVSWNSGALT |
| | SGVHTFPAVLQSSGLYSLSSVVTVPSSSLGTQTYICNVNHKP |
| | SNTKVDKKVEPKSCDKTHTCPPCPAPELLGGPDVFLFPPKP |
| | KDTLMISRTPEVTCVVVDVSHEDPEVKFNWYVDGVEVHN |
| | AKTKPREEQYNSTYRVVSVLTVLHQDWLNGKEYKCKVSN |
| | KALPAPEEKTISKAKGQPREPQVYTLPPSREEMTKNQVSLT |
| | CLVKGFYPSDIAVEWESNGQPENNYKTTPPVLDSDGSFFLY |
| | SKLTVDKSRWQQGNVFSCSVMHEALHNHYTQKSLSLSPG |
| | (SEQ ID NO: 63) |
| T26B611 | QLQLQESGPGLVKPSETLSLTCTVSGGSISSSPYYWGWIRQP |
| | PGKGLEWIGSFYYGGSNYYNPSLKSRVTISADTSKNQFSLK |
| | LSSVTAADTAVYYCARQAGDWDYWGQGTLVTVSSASTKG |
| | PSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCDKTHTCPPCPAPELLGGPDVFLFPPKP |
| | KDTLMISRTPEVTCVVVDVSHEDPEVKFNWYVDGVEVHN |
| | AKTKPREEQYNSTYRVVSVLTVLHQDWLNGKEYKCKVSN |
| | KALPAPEEKTISKAKGQPREPQVYTLPPSREEMTKNQVSLT |
| | CLVKGFYPSDIAVEWESNGQPENNYKTTPPVLDSDGSFFLY |
| | SKLTVDKSRWQQGNVFSCSVMHEALHNHYTQKSLSLSPG |
| | (SEQ ID NO: 64) |
| T26B612 | QLQLQESGPGLVKPSETLSLTCTVSGGSISSGIYNWGWIRQP |
| | PGKGLEWIGNFQYSGITYYNPSLKSRVTISVDTSKNQFSLQL |
| | SSVTAADTAVYYCARQAGDFDYWGQGTLVTVSSASTKGP |
| | SVFPLAPSSKSTSGGTAALGCLVKDYFPEPVTVSWNSGALT |
| | SGVHTFPAVLQSSGLYSLSSVVTVPSSSLGTQTYICNVNHKP |
| | SNTKVDKKVEPKSCDKTHTCPPCPAPELLGGPDVFLFPPKP |
| | KDTLMISRTPEVTCVVVDVSHEDPEVKFNWYVDGVEVHN |
| | AKTKPREEQYNSTYRVVSVLTVLHQDWLNGKEYKCKVSN |
| | KALPAPEEKTISKAKGQPREPQVYTLPPSREEMTKNQVSLT |
| | CLVKGFYPSDIAVEWESNGQPENNYKTTPPVLDSDGSFFLY |
| | SKLTVDKSRWQQGNVFSCSVMHEALHNHYTQKSLSLSPG |
| | (SEQ ID NO: 65) |
| T26B613 | QVQLVESGGGLVKPGGSLRLSCAASGFTFSDYYMTWIRQA |
| | PGKGLEWVSYISSSGITKYYADFVKGRFTISRDNAKNSLYL |
| | QMNSLRAEDTAVYYCARDRDRQWLLEFDYWGQGTLVTVS |
| | SASTKGPSVFPLAPSSKSTSGGTAALGCLVKDYFPEPVTVS |
| | WNSGALTSGVHTFPAVLQSSGLYSLSSVVTVPSSSLGTQTYI |
| | CNVNHKPSNTKVDKKVEPKSCDKTHTCPPCPAPELLGGPD |
| | VFLFPPKPKDTLMISRTPEVTCVVVDVSHEDPEVKFNWYVD |
| | GVEVHNAKTKPREEQYNSTYRVVSVLTVLHQDWLNGKEY |
| | KCKVSNKALPAPEEKTISKAKGQPREPQVYTLPPSREEMTK |
| | NQVSLTCLVKGFYPSDIAVEWESNGQPENNYKTTPPVLDSD |
| | GSFFLYSKLTVDKSRWQQGNVFSCSVMHEALHNHYTQKSL |
| | SLSPG (SEQ ID NO: 66) |
| T26B614 | QVQLQESGPGLVKPSETLSLSCTVSGGSLNSYNYYWGWVR |
| | QPPGKGLEWIGTIYSSGSAYYNPSLKSRFTISVATSKNQFSL |
| | RLSSVTAADTAVYYCARGYRNSWYALFEYWGQGTLVTVS |
| | SASTKGPSVFPLAPSSKSTSGGTAALGCLVKDYFPEPVTVS |
| | WNSGALTSGVHTFPAVLQSSGLYSLSSVVTVPSSSLGTQTYI |
| | CNVNHKPSNTKVDKKVEPKSCDKTHTCPPCPAPELLGGPD |
| | VFLFPPKPKDTLMISRTPEVTCVVVDVSHEDPEVKFNWYVD |
| | GVEVHNAKTKPREEQYNSTYRVVSVLTVLHQDWLNGKEY |
| | KCKVSNKALPAPEEKTISKAKGQPREPQVYTLPPSREEMTK |
| | NQVSLTCLVKGFYPSDIAVEWESNGQPENNYKTTPPVLDSD |
| | GSFFLYSKLTVDKSRWQQGNVFSCSVMHEALHNHYTQKSL |
| | SLSPG (SEQ ID NO: 67) |
| T26B615 | QVQLQESGPGLVKPSETLSLTCTVSGGSISSSIYYWAWIRQP |
| | PGKGLEWIGTIYYGGSPYYSPSLKSRVTISIDTSKSQFSLRLT |
| | SVTVADTAVYYCARRAGDFDYWGQGTLVTVSSASTKGPS |
| | VFPLAPSSKSTSGGTAALGCLVKDYFPEPVTVSWNSGALTS |
| | GVHTFPAVLQSSGLYSLSSVVTVPSSSLGTQTYICNVNHKPS |
| | NTKVDKKVEPKSCDKTHTCPPCPAPELLGGPDVFLFPPKPK |
| | DTLMISRTPEVTCVVVDVSHEDPEVKFNWYVDGVEVHNA |
| | KTKPREEQYNSTYRVVSVLTVLHQDWLNGKEYKCKVSNK |
| | ALPAPEEKTISKAKGQPREPQVYTLPPSREEMTKNQVSLTC |
| | LVKGFYPSDIAVEWESNGQPENNYKTTPPVLDSDGSFFLYS |
| | KLTVDKSRWQQGNVFSCSVMHEALHNHYTQKSLSLSPG |
| | (SEQ ID NO: 68) |

The VL and VH nucleotide sequences of exemplary anti-CD37 antibodies are summarized in Table 9 and Table 10 below.

TABLE 9
VL nucleotide sequences of exemplary anti-CD37 antibodies

| Antibody | Variable Sequence |
|---|---|
| Name | (SEQ ID NO) |
| T26B373 | GACATCCAGATGACCCAGAGCCCTAGCACCCTGAGCGCTAGCG |
| | TGGGCGACAGGGTGACCATCACCTGCAGAGCCAGCCAGAGCA |
| | CCAGCAGCTGGCTGGCCTGGTACCAGCAGAAGCCCGGCAAGG |
| | CCCCCAAGCTGCTGATCTACAAGACCAGCAGCCTGGAGTCCGG |
| | CGTGCCTAGCAGGTTTAGCGGCAGCGGCAGCGGCACCGAGTTT |
| | ACCCTGACCATCAGCAGCCTCCAGCCCGACGACTTCGCCACCT |
| | ACTACTGCCAGCAGTACAACAGCTACAGCTTCGGCCAGGGCAC |
| | AAAGGTGGAGATCAAG (SEQ ID NO: 17) |
| T26B374 | GACATCCAGATGACCCAGAGCCCTAGCACACTGAGCGCCAGC |
| | GTGGGCGACAGAGTGACCATCACCTGCAGGGCCAGCCAGTCC |
| | ATCAGCAGCTGGCTGGCCTGGTACCAGCAGAAGCCCGGCAAG |
| | GCCCCCAAGCTGCTGATCTACAAGGCCAGCAGCCTGGAGAGC |
| | GGCGTGCCTAGCAGGTTCAGCGGCAGCAGGAGCGGCACCGAG |
| | TTCACCCTGACCATCAGCAGCCTGCAGCCCGACGACTTCGCCA |
| | CCTACTACTGCCAGCAGTACAACAGCTGGACCTTCGGCCAGGG |
| | CACCAAGGTGGAGATCAAG (SEQ ID NO: 18) |
| T26B375 | GACATCCAGATGACCCAGAGCCCTAGCACACTGTCAGCTAGCG |
| | TCGGCGACAGGGTGACCATCACATGCAGGGCTAGTCAGAGTAT |
| | CAGTAGCTGGCTGGCCTGGTACCAGCAGAAGCCCGGCAAGGC |
| | CCCCAAGCTGCTGATCTACAAGGCCAGCAGCCTGGAGAGCGG |
| | AGTGCCCAGCAGATTCAGTGGTAGCGGCAGTGGCACCGAGTTC |
| | ACCCTGACAATCAGCAGCCTGCAGCCCGACGATTTCGCCACCT |
| | ACTACTGCCAGCAGTACAACAGCTACACCTTCGGCCAGGGCAC |
| | CAAGCTGGAGATCAAG (SEQ ID NO: 19) |
| T26B379 | GACATCCAGATGACCCAGAGCCCTAGCACACTGAGCGCCAGC |
| | GTGGGCGACAGGGTGACCATCACCTGCAGGGCCAGCCAGAGC |
| | ATCAGCAGCTGGCTGGCCTGGTACCAGCAGAAGCCCGGCAAG |
| | GCCCCCAAGCTGCTGATCTTCAAGACCAGCAGCCTGGAGAGCG |
| | GCGTGCCTAGCAGATTTAGCGGCAGCGGCAGCGGCACCGAGTT |
| | CACCCTGACCATCAGCAGCCTGCAGCCCGACGACTTCGCCACC |
| | TACTACTGCCAGCAGTACAACAGCTACAGCTTCGGCGGCGGCA |
| | CCAAGGTGGAGATCAAG (SEQ ID NO: 20) |
| T26B382 | CAGAGCGCCCTGACCCAGCCTGCTAGCGTGAGCGGAAGCCCTG |
| | GCCAGAGCATCACCATCAGCTGCACAGGCACCAGCAGCGATG |
| | TGGGCGGCTACAACTACGTGAGCTGGTACCAGCAGCACCCCAG |
| | CAAGGCCCCCAAGCTGATGATCTACGATGTGAGCAACAGGCCC |
| | AGCGGCGTGAGCAATAGGTTCAGCGGCAGCAAGAGCGGCAAC |
| | ACCGCCTCCCTGACAATCAGCGGCCTGCAGGCCGAGGATGAG |
| | GCCGACTACTACTGCAGCAGCTACACCAGCAGCTCCACCCTGG |
| | TGGTGTTTGGCGGCGGCACCAAGCTGACCGTGCTG (SEQ ID |
| | NO: 21) |
| T26B385 | GACATCCAGATGACCCAGAGCCCTAGCACACTGAGCGCCAGC |
| | GTGGGAGACAGGGTGACCATCACCTGCAGAGCCAGCCAGTCC |
| | CTGAGCAGCTGGCTGGCCTGGTACCAGCAGAAGCCCGGCAAG |
| | GCCCCCAAGCTGCTGATCTTCAAGACAAGCAGCCTGGAGAGCG |
| | GCGTGCCCTCCAGATTCAGCGGCAGCGGAAGCGGCACCGAGTT |
| | CACCCTGACCATCAGCAGCCTGCAGCCCGACGACTTCGCCATC |
| | TACTACTGCCAGCAGTACAACTCCTACATCTTCGGCCAGGGCA |
| | CCAGGCTGGAGATCAAG (SEQ ID NO: 22) |
| T26B386 | AGCAGCGAGCTGACCCAGGATCCTGCCGTGAGCGTGGCCCTGG |
| | GACAGACCGTGAGGATCACCTGCCAGGGCGACAGCCTGAGGA |
| | ACTACTACGCCAGCTGGTACCAGCAGAAACCCGGACAGGCCC |
| | CCGTGCTGGTGTTCTACGGCAAGGACAACAGACCCAGCGGCAT |
| | CCCCGACAGGTTCAGCGGAAGCACCAGCGGCAACACCGCCAG |
| | CCTGACCATTACCGGCGCTCAGGCCCAGGACGAGGCCGACTAC |
| | TACTGCAACAGCAGGGACAGCAGCGGCGATCACCTGGTGTTTG |
| | GCGGCGGCACCAAGCTGACCGTGCTG (SEQ ID NO: 23) |
| T26B388 | GACATCCAGATGACCCAGAGCCCTAGCAGCCTGAGCGCCAGC |
| | GTGGGAGACAGGGTGACCATCACCTGCAGGGCCAGCCAGGGC |
| | ATCAGGAACGACCTGGGCTGGTACCAGCAGAAGCCCGGCAAG |
| | GCCCCCAAGAGGCTGATCTACGCTGCCAGCAGCCTGCAGGGCG |
| | GAGTGCCTAGCAGGTTTAGCGGCTCCGGCAGCGGCACAGAGTT |
| | CACCCTGACCATCTCCAGCCTGCAGCCCGAGGACTTCGCCACC |
| | TACTACTGCCTGCAGCACTACACCTACCCTCTGACCTTCGGCG |
| | GCGGCACCAAGGTGGAGATCAAG (SEQ ID NO: 24) |
| T26B459 | GACATCCAGATGACCCAGAGCCCTAGCACCCTGAGCGCTAGCG |
| | TGGGCGACAGGGTGACCATCACCTGCAGAGCCAGCCAGAGCA |
| | CCAGCAGCTGGCTGGCCTGGTACCAGCAGAAGCCCGGCAAGG |
| | CCCCCAAGCTGCTGATCTACAAGACCAGCAGCCTGGAGTCCGG |
| | CGTGCCTAGCAGGTTTAGCGGCAGCGGCAGCGGCACCGAGTTT |
| | ACCCTGACCATCAGCAGCCTCCAGCCCGACGACTTCGCCACCT |
| | ACTACTGCCAGCAGTACAACAGCTACAGCTTCGGCCAGGGCAC |
| | AAAGGTGGAGATCAAG (SEQ ID NO: 17) |
| T26B460 | GACATCCAGATGACCCAGAGCCCTAGCACACTGAGCGCCAGC |
| | GTGGGCGACAGAGTGACCATCACCTGCAGGGCCAGCCAGTCC |
| | ATCAGCAGCTGGCTGGCCTGGTACCAGCAGAAGCCCGGCAAG |
| | GCCCCCAAGCTGCTGATCTACAAGGCCAGCAGCCTGGAGAGC |
| | GGCGTGCCTAGCAGGTTCAGCGGCAGCAGGAGCGGCACCGAG |
| | TTCACCCTGACCATCAGCAGCCTGCAGCCCGACGACTTCGCCA |
| | CCTACTACTGCCAGCAGTACAACAGCTGGACCTTCGGCCAGGG |
| | CACCAAGGTGGAGATCAAG (SEQ ID NO: 18) |
| T26B461 | GACATCCAGATGACCCAGAGCCCTAGCACACTGTCAGCTAGCG |
| | TCGGCGACAGGGTGACCATCACATGCAGGGCTAGTCAGAGTAT |
| | CAGTAGCTGGCTGGCCTGGTACCAGCAGAAGCCCGGCAAGGC |
| | CCCCAAGCTGCTGATCTACAAGGCCAGCAGCCTGGAGAGCGG |
| | AGTGCCCAGCAGATTCAGTGGTAGCGGCAGTGGCACCGAGTTC |
| | ACCCTGACAATCAGCAGCCTGCAGCCCGACGATTTCGCCACCT |
| | ACTACTGCCAGCAGTACAACAGCTACACCTTCGGCCAGGGCAC |
| | CAAGCTGGAGATCAAG (SEQ ID NO: 19) |
| T26B462 | GACATCCAGATGACCCAGAGCCCTAGCACACTGAGCGCCAGC |
| | GTGGGCGACAGGGTGACCATCACCTGCAGGGCCAGCCAGAGC |
| | ATCAGCAGCTGGCTGGCCTGGTACCAGCAGAAGCCCGGCAAG |
| | GCCCCCAAGCTGCTGATCTTCAAGACCAGCAGCCTGGAGAGCG |
| | GCGTGCCTAGCAGATTTAGCGGCAGCGGCAGCGGCACCGAGTT |
| | CACCCTGACCATCAGCAGCCTGCAGCCCGACGACTTCGCCACC |
| | TACTACTGCCAGCAGTACAACAGCTACAGCTTCGGCGGCGGCA |
| | CCAAGGTGGAGATCAAG (SEQ ID NO: 20) |
| T26B463 | CAGAGCGCCCTGACCCAGCCTGCTAGCGTGAGCGGAAGCCCTG |
| | GCCAGAGCATCACCATCAGCTGCACAGGCACCAGCAGCGATG |
| | TGGGCGGCTACAACTACGTGAGCTGGTACCAGCAGCACCCCAG |
| | CAAGGCCCCCAAGCTGATGATCTACGATGTGAGCAACAGGCCC |
| | AGCGGCGTGAGCAATAGGTTCAGCGGCAGCAAGAGCGGCAAC |
| | ACCGCCTCCCTGACAATCAGCGGCCTGCAGGCCGAGGATGAG |
| | GCCGACTACTACTGCAGCAGCTACACCAGCAGCTCCACCCTGG |
| | TGGTGTTTGGCGGCGGCACCAAGCTGACCGTGCTG (SEQ ID |
| | NO: 21) |
| T26B464 | GACATCCAGATGACCCAGAGCCCTAGCACACTGAGCGCCAGC |
| | GTGGGAGACAGGGTGACCATCACCTGCAGAGCCAGCCAGTCC |
| | CTGAGCAGCTGGCTGGCCTGGTACCAGCAGAAGCCCGGCAAG |
| | GCCCCCAAGCTGCTGATCTTCAAGACAAGCAGCCTGGAGAGCG |
| | GCGTGCCCTCCAGATTCAGCGGCAGCGGAAGCGGCACCGAGTT |
| | CACCCTGACCATCAGCAGCCTGCAGCCCGACGACTTCGCCATC |
| | TACTACTGCCAGCAGTACAACTCCTACATCTTCGGCCAGGGCA |
| | CCAGGCTGGAGATCAAG (SEQ ID NO: 22) |
| T26B465 | AGCAGCGAGCTGACCCAGGATCCTGCCGTGAGCGTGGCCCTGG |
| | GACAGACCGTGAGGATCACCTGCCAGGGCGACAGCCTGAGGA |
| | ACTACTACGCCAGCTGGTACCAGCAGAAACCCGGACAGGCCC |
| | CCGTGCTGGTGTTCTACGGCAAGGACAACAGACCCAGCGGCAT |
| | CCCCGACAGGTTCAGCGGAAGCACCAGCGGCAACACCGCCAG |
| | CCTGACCATTACCGGCGCTCAGGCCCAGGACGAGGCCGACTAC |
| | TACTGCAACAGCAGGGACAGCAGCGGCGATCACCTGGTGTTTG |
| | GCGGCGGCACCAAGCTGACCGTGCTG (SEQ ID NO: 23) |
| T26B466 | GACATCCAGATGACCCAGAGCCCTAGCAGCCTGAGCGCCAGC |
| | GTGGGAGACAGGGTGACCATCACCTGCAGGGCCAGCCAGGGC |
| | ATCAGGAACGACCTGGGCTGGTACCAGCAGAAGCCCGGCAAG |
| | GCCCCCAAGAGGCTGATCTACGCTGCCAGCAGCCTGCAGGGCG |
| | GAGTGCCTAGCAGGTTTAGCGGCTCCGGCAGCGGCACAGAGTT |
| | CACCCTGACCATCTCCAGCCTGCAGCCCGAGGACTTCGCCACC |
| | TACTACTGCCTGCAGCACTACACCTACCCTCTGACCTTCGGCG |
| | GCGGCACCAAGGTGGAGATCAAG (SEQ ID NO: 24) |
| T26B608 | GACATCCAGATGACCCAGAGCCCTAGCACCCTGAGCGCTAGCG |
| | TGGGCGACAGGGTGACCATCACCTGCAGAGCCAGCCAGAGCA |
| | CCAGCAGCTGGCTGGCCTGGTACCAGCAGAAGCCCGGCAAGG |
| | CCCCCAAGCTGCTGATCTACAAGACCAGCAGCCTGGAGTCCGG |
| | CGTGCCTAGCAGGTTTAGCGGCAGCGGCAGCGGCACCGAGTTT |
| | ACCCTGACCATCAGCAGCCTCCAGCCCGACGACTTCGCCACCT |
| | ACTACTGCCAGCAGTACAACAGCTACAGCTTCGGCCAGGGCAC |
| | AAAGGTGGAGATCAAG (SEQ ID NO: 17) |
| T26B609 | GACATCCAGATGACCCAGAGCCCTAGCAGCCTGAGCGCCAGC |
| | GTGGGAGACAGGGTGACCATCACCTGCAGGGCCAGCCAGGGC |
| | ATCAGGAACGACCTGGGCTGGTACCAGCAGAAGCCCGGCAAG |
| | GCCCCCAAGAGGCTGATCTACGCTGCCAGCAGCCTGCAGGGCG |
| | GAGTGCCTAGCAGGTTTAGCGGCTCCGGCAGCGGCACAGAGTT |
| | CACCCTGACCATCTCCAGCCTGCAGCCCGAGGACTTCGCCACC |
| | TACTACTGCCTGCAGCACTACACCTACCCTCTGACCTTCGGCG |
| | GCGGCACCAAGGTGGAGATCAAG (SEQ ID NO: 24) |
| T26B610 | GACATCCAGATGACCCAGAGCCCTAGCACACTGAGCGCCAGC |
| | GTGGGAGACAGGGTGACCATCACCTGCAGAGCCAGCCAGTCC |
| | CTGAGCAGCTGGCTGGCCTGGTACCAGCAGAAGCCCGGCAAG |
| | GCCCCCAAGCTGCTGATCTTCAAGACAAGCAGCCTGGAGAGCG |
| | GCGTGCCCTCCAGATTCAGCGGCAGCGGAAGCGGCACCGAGTT |
| | CACCCTGACCATCAGCAGCCTGCAGCCCGACGACTTCGCCATC |
| | TACTACTGCCAGCAGTACAACTCCTACATCTTCGGCCAGGGCA |
| | CCAGGCTGGAGATCAAG (SEQ ID NO: 22) |
| T26B611 | GACATCCAGATGACCCAGAGCCCTAGCACACTGAGCGCCAGC |
| | GTGGGCGACAGAGTGACCATCACCTGCAGGGCCAGCCAGTCC |
| | ATCAGCAGCTGGCTGGCCTGGTACCAGCAGAAGCCCGGCAAG |
| | GCCCCCAAGCTGCTGATCTACAAGGCCAGCAGCCTGGAGAGC |
| | GGCGTGCCTAGCAGGTTCAGCGGCAGCAGGAGCGGCACCGAG |
| | TTCACCCTGACCATCAGCAGCCTGCAGCCCGACGACTTCGCCA |
| | CCTACTACTGCCAGCAGTACAACAGCTGGACCTTCGGCCAGGG |
| | CACCAAGGTGGAGATCAAG (SEQ ID NO: 18) |
| T26B612 | GACATCCAGATGACCCAGAGCCCTAGCACACTGTCAGCTAGCG |
| | TCGGCGACAGGGTGACCATCACATGCAGGGCTAGTCAGAGTAT |
| | CAGTAGCTGGCTGGCCTGGTACCAGCAGAAGCCCGGCAAGGC |
| | CCCCAAGCTGCTGATCTACAAGGCCAGCAGCCTGGAGAGCGG |
| | AGTGCCCAGCAGATTCAGTGGTAGCGGCAGTGGCACCGAGTTC |
| | ACCCTGACAATCAGCAGCCTGCAGCCCGACGATTTCGCCACCT |
| | ACTACTGCCAGCAGTACAACAGCTACACCTTCGGCCAGGGCAC |
| | CAAGCTGGAGATCAAG (SEQ ID NO: 19) |
| T26B613 | CAGAGCGCCCTGACCCAGCCTGCTAGCGTGAGCGGAAGCCCTG |
| | GCCAGAGCATCACCATCAGCTGCACAGGCACCAGCAGCGATG |
| | TGGGCGGCTACAACTACGTGAGCTGGTACCAGCAGCACCCCAG |
| | CAAGGCCCCCAAGCTGATGATCTACGATGTGAGCAACAGGCCC |
| | AGCGGCGTGAGCAATAGGTTCAGCGGCAGCAAGAGCGGCAAC |
| | ACCGCCTCCCTGACAATCAGCGGCCTGCAGGCCGAGGATGAG |
| | GCCGACTACTACTGCAGCAGCTACACCAGCAGCTCCACCCTGG |
| | TGGTGTTTGGCGGCGGCACCAAGCTGACCGTGCTG (SEQ ID |
| | NO: 21) |
| T26B614 | AGCAGCGAGCTGACCCAGGATCCTGCCGTGAGCGTGGCCCTGG |
| | GACAGACCGTGAGGATCACCTGCCAGGGCGACAGCCTGAGGA |
| | ACTACTACGCCAGCTGGTACCAGCAGAAACCCGGACAGGCCC |
| | CCGTGCTGGTGTTCTACGGCAAGGACAACAGACCCAGCGGCAT |
| | CCCCGACAGGTTCAGCGGAAGCACCAGCGGCAACACCGCCAG |
| | CCTGACCATTACCGGCGCTCAGGCCCAGGACGAGGCCGACTAC |
| | TACTGCAACAGCAGGGACAGCAGCGGCGATCACCTGGTGTTTG |
| | GCGGCGGCACCAAGCTGACCGTGCTG (SEQ ID NO: 23) |
| T26B615 | GACATCCAGATGACCCAGAGCCCTAGCACACTGAGCGCCAGC |
| | GTGGGCGACAGGGTGACCATCACCTGCAGGGCCAGCCAGAGC |
| | ATCAGCAGCTGGCTGGCCTGGTACCAGCAGAAGCCCGGCAAG |
| | GCCCCCAAGCTGCTGATCTTCAAGACCAGCAGCCTGGAGAGCG |
| | GCGTGCCTAGCAGATTTAGCGGCAGCGGCAGCGGCACCGAGTT |
| | CACCCTGACCATCAGCAGCCTGCAGCCCGACGACTTCGCCACC |
| | TACTACTGCCAGCAGTACAACAGCTACAGCTTCGGCGGCGGCA |
| | CCAAGGTGGAGATCAAG (SEQ ID NO: 20) |

TABLE 10
VH nucleotide sequences of exemplary anti-CD37 antibodies

| Antibody | Variable Sequence |
|---|---|
| Name | (SEQ ID NO) |
| T26B373 | CAGGTGCAGCTGCAGGAAAGCGGCCCCGGACTGGTGAAGCCCA |
| | GCGAAACCCTGAGCCTGACCTGTACCGTGAGCGGCGGCAGCAT |
| | CAGCAGCGGCGTGTACTACTGGGCCTGGATCAGACAGCCCCCT |
| | GGCAAGGGCCTGGAGCTGATCGGCACCTTCTACTACAGCGGCA |
| | GCACCTACTACGACAGCAGCCTGAGGAGCAGGGTGACCATCAG |
| | CGTGGACACCAGCAAGAACCAGTTCAGCCTGAAGCTGAGCAGC |
| | GTGACCGCCGCCGATACAGCCGTGTATTACTGCGCCAGGCAGG |
| | CCGGCGACTTCGACTACTGGGGCCAGGGAACCCTGGTGACCGT |
| | GAGCAGC (SEQ ID NO: 25) |
| T26B374 | CAGCTGCAGCTGCAGGAGAGCGGACCCGGACTGGTGAAGCCCA |
| | GCGAGACCCTGAGCCTGACCTGCACCGTGAGCGGCGGAAGCAT |
| | CAGCAGCAGCCCCTACTATTGGGGCTGGATCAGGCAGCCTCCC |
| | GGAAAGGGCCTGGAGTGGATCGGCAGCTTCTACTACGGCGGCA |
| | GCAACTACTACAACCCCAGCCTGAAGAGCAGGGTGACCATCAG |
| | CGCCGACACCAGCAAGAACCAGTTCAGCCTGAAGCTGTCCAGC |
| | GTGACCGCCGCTGATACCGCCGTGTACTACTGCGCCAGACAGG |
| | CCGGCGACTGGGATTACTGGGGCCAGGGCACACTGGTGACCGT |
| | GAGCAGC (SEQ ID NO: 26) |
| T26B375 | CAGCTGCAGCTGCAGGAGAGCGGACCTGGCCTGGTGAAGCCCA |
| | GCGAGACCCTGAGCCTGACCTGCACCGTGAGCGGCGGAAGCAT |
| | CAGCAGCGGCATCTACAACTGGGGCTGGATCAGGCAGCCCCCT |
| | GGCAAAGGCCTGGAGTGGATCGGCAACTTCCAGTACAGCGGCA |
| | TCACCTACTACAACCCCAGCCTGAAGAGCAGGGTGACCATCAG |
| | CGTGGACACCAGCAAGAACCAGTTCAGCCTGCAGCTGAGCAGC |
| | GTGACAGCTGCCGACACCGCCGTGTACTACTGTGCCAGGCAGG |
| | CCGGCGACTTCGATTACTGGGGCCAGGGCACCCTGGTGACAGT |
| | GAGCAGC (SEQ ID NO: 27) |
| T26B379 | CAGGTGCAGCTGCAGGAATCCGGCCCTGGCCTGGTGAAACCCA |
| | GCGAGACCCTGAGCCTGACCTGCACAGTGAGTGGAGGTAGCAT |
| | CAGCAGCAGCATCTACTACTGGGCCTGGATCAGGCAGCCTCCC |
| | GGCAAAGGACTGGAGTGGATCGGTACCATCTACTACGGCGGCA |
| | GCCCCTATTACAGTCCCTCACTGAAGAGCAGGGTGACCATCAG |
| | CATCGACACCAGCAAGTCCCAGTTCAGCCTGAGGCTGACCAGC |
| | GTGACAGTGGCCGACACCGCCGTGTATTACTGCGCCAGGAGGG |
| | CCGGCGACTTCGACTACTGGGGACAGGGCACCCTGGTGACCGT |
| | GAGCAGC (SEQ ID NO: 28) |
| T26B382 | CAGGTGCAGCTGGTGGAGAGCGGCGGAGGACTGGTGAAGCCTG |
| | GAGGCTCCCTGAGACTGAGCTGTGCCGCCTCCGGCTTCACCTTC |
| | AGCGACTACTATATGACCTGGATCAGGCAGGCCCCTGGCAAGG |
| | GACTGGAGTGGGTGAGCTACATCAGCAGCAGCGGCATCACCAA |
| | GTACTACGCCGACTTCGTGAAGGGCAGGTTCACCATCAGCAGG |
| | GACAACGCCAAGAACAGCCTGTACCTGCAGATGAACAGCCTGA |
| | GGGCCGAGGACACCGCCGTGTACTACTGCGCCAGGGACAGAGA |
| | CAGGCAGTGGCTGCTGGAGTTTGACTACTGGGGCCAGGGCACC |
| | CTGGTGACAGTGTCCAGC (SEQ ID NO: 29) |
| T26B385 | CAGGTGCAGCTGCAGGAGAGCGGCCCTGGACTGGTGAAGCCCA |
| | GCGAGACCCTGAGCCTGACCTGCATCGTGAGCGGCGGCAGCGT |
| | GAGCAGCAGAAACTACTACTGGGGCTGGATTAGGCAGCCCCCT |
| | GGCAAAGGCCTGGAGTGGATCGGCAGGATCTACTACAGCGGCA |
| | ACACCAACTACAACCCCAGCCTGAAGAGCAGAGTGACCATCTC |
| | CGTGGACACCAGCAAGAACCAGTTCAGCCTGAAGCTGAGCAGC |
| | GTGACAGCCGCCGATACCGCCGTGTACTACTGCGCCAGATGGG |
| | CCGGCGAAATCGACTACTGGGGCCAGGGCACACTGGTGACCGT |
| | GAGCAGC (SEQ ID NO: 30) |
| T26B386 | CAGGTGCAGCTGCAGGAGAGCGGCCCTGGACTGGTGAAACCCA |
| | GCGAGACCCTGAGCCTGAGCTGCACAGTGAGCGGCGGCAGCCT |
| | GAACAGCTACAACTACTACTGGGGCTGGGTGAGACAGCCTCCC |
| | GGCAAGGGACTGGAGTGGATCGGCACCATCTACAGCAGCGGCT |
| | CCGCCTACTACAACCCCAGCCTGAAGAGCAGGTTCACCATCAG |
| | CGTGGCCACCAGCAAGAACCAGTTCAGCCTGAGACTGAGCAGC |
| | GTGACAGCCGCCGACACCGCTGTGTACTACTGCGCCAGGGGCT |
| | ACAGGAACAGCTGGTACGCCCTGTTCGAGTACTGGGGACAGGG |
| | CACCCTGGTGACCGTGAGCAGC (SEQ ID NO: 31) |
| T26B388 | CAGGTGCAGCTGCAGCAGTGGGGCGCTGGACTGCTGAAGCCCA |
| | GCGAGACCCTGTCCCTGACCTGTGCCGTGTACGGCGGCAGCTTC |
| | AGCGACTACTACTGGAGCTGGATCAGACAGCCTCCTGGCAAGG |
| | GCCTGGAGTGGATCGGCGAGATCGACCACAGCGGCTCCACCGA |
| | CTACAACCCCAGCCTGAAGTCCAGGGTGACCATCAGCGTGGAC |
| | ACCAGCAAGAACCAGTTCAGCCTGAAGCTGAGCAGCGTGACAG |
| | CCGCCGATACCGCCGTGTACTACTGCGCCAGGAGCATGTACTA |
| | CGACATCTGGACCGGCTACCACGGCGCCTTTGACATCTGGGGC |
| | CAGGGCACCATGGTGACCGTGAGCAGC (SEQ ID NO: 32) |
| T26B459 | CAGGTGCAGCTGCAGGAAAGCGGCCCCGGACTGGTGAAGCCCA |
| | GCGAAACCCTGAGCCTGACCTGTACCGTGAGCGGCGGCAGCAT |
| | CAGCAGCGGCGTGTACTACTGGGCCTGGATCAGACAGCCCCCT |
| | GGCAAGGGCCTGGAGCTGATCGGCACCTTCTACTACAGCGGCA |
| | GCACCTACTACGACAGCAGCCTGAGGAGCAGGGTGACCATCAG |
| | CGTGGACACCAGCAAGAACCAGTTCAGCCTGAAGCTGAGCAGC |
| | GTGACCGCCGCCGATACAGCCGTGTATTACTGCGCCAGGCAGG |
| | CCGGCGACTTCGACTACTGGGGCCAGGGAACCCTGGTGACCGT |
| | GAGCAGC (SEQ ID NO: 25) |
| T26B460 | CAGCTGCAGCTGCAGGAGAGCGGACCCGGACTGGTGAAGCCCA |
| | GCGAGACCCTGAGCCTGACCTGCACCGTGAGCGGCGGAAGCAT |
| | CAGCAGCAGCCCCTACTATTGGGGCTGGATCAGGCAGCCTCCC |
| | GGAAAGGGCCTGGAGTGGATCGGCAGCTTCTACTACGGCGGCA |
| | GCAACTACTACAACCCCAGCCTGAAGAGCAGGGTGACCATCAG |
| | CGCCGACACCAGCAAGAACCAGTTCAGCCTGAAGCTGTCCAGC |
| | GTGACCGCCGCTGATACCGCCGTGTACTACTGCGCCAGACAGG |
| | CCGGCGACTGGGATTACTGGGGCCAGGGCACACTGGTGACCGT |
| | GAGCAGC (SEQ ID NO: 26) |
| T26B461 | CAGCTGCAGCTGCAGGAGAGCGGACCTGGCCTGGTGAAGCCCA |
| | GCGAGACCCTGAGCCTGACCTGCACCGTGAGCGGCGGAAGCAT |
| | CAGCAGCGGCATCTACAACTGGGGCTGGATCAGGCAGCCCCCT |
| | GGCAAAGGCCTGGAGTGGATCGGCAACTTCCAGTACAGCGGCA |
| | TCACCTACTACAACCCCAGCCTGAAGAGCAGGGTGACCATCAG |
| | CGTGGACACCAGCAAGAACCAGTTCAGCCTGCAGCTGAGCAGC |
| | GTGACAGCTGCCGACACCGCCGTGTACTACTGTGCCAGGCAGG |
| | CCGGCGACTTCGATTACTGGGGCCAGGGCACCCTGGTGACAGT |
| | GAGCAGC (SEQ ID NO: 27) |
| T26B462 | CAGGTGCAGCTGCAGGAATCCGGCCCTGGCCTGGTGAAACCCA |
| | GCGAGACCCTGAGCCTGACCTGCACAGTGAGTGGAGGTAGCAT |
| | CAGCAGCAGCATCTACTACTGGGCCTGGATCAGGCAGCCTCCC |
| | GGCAAAGGACTGGAGTGGATCGGTACCATCTACTACGGCGGCA |
| | GCCCCTATTACAGTCCCTCACTGAAGAGCAGGGTGACCATCAG |
| | CATCGACACCAGCAAGTCCCAGTTCAGCCTGAGGCTGACCAGC |
| | GTGACAGTGGCCGACACCGCCGTGTATTACTGCGCCAGGAGGG |
| | CCGGCGACTTCGACTACTGGGGACAGGGCACCCTGGTGACCGT |
| | GAGCAGC (SEQ ID NO: 28) |
| T26B463 | CAGGTGCAGCTGGTGGAGAGCGGCGGAGGACTGGTGAAGCCTG |
| | GAGGCTCCCTGAGACTGAGCTGTGCCGCCTCCGGCTTCACCTTC |
| | AGCGACTACTATATGACCTGGATCAGGCAGGCCCCTGGCAAGG |
| | GACTGGAGTGGGTGAGCTACATCAGCAGCAGCGGCATCACCAA |
| | GTACTACGCCGACTTCGTGAAGGGCAGGTTCACCATCAGCAGG |
| | GACAACGCCAAGAACAGCCTGTACCTGCAGATGAACAGCCTGA |
| | GGGCCGAGGACACCGCCGTGTACTACTGCGCCAGGGACAGAGA |
| | CAGGCAGTGGCTGCTGGAGTTTGACTACTGGGGCCAGGGCACC |
| | CTGGTGACAGTGTCCAGC (SEQ ID NO: 29) |
| T26B464 | CAGGTGCAGCTGCAGGAGAGCGGCCCTGGACTGGTGAAGCCCA |
| | GCGAGACCCTGAGCCTGACCTGCATCGTGAGCGGCGGCAGCGT |
| | GAGCAGCAGAAACTACTACTGGGGCTGGATTAGGCAGCCCCCT |
| | GGCAAAGGCCTGGAGTGGATCGGCAGGATCTACTACAGCGGCA |
| | ACACCAACTACAACCCCAGCCTGAAGAGCAGAGTGACCATCTC |
| | CGTGGACACCAGCAAGAACCAGTTCAGCCTGAAGCTGAGCAGC |
| | GTGACAGCCGCCGATACCGCCGTGTACTACTGCGCCAGATGGG |
| | CCGGCGAAATCGACTACTGGGGCCAGGGCACACTGGTGACCGT |
| | GAGCAGC (SEQ ID NO: 30) |
| T26B465 | CAGGTGCAGCTGCAGGAGAGCGGCCCTGGACTGGTGAAACCCA |
| | GCGAGACCCTGAGCCTGAGCTGCACAGTGAGCGGCGGCAGCCT |
| | GAACAGCTACAACTACTACTGGGGCTGGGTGAGACAGCCTCCC |
| | GGCAAGGGACTGGAGTGGATCGGCACCATCTACAGCAGCGGCT |
| | CCGCCTACTACAACCCCAGCCTGAAGAGCAGGTTCACCATCAG |
| | CGTGGCCACCAGCAAGAACCAGTTCAGCCTGAGACTGAGCAGC |
| | GTGACAGCCGCCGACACCGCTGTGTACTACTGCGCCAGGGGCT |
| | ACAGGAACAGCTGGTACGCCCTGTTCGAGTACTGGGGACAGGG |
| | CACCCTGGTGACCGTGAGCAGC (SEQ ID NO: 31) |
| T26B466 | CAGGTGCAGCTGCAGCAGTGGGGCGCTGGACTGCTGAAGCCCA |
| | GCGAGACCCTGTCCCTGACCTGTGCCGTGTACGGCGGCAGCTTC |
| | AGCGACTACTACTGGAGCTGGATCAGACAGCCTCCTGGCAAGG |
| | GCCTGGAGTGGATCGGCGAGATCGACCACAGCGGCTCCACCGA |
| | CTACAACCCCAGCCTGAAGTCCAGGGTGACCATCAGCGTGGAC |
| | ACCAGCAAGAACCAGTTCAGCCTGAAGCTGAGCAGCGTGACAG |
| | CCGCCGATACCGCCGTGTACTACTGCGCCAGGAGCATGTACTA |
| | CGACATCTGGACCGGCTACCACGGCGCCTTTGACATCTGGGGC |
| | CAGGGCACCATGGTGACCGTGAGCAGC (SEQ ID NO: 32) |
| T26B608 | CAGGTGCAGCTGCAGGAAAGCGGCCCCGGACTGGTGAAGCCCA |
| | GCGAAACCCTGAGCCTGACCTGTACCGTGAGCGGCGGCAGCAT |
| | CAGCAGCGGCGTGTACTACTGGGCCTGGATCAGACAGCCCCCT |
| | GGCAAGGGCCTGGAGCTGATCGGCACCTTCTACTACAGCGGCA |
| | GCACCTACTACGACAGCAGCCTGAGGAGCAGGGTGACCATCAG |
| | CGTGGACACCAGCAAGAACCAGTTCAGCCTGAAGCTGAGCAGC |
| | GTGACCGCCGCCGATACAGCCGTGTATTACTGCGCCAGGCAGG |
| | CCGGCGACTTCGACTACTGGGGCCAGGGAACCCTGGTGACCGT |
| | GAGCAGC (SEQ ID NO: 25) |
| T26B609 | CAGGTGCAGCTGCAGCAGTGGGGCGCTGGACTGCTGAAGCCCA |
| | GCGAGACCCTGTCCCTGACCTGTGCCGTGTACGGCGGCAGCTTC |
| | AGCGACTACTACTGGAGCTGGATCAGACAGCCTCCTGGCAAGG |
| | GCCTGGAGTGGATCGGCGAGATCGACCACAGCGGCTCCACCGA |
| | CTACAACCCCAGCCTGAAGTCCAGGGTGACCATCAGCGTGGAC |
| | ACCAGCAAGAACCAGTTCAGCCTGAAGCTGAGCAGCGTGACAG |
| | CCGCCGATACCGCCGTGTACTACTGCGCCAGGAGCATGTACTA |
| | CGACATCTGGACCGGCTACCACGGCGCCTTTGACATCTGGGGC |
| | CAGGGCACCATGGTGACCGTGAGCAGC (SEQ ID NO: 32) |
| T26B610 | CAGGTGCAGCTGCAGGAGAGCGGCCCTGGACTGGTGAAGCCCA |
| | GCGAGACCCTGAGCCTGACCTGCATCGTGAGCGGCGGCAGCGT |
| | GAGCAGCAGAAACTACTACTGGGGCTGGATTAGGCAGCCCCCT |
| | GGCAAAGGCCTGGAGTGGATCGGCAGGATCTACTACAGCGGCA |
| | ACACCAACTACAACCCCAGCCTGAAGAGCAGAGTGACCATCTC |
| | CGTGGACACCAGCAAGAACCAGTTCAGCCTGAAGCTGAGCAGC |
| | GTGACAGCCGCCGATACCGCCGTGTACTACTGCGCCAGATGGG |
| | CCGGCGAAATCGACTACTGGGGCCAGGGCACACTGGTGACCGT |
| | GAGCAGC (SEQ ID NO: 30) |
| T26B611 | CAGCTGCAGCTGCAGGAGAGCGGACCCGGACTGGTGAAGCCCA |
| | GCGAGACCCTGAGCCTGACCTGCACCGTGAGCGGCGGAAGCAT |
| | CAGCAGCAGCCCCTACTATTGGGGCTGGATCAGGCAGCCTCCC |
| | GGAAAGGGCCTGGAGTGGATCGGCAGCTTCTACTACGGCGGCA |
| | GCAACTACTACAACCCCAGCCTGAAGAGCAGGGTGACCATCAG |
| | CGCCGACACCAGCAAGAACCAGTTCAGCCTGAAGCTGTCCAGC |
| | GTGACCGCCGCTGATACCGCCGTGTACTACTGCGCCAGACAGG |
| | CCGGCGACTGGGATTACTGGGGCCAGGGCACACTGGTGACCGT |
| | GAGCAGC (SEQ ID NO: 26) |
| T26B612 | CAGCTGCAGCTGCAGGAGAGCGGACCTGGCCTGGTGAAGCCCA |
| | GCGAGACCCTGAGCCTGACCTGCACCGTGAGCGGCGGAAGCAT |
| | CAGCAGCGGCATCTACAACTGGGGCTGGATCAGGCAGCCCCCT |
| | GGCAAAGGCCTGGAGTGGATCGGCAACTTCCAGTACAGCGGCA |
| | TCACCTACTACAACCCCAGCCTGAAGAGCAGGGTGACCATCAG |
| | CGTGGACACCAGCAAGAACCAGTTCAGCCTGCAGCTGAGCAGC |
| | GTGACAGCTGCCGACACCGCCGTGTACTACTGTGCCAGGCAGG |
| | CCGGCGACTTCGATTACTGGGGCCAGGGCACCCTGGTGACAGT |
| | GAGCAGC (SEQ ID NO: 27) |
| T26B613 | CAGGTGCAGCTGGTGGAGAGCGGCGGAGGACTGGTGAAGCCTG |
| | GAGGCTCCCTGAGACTGAGCTGTGCCGCCTCCGGCTTCACCTTC |
| | AGCGACTACTATATGACCTGGATCAGGCAGGCCCCTGGCAAGG |
| | GACTGGAGTGGGTGAGCTACATCAGCAGCAGCGGCATCACCAA |
| | GTACTACGCCGACTTCGTGAAGGGCAGGTTCACCATCAGCAGG |
| | GACAACGCCAAGAACAGCCTGTACCTGCAGATGAACAGCCTGA |
| | GGGCCGAGGACACCGCCGTGTACTACTGCGCCAGGGACAGAGA |
| | CAGGCAGTGGCTGCTGGAGTTTGACTACTGGGGCCAGGGCACC |
| | CTGGTGACAGTGTCCAGC (SEQ ID NO: 29) |
| T26B614 | CAGGTGCAGCTGCAGGAGAGCGGCCCTGGACTGGTGAAACCCA |
| | GCGAGACCCTGAGCCTGAGCTGCACAGTGAGCGGCGGCAGCCT |
| | GAACAGCTACAACTACTACTGGGGCTGGGTGAGACAGCCTCCC |
| | GGCAAGGGACTGGAGTGGATCGGCACCATCTACAGCAGCGGCT |
| | CCGCCTACTACAACCCCAGCCTGAAGAGCAGGTTCACCATCAG |
| | CGTGGCCACCAGCAAGAACCAGTTCAGCCTGAGACTGAGCAGC |
| | GTGACAGCCGCCGACACCGCTGTGTACTACTGCGCCAGGGGCT |
| | ACAGGAACAGCTGGTACGCCCTGTTCGAGTACTGGGGACAGGG |
| | CACCCTGGTGACCGTGAGCAGC (SEQ ID NO: 31) |
| T26B615 | CAGGTGCAGCTGCAGGAATCCGGCCCTGGCCTGGTGAAACCCA |
| | GCGAGACCCTGAGCCTGACCTGCACAGTGAGTGGAGGTAGCAT |
| | CAGCAGCAGCATCTACTACTGGGCCTGGATCAGGCAGCCTCCC |
| | GGCAAAGGACTGGAGTGGATCGGTACCATCTACTACGGCGGCA |
| | GCCCCTATTACAGTCCCTCACTGAAGAGCAGGGTGACCATCAG |
| | CATCGACACCAGCAAGTCCCAGTTCAGCCTGAGGCTGACCAGC |
| | GTGACAGTGGCCGACACCGCCGTGTATTACTGCGCCAGGAGGG |
| | CCGGCGACTTCGACTACTGGGGACAGGGCACCCTGGTGACCGT |
| | GAGCAGC (SEQ ID NO: 28) |

The complete nucleotide sequences of the light chains and heavy chains of exemplary anti-CD37 antibodies are summarized in Table 11 and Table 12 below.

TABLE 11
Complete nucleotide sequences of the light chains of exemplary
anti-CD37 antibodies

| Antibody | Complete Sequence |
|---|---|
| Name | (SEQ ID NO) |
| T26B373 | GACATCCAGATGACCCAGAGCCCTAGCACCCTGAGCGCTAGCG |
| | TGGGCGACAGGGTGACCATCACCTGCAGAGCCAGCCAGAGCAC |
| | CAGCAGCTGGCTGGCCTGGTACCAGCAGAAGCCCGGCAAGGCC |
| | CCCAAGCTGCTGATCTACAAGACCAGCAGCCTGGAGTCCGGCG |
| | TGCCTAGCAGGTTTAGCGGCAGCGGCAGCGGCACCGAGTTTAC |
| | CCTGACCATCAGCAGCCTCCAGCCCGACGACTTCGCCACCTACT |
| | ACTGCCAGCAGTACAACAGCTACAGCTTCGGCCAGGGCACAAA |
| | GGTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 69) |
| T26B374 | GACATCCAGATGACCCAGAGCCCTAGCACACTGAGCGCCAGCG |
| | TGGGCGACAGAGTGACCATCACCTGCAGGGCCAGCCAGTCCAT |
| | CAGCAGCTGGCTGGCCTGGTACCAGCAGAAGCCCGGCAAGGCC |
| | CCCAAGCTGCTGATCTACAAGGCCAGCAGCCTGGAGAGCGGCG |
| | TGCCTAGCAGGTTCAGCGGCAGCAGGAGCGGCACCGAGTTCAC |
| | CCTGACCATCAGCAGCCTGCAGCCCGACGACTTCGCCACCTACT |
| | ACTGCCAGCAGTACAACAGCTGGACCTTCGGCCAGGGCACCAA |
| | GGTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 70) |
| T26B375 | GACATCCAGATGACCCAGAGCCCTAGCACACTGTCAGCTAGCG |
| | TCGGCGACAGGGTGACCATCACATGCAGGGCTAGTCAGAGTAT |
| | CAGTAGCTGGCTGGCCTGGTACCAGCAGAAGCCCGGCAAGGCC |
| | CCCAAGCTGCTGATCTACAAGGCCAGCAGCCTGGAGAGCGGAG |
| | TGCCCAGCAGATTCAGTGGTAGCGGCAGTGGCACCGAGTTCAC |
| | CCTGACAATCAGCAGCCTGCAGCCCGACGATTTCGCCACCTACT |
| | ACTGCCAGCAGTACAACAGCTACACCTTCGGCCAGGGCACCAA |
| | GCTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 71) |
| T26B379 | GACATCCAGATGACCCAGAGCCCTAGCACACTGAGCGCCAGCG |
| | TGGGCGACAGGGTGACCATCACCTGCAGGGCCAGCCAGAGCAT |
| | CAGCAGCTGGCTGGCCTGGTACCAGCAGAAGCCCGGCAAGGCC |
| | CCCAAGCTGCTGATCTTCAAGACCAGCAGCCTGGAGAGCGGCG |
| | TGCCTAGCAGATTTAGCGGCAGCGGCAGCGGCACCGAGTTCAC |
| | CCTGACCATCAGCAGCCTGCAGCCCGACGACTTCGCCACCTACT |
| | ACTGCCAGCAGTACAACAGCTACAGCTTCGGCGGCGGCACCAA |
| | GGTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 72) |
| T26B382 | CAGAGCGCCCTGACCCAGCCTGCTAGCGTGAGCGGAAGCCCTG |
| | GCCAGAGCATCACCATCAGCTGCACAGGCACCAGCAGCGATGT |
| | GGGCGGCTACAACTACGTGAGCTGGTACCAGCAGCACCCCAGC |
| | AAGGCCCCCAAGCTGATGATCTACGATGTGAGCAACAGGCCCA |
| | GCGGCGTGAGCAATAGGTTCAGCGGCAGCAAGAGCGGCAACA |
| | CCGCCTCCCTGACAATCAGCGGCCTGCAGGCCGAGGATGAGGC |
| | CGACTACTACTGCAGCAGCTACACCAGCAGCTCCACCCTGGTG |
| | GTGTTTGGCGGCGGCACCAAGCTGACCGTGCTGGGTCAGCCCA |
| | AGGCTGCACCCAGTGTCACTCTGTTCCCGCCCTCCTCTGAGGAG |
| | CTTCAAGCCAACAAGGCCACACTGGTGTGTCTCATAAGTGACTT |
| | CTACCCGGGAGCCGTGACAGTGGCCTGGAAGGCCGATAGCAGC |
| | CCCGTCAAGGCGGGAGTGGAGACCACCACACCCTCCAAACAAA |
| | GCAACAACAAGTACGCGGCCAGCAGCTATCTGAGCCTGACGCC |
| | TGAGCAGTGGAAGTCCCACAGAAGCTACAGCTGCCAGGTCACG |
| | CATGAAGGGAGCACCGTGGAGAAGACAGTGGCCCCTACAGAAT |
| | GTTCA (SEQ ID NO: 73) |
| T26B385 | GACATCCAGATGACCCAGAGCCCTAGCACACTGAGCGCCAGCG |
| | TGGGAGACAGGGTGACCATCACCTGCAGAGCCAGCCAGTCCCT |
| | GAGCAGCTGGCTGGCCTGGTACCAGCAGAAGCCCGGCAAGGCC |
| | CCCAAGCTGCTGATCTTCAAGACAAGCAGCCTGGAGAGCGGCG |
| | TGCCCTCCAGATTCAGCGGCAGCGGAAGCGGCACCGAGTTCAC |
| | CCTGACCATCAGCAGCCTGCAGCCCGACGACTTCGCCATCTACT |
| | ACTGCCAGCAGTACAACTCCTACATCTTCGGCCAGGGCACCAG |
| | GCTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 74) |
| T26B386 | AGCAGCGAGCTGACCCAGGATCCTGCCGTGAGCGTGGCCCTGG |
| | GACAGACCGTGAGGATCACCTGCCAGGGCGACAGCCTGAGGAA |
| | CTACTACGCCAGCTGGTACCAGCAGAAACCCGGACAGGCCCCC |
| | GTGCTGGTGTTCTACGGCAAGGACAACAGACCCAGCGGCATCC |
| | CCGACAGGTTCAGCGGAAGCACCAGCGGCAACACCGCCAGCCT |
| | GACCATTACCGGCGCTCAGGCCCAGGACGAGGCCGACTACTAC |
| | TGCAACAGCAGGGACAGCAGCGGCGATCACCTGGTGTTTGGCG |
| | GCGGCACCAAGCTGACCGTGCTGGGTCAGCCCAAGGCTGCACC |
| | CAGTGTCACTCTGTTCCCGCCCTCCTCTGAGGAGCTTCAAGCCA |
| | ACAAGGCCACACTGGTGTGTCTCATAAGTGACTTCTACCCGGGA |
| | GCCGTGACAGTGGCCTGGAAGGCCGATAGCAGCCCCGTCAAGG |
| | CGGGAGTGGAGACCACCACACCCTCCAAACAAAGCAACAACA |
| | AGTACGCGGCCAGCAGCTATCTGAGCCTGACGCCTGAGCAGTG |
| | GAAGTCCCACAGAAGCTACAGCTGCCAGGTCACGCATGAAGGG |
| | AGCACCGTGGAGAAGACAGTGGCCCCTACAGAATGTTCA (SEQ |
| | ID NO: 75) |
| T26B388 | GACATCCAGATGACCCAGAGCCCTAGCAGCCTGAGCGCCAGCG |
| | TGGGAGACAGGGTGACCATCACCTGCAGGGCCAGCCAGGGCAT |
| | CAGGAACGACCTGGGCTGGTACCAGCAGAAGCCCGGCAAGGCC |
| | CCCAAGAGGCTGATCTACGCTGCCAGCAGCCTGCAGGGCGGAG |
| | TGCCTAGCAGGTTTAGCGGCTCCGGCAGCGGCACAGAGTTCAC |
| | CCTGACCATCTCCAGCCTGCAGCCCGAGGACTTCGCCACCTACT |
| | ACTGCCTGCAGCACTACACCTACCCTCTGACCTTCGGCGGCGGC |
| | ACCAAGGTGGAGATCAAGCGTACGGTGGCTGCACCATCTGTCT |
| | TCATCTTCCCGCCATCTGATGAGCAGTTGAAATCTGGAACTGCC |
| | TCTGTTGTGTGCCTGCTGAATAACTTCTATCCCAGAGAGGCCAA |
| | AGTACAGTGGAAGGTGGATAACGCCCTCCAATCGGGTAACTCC |
| | CAGGAGAGTGTCACAGAGCAGGACAGCAAGGACAGCACCTAC |
| | AGCCTCAGCAGCACCCTGACGCTGAGCAAAGCAGACTACGAGA |
| | AACACAAAGTCTACGCCTGCGAAGTCACCCATCAGGGCCTGAG |
| | CTCGCCCGTCACAAAGAGCTTCAACAGGGGAGAGTGT (SEQ ID |
| | NO: 76) |
| T26B459 | GACATCCAGATGACCCAGAGCCCTAGCACCCTGAGCGCTAGCG |
| | TGGGCGACAGGGTGACCATCACCTGCAGAGCCAGCCAGAGCAC |
| | CAGCAGCTGGCTGGCCTGGTACCAGCAGAAGCCCGGCAAGGCC |
| | CCCAAGCTGCTGATCTACAAGACCAGCAGCCTGGAGTCCGGCG |
| | TGCCTAGCAGGTTTAGCGGCAGCGGCAGCGGCACCGAGTTTAC |
| | CCTGACCATCAGCAGCCTCCAGCCCGACGACTTCGCCACCTACT |
| | ACTGCCAGCAGTACAACAGCTACAGCTTCGGCCAGGGCACAAA |
| | GGTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 69) |
| T26B460 | GACATCCAGATGACCCAGAGCCCTAGCACACTGAGCGCCAGCG |
| | TGGGCGACAGAGTGACCATCACCTGCAGGGCCAGCCAGTCCAT |
| | CAGCAGCTGGCTGGCCTGGTACCAGCAGAAGCCCGGCAAGGCC |
| | CCCAAGCTGCTGATCTACAAGGCCAGCAGCCTGGAGAGCGGCG |
| | TGCCTAGCAGGTTCAGCGGCAGCAGGAGCGGCACCGAGTTCAC |
| | CCTGACCATCAGCAGCCTGCAGCCCGACGACTTCGCCACCTACT |
| | ACTGCCAGCAGTACAACAGCTGGACCTTCGGCCAGGGCACCAA |
| | GGTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 70) |
| T26B461 | GACATCCAGATGACCCAGAGCCCTAGCACACTGTCAGCTAGCG |
| | TCGGCGACAGGGTGACCATCACATGCAGGGCTAGTCAGAGTAT |
| | CAGTAGCTGGCTGGCCTGGTACCAGCAGAAGCCCGGCAAGGCC |
| | CCCAAGCTGCTGATCTACAAGGCCAGCAGCCTGGAGAGCGGAG |
| | TGCCCAGCAGATTCAGTGGTAGCGGCAGTGGCACCGAGTTCAC |
| | CCTGACAATCAGCAGCCTGCAGCCCGACGATTTCGCCACCTACT |
| | ACTGCCAGCAGTACAACAGCTACACCTTCGGCCAGGGCACCAA |
| | GCTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 71) |
| T26B462 | GACATCCAGATGACCCAGAGCCCTAGCACACTGAGCGCCAGCG |
| | TGGGCGACAGGGTGACCATCACCTGCAGGGCCAGCCAGAGCAT |
| | CAGCAGCTGGCTGGCCTGGTACCAGCAGAAGCCCGGCAAGGCC |
| | CCCAAGCTGCTGATCTTCAAGACCAGCAGCCTGGAGAGCGGCG |
| | TGCCTAGCAGATTTAGCGGCAGCGGCAGCGGCACCGAGTTCAC |
| | CCTGACCATCAGCAGCCTGCAGCCCGACGACTTCGCCACCTACT |
| | ACTGCCAGCAGTACAACAGCTACAGCTTCGGCGGCGGCACCAA |
| | GGTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 72) |
| T26B463 | CAGAGCGCCCTGACCCAGCCTGCTAGCGTGAGCGGAAGCCCTG |
| | GCCAGAGCATCACCATCAGCTGCACAGGCACCAGCAGCGATGT |
| | GGGCGGCTACAACTACGTGAGCTGGTACCAGCAGCACCCCAGC |
| | AAGGCCCCCAAGCTGATGATCTACGATGTGAGCAACAGGCCCA |
| | GCGGCGTGAGCAATAGGTTCAGCGGCAGCAAGAGCGGCAACA |
| | CCGCCTCCCTGACAATCAGCGGCCTGCAGGCCGAGGATGAGGC |
| | CGACTACTACTGCAGCAGCTACACCAGCAGCTCCACCCTGGTG |
| | GTGTTTGGCGGCGGCACCAAGCTGACCGTGCTGGGTCAGCCCA |
| | AGGCTGCACCCAGTGTCACTCTGTTCCCGCCCTCCTCTGAGGAG |
| | CTTCAAGCCAACAAGGCCACACTGGTGTGTCTCATAAGTGACTT |
| | CTACCCGGGAGCCGTGACAGTGGCCTGGAAGGCCGATAGCAGC |
| | CCCGTCAAGGCGGGAGTGGAGACCACCACACCCTCCAAACAAA |
| | GCAACAACAAGTACGCGGCCAGCAGCTATCTGAGCCTGACGCC |
| | TGAGCAGTGGAAGTCCCACAGAAGCTACAGCTGCCAGGTCACG |
| | CATGAAGGGAGCACCGTGGAGAAGACAGTGGCCCCTACAGAAT |
| | GTTCA (SEQ ID NO: 73) |
| T26B464 | GACATCCAGATGACCCAGAGCCCTAGCACACTGAGCGCCAGCG |
| | TGGGAGACAGGGTGACCATCACCTGCAGAGCCAGCCAGTCCCT |
| | GAGCAGCTGGCTGGCCTGGTACCAGCAGAAGCCCGGCAAGGCC |
| | CCCAAGCTGCTGATCTTCAAGACAAGCAGCCTGGAGAGCGGCG |
| | TGCCCTCCAGATTCAGCGGCAGCGGAAGCGGCACCGAGTTCAC |
| | CCTGACCATCAGCAGCCTGCAGCCCGACGACTTCGCCATCTACT |
| | ACTGCCAGCAGTACAACTCCTACATCTTCGGCCAGGGCACCAG |
| | GCTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 74) |
| T26B465 | AGCAGCGAGCTGACCCAGGATCCTGCCGTGAGCGTGGCCCTGG |
| | GACAGACCGTGAGGATCACCTGCCAGGGCGACAGCCTGAGGAA |
| | CTACTACGCCAGCTGGTACCAGCAGAAACCCGGACAGGCCCCC |
| | GTGCTGGTGTTCTACGGCAAGGACAACAGACCCAGCGGCATCC |
| | CCGACAGGTTCAGCGGAAGCACCAGCGGCAACACCGCCAGCCT |
| | GACCATTACCGGCGCTCAGGCCCAGGACGAGGCCGACTACTAC |
| | TGCAACAGCAGGGACAGCAGCGGCGATCACCTGGTGTTTGGCG |
| | GCGGCACCAAGCTGACCGTGCTGGGTCAGCCCAAGGCTGCACC |
| | CAGTGTCACTCTGTTCCCGCCCTCCTCTGAGGAGCTTCAAGCCA |
| | ACAAGGCCACACTGGTGTGTCTCATAAGTGACTTCTACCCGGGA |
| | GCCGTGACAGTGGCCTGGAAGGCCGATAGCAGCCCCGTCAAGG |
| | CGGGAGTGGAGACCACCACACCCTCCAAACAAAGCAACAACA |
| | AGTACGCGGCCAGCAGCTATCTGAGCCTGACGCCTGAGCAGTG |
| | GAAGTCCCACAGAAGCTACAGCTGCCAGGTCACGCATGAAGGG |
| | AGCACCGTGGAGAAGACAGTGGCCCCTACAGAATGTTCA (SEQ |
| | ID NO: 75) |
| T26B466 | GACATCCAGATGACCCAGAGCCCTAGCAGCCTGAGCGCCAGCG |
| | TGGGAGACAGGGTGACCATCACCTGCAGGGCCAGCCAGGGCAT |
| | CAGGAACGACCTGGGCTGGTACCAGCAGAAGCCCGGCAAGGCC |
| | CCCAAGAGGCTGATCTACGCTGCCAGCAGCCTGCAGGGCGGAG |
| | TGCCTAGCAGGTTTAGCGGCTCCGGCAGCGGCACAGAGTTCAC |
| | CCTGACCATCTCCAGCCTGCAGCCCGAGGACTTCGCCACCTACT |
| | ACTGCCTGCAGCACTACACCTACCCTCTGACCTTCGGCGGCGGC |
| | ACCAAGGTGGAGATCAAGCGTACGGTGGCTGCACCATCTGTCT |
| | TCATCTTCCCGCCATCTGATGAGCAGTTGAAATCTGGAACTGCC |
| | TCTGTTGTGTGCCTGCTGAATAACTTCTATCCCAGAGAGGCCAA |
| | AGTACAGTGGAAGGTGGATAACGCCCTCCAATCGGGTAACTCC |
| | CAGGAGAGTGTCACAGAGCAGGACAGCAAGGACAGCACCTAC |
| | AGCCTCAGCAGCACCCTGACGCTGAGCAAAGCAGACTACGAGA |
| | AACACAAAGTCTACGCCTGCGAAGTCACCCATCAGGGCCTGAG |
| | CTCGCCCGTCACAAAGAGCTTCAACAGGGGAGAGTGT (SEQ ID |
| | NO: 76) |
| T26B608 | GACATCCAGATGACCCAGAGCCCTAGCACCCTGAGCGCTAGCG |
| | TGGGCGACAGGGTGACCATCACCTGCAGAGCCAGCCAGAGCAC |
| | CAGCAGCTGGCTGGCCTGGTACCAGCAGAAGCCCGGCAAGGCC |
| | CCCAAGCTGCTGATCTACAAGACCAGCAGCCTGGAGTCCGGCG |
| | TGCCTAGCAGGTTTAGCGGCAGCGGCAGCGGCACCGAGTTTAC |
| | CCTGACCATCAGCAGCCTCCAGCCCGACGACTTCGCCACCTACT |
| | ACTGCCAGCAGTACAACAGCTACAGCTTCGGCCAGGGCACAAA |
| | GGTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 69) |
| T26B609 | GACATCCAGATGACCCAGAGCCCTAGCAGCCTGAGCGCCAGCG |
| | TGGGAGACAGGGTGACCATCACCTGCAGGGCCAGCCAGGGCAT |
| | CAGGAACGACCTGGGCTGGTACCAGCAGAAGCCCGGCAAGGCC |
| | CCCAAGAGGCTGATCTACGCTGCCAGCAGCCTGCAGGGCGGAG |
| | TGCCTAGCAGGTTTAGCGGCTCCGGCAGCGGCACAGAGTTCAC |
| | CCTGACCATCTCCAGCCTGCAGCCCGAGGACTTCGCCACCTACT |
| | ACTGCCTGCAGCACTACACCTACCCTCTGACCTTCGGCGGCGGC |
| | ACCAAGGTGGAGATCAAGCGTACGGTGGCTGCACCATCTGTCT |
| | TCATCTTCCCGCCATCTGATGAGCAGTTGAAATCTGGAACTGCC |
| | TCTGTTGTGTGCCTGCTGAATAACTTCTATCCCAGAGAGGCCAA |
| | AGTACAGTGGAAGGTGGATAACGCCCTCCAATCGGGTAACTCC |
| | CAGGAGAGTGTCACAGAGCAGGACAGCAAGGACAGCACCTAC |
| | AGCCTCAGCAGCACCCTGACGCTGAGCAAAGCAGACTACGAGA |
| | AACACAAAGTCTACGCCTGCGAAGTCACCCATCAGGGCCTGAG |
| | CTCGCCCGTCACAAAGAGCTTCAACAGGGGAGAGTGT (SEQ ID |
| | NO: 76) |
| T26B610 | GACATCCAGATGACCCAGAGCCCTAGCACACTGAGCGCCAGCG |
| | TGGGAGACAGGGTGACCATCACCTGCAGAGCCAGCCAGTCCCT |
| | GAGCAGCTGGCTGGCCTGGTACCAGCAGAAGCCCGGCAAGGCC |
| | CCCAAGCTGCTGATCTTCAAGACAAGCAGCCTGGAGAGCGGCG |
| | TGCCCTCCAGATTCAGCGGCAGCGGAAGCGGCACCGAGTTCAC |
| | CCTGACCATCAGCAGCCTGCAGCCCGACGACTTCGCCATCTACT |
| | ACTGCCAGCAGTACAACTCCTACATCTTCGGCCAGGGCACCAG |
| | GCTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 74) |
| T26B611 | GACATCCAGATGACCCAGAGCCCTAGCACACTGAGCGCCAGCG |
| | TGGGCGACAGAGTGACCATCACCTGCAGGGCCAGCCAGTCCAT |
| | CAGCAGCTGGCTGGCCTGGTACCAGCAGAAGCCCGGCAAGGCC |
| | CCCAAGCTGCTGATCTACAAGGCCAGCAGCCTGGAGAGCGGCG |
| | TGCCTAGCAGGTTCAGCGGCAGCAGGAGCGGCACCGAGTTCAC |
| | CCTGACCATCAGCAGCCTGCAGCCCGACGACTTCGCCACCTACT |
| | ACTGCCAGCAGTACAACAGCTGGACCTTCGGCCAGGGCACCAA |
| | GGTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 70) |
| T26B612 | GACATCCAGATGACCCAGAGCCCTAGCACACTGTCAGCTAGCG |
| | TCGGCGACAGGGTGACCATCACATGCAGGGCTAGTCAGAGTAT |
| | CAGTAGCTGGCTGGCCTGGTACCAGCAGAAGCCCGGCAAGGCC |
| | CCCAAGCTGCTGATCTACAAGGCCAGCAGCCTGGAGAGCGGAG |
| | TGCCCAGCAGATTCAGTGGTAGCGGCAGTGGCACCGAGTTCAC |
| | CCTGACAATCAGCAGCCTGCAGCCCGACGATTTCGCCACCTACT |
| | ACTGCCAGCAGTACAACAGCTACACCTTCGGCCAGGGCACCAA |
| | GCTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 71) |
| T26B613 | CAGAGCGCCCTGACCCAGCCTGCTAGCGTGAGCGGAAGCCCTG |
| | GCCAGAGCATCACCATCAGCTGCACAGGCACCAGCAGCGATGT |
| | GGGCGGCTACAACTACGTGAGCTGGTACCAGCAGCACCCCAGC |
| | AAGGCCCCCAAGCTGATGATCTACGATGTGAGCAACAGGCCCA |
| | GCGGCGTGAGCAATAGGTTCAGCGGCAGCAAGAGCGGCAACA |
| | CCGCCTCCCTGACAATCAGCGGCCTGCAGGCCGAGGATGAGGC |
| | CGACTACTACTGCAGCAGCTACACCAGCAGCTCCACCCTGGTG |
| | GTGTTTGGCGGCGGCACCAAGCTGACCGTGCTGGGTCAGCCCA |
| | AGGCTGCACCCAGTGTCACTCTGTTCCCGCCCTCCTCTGAGGAG |
| | CTTCAAGCCAACAAGGCCACACTGGTGTGTCTCATAAGTGACTT |
| | CTACCCGGGAGCCGTGACAGTGGCCTGGAAGGCCGATAGCAGC |
| | CCCGTCAAGGCGGGAGTGGAGACCACCACACCCTCCAAACAAA |
| | GCAACAACAAGTACGCGGCCAGCAGCTATCTGAGCCTGACGCC |
| | TGAGCAGTGGAAGTCCCACAGAAGCTACAGCTGCCAGGTCACG |
| | CATGAAGGGAGCACCGTGGAGAAGACAGTGGCCCCTACAGAAT |
| | GTTCA (SEQ ID NO: 73) |
| T26B614 | AGCAGCGAGCTGACCCAGGATCCTGCCGTGAGCGTGGCCCTGG |
| | GACAGACCGTGAGGATCACCTGCCAGGGCGACAGCCTGAGGAA |
| | CTACTACGCCAGCTGGTACCAGCAGAAACCCGGACAGGCCCCC |
| | GTGCTGGTGTTCTACGGCAAGGACAACAGACCCAGCGGCATCC |
| | CCGACAGGTTCAGCGGAAGCACCAGCGGCAACACCGCCAGCCT |
| | GACCATTACCGGCGCTCAGGCCCAGGACGAGGCCGACTACTAC |
| | TGCAACAGCAGGGACAGCAGCGGCGATCACCTGGTGTTTGGCG |
| | GCGGCACCAAGCTGACCGTGCTGGGTCAGCCCAAGGCTGCACC |
| | CAGTGTCACTCTGTTCCCGCCCTCCTCTGAGGAGCTTCAAGCCA |
| | ACAAGGCCACACTGGTGTGTCTCATAAGTGACTTCTACCCGGGA |
| | GCCGTGACAGTGGCCTGGAAGGCCGATAGCAGCCCCGTCAAGG |
| | CGGGAGTGGAGACCACCACACCCTCCAAACAAAGCAACAACA |
| | AGTACGCGGCCAGCAGCTATCTGAGCCTGACGCCTGAGCAGTG |
| | GAAGTCCCACAGAAGCTACAGCTGCCAGGTCACGCATGAAGGG |
| | AGCACCGTGGAGAAGACAGTGGCCCCTACAGAATGTTCA (SEQ |
| | ID NO: 75) |
| T26B615 | GACATCCAGATGACCCAGAGCCCTAGCACACTGAGCGCCAGCG |
| | TGGGCGACAGGGTGACCATCACCTGCAGGGCCAGCCAGAGCAT |
| | CAGCAGCTGGCTGGCCTGGTACCAGCAGAAGCCCGGCAAGGCC |
| | CCCAAGCTGCTGATCTTCAAGACCAGCAGCCTGGAGAGCGGCG |
| | TGCCTAGCAGATTTAGCGGCAGCGGCAGCGGCACCGAGTTCAC |
| | CCTGACCATCAGCAGCCTGCAGCCCGACGACTTCGCCACCTACT |
| | ACTGCCAGCAGTACAACAGCTACAGCTTCGGCGGCGGCACCAA |
| | GGTGGAGATCAAGCGTACGGTGGCTGCACCATCTGTCTTCATCT |
| | TCCCGCCATCTGATGAGCAGTTGAAATCTGGAACTGCCTCTGTT |
| | GTGTGCCTGCTGAATAACTTCTATCCCAGAGAGGCCAAAGTAC |
| | AGTGGAAGGTGGATAACGCCCTCCAATCGGGTAACTCCCAGGA |
| | GAGTGTCACAGAGCAGGACAGCAAGGACAGCACCTACAGCCTC |
| | AGCAGCACCCTGACGCTGAGCAAAGCAGACTACGAGAAACAC |
| | AAAGTCTACGCCTGCGAAGTCACCCATCAGGGCCTGAGCTCGC |
| | CCGTCACAAAGAGCTTCAACAGGGGAGAGTGT (SEQ ID NO: 72) |

TABLE 12
Complete nucleotide sequences of the heavy chains of exemplary
anti-CD37 antibodies

| Antibody | Complete Sequence |
|---|---|
| Name | (SEQ ID NO) |
| T26B373 | CAGGTGCAGCTGCAGGAAAGCGGCCCCGGACTGGTGAAGCCCA |
| | GCGAAACCCTGAGCCTGACCTGTACCGTGAGCGGCGGCAGCAT |
| | CAGCAGCGGCGTGTACTACTGGGCCTGGATCAGACAGCCCCCT |
| | GGCAAGGGCCTGGAGCTGATCGGCACCTTCTACTACAGCGGCA |
| | GCACCTACTACGACAGCAGCCTGAGGAGCAGGGTGACCATCAG |
| | CGTGGACACCAGCAAGAACCAGTTCAGCCTGAAGCTGAGCAGC |
| | GTGACCGCCGCCGATACAGCCGTGTATTACTGCGCCAGGCAGG |
| | CCGGCGACTTCGACTACTGGGGCCAGGGAACCCTGGTGACCGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGCCCACCGTGCCC |
| | AGCACCTGAACTCCTGGGGGGACCGTCAGTCTTCCTCTTCCCCC |
| | CAAAACCCAAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCATCGAGAAAACCATCTCCAAAGCCAAAGGGCAGCCCCGAGA |
| | ACCACAGGTGTACACCCTGCCCCCATCCCGGGAGGAGATGACC |
| | AAGAACCAGGTCAGCCTGACCTGCCTGGTCAAAGGCTTCTATC |
| | CCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGAGCAGAT |
| | GGCAGCAGGGGAACGTCTTCTCATGCTCCGTGATGCATGAGGC |
| | TCTGCACAACCACTACACGCAGAAGAGCCTCTCCCTGTCTCCGG |
| | GTAAA (SEQ ID NO: 77) |
| T26B374 | CAGCTGCAGCTGCAGGAGAGCGGACCCGGACTGGTGAAGCCCA |
| | GCGAGACCCTGAGCCTGACCTGCACCGTGAGCGGCGGAAGCAT |
| | CAGCAGCAGCCCCTACTATTGGGGCTGGATCAGGCAGCCTCCC |
| | GGAAAGGGCCTGGAGTGGATCGGCAGCTTCTACTACGGCGGCA |
| | GCAACTACTACAACCCCAGCCTGAAGAGCAGGGTGACCATCAG |
| | CGCCGACACCAGCAAGAACCAGTTCAGCCTGAAGCTGTCCAGC |
| | GTGACCGCCGCTGATACCGCCGTGTACTACTGCGCCAGACAGG |
| | CCGGCGACTGGGATTACTGGGGCCAGGGCACACTGGTGACCGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGCCCACCGTGCCC |
| | AGCACCTGAACTCCTGGGGGGACCGTCAGTCTTCCTCTTCCCCC |
| | CAAAACCCAAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCATCGAGAAAACCATCTCCAAAGCCAAAGGGCAGCCCCGAGA |
| | ACCACAGGTGTACACCCTGCCCCCATCCCGGGAGGAGATGACC |
| | AAGAACCAGGTCAGCCTGACCTGCCTGGTCAAAGGCTTCTATC |
| | CCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGAGCAGAT |
| | GGCAGCAGGGGAACGTCTTCTCATGCTCCGTGATGCATGAGGC |
| | TCTGCACAACCACTACACGCAGAAGAGCCTCTCCCTGTCTCCGG |
| | GTAAA (SEQ ID NO: 78) |
| T26B375 | CAGCTGCAGCTGCAGGAGAGCGGACCTGGCCTGGTGAAGCCCA |
| | GCGAGACCCTGAGCCTGACCTGCACCGTGAGCGGCGGAAGCAT |
| | CAGCAGCGGCATCTACAACTGGGGCTGGATCAGGCAGCCCCCT |
| | GGCAAAGGCCTGGAGTGGATCGGCAACTTCCAGTACAGCGGCA |
| | TCACCTACTACAACCCCAGCCTGAAGAGCAGGGTGACCATCAG |
| | CGTGGACACCAGCAAGAACCAGTTCAGCCTGCAGCTGAGCAGC |
| | GTGACAGCTGCCGACACCGCCGTGTACTACTGTGCCAGGCAGG |
| | CCGGCGACTTCGATTACTGGGGCCAGGGCACCCTGGTGACAGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGCCCACCGTGCCC |
| | AGCACCTGAACTCCTGGGGGGACCGTCAGTCTTCCTCTTCCCCC |
| | CAAAACCCAAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCATCGAGAAAACCATCTCCAAAGCCAAAGGGCAGCCCCGAGA |
| | ACCACAGGTGTACACCCTGCCCCCATCCCGGGAGGAGATGACC |
| | AAGAACCAGGTCAGCCTGACCTGCCTGGTCAAAGGCTTCTATC |
| | CCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGAGCAGAT |
| | GGCAGCAGGGGAACGTCTTCTCATGCTCCGTGATGCATGAGGC |
| | TCTGCACAACCACTACACGCAGAAGAGCCTCTCCCTGTCTCCGG |
| | GTAAA (SEQ ID NO: 79) |
| T26B379 | CAGGTGCAGCTGCAGGAATCCGGCCCTGGCCTGGTGAAACCCA |
| | GCGAGACCCTGAGCCTGACCTGCACAGTGAGTGGAGGTAGCAT |
| | CAGCAGCAGCATCTACTACTGGGCCTGGATCAGGCAGCCTCCC |
| | GGCAAAGGACTGGAGTGGATCGGTACCATCTACTACGGCGGCA |
| | GCCCCTATTACAGTCCCTCACTGAAGAGCAGGGTGACCATCAG |
| | CATCGACACCAGCAAGTCCCAGTTCAGCCTGAGGCTGACCAGC |
| | GTGACAGTGGCCGACACCGCCGTGTATTACTGCGCCAGGAGGG |
| | CCGGCGACTTCGACTACTGGGGACAGGGCACCCTGGTGACCGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGCCCACCGTGCCC |
| | AGCACCTGAACTCCTGGGGGGACCGTCAGTCTTCCTCTTCCCCC |
| | CAAAACCCAAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCATCGAGAAAACCATCTCCAAAGCCAAAGGGCAGCCCCGAGA |
| | ACCACAGGTGTACACCCTGCCCCCATCCCGGGAGGAGATGACC |
| | AAGAACCAGGTCAGCCTGACCTGCCTGGTCAAAGGCTTCTATC |
| | CCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGAGCAGAT |
| | GGCAGCAGGGGAACGTCTTCTCATGCTCCGTGATGCATGAGGC |
| | TCTGCACAACCACTACACGCAGAAGAGCCTCTCCCTGTCTCCGG |
| | GTAAA (SEQ ID NO: 80) |
| T26B382 | CAGGTGCAGCTGGTGGAGAGCGGCGGAGGACTGGTGAAGCCTG |
| | GAGGCTCCCTGAGACTGAGCTGTGCCGCCTCCGGCTTCACCTTC |
| | AGCGACTACTATATGACCTGGATCAGGCAGGCCCCTGGCAAGG |
| | GACTGGAGTGGGTGAGCTACATCAGCAGCAGCGGCATCACCAA |
| | GTACTACGCCGACTTCGTGAAGGGCAGGTTCACCATCAGCAGG |
| | GACAACGCCAAGAACAGCCTGTACCTGCAGATGAACAGCCTGA |
| | GGGCCGAGGACACCGCCGTGTACTACTGCGCCAGGGACAGAGA |
| | CAGGCAGTGGCTGCTGGAGTTTGACTACTGGGGCCAGGGCACC |
| | CTGGTGACAGTGTCCAGCGCCTCCACCAAGGGCCCATCGGTCTT |
| | CCCCCTGGCACCCTCCTCCAAGAGCACCTCTGGGGGCACAGCG |
| | GCCCTGGGCTGCCTGGTCAAGGACTACTTCCCCGAACCGGTGA |
| | CGGTGTCGTGGAACTCAGGCGCCCTGACCAGCGGCGTGCACAC |
| | CTTCCCGGCTGTCCTACAGTCCTCAGGACTCTACTCCCTCAGCA |
| | GCGTGGTGACCGTGCCCTCCAGCAGCTTGGGCACCCAGACCTA |
| | CATCTGCAACGTGAATCACAAGCCCAGCAACACCAAGGTGGAC |
| | AAGAAAGTTGAGCCCAAATCTTGTGACAAAACTCACACATGCC |
| | CACCGTGCCCAGCACCTGAACTCCTGGGGGGACCGTCAGTCTTC |
| | CTCTTCCCCCCAAAACCCAAGGACACCCTCATGATCTCCCGGAC |
| | CCCTGAGGTCACATGCGTGGTGGTGGACGTGAGCCACGAAGAC |
| | CCTGAGGTCAAGTTCAACTGGTACGTGGACGGCGTGGAGGTGC |
| | ATAATGCCAAGACAAAGCCGCGGGAGGAGCAGTACAACAGCA |
| | CGTACCGTGTGGTCAGCGTCCTCACCGTCCTGCACCAGGACTGG |
| | CTGAATGGCAAGGAGTACAAGTGCAAGGTCTCCAACAAAGCCC |
| | TCCCAGCCCCCATCGAGAAAACCATCTCCAAAGCCAAAGGGCA |
| | GCCCCGAGAACCACAGGTGTACACCCTGCCCCCATCCCGGGAG |
| | GAGATGACCAAGAACCAGGTCAGCCTGACCTGCCTGGTCAAAG |
| | GCTTCTATCCCAGCGACATCGCCGTGGAGTGGGAGAGCAATGG |
| | GCAGCCGGAGAACAACTACAAGACCACGCCTCCCGTGCTGGAC |
| | TCCGACGGCTCCTTCTTCCTCTACAGCAAGCTCACCGTGGACAA |
| | GAGCAGATGGCAGCAGGGGAACGTCTTCTCATGCTCCGTGATG |
| | CATGAGGCTCTGCACAACCACTACACGCAGAAGAGCCTCTCCC |
| | TGTCTCCGGGTAAA (SEQ ID NO: 81) |
| T26B385 | CAGGTGCAGCTGCAGGAGAGCGGCCCTGGACTGGTGAAGCCCA |
| | GCGAGACCCTGAGCCTGACCTGCATCGTGAGCGGCGGCAGCGT |
| | GAGCAGCAGAAACTACTACTGGGGCTGGATTAGGCAGCCCCCT |
| | GGCAAAGGCCTGGAGTGGATCGGCAGGATCTACTACAGCGGCA |
| | ACACCAACTACAACCCCAGCCTGAAGAGCAGAGTGACCATCTC |
| | CGTGGACACCAGCAAGAACCAGTTCAGCCTGAAGCTGAGCAGC |
| | GTGACAGCCGCCGATACCGCCGTGTACTACTGCGCCAGATGGG |
| | CCGGCGAAATCGACTACTGGGGCCAGGGCACACTGGTGACCGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGCCCACCGTGCCC |
| | AGCACCTGAACTCCTGGGGGGACCGTCAGTCTTCCTCTTCCCCC |
| | CAAAACCCAAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCATCGAGAAAACCATCTCCAAAGCCAAAGGGCAGCCCCGAGA |
| | ACCACAGGTGTACACCCTGCCCCCATCCCGGGAGGAGATGACC |
| | AAGAACCAGGTCAGCCTGACCTGCCTGGTCAAAGGCTTCTATC |
| | CCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGAGCAGAT |
| | GGCAGCAGGGGAACGTCTTCTCATGCTCCGTGATGCATGAGGC |
| | TCTGCACAACCACTACACGCAGAAGAGCCTCTCCCTGTCTCCGG |
| | GTAAA (SEQ ID NO: 82) |
| T26B386 | CAGGTGCAGCTGCAGGAGAGCGGCCCTGGACTGGTGAAACCCA |
| | GCGAGACCCTGAGCCTGAGCTGCACAGTGAGCGGCGGCAGCCT |
| | GAACAGCTACAACTACTACTGGGGCTGGGTGAGACAGCCTCCC |
| | GGCAAGGGACTGGAGTGGATCGGCACCATCTACAGCAGCGGCT |
| | CCGCCTACTACAACCCCAGCCTGAAGAGCAGGTTCACCATCAG |
| | CGTGGCCACCAGCAAGAACCAGTTCAGCCTGAGACTGAGCAGC |
| | GTGACAGCCGCCGACACCGCTGTGTACTACTGCGCCAGGGGCT |
| | ACAGGAACAGCTGGTACGCCCTGTTCGAGTACTGGGGACAGGG |
| | CACCCTGGTGACCGTGAGCAGCGCCTCCACCAAGGGCCCATCG |
| | GTCTTCCCCCTGGCACCCTCCTCCAAGAGCACCTCTGGGGGCAC |
| | AGCGGCCCTGGGCTGCCTGGTCAAGGACTACTTCCCCGAACCG |
| | GTGACGGTGTCGTGGAACTCAGGCGCCCTGACCAGCGGCGTGC |
| | ACACCTTCCCGGCTGTCCTACAGTCCTCAGGACTCTACTCCCTC |
| | AGCAGCGTGGTGACCGTGCCCTCCAGCAGCTTGGGCACCCAGA |
| | CCTACATCTGCAACGTGAATCACAAGCCCAGCAACACCAAGGT |
| | GGACAAGAAAGTTGAGCCCAAATCTTGTGACAAAACTCACACA |
| | TGCCCACCGTGCCCAGCACCTGAACTCCTGGGGGGACCGTCAG |
| | TCTTCCTCTTCCCCCCAAAACCCAAGGACACCCTCATGATCTCC |
| | CGGACCCCTGAGGTCACATGCGTGGTGGTGGACGTGAGCCACG |
| | AAGACCCTGAGGTCAAGTTCAACTGGTACGTGGACGGCGTGGA |
| | GGTGCATAATGCCAAGACAAAGCCGCGGGAGGAGCAGTACAA |
| | CAGCACGTACCGTGTGGTCAGCGTCCTCACCGTCCTGCACCAGG |
| | ACTGGCTGAATGGCAAGGAGTACAAGTGCAAGGTCTCCAACAA |
| | AGCCCTCCCAGCCCCCATCGAGAAAACCATCTCCAAAGCCAAA |
| | GGGCAGCCCCGAGAACCACAGGTGTACACCCTGCCCCCATCCC |
| | GGGAGGAGATGACCAAGAACCAGGTCAGCCTGACCTGCCTGGT |
| | CAAAGGCTTCTATCCCAGCGACATCGCCGTGGAGTGGGAGAGC |
| | AATGGGCAGCCGGAGAACAACTACAAGACCACGCCTCCCGTGC |
| | TGGACTCCGACGGCTCCTTCTTCCTCTACAGCAAGCTCACCGTG |
| | GACAAGAGCAGATGGCAGCAGGGGAACGTCTTCTCATGCTCCG |
| | TGATGCATGAGGCTCTGCACAACCACTACACGCAGAAGAGCCT |
| | CTCCCTGTCTCCGGGTAAA (SEQ ID NO: 83) |
| T26B388 | CAGGTGCAGCTGCAGCAGTGGGGCGCTGGACTGCTGAAGCCCA |
| | GCGAGACCCTGTCCCTGACCTGTGCCGTGTACGGCGGCAGCTTC |
| | AGCGACTACTACTGGAGCTGGATCAGACAGCCTCCTGGCAAGG |
| | GCCTGGAGTGGATCGGCGAGATCGACCACAGCGGCTCCACCGA |
| | CTACAACCCCAGCCTGAAGTCCAGGGTGACCATCAGCGTGGAC |
| | ACCAGCAAGAACCAGTTCAGCCTGAAGCTGAGCAGCGTGACAG |
| | CCGCCGATACCGCCGTGTACTACTGCGCCAGGAGCATGTACTA |
| | CGACATCTGGACCGGCTACCACGGCGCCTTTGACATCTGGGGC |
| | CAGGGCACCATGGTGACCGTGAGCAGCGCCTCCACCAAGGGCC |
| | CATCGGTCTTCCCCCTGGCACCCTCCTCCAAGAGCACCTCTGGG |
| | GGCACAGCGGCCCTGGGCTGCCTGGTCAAGGACTACTTCCCCG |
| | AACCGGTGACGGTGTCGTGGAACTCAGGCGCCCTGACCAGCGG |
| | CGTGCACACCTTCCCGGCTGTCCTACAGTCCTCAGGACTCTACT |
| | CCCTCAGCAGCGTGGTGACCGTGCCCTCCAGCAGCTTGGGCAC |
| | CCAGACCTACATCTGCAACGTGAATCACAAGCCCAGCAACACC |
| | AAGGTGGACAAGAAAGTTGAGCCCAAATCTTGTGACAAAACTC |
| | ACACATGCCCACCGTGCCCAGCACCTGAACTCCTGGGGGGACC |
| | GTCAGTCTTCCTCTTCCCCCCAAAACCCAAGGACACCCTCATGA |
| | TCTCCCGGACCCCTGAGGTCACATGCGTGGTGGTGGACGTGAG |
| | CCACGAAGACCCTGAGGTCAAGTTCAACTGGTACGTGGACGGC |
| | GTGGAGGTGCATAATGCCAAGACAAAGCCGCGGGAGGAGCAG |
| | TACAACAGCACGTACCGTGTGGTCAGCGTCCTCACCGTCCTGCA |
| | CCAGGACTGGCTGAATGGCAAGGAGTACAAGTGCAAGGTCTCC |
| | AACAAAGCCCTCCCAGCCCCCATCGAGAAAACCATCTCCAAAG |
| | CCAAAGGGCAGCCCCGAGAACCACAGGTGTACACCCTGCCCCC |
| | ATCCCGGGAGGAGATGACCAAGAACCAGGTCAGCCTGACCTGC |
| | CTGGTCAAAGGCTTCTATCCCAGCGACATCGCCGTGGAGTGGG |
| | AGAGCAATGGGCAGCCGGAGAACAACTACAAGACCACGCCTCC |
| | CGTGCTGGACTCCGACGGCTCCTTCTTCCTCTACAGCAAGCTCA |
| | CCGTGGACAAGAGCAGATGGCAGCAGGGGAACGTCTTCTCATG |
| | CTCCGTGATGCATGAGGCTCTGCACAACCACTACACGCAGAAG |
| | AGCCTCTCCCTGTCTCCGGGTAAA (SEQ ID NO: 84) |
| T26B459 | CAGGTGCAGCTGCAGGAAAGCGGCCCCGGACTGGTGAAGCCCA |
| | GCGAAACCCTGAGCCTGACCTGTACCGTGAGCGGCGGCAGCAT |
| | CAGCAGCGGCGTGTACTACTGGGCCTGGATCAGACAGCCCCCT |
| | GGCAAGGGCCTGGAGCTGATCGGCACCTTCTACTACAGCGGCA |
| | GCACCTACTACGACAGCAGCCTGAGGAGCAGGGTGACCATCAG |
| | CGTGGACACCAGCAAGAACCAGTTCAGCCTGAAGCTGAGCAGC |
| | GTGACCGCCGCCGATACAGCCGTGTATTACTGCGCCAGGCAGG |
| | CCGGCGACTTCGACTACTGGGGCCAGGGAACCCTGGTGACCGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGCCCACCGTGCCC |
| | AGCACCTGAACTCCTGGGGGGACCGTCAGTCTTCCTCTTCCCCC |
| | CAAAACCCGAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCATCGAGAAAACCATCTCCAAAGCCAAAGGGCAGCCCCGAGA |
| | ACCACAGGTGTACACCCTGCCCCCATCCCGGGAGGAGATGACC |
| | AAGAACCAGGTCAGCCTGACCTGCCTGGTCAAAGGCTTCTATC |
| | CCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGAGCAGGT |
| | GGCAGCAGGGGAACGTCTTCTCATGCTCCGTGATGCATGAGGC |
| | TCTGCACAACCACTACCGGCAGAAGAGCCTCTCCCTGTCTCCGG |
| | GTAAA (SEQ ID NO: 85) |
| T26B460 | CAGCTGCAGCTGCAGGAGAGCGGACCCGGACTGGTGAAGCCCA |
| | GCGAGACCCTGAGCCTGACCTGCACCGTGAGCGGCGGAAGCAT |
| | CAGCAGCAGCCCCTACTATTGGGGCTGGATCAGGCAGCCTCCC |
| | GGAAAGGGCCTGGAGTGGATCGGCAGCTTCTACTACGGCGGCA |
| | GCAACTACTACAACCCCAGCCTGAAGAGCAGGGTGACCATCAG |
| | CGCCGACACCAGCAAGAACCAGTTCAGCCTGAAGCTGTCCAGC |
| | GTGACCGCCGCTGATACCGCCGTGTACTACTGCGCCAGACAGG |
| | CCGGCGACTGGGATTACTGGGGCCAGGGCACACTGGTGACCGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGCCCACCGTGCCC |
| | AGCACCTGAACTCCTGGGGGGACCGTCAGTCTTCCTCTTCCCCC |
| | CAAAACCCGAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCATCGAGAAAACCATCTCCAAAGCCAAAGGGCAGCCCCGAGA |
| | ACCACAGGTGTACACCCTGCCCCCATCCCGGGAGGAGATGACC |
| | AAGAACCAGGTCAGCCTGACCTGCCTGGTCAAAGGCTTCTATC |
| | CCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGAGCAGGT |
| | GGCAGCAGGGGAACGTCTTCTCATGCTCCGTGATGCATGAGGC |
| | TCTGCACAACCACTACCGGCAGAAGAGCCTCTCCCTGTCTCCGG |
| | GTAAA (SEQ ID NO: 86) |
| T26B461 | CAGCTGCAGCTGCAGGAGAGCGGACCTGGCCTGGTGAAGCCCA |
| | GCGAGACCCTGAGCCTGACCTGCACCGTGAGCGGCGGAAGCAT |
| | CAGCAGCGGCATCTACAACTGGGGCTGGATCAGGCAGCCCCCT |
| | GGCAAAGGCCTGGAGTGGATCGGCAACTTCCAGTACAGCGGCA |
| | TCACCTACTACAACCCCAGCCTGAAGAGCAGGGTGACCATCAG |
| | CGTGGACACCAGCAAGAACCAGTTCAGCCTGCAGCTGAGCAGC |
| | GTGACAGCTGCCGACACCGCCGTGTACTACTGTGCCAGGCAGG |
| | CCGGCGACTTCGATTACTGGGGCCAGGGCACCCTGGTGACAGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGCCCACCGTGCCC |
| | AGCACCTGAACTCCTGGGGGGACCGTCAGTCTTCCTCTTCCCCC |
| | CAAAACCCGAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCATCGAGAAAACCATCTCCAAAGCCAAAGGGCAGCCCCGAGA |
| | ACCACAGGTGTACACCCTGCCCCCATCCCGGGAGGAGATGACC |
| | AAGAACCAGGTCAGCCTGACCTGCCTGGTCAAAGGCTTCTATC |
| | CCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGAGCAGGT |
| | GGCAGCAGGGGAACGTCTTCTCATGCTCCGTGATGCATGAGGC |
| | TCTGCACAACCACTACCGGCAGAAGAGCCTCTCCCTGTCTCCGG |
| | GTAAA (SEQ ID NO: 87) |
| T26B462 | CAGGTGCAGCTGCAGGAATCCGGCCCTGGCCTGGTGAAACCCA |
| | GCGAGACCCTGAGCCTGACCTGCACAGTGAGTGGAGGTAGCAT |
| | CAGCAGCAGCATCTACTACTGGGCCTGGATCAGGCAGCCTCCC |
| | GGCAAAGGACTGGAGTGGATCGGTACCATCTACTACGGCGGCA |
| | GCCCCTATTACAGTCCCTCACTGAAGAGCAGGGTGACCATCAG |
| | CATCGACACCAGCAAGTCCCAGTTCAGCCTGAGGCTGACCAGC |
| | GTGACAGTGGCCGACACCGCCGTGTATTACTGCGCCAGGAGGG |
| | CCGGCGACTTCGACTACTGGGGACAGGGCACCCTGGTGACCGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGCCCACCGTGCCC |
| | AGCACCTGAACTCCTGGGGGGACCGTCAGTCTTCCTCTTCCCCC |
| | CAAAACCCGAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCATCGAGAAAACCATCTCCAAAGCCAAAGGGCAGCCCCGAGA |
| | ACCACAGGTGTACACCCTGCCCCCATCCCGGGAGGAGATGACC |
| | AAGAACCAGGTCAGCCTGACCTGCCTGGTCAAAGGCTTCTATC |
| | CCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGAGCAGGT |
| | GGCAGCAGGGGAACGTCTTCTCATGCTCCGTGATGCATGAGGC |
| | TCTGCACAACCACTACCGGCAGAAGAGCCTCTCCCTGTCTCCGG |
| | GTAAA (SEQ ID NO: 88) |
| T26B463 | CAGGTGCAGCTGGTGGAGAGCGGCGGAGGACTGGTGAAGCCTG |
| | GAGGCTCCCTGAGACTGAGCTGTGCCGCCTCCGGCTTCACCTTC |
| | AGCGACTACTATATGACCTGGATCAGGCAGGCCCCTGGCAAGG |
| | GACTGGAGTGGGTGAGCTACATCAGCAGCAGCGGCATCACCAA |
| | GTACTACGCCGACTTCGTGAAGGGCAGGTTCACCATCAGCAGG |
| | GACAACGCCAAGAACAGCCTGTACCTGCAGATGAACAGCCTGA |
| | GGGCCGAGGACACCGCCGTGTACTACTGCGCCAGGGACAGAGA |
| | CAGGCAGTGGCTGCTGGAGTTTGACTACTGGGGCCAGGGCACC |
| | CTGGTGACAGTGTCCAGCGCCTCCACCAAGGGCCCATCGGTCTT |
| | CCCCCTGGCACCCTCCTCCAAGAGCACCTCTGGGGGCACAGCG |
| | GCCCTGGGCTGCCTGGTCAAGGACTACTTCCCCGAACCGGTGA |
| | CGGTGTCGTGGAACTCAGGCGCCCTGACCAGCGGCGTGCACAC |
| | CTTCCCGGCTGTCCTACAGTCCTCAGGACTCTACTCCCTCAGCA |
| | GCGTGGTGACCGTGCCCTCCAGCAGCTTGGGCACCCAGACCTA |
| | CATCTGCAACGTGAATCACAAGCCCAGCAACACCAAGGTGGAC |
| | AAGAAAGTTGAGCCCAAATCTTGTGACAAAACTCACACATGCC |
| | CACCGTGCCCAGCACCTGAACTCCTGGGGGGACCGTCAGTCTTC |
| | CTCTTCCCCCCAAAACCCGAGGACACCCTCATGATCTCCCGGAC |
| | CCCTGAGGTCACATGCGTGGTGGTGGACGTGAGCCACGAAGAC |
| | CCTGAGGTCAAGTTCAACTGGTACGTGGACGGCGTGGAGGTGC |
| | ATAATGCCAAGACAAAGCCGCGGGAGGAGCAGTACAACAGCA |
| | CGTACCGTGTGGTCAGCGTCCTCACCGTCCTGCACCAGGACTGG |
| | CTGAATGGCAAGGAGTACAAGTGCAAGGTCTCCAACAAAGCCC |
| | TCCCAGCCCCCATCGAGAAAACCATCTCCAAAGCCAAAGGGCA |
| | GCCCCGAGAACCACAGGTGTACACCCTGCCCCCATCCCGGGAG |
| | GAGATGACCAAGAACCAGGTCAGCCTGACCTGCCTGGTCAAAG |
| | GCTTCTATCCCAGCGACATCGCCGTGGAGTGGGAGAGCAATGG |
| | GCAGCCGGAGAACAACTACAAGACCACGCCTCCCGTGCTGGAC |
| | TCCGACGGCTCCTTCTTCCTCTACAGCAAGCTCACCGTGGACAA |
| | GAGCAGGTGGCAGCAGGGGAACGTCTTCTCATGCTCCGTGATG |
| | CATGAGGCTCTGCACAACCACTACCGGCAGAAGAGCCTCTCCC |
| | TGTCTCCGGGTAAA (SEQ ID NO: 89) |
| T26B464 | CAGGTGCAGCTGCAGGAGAGCGGCCCTGGACTGGTGAAGCCCA |
| | GCGAGACCCTGAGCCTGACCTGCATCGTGAGCGGCGGCAGCGT |
| | GAGCAGCAGAAACTACTACTGGGGCTGGATTAGGCAGCCCCCT |
| | GGCAAAGGCCTGGAGTGGATCGGCAGGATCTACTACAGCGGCA |
| | ACACCAACTACAACCCCAGCCTGAAGAGCAGAGTGACCATCTC |
| | CGTGGACACCAGCAAGAACCAGTTCAGCCTGAAGCTGAGCAGC |
| | GTGACAGCCGCCGATACCGCCGTGTACTACTGCGCCAGATGGG |
| | CCGGCGAAATCGACTACTGGGGCCAGGGCACACTGGTGACCGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGCCCACCGTGCCC |
| | AGCACCTGAACTCCTGGGGGGACCGTCAGTCTTCCTCTTCCCCC |
| | CAAAACCCGAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCATCGAGAAAACCATCTCCAAAGCCAAAGGGCAGCCCCGAGA |
| | ACCACAGGTGTACACCCTGCCCCCATCCCGGGAGGAGATGACC |
| | AAGAACCAGGTCAGCCTGACCTGCCTGGTCAAAGGCTTCTATC |
| | CCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGAGCAGGT |
| | GGCAGCAGGGGAACGTCTTCTCATGCTCCGTGATGCATGAGGC |
| | TCTGCACAACCACTACCGGCAGAAGAGCCTCTCCCTGTCTCCGG |
| | GTAAA (SEQ ID NO: 90) |
| T26B465 | CAGGTGCAGCTGCAGGAGAGCGGCCCTGGACTGGTGAAACCCA |
| | GCGAGACCCTGAGCCTGAGCTGCACAGTGAGCGGCGGCAGCCT |
| | GAACAGCTACAACTACTACTGGGGCTGGGTGAGACAGCCTCCC |
| | GGCAAGGGACTGGAGTGGATCGGCACCATCTACAGCAGCGGCT |
| | CCGCCTACTACAACCCCAGCCTGAAGAGCAGGTTCACCATCAG |
| | CGTGGCCACCAGCAAGAACCAGTTCAGCCTGAGACTGAGCAGC |
| | GTGACAGCCGCCGACACCGCTGTGTACTACTGCGCCAGGGGCT |
| | ACAGGAACAGCTGGTACGCCCTGTTCGAGTACTGGGGACAGGG |
| | CACCCTGGTGACCGTGAGCAGCGCCTCCACCAAGGGCCCATCG |
| | GTCTTCCCCCTGGCACCCTCCTCCAAGAGCACCTCTGGGGGCAC |
| | AGCGGCCCTGGGCTGCCTGGTCAAGGACTACTTCCCCGAACCG |
| | GTGACGGTGTCGTGGAACTCAGGCGCCCTGACCAGCGGCGTGC |
| | ACACCTTCCCGGCTGTCCTACAGTCCTCAGGACTCTACTCCCTC |
| | AGCAGCGTGGTGACCGTGCCCTCCAGCAGCTTGGGCACCCAGA |
| | CCTACATCTGCAACGTGAATCACAAGCCCAGCAACACCAAGGT |
| | GGACAAGAAAGTTGAGCCCAAATCTTGTGACAAAACTCACACA |
| | TGCCCACCGTGCCCAGCACCTGAACTCCTGGGGGGACCGTCAG |
| | TCTTCCTCTTCCCCCCAAAACCCGAGGACACCCTCATGATCTCC |
| | CGGACCCCTGAGGTCACATGCGTGGTGGTGGACGTGAGCCACG |
| | AAGACCCTGAGGTCAAGTTCAACTGGTACGTGGACGGCGTGGA |
| | GGTGCATAATGCCAAGACAAAGCCGCGGGAGGAGCAGTACAA |
| | CAGCACGTACCGTGTGGTCAGCGTCCTCACCGTCCTGCACCAGG |
| | ACTGGCTGAATGGCAAGGAGTACAAGTGCAAGGTCTCCAACAA |
| | AGCCCTCCCAGCCCCCATCGAGAAAACCATCTCCAAAGCCAAA |
| | GGGCAGCCCCGAGAACCACAGGTGTACACCCTGCCCCCATCCC |
| | GGGAGGAGATGACCAAGAACCAGGTCAGCCTGACCTGCCTGGT |
| | CAAAGGCTTCTATCCCAGCGACATCGCCGTGGAGTGGGAGAGC |
| | AATGGGCAGCCGGAGAACAACTACAAGACCACGCCTCCCGTGC |
| | TGGACTCCGACGGCTCCTTCTTCCTCTACAGCAAGCTCACCGTG |
| | GACAAGAGCAGGTGGCAGCAGGGGAACGTCTTCTCATGCTCCG |
| | TGATGCATGAGGCTCTGCACAACCACTACCGGCAGAAGAGCCT |
| | CTCCCTGTCTCCGGGTAAA (SEQ ID NO: 91) |
| T26B466 | CAGGTGCAGCTGCAGCAGTGGGGCGCTGGACTGCTGAAGCCCA |
| | GCGAGACCCTGTCCCTGACCTGTGCCGTGTACGGCGGCAGCTTC |
| | AGCGACTACTACTGGAGCTGGATCAGACAGCCTCCTGGCAAGG |
| | GCCTGGAGTGGATCGGCGAGATCGACCACAGCGGCTCCACCGA |
| | CTACAACCCCAGCCTGAAGTCCAGGGTGACCATCAGCGTGGAC |
| | ACCAGCAAGAACCAGTTCAGCCTGAAGCTGAGCAGCGTGACAG |
| | CCGCCGATACCGCCGTGTACTACTGCGCCAGGAGCATGTACTA |
| | CGACATCTGGACCGGCTACCACGGCGCCTTTGACATCTGGGGC |
| | CAGGGCACCATGGTGACCGTGAGCAGCGCCTCCACCAAGGGCC |
| | CATCGGTCTTCCCCCTGGCACCCTCCTCCAAGAGCACCTCTGGG |
| | GGCACAGCGGCCCTGGGCTGCCTGGTCAAGGACTACTTCCCCG |
| | AACCGGTGACGGTGTCGTGGAACTCAGGCGCCCTGACCAGCGG |
| | CGTGCACACCTTCCCGGCTGTCCTACAGTCCTCAGGACTCTACT |
| | CCCTCAGCAGCGTGGTGACCGTGCCCTCCAGCAGCTTGGGCAC |
| | CCAGACCTACATCTGCAACGTGAATCACAAGCCCAGCAACACC |
| | AAGGTGGACAAGAAAGTTGAGCCCAAATCTTGTGACAAAACTC |
| | ACACATGCCCACCGTGCCCAGCACCTGAACTCCTGGGGGGACC |
| | GTCAGTCTTCCTCTTCCCCCCAAAACCCGAGGACACCCTCATGA |
| | TCTCCCGGACCCCTGAGGTCACATGCGTGGTGGTGGACGTGAG |
| | CCACGAAGACCCTGAGGTCAAGTTCAACTGGTACGTGGACGGC |
| | GTGGAGGTGCATAATGCCAAGACAAAGCCGCGGGAGGAGCAG |
| | TACAACAGCACGTACCGTGTGGTCAGCGTCCTCACCGTCCTGCA |
| | CCAGGACTGGCTGAATGGCAAGGAGTACAAGTGCAAGGTCTCC |
| | AACAAAGCCCTCCCAGCCCCCATCGAGAAAACCATCTCCAAAG |
| | CCAAAGGGCAGCCCCGAGAACCACAGGTGTACACCCTGCCCCC |
| | ATCCCGGGAGGAGATGACCAAGAACCAGGTCAGCCTGACCTGC |
| | CTGGTCAAAGGCTTCTATCCCAGCGACATCGCCGTGGAGTGGG |
| | AGAGCAATGGGCAGCCGGAGAACAACTACAAGACCACGCCTCC |
| | CGTGCTGGACTCCGACGGCTCCTTCTTCCTCTACAGCAAGCTCA |
| | CCGTGGACAAGAGCAGGTGGCAGCAGGGGAACGTCTTCTCATG |
| | CTCCGTGATGCATGAGGCTCTGCACAACCACTACCGGCAGAAG |
| | AGCCTCTCCCTGTCTCCGGGTAAA (SEQ ID NO: 92) |
| T26B608 | CAGGTGCAGCTGCAGGAAAGCGGCCCCGGACTGGTGAAGCCCA |
| | GCGAAACCCTGAGCCTGACCTGTACCGTGAGCGGCGGCAGCAT |
| | CAGCAGCGGCGTGTACTACTGGGCCTGGATCAGACAGCCCCCT |
| | GGCAAGGGCCTGGAGCTGATCGGCACCTTCTACTACAGCGGCA |
| | GCACCTACTACGACAGCAGCCTGAGGAGCAGGGTGACCATCAG |
| | CGTGGACACCAGCAAGAACCAGTTCAGCCTGAAGCTGAGCAGC |
| | GTGACCGCCGCCGATACAGCCGTGTATTACTGCGCCAGGCAGG |
| | CCGGCGACTTCGACTACTGGGGCCAGGGAACCCTGGTGACCGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGTCCACCGTGCCC |
| | AGCACCTGAACTGCTGGGGGGACCGGATGTCTTCCTCTTCCCCC |
| | CAAAACCCAAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCGAGGAGAAAACCATCTCCAAAGCCAAAGGGCAGCCCCGAG |
| | AACCACAGGTGTACACCCTGCCCCCATCCCGGGAGGAGATGAC |
| | CAAGAACCAGGTCAGCCTGACCTGCCTGGTCAAAGGCTTCTAT |
| | CCCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGTCTAGAT |
| | GGCAGCAGGGGAACGTCTTCTCATGCTCCGTGATGCATGAGGC |
| | TCTGCACAACCACTACACGCAGAAGAGCCTCTCCCTGTCTCCGG |
| | GT (SEQ ID NO: 93) |
| T26B609 | CAGGTGCAGCTGCAGCAGTGGGGCGCTGGACTGCTGAAGCCCA |
| | GCGAGACCCTGTCCCTGACCTGTGCCGTGTACGGCGGCAGCTTC |
| | AGCGACTACTACTGGAGCTGGATCAGACAGCCTCCTGGCAAGG |
| | GCCTGGAGTGGATCGGCGAGATCGACCACAGCGGCTCCACCGA |
| | CTACAACCCCAGCCTGAAGTCCAGGGTGACCATCAGCGTGGAC |
| | ACCAGCAAGAACCAGTTCAGCCTGAAGCTGAGCAGCGTGACAG |
| | CCGCCGATACCGCCGTGTACTACTGCGCCAGGAGCATGTACTA |
| | CGACATCTGGACCGGCTACCACGGCGCCTTTGACATCTGGGGC |
| | CAGGGCACCATGGTGACCGTGAGCAGCGCCTCCACCAAGGGCC |
| | CATCGGTCTTCCCCCTGGCACCCTCCTCCAAGAGCACCTCTGGG |
| | GGCACAGCGGCCCTGGGCTGCCTGGTCAAGGACTACTTCCCCG |
| | AACCGGTGACGGTGTCGTGGAACTCAGGCGCCCTGACCAGCGG |
| | CGTGCACACCTTCCCGGCTGTCCTACAGTCCTCAGGACTCTACT |
| | CCCTCAGCAGCGTGGTGACCGTGCCCTCCAGCAGCTTGGGCAC |
| | CCAGACCTACATCTGCAACGTGAATCACAAGCCCAGCAACACC |
| | AAGGTGGACAAGAAAGTTGAGCCCAAATCTTGTGACAAAACTC |
| | ACACATGTCCACCGTGCCCAGCACCTGAACTGCTGGGGGGACC |
| | GGATGTCTTCCTCTTCCCCCCAAAACCCAAGGACACCCTCATGA |
| | TCTCCCGGACCCCTGAGGTCACATGCGTGGTGGTGGACGTGAG |
| | CCACGAAGACCCTGAGGTCAAGTTCAACTGGTACGTGGACGGC |
| | GTGGAGGTGCATAATGCCAAGACAAAGCCGCGGGAGGAGCAG |
| | TACAACAGCACGTACCGTGTGGTCAGCGTCCTCACCGTCCTGCA |
| | CCAGGACTGGCTGAATGGCAAGGAGTACAAGTGCAAGGTCTCC |
| | AACAAAGCCCTCCCAGCCCCCGAGGAGAAAACCATCTCCAAAG |
| | CCAAAGGGCAGCCCCGAGAACCACAGGTGTACACCCTGCCCCC |
| | ATCCCGGGAGGAGATGACCAAGAACCAGGTCAGCCTGACCTGC |
| | CTGGTCAAAGGCTTCTATCCCAGCGACATCGCCGTGGAGTGGG |
| | AGAGCAATGGGCAGCCGGAGAACAACTACAAGACCACGCCTCC |
| | CGTGCTGGACTCCGACGGCTCCTTCTTCCTCTACAGCAAGCTCA |
| | CCGTGGACAAGTCTAGATGGCAGCAGGGGAACGTCTTCTCATG |
| | CTCCGTGATGCATGAGGCTCTGCACAACCACTACACGCAGAAG |
| | AGCCTCTCCCTGTCTCCGGGT (SEQ ID NO: 94) |
| T26B610 | CAGGTGCAGCTGCAGGAGAGCGGCCCTGGACTGGTGAAGCCCA |
| | GCGAGACCCTGAGCCTGACCTGCATCGTGAGCGGCGGCAGCGT |
| | GAGCAGCAGAAACTACTACTGGGGCTGGATTAGGCAGCCCCCT |
| | GGCAAAGGCCTGGAGTGGATCGGCAGGATCTACTACAGCGGCA |
| | ACACCAACTACAACCCCAGCCTGAAGAGCAGAGTGACCATCTC |
| | CGTGGACACCAGCAAGAACCAGTTCAGCCTGAAGCTGAGCAGC |
| | GTGACAGCCGCCGATACCGCCGTGTACTACTGCGCCAGATGGG |
| | CCGGCGAAATCGACTACTGGGGCCAGGGCACACTGGTGACCGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGTCCACCGTGCCC |
| | AGCACCTGAACTGCTGGGGGGACCGGATGTCTTCCTCTTCCCCC |
| | CAAAACCCAAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCGAGGAGAAAACCATCTCCAAAGCCAAAGGGCAGCCCCGAG |
| | AACCACAGGTGTACACCCTGCCCCCATCCCGGGAGGAGATGAC |
| | CAAGAACCAGGTCAGCCTGACCTGCCTGGTCAAAGGCTTCTAT |
| | CCCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGTCTAGAT |
| | GGCAGCAGGGGAACGTCTTCTCATGCTCCGTGATGCATGAGGC |
| | TCTGCACAACCACTACACGCAGAAGAGCCTCTCCCTGTCTCCGG |
| | GT (SEQ ID NO: 95) |
| T26B611 | CAGCTGCAGCTGCAGGAGAGCGGACCCGGACTGGTGAAGCCCA |
| | GCGAGACCCTGAGCCTGACCTGCACCGTGAGCGGCGGAAGCAT |
| | CAGCAGCAGCCCCTACTATTGGGGCTGGATCAGGCAGCCTCCC |
| | GGAAAGGGCCTGGAGTGGATCGGCAGCTTCTACTACGGCGGCA |
| | GCAACTACTACAACCCCAGCCTGAAGAGCAGGGTGACCATCAG |
| | CGCCGACACCAGCAAGAACCAGTTCAGCCTGAAGCTGTCCAGC |
| | GTGACCGCCGCTGATACCGCCGTGTACTACTGCGCCAGACAGG |
| | CCGGCGACTGGGATTACTGGGGCCAGGGCACACTGGTGACCGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGTCCACCGTGCCC |
| | AGCACCTGAACTGCTGGGGGGACCGGATGTCTTCCTCTTCCCCC |
| | CAAAACCCAAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCGAGGAGAAAACCATCTCCAAAGCCAAAGGGCAGCCCCGAG |
| | AACCACAGGTGTACACCCTGCCCCCATCCCGGGAGGAGATGAC |
| | CAAGAACCAGGTCAGCCTGACCTGCCTGGTCAAAGGCTTCTAT |
| | CCCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGTCTAGAT |
| | GGCAGCAGGGGAACGTCTTCTCATGCTCCGTGATGCATGAGGC |
| | TCTGCACAACCACTACACGCAGAAGAGCCTCTCCCTGTCTCCGG |
| | GT (SEQ ID NO: 96) |
| T26B612 | CAGCTGCAGCTGCAGGAGAGCGGACCTGGCCTGGTGAAGCCCA |
| | GCGAGACCCTGAGCCTGACCTGCACCGTGAGCGGCGGAAGCAT |
| | CAGCAGCGGCATCTACAACTGGGGCTGGATCAGGCAGCCCCCT |
| | GGCAAAGGCCTGGAGTGGATCGGCAACTTCCAGTACAGCGGCA |
| | TCACCTACTACAACCCCAGCCTGAAGAGCAGGGTGACCATCAG |
| | CGTGGACACCAGCAAGAACCAGTTCAGCCTGCAGCTGAGCAGC |
| | GTGACAGCTGCCGACACCGCCGTGTACTACTGTGCCAGGCAGG |
| | CCGGCGACTTCGATTACTGGGGCCAGGGCACCCTGGTGACAGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGTCCACCGTGCCC |
| | AGCACCTGAACTGCTGGGGGGACCGGATGTCTTCCTCTTCCCCC |
| | CAAAACCCAAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCGAGGAGAAAACCATCTCCAAAGCCAAAGGGCAGCCCCGAG |
| | AACCACAGGTGTACACCCTGCCCCCATCCCGGGAGGAGATGAC |
| | CAAGAACCAGGTCAGCCTGACCTGCCTGGTCAAAGGCTTCTAT |
| | CCCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGTCTAGAT |
| | GGCAGCAGGGGAACGTCTTCTCATGCTCCGTGATGCATGAGGC |
| | TCTGCACAACCACTACACGCAGAAGAGCCTCTCCCTGTCTCCGG |
| | GT (SEQ ID NO: 97) |
| T26B613 | CAGGTGCAGCTGGTGGAGAGCGGCGGAGGACTGGTGAAGCCTG |
| | GAGGCTCCCTGAGACTGAGCTGTGCCGCCTCCGGCTTCACCTTC |
| | AGCGACTACTATATGACCTGGATCAGGCAGGCCCCTGGCAAGG |
| | GACTGGAGTGGGTGAGCTACATCAGCAGCAGCGGCATCACCAA |
| | GTACTACGCCGACTTCGTGAAGGGCAGGTTCACCATCAGCAGG |
| | GACAACGCCAAGAACAGCCTGTACCTGCAGATGAACAGCCTGA |
| | GGGCCGAGGACACCGCCGTGTACTACTGCGCCAGGGACAGAGA |
| | CAGGCAGTGGCTGCTGGAGTTTGACTACTGGGGCCAGGGCACC |
| | CTGGTGACAGTGTCCAGCGCCTCCACCAAGGGCCCATCGGTCTT |
| | CCCCCTGGCACCCTCCTCCAAGAGCACCTCTGGGGGCACAGCG |
| | GCCCTGGGCTGCCTGGTCAAGGACTACTTCCCCGAACCGGTGA |
| | CGGTGTCGTGGAACTCAGGCGCCCTGACCAGCGGCGTGCACAC |
| | CTTCCCGGCTGTCCTACAGTCCTCAGGACTCTACTCCCTCAGCA |
| | GCGTGGTGACCGTGCCCTCCAGCAGCTTGGGCACCCAGACCTA |
| | CATCTGCAACGTGAATCACAAGCCCAGCAACACCAAGGTGGAC |
| | AAGAAAGTTGAGCCCAAATCTTGTGACAAAACTCACACATGTC |
| | CACCGTGCCCAGCACCTGAACTGCTGGGGGGACCGGATGTCTT |
| | CCTCTTCCCCCCAAAACCCAAGGACACCCTCATGATCTCCCGGA |
| | CCCCTGAGGTCACATGCGTGGTGGTGGACGTGAGCCACGAAGA |
| | CCCTGAGGTCAAGTTCAACTGGTACGTGGACGGCGTGGAGGTG |
| | CATAATGCCAAGACAAAGCCGCGGGAGGAGCAGTACAACAGC |
| | ACGTACCGTGTGGTCAGCGTCCTCACCGTCCTGCACCAGGACTG |
| | GCTGAATGGCAAGGAGTACAAGTGCAAGGTCTCCAACAAAGCC |
| | CTCCCAGCCCCCGAGGAGAAAACCATCTCCAAAGCCAAAGGGC |
| | AGCCCCGAGAACCACAGGTGTACACCCTGCCCCCATCCCGGGA |
| | GGAGATGACCAAGAACCAGGTCAGCCTGACCTGCCTGGTCAAA |
| | GGCTTCTATCCCAGCGACATCGCCGTGGAGTGGGAGAGCAATG |
| | GGCAGCCGGAGAACAACTACAAGACCACGCCTCCCGTGCTGGA |
| | CTCCGACGGCTCCTTCTTCCTCTACAGCAAGCTCACCGTGGACA |
| | AGTCTAGATGGCAGCAGGGGAACGTCTTCTCATGCTCCGTGATG |
| | CATGAGGCTCTGCACAACCACTACACGCAGAAGAGCCTCTCCC |
| | TGTCTCCGGGT (SEQ ID NO: 98) |
| T26B614 | CAGGTGCAGCTGCAGGAGAGCGGCCCTGGACTGGTGAAACCCA |
| | GCGAGACCCTGAGCCTGAGCTGCACAGTGAGCGGCGGCAGCCT |
| | GAACAGCTACAACTACTACTGGGGCTGGGTGAGACAGCCTCCC |
| | GGCAAGGGACTGGAGTGGATCGGCACCATCTACAGCAGCGGCT |
| | CCGCCTACTACAACCCCAGCCTGAAGAGCAGGTTCACCATCAG |
| | CGTGGCCACCAGCAAGAACCAGTTCAGCCTGAGACTGAGCAGC |
| | GTGACAGCCGCCGACACCGCTGTGTACTACTGCGCCAGGGGCT |
| | ACAGGAACAGCTGGTACGCCCTGTTCGAGTACTGGGGACAGGG |
| | CACCCTGGTGACCGTGAGCAGCGCCTCCACCAAGGGCCCATCG |
| | GTCTTCCCCCTGGCACCCTCCTCCAAGAGCACCTCTGGGGGCAC |
| | AGCGGCCCTGGGCTGCCTGGTCAAGGACTACTTCCCCGAACCG |
| | GTGACGGTGTCGTGGAACTCAGGCGCCCTGACCAGCGGCGTGC |
| | ACACCTTCCCGGCTGTCCTACAGTCCTCAGGACTCTACTCCCTC |
| | AGCAGCGTGGTGACCGTGCCCTCCAGCAGCTTGGGCACCCAGA |
| | CCTACATCTGCAACGTGAATCACAAGCCCAGCAACACCAAGGT |
| | GGACAAGAAAGTTGAGCCCAAATCTTGTGACAAAACTCACACA |
| | TGTCCACCGTGCCCAGCACCTGAACTGCTGGGGGGACCGGATG |
| | TCTTCCTCTTCCCCCCAAAACCCAAGGACACCCTCATGATCTCC |
| | CGGACCCCTGAGGTCACATGCGTGGTGGTGGACGTGAGCCACG |
| | AAGACCCTGAGGTCAAGTTCAACTGGTACGTGGACGGCGTGGA |
| | GGTGCATAATGCCAAGACAAAGCCGCGGGAGGAGCAGTACAA |
| | CAGCACGTACCGTGTGGTCAGCGTCCTCACCGTCCTGCACCAGG |
| | ACTGGCTGAATGGCAAGGAGTACAAGTGCAAGGTCTCCAACAA |
| | AGCCCTCCCAGCCCCCGAGGAGAAAACCATCTCCAAAGCCAAA |
| | GGGCAGCCCCGAGAACCACAGGTGTACACCCTGCCCCCATCCC |
| | GGGAGGAGATGACCAAGAACCAGGTCAGCCTGACCTGCCTGGT |
| | CAAAGGCTTCTATCCCAGCGACATCGCCGTGGAGTGGGAGAGC |
| | AATGGGCAGCCGGAGAACAACTACAAGACCACGCCTCCCGTGC |
| | TGGACTCCGACGGCTCCTTCTTCCTCTACAGCAAGCTCACCGTG |
| | GACAAGTCTAGATGGCAGCAGGGGAACGTCTTCTCATGCTCCG |
| | TGATGCATGAGGCTCTGCACAACCACTACACGCAGAAGAGCCT |
| | CTCCCTGTCTCCGGGT (SEQ ID NO: 99) |
| T26B615 | CAGGTGCAGCTGCAGGAATCCGGCCCTGGCCTGGTGAAACCCA |
| | GCGAGACCCTGAGCCTGACCTGCACAGTGAGTGGAGGTAGCAT |
| | CAGCAGCAGCATCTACTACTGGGCCTGGATCAGGCAGCCTCCC |
| | GGCAAAGGACTGGAGTGGATCGGTACCATCTACTACGGCGGCA |
| | GCCCCTATTACAGTCCCTCACTGAAGAGCAGGGTGACCATCAG |
| | CATCGACACCAGCAAGTCCCAGTTCAGCCTGAGGCTGACCAGC |
| | GTGACAGTGGCCGACACCGCCGTGTATTACTGCGCCAGGAGGG |
| | CCGGCGACTTCGACTACTGGGGACAGGGCACCCTGGTGACCGT |
| | GAGCAGCGCCTCCACCAAGGGCCCATCGGTCTTCCCCCTGGCA |
| | CCCTCCTCCAAGAGCACCTCTGGGGGCACAGCGGCCCTGGGCT |
| | GCCTGGTCAAGGACTACTTCCCCGAACCGGTGACGGTGTCGTG |
| | GAACTCAGGCGCCCTGACCAGCGGCGTGCACACCTTCCCGGCT |
| | GTCCTACAGTCCTCAGGACTCTACTCCCTCAGCAGCGTGGTGAC |
| | CGTGCCCTCCAGCAGCTTGGGCACCCAGACCTACATCTGCAAC |
| | GTGAATCACAAGCCCAGCAACACCAAGGTGGACAAGAAAGTTG |
| | AGCCCAAATCTTGTGACAAAACTCACACATGTCCACCGTGCCC |
| | AGCACCTGAACTGCTGGGGGGACCGGATGTCTTCCTCTTCCCCC |
| | CAAAACCCAAGGACACCCTCATGATCTCCCGGACCCCTGAGGT |
| | CACATGCGTGGTGGTGGACGTGAGCCACGAAGACCCTGAGGTC |
| | AAGTTCAACTGGTACGTGGACGGCGTGGAGGTGCATAATGCCA |
| | AGACAAAGCCGCGGGAGGAGCAGTACAACAGCACGTACCGTG |
| | TGGTCAGCGTCCTCACCGTCCTGCACCAGGACTGGCTGAATGGC |
| | AAGGAGTACAAGTGCAAGGTCTCCAACAAAGCCCTCCCAGCCC |
| | CCGAGGAGAAAACCATCTCCAAAGCCAAAGGGCAGCCCCGAG |
| | AACCACAGGTGTACACCCTGCCCCCATCCCGGGAGGAGATGAC |
| | CAAGAACCAGGTCAGCCTGACCTGCCTGGTCAAAGGCTTCTAT |
| | CCCAGCGACATCGCCGTGGAGTGGGAGAGCAATGGGCAGCCGG |
| | AGAACAACTACAAGACCACGCCTCCCGTGCTGGACTCCGACGG |
| | CTCCTTCTTCCTCTACAGCAAGCTCACCGTGGACAAGTCTAGAT |
| | GGCAGCAGGGGAACGTCTTCTCATGCTCCGTGATGCATGAGGC |
| | TCTGCACAACCACTACACGCAGAAGAGCCTCTCCCTGTCTCCGG |
| | GT (SEQ ID NO: 100) |

### Example 3—CDC Functional Analysis of Anti-GPRC5D Antibodies

To further confirm that the CDC potentiation characteristics of the RE mutations in the Fc region are general properties of the mutations, effects of the RE mutations were investigated in a third target cell line, H929, expressing GPRC5D. Briefly, a wildtype IgG1 antibody with low fucosylation, GC5B747.CLF, and its counterpart with RE mutations, GC5B752.CLF, were added to the H929 target cells and incubated for 30 minutes at 37° C. Baby rabbit serum was then added to target cells to a final concentration of 10% to provide a source of complement components for CDC. The mixture was incubated for 4 hours at 37° C. 100 μl of CellTiter-Glo reagent (Promega) was added to the mixture, followed by incubation for 10 minutes at room temperature. Target cell viability was determined by measuring luminescence with a Tecan SPARK Reader and reported in Relative Luminescence Units (RLU).

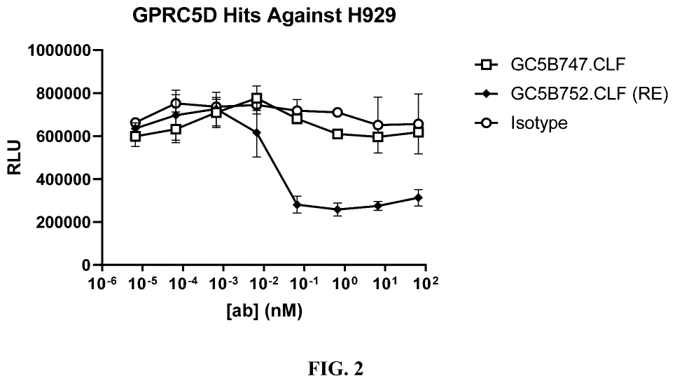

The wildtype GC5B747.CLF antibody did not show any CDC activity against H929 cells, while the GC5B752.CLF antibody comprising RE mutations showed significantly enhanced CDC activity against H929 cells (FIG. 2).

These results showed that the RE mutations in the Fc region consistently potentiate CDC activity of antibodies across multiple target cell lines. Such CDC potentiation is therefore not a target-specific phenomenon but a general characteristic of the RE mutations that can be applied to enhance Fc effector function in general.

The VL and VH amino acid sequences of exemplary anti-GPRC5D antibodies are summarized in Table 13 below.

TABLE 13
VL and VH amino acid sequences of exemplary anti-GPRC5D
antibodies

| Antibody | | Variable Sequence |
|---|---|---|
| Name | VL/VH | (SEQ ID NO) |
| GC5B747 | VL | EIVLTQSPATLSLSPGERATLSCRASQSVSSYLAWYQQ |
| | | KPGQAPRLLIYDASNRATGIPARFSGSGSGTDFTLTISS |
| | | LEPEDFAVYYCQQRSNWPPTFGQGTKVEIK (SEQ ID |
| | | NO: 33) |
| GC5B747 | VH | QLQLQESGPGLVKPSETLSLTCTVSGGSLSSSSYWWG |
| | | WTRQPPGRGLEWIGTMYYSGNIYYNPSLQSRATISVD |
| | | TSKNQFSLKLSSVTAADTAVYYCARHVGYSYGRRFW |
| | | YFDLWGRGTLVTVSS (SEQ ID NO: 34) |
| GC5B752 | VL | EIVLTQSPATLSLSPGERATLSCRASQSVSSYLAWYQQ |
| | | KPGQAPRLLIYDASNRATGIPARFSGSGSGTDFTLTISS |
| | | LEPEDFAVYYCQQRSNWPPTFGQGTKVEIK (SEQ ID |
| | | NO: 33) |
| GC5B752 | VH | QLQLQESGPGLVKPSETLSLTCTVSGGSLSSSSYWWG |
| | | WTRQPPGRGLEWIGTMYYSGNIYYNPSLQSRATISVD |
| | | TSKNQFSLKLSSVTAADTAVYYCARHVGYSYGRRFW |
| | | YFDLWGRGTLVTVSS (SEQ ID NO: 34) |

The complete amino acid sequences of the light chains and heavy chains of exemplary anti-GPRC5D antibodies are summarized in Table 14 below.

TABLE 14
Complete amino acid sequences of the light chains and
heavy chains of exemplary anti-GPRC5D antibodies

| | Light | |
|---|---|---|
| | Chain/ | |
| Antibody | Heavy | Complete Sequence |
| Name | Chain | (SEQ ID NO) |
| GC5B747 | Light | EIVLTQSPATLSLSPGERATLSCRASQSVSSYLAWYQ |
| | chain | QKPGQAPRLLIYDASNRATGIPARFSGSGSGTDFTLTI |
| | | SSLEPEDFAVYYCQQRSNWPPTFGQGTKVEIKRTVA |
| | | APSVFIFPPSDEQLKSGTASVVCLLNNFYPREAKVQW |
| | | KVDNALQSGNSQESVTEQDSKDSTYSLSSTLTLSKA |
| | | DYEKHKVYACEVTHQGLSSPVTKSFNRGEC (SEQ ID |
| | | NO: 101) |
| GC5B747 | Heavy | QLQLQESGPGLVKPSETLSLTCTVSGGSLSSSSYWW |
| | chain | GWTRQPPGRGLEWIGTMYYSGNIYYNPSLQSRATIS |
| | | VDTSKNQFSLKLSSVTAADTAVYYCARHVGYSYGR |
| | | RFWYFDLWGRGTLVTVSSASTKGPSVFPLAPSSKSTS |
| | | GGTAALGCLVKDYFPEPVTVSWNSGALTSGVHTFPA |
| | | VLQSSGLYSLSSVVTVPSSSLGTQTYICNVNHKPSNT |
| | | KVDKKVEPKSCDKTHTCPPCPAPELLGGPSVFLFPPK |
| | | PKDTLMISRTPEVTCVVVDVSHEDPEVKFNWYVDG |
| | | VEVHNAKTKPREEQYNSTYRVVSVLTVLHQDWLNG |
| | | KEYKCKVSNKALPAPIEKTISKAKGQPREPQVYTLPP |
| | | SREEMTKNQVSLTCLVKGFYPSDIAVEWESNGQPEN |
| | | NYKTTPPVLDSDGSFFLYSKLTVDKSRWQQGNVFSC |
| | | SVMHEALHNHYTQKSLSLSPG (SEQ ID NO: 102) |
| GC5B752 | Light | EIVLTQSPATLSLSPGERATLSCRASQSVSSYLAWYQ |
| | chain | QKPGQAPRLLIYDASNRATGIPARFSGSGSGTDFTLTI |
| | | SSLEPEDFAVYYCQQRSNWPPTFGQGTKVEIKRTVA |
| | | APSVFIFPPSDEQLKSGTASVVCLLNNFYPREAKVQW |
| | | KVDNALQSGNSQESVTEQDSKDSTYSLSSTLTLSKA |
| | | DYEKHKVYACEVTHQGLSSPVTKSFNRGEC (SEQ ID |
| | | NO: 101) |
| GC5B752 | Heavy | QLQLQESGPGLVKPSETLSLTCTVSGGSLSSSSYWW |
| | chain | GWTRQPPGRGLEWIGTMYYSGNIYYNPSLQSRATIS |
| | | VDTSKNQFSLKLSSVTAADTAVYYCARHVGYSYGR |
| | | RFWYFDLWGRGTLVTVSSASTKGPSVFPLAPSSKSTS |
| | | GGTAALGCLVKDYFPEPVTVSWNSGALTSGVHTFPA |
| | | VLQSSGLYSLSSVVTVPSSSLGTQTYICNVNHKPSNT |
| | | KVDKKVEPKSCDKTHTCPPCPAPELLGGPSVFLFPPK |
| | | PEDTLMISRTPEVTCVVVDVSHEDPEVKFNWYVDG |
| | | VEVHNAKTKPREEQYNSTYRVVSVLTVLHQDWLNG |
| | | KEYKCKVSNKALPAPIEKTISKAKGQPREPQVYTLPP |
| | | SREEMTKNQVSLTCLVKGFYPSDIAVEWESNGQPEN |
| | | NYKTTPPVLDSDGSFFLYSKLTVDKSRWQQGNVFSC |
| | | SVMHEALHNHYRQKSLSLSPG (SEQ ID NO: 103) |

The VL and VH nucleotide sequences of exemplary anti-GPRC5D antibodies are summarized in Table 15 below.

TABLE 15
VL and VH nucleotide sequences of exemplary
anti-GPRC5D antibodies

| Antibody | VL/ | Variable Sequence |
|---|---|---|
| Name | VH | (SEQ ID NO) |
| GC5B747 | VL | GAAATCGTATTGACCCAATCTCCTGCTACTCTCAGC |
| | | TTGAGCCCCGGTGAGAGAGCAACCCTGTCTTGTCGA |
| | | GCCTCTCAAAGTGTGTCTTCCTACCTCGCCTGGTATC |
| | | AACAGAAGCCTGGCCAAGCCCCACGACTGCTTATTT |
| | | ATGACGCTTCCAATCGGGCTACTGGAATCCCTGCCA |
| | | GGTTCTCCGGGAGTGGTTCTGGAACAGACTTCACTC |
| | | TGACCATCTCCTCACTGGAACCCGAGGACTTCGCAG |
| | | TCTATTATTGCCAACAGAGGAGTAATTGGCCCCCTA |
| | | CATTTGGCCAAGGCACAAAAGTCGAGATTAAG (SEQ |
| | | ID NO: 35) |
| GC5B747 | VH | CAACTTCAACTTCAGGAGAGTGGCCCAGGACTCGTT |
| | | AAACCTAGTGAGACCCTGTCCCTGACCTGTACTGTG |
| | | TCCGGTGGATCACTGTCATCCTCCTCATATTGGTGG |
| | | GGCTGGACTCGCCAGCCACCAGGCAGGGGATTGGA |
| | | GTGGATTGGCACTATGTATTACTCAGGCAATATTTA |
| | | CTATAATCCCTCTCTCCAGAGTCGGGCTACAATATC |
| | | CGTCGATACTAGCAAAAATCAATTTTCCCTGAAATT |
| | | GAGTAGCGTAACTGCCGCCGACACAGCCGTCTATTA |
| | | CTGTGCACGACACGTTGGGTATTCATACGGTAGGCG |
| | | ATTTTGGTACTTCGATTTGTGGGGGAGAGGAACCCT |
| | | TGTGACTGTAAGCAGC (SEQ ID NO: 36) |
| GC5B752 | VL | GAAATCGTATTGACCCAATCTCCTGCTACTCTCAGC |
| | | TTGAGCCCCGGTGAGAGAGCAACCCTGTCTTGTCGA |
| | | GCCTCTCAAAGTGTGTCTTCCTACCTCGCCTGGTATC |
| | | AACAGAAGCCTGGCCAAGCCCCACGACTGCTTATTT |
| | | ATGACGCTTCCAATCGGGCTACTGGAATCCCTGCCA |
| | | GGTTCTCCGGGAGTGGTTCTGGAACAGACTTCACTC |
| | | TGACCATCTCCTCACTGGAACCCGAGGACTTCGCAG |
| | | TCTATTATTGCCAACAGAGGAGTAATTGGCCCCCTA |
| | | CATTTGGCCAAGGCACAAAAGTCGAGATTAAG (SEQ |
| | | ID NO: 35) |
| GC5B752 | VH | CAACTTCAACTTCAGGAGAGTGGCCCAGGACTCGTT |
| | | AAACCTAGTGAGACCCTGTCCCTGACCTGTACTGTG |
| | | TCCGGTGGATCACTGTCATCCTCCTCATATTGGTGG |
| | | GGCTGGACTCGCCAGCCACCAGGCAGGGGATTGGA |
| | | GTGGATTGGCACTATGTATTACTCAGGCAATATTTA |
| | | CTATAATCCCTCTCTCCAGAGTCGGGCTACAATATC |
| | | CGTCGATACTAGCAAAAATCAATTTTCCCTGAAATT |
| | | GAGTAGCGTAACTGCCGCCGACACAGCCGTCTATTA |
| | | CTGTGCACGACACGTTGGGTATTCATACGGTAGGCG |
| | | ATTTTGGTACTTCGATTTGTGGGGGAGAGGAACCCT |
| | | TGTGACTGTAAGCAGC (SEQ ID NO: 36) |

The complete nucleotide sequences of the light chains and heavy chains of exemplary anti-GPRC5D antibodies are summarized in Table 16 below.

TABLE 16
Complete nucleotide sequences of the light chains
and heavy chains of exemplary anti-GPRC5D antibodies

| | Light | |
|---|---|---|
| | Chain/ | |
| Antibody | Heavy | Complete Sequence |
| Name | Chain | (SEQ ID NO) |
| GC5B747 | Light | GAAATCGTATTGACCCAATCTCCTGCTACTCTCAG |
| | chain | CTTGAGCCCCGGTGAGAGAGCAACCCTGTCTTGTC |
| | | GAGCCTCTCAAAGTGTGTCTTCCTACCTCGCCTGG |
| | | TATCAACAGAAGCCTGGCCAAGCCCCACGACTGC |
| | | TTATTTATGACGCTTCCAATCGGGCTACTGGAATC |
| | | CCTGCCAGGTTCTCCGGGAGTGGTTCTGGAACAGA |
| | | CTTCACTCTGACCATCTCCTCACTGGAACCCGAGG |
| | | ACTTCGCAGTCTATTATTGCCAACAGAGGAGTAAT |
| | | TGGCCCCCTACATTTGGCCAAGGCACAAAAGTCG |
| | | AGATTAAGCGGACAGTGGCCGCTCCTTCCGTGTTC |
| | | ATCTTCCCACCTTCCGACGAGCAGCTGAAGTCCGG |
| | | CACAGCTTCTGTCGTGTGCCTGCTGAACAACTTCT |
| | | ACCCTCGGGAAGCCAAGGTGCAGTGGAAGGTGGA |
| | | CAATGCCCTGCAGTCCGGCAACTCCCAAGAGTCTG |
| | | TGACCGAGCAGGACTCCAAGGACAGCACCTACAG |
| | | CCTGTCCTCCACACTGACCCTGTCCAAGGCCGACT |
| | | ACGAGAAGCACAAGGTGTACGCCTGCGAAGTGAC |
| | | CCATCAGGGCCTGTCTAGCCCTGTGACCAAGTCTT |
| | | TCAACCGGGGCGAGTGT (SEQ ID NO: 104) |
| GC5B747 | Heavy | CAACTTCAACTTCAGGAGAGTGGCCCAGGACTCGT |
| | chain | TAAACCTAGTGAGACCCTGTCCCTGACCTGTACTG |
| | | TGTCCGGTGGATCACTGTCATCCTCCTCATATTGGT |
| | | GGGGCTGGACTCGCCAGCCACCAGGCAGGGGATT |
| | | GGAGTGGATTGGCACTATGTATTACTCAGGCAATA |
| | | TTTACTATAATCCCTCTCTCCAGAGTCGGGCTACA |
| | | ATATCCGTCGATACTAGCAAAAATCAATTTTCCCT |
| | | GAAATTGAGTAGCGTAACTGCCGCCGACACAGCC |
| | | GTCTATTACTGTGCACGACACGTTGGGTATTCATA |
| | | CGGTAGGCGATTTTGGTACTTCGATTTGTGGGGGA |
| | | GAGGAACCCTTGTGACTGTAAGCAGCGCCTCCACC |
| | | AAGGGCCCATCGGTCTTCCCCCTGGCACCCTCCTC |
| | | CAAGAGCACCTCTGGGGGCACAGCGGCCCTGGGC |
| | | TGCCTGGTCAAGGACTACTTCCCCGAACCGGTGAC |
| | | GGTGTCGTGGAACTCAGGCGCCCTGACCAGCGGC |
| | | GTGCACACCTTCCCGGCTGTCCTACAGTCCTCAGG |
| | | ACTCTACTCCCTCAGCAGCGTGGTGACCGTGCCCT |
| | | CCAGCAGCTTGGGCACCCAGACCTACATCTGCAAC |
| | | GTGAATCACAAGCCCAGCAACACCAAGGTGGACA |
| | | AGAAAGTTGAGCCCAAATCTTGTGACAAAACTCA |
| | | CACATGTCCACCGTGCCCAGCACCTGAACTGCTGG |
| | | GGGGACCGTCAGTCTTCCTCTTCCCCCCAAAACCC |
| | | AAGGACACCCTCATGATCTCCCGGACCCCTGAGGT |
| | | CACATGCGTGGTGGTGGACGTGAGCCACGAAGAC |
| | | CCTGAGGTCAAGTTCAACTGGTACGTGGACGGCGT |
| | | GGAGGTGCATAATGCCAAGACAAAGCCGCGGGAG |
| | | GAGCAGTACAACAGCACGTACCGTGTGGTCAGCG |
| | | TCCTCACCGTCCTGCACCAGGACTGGCTGAATGGC |
| | | AAGGAGTACAAGTGCAAGGTCTCCAACAAAGCCC |
| | | TCCCAGCCCCCATCGAGAAAACCATCTCCAAAGCC |
| | | AAAGGGCAGCCCCGAGAACCACAGGTGTACACCC |
| | | TGCCCCCATCCCGGGAGGAGATGACCAAGAACCA |
| | | GGTCAGCCTGACCTGCCTGGTCAAAGGCTTCTATC |
| | | CCAGCGACATCGCCGTGGAGTGGGAGAGCAATGG |
| | | GCAGCCGGAGAACAACTACAAGACCACGCCTCCC |
| | | GTGCTGGACTCCGACGGCTCCTTCTTCCTCTACAG |
| | | CAAGCTCACCGTGGACAAGTCTAGATGGCAGCAG |
| | | GGGAACGTCTTCTCATGCTCCGTGATGCATGAGGC |
| | | TCTGCACAACCACTACACGCAGAAGAGCCTCTCCC |
| | | TGTCTCCGGGT (SEQ ID NO: 105) |
| GC5B752 | Light | GAAATCGTATTGACCCAATCTCCTGCTACTCTCAG |
| | chain | CTTGAGCCCCGGTGAGAGAGCAACCCTGTCTTGTC |
| | | GAGCCTCTCAAAGTGTGTCTTCCTACCTCGCCTGG |
| | | TATCAACAGAAGCCTGGCCAAGCCCCACGACTGC |
| | | TTATTTATGACGCTTCCAATCGGGCTACTGGAATC |
| | | CCTGCCAGGTTCTCCGGGAGTGGTTCTGGAACAGA |
| | | CTTCACTCTGACCATCTCCTCACTGGAACCCGAGG |
| | | ACTTCGCAGTCTATTATTGCCAACAGAGGAGTAAT |
| | | TGGCCCCCTACATTTGGCCAAGGCACAAAAGTCG |
| | | AGATTAAGCGGACAGTGGCCGCTCCTTCCGTGTTC |
| | | ATCTTCCCACCTTCCGACGAGCAGCTGAAGTCCGG |
| | | CACAGCTTCTGTCGTGTGCCTGCTGAACAACTTCT |
| | | ACCCTCGGGAAGCCAAGGTGCAGTGGAAGGTGGA |
| | | CAATGCCCTGCAGTCCGGCAACTCCCAAGAGTCTG |
| | | TGACCGAGCAGGACTCCAAGGACAGCACCTACAG |
| | | CCTGTCCTCCACACTGACCCTGTCCAAGGCCGACT |
| | | ACGAGAAGCACAAGGTGTACGCCTGCGAAGTGAC |
| | | CCATCAGGGCCTGTCTAGCCCTGTGACCAAGTCTT |
| | | TCAACCGGGGCGAGTGT (SEQ ID NO: 104) |
| GC5B752 | Heavy | CAACTTCAACTTCAGGAGAGTGGCCCAGGACTCGT |
| | chain | TAAACCTAGTGAGACCCTGTCCCTGACCTGTACTG |
| | | TGTCCGGTGGATCACTGTCATCCTCCTCATATTGGT |
| | | GGGGCTGGACTCGCCAGCCACCAGGCAGGGGATT |
| | | GGAGTGGATTGGCACTATGTATTACTCAGGCAATA |
| | | TTTACTATAATCCCTCTCTCCAGAGTCGGGCTACA |
| | | ATATCCGTCGATACTAGCAAAAATCAATTTTCCCT |
| | | GAAATTGAGTAGCGTAACTGCCGCCGACACAGCC |
| | | GTCTATTACTGTGCACGACACGTTGGGTATTCATA |
| | | CGGTAGGCGATTTTGGTACTTCGATTTGTGGGGGA |
| | | GAGGAACCCTTGTGACTGTAAGCAGCGCCTCCACC |
| | | AAGGGCCCATCGGTCTTCCCCCTGGCACCCTCCTC |
| | | CAAGAGCACCTCTGGGGGCACAGCGGCCCTGGGC |
| | | TGCCTGGTCAAGGACTACTTCCCCGAACCGGTGAC |
| | | GGTGTCGTGGAACTCAGGCGCCCTGACCAGCGGC |
| | | GTGCACACCTTCCCGGCTGTCCTACAGTCCTCAGG |
| | | ACTCTACTCCCTCAGCAGCGTGGTGACCGTGCCCT |
| | | CCAGCAGCTTGGGCACCCAGACCTACATCTGCAAC |
| | | GTGAATCACAAGCCCAGCAACACCAAGGTGGACA |
| | | AGAAAGTTGAGCCCAAATCTTGTGACAAAACTCA |
| | | CACATGTCCACCGTGCCCAGCACCTGAACTGCTGG |
| | | GGGGACCGTCAGTCTTCCTCTTCCCCCCAAAACCC |
| | | GAGGACACCCTCATGATCTCCCGGACCCCTGAGGT |
| | | CACATGCGTGGTGGTGGACGTGAGCCACGAAGAC |
| | | CCTGAGGTCAAGTTCAACTGGTACGTGGACGGCGT |
| | | GGAGGTGCATAATGCCAAGACAAAGCCGCGGGAG |
| | | GAGCAGTACAACAGCACGTACCGTGTGGTCAGCG |
| | | TCCTCACCGTCCTGCACCAGGACTGGCTGAATGGC |
| | | AAGGAGTACAAGTGCAAGGTCTCCAACAAAGCCC |
| | | TCCCAGCCCCCATCGAGAAAACCATCTCCAAAGCC |
| | | AAAGGGCAGCCCCGAGAACCACAGGTGTACACCC |
| | | TGCCCCCATCCCGGGAGGAGATGACCAAGAACCA |
| | | GGTCAGCCTGACCTGCCTGGTCAAAGGCTTCTATC |
| | | CCAGCGACATCGCCGTGGAGTGGGAGAGCAATGG |
| | | GCAGCCGGAGAACAACTACAAGACCACGCCTCCC |
| | | GTGCTGGACTCCGACGGCTCCTTCTTCCTCTACAG |
| | | CAAGCTCACCGTGGACAAGTCTAGATGGCAGCAG |
| | | GGGAACGTCTTCTCATGCTCCGTGATGCATGAGGC |
| | | TCTGCACAACCACTACCGCCAGAAGAGCCTCTCCC |
| | | TGTCTCCGGGT (SEQ ID NO: 106) |

### Example 4—Generation of Anti-KLK2 Antibodies

### Antibody Generation Using Transgenic Mice (Ablexis®) and Transgenic Rats (OmniRat®) Expressing Human Immunoglobulin Loci

The OmniRat® contains a chimeric human/rat IgH locus (comprising 22 human VHs, all human D and JH segments in natural configuration linked to the rat CH locus) together with fully human IgL loci (12 Vκs linked to Jκ-Cκ and 16 Vλs linked to Jλ-Cλ) (see, e.g., Osborn, et al., J Immunol, 2013, 190(4): 1481-90). Accordingly, the rats exhibit reduced expression of rat immunoglobulin, and in response to immunization, the introduced human heavy and light chain transgenes undergo class switching and somatic mutation to generate high affinity chimeric human/rat IgG monoclonal antibodies with fully human variable regions. The preparation and use of OmniRat®, and the genomic modifications carried by such rats, is described in International Publication No. WO14/093908.

Ablexis® mice generate antibodies having human variable domains linked to human CH1 and CL domains, chimeric human/mouse hinge regions, and mouse Fc regions. Ablexis Kappa Mouse and Lambda Mouse strains are distinguished by which of their heavy chains are human or mouse as noted below. Antibodies produced by the Kappa Mouse lack sequences derived from mouse VH, DH and JH exons and mouse Vκ, Jκ and Cκ exons. The endogenous mouse Igλ is active in the Kappa Mouse. The human Igκ chains comprise approximately 90-95% of the naïve repertoire and mouse Igλ chains comprise approximately 5-10% of the naïve repertoire in this strain. Antibodies produced by the Lambda Mouse lack sequences derived from mouse VH, DH and JH exons and mouse Vλ, Jλ and Cλ exons. The endogenous mouse Igκ is active in the Lambda Mouse. The human Igλ chains comprise approximately 40% of the naïve repertoire and mouse Igκ chains comprise approximately 60% of the naïve repertoire. The preparation and use of Ablexis®, and the genomic modifications carried by such mice, is described in International Publication No. WO11/123708.

Ablexis® mice and OmniRats® rats were immunized with soluble full length KLK2 protein (human kallikrein-2 6-His protein, with an amino acid sequence of

(SEQ ID NO: 167)
VPLIEGRIVGGWECEKHSQPWQVAVYSHGWAHCGGVLVHPQWVLTAAHCL
KKNSQVWLGRHNLFEPEDTGQRVPVSHSFPHPLYNMSLLKHQSLRPDEDS
SHDLMLLRLSEPAKITDVVKVLGLPTQEPALGTTCYASGWGSIEPEEFLR
PRSLQCVSLHYSEKVTEFMLCAGLWTGGKDTCGGDSGGPLVCNGVLQGIT
SWGPEPCALPEKPAVYTKVVHYRKWIKDTIAANPHHEIHHH.

Lymphocytes from Ablexis® mice and OmniRats® rats were extracted from lymph nodes and fusions performed by cohorts. Cells were combined and sorted for CD138 expression. Hybridoma screening was performed in high throughput miniaturized MSD format using soluble hKLK2 antigen. Approximately more than 300 samples were identified to be KLK2 binders. The binding of more than 300 anti-hKLK2 supernatant samples to human KLK2 protein was measured by a single cycle kinetics method using the Biacore™ 8K SPR System. Additionally the supernatant samples were tested for binding to human KLK3 protein, and to KLK2 expressing cell lines VCap and negative control cell line DU145 by flow cytometry.

Selected binders were subject to scFv conversion in both VH-VL and VL-VH orientation and tested for thermostability. Binary combinatorial scFv libraries were generated in the orientation VH-linker-VL in which one of the variable regions comprised sequences of the combinatorial library and the other variable region comprised the sequence of the parental KL2B30 VH or VL. A linker with a sequence of GGSEGKSSGSGSESKSTGGS (SEQ ID NO: 166) was used to conjugate the VH/VL regions. The engineered scFvs were cloned and expressed in E. coli. The scFvs resulting from the supernatants were tested for binding to human KLK2 by ELISA, which binding was compared to that of the KL2B30 antibody. Any new variants exhibiting binding affinities comparable to that of KL2B30 were consolidated and further tested for binding to human KLK2 after incubating the E. coli culture supernatants at 55° C., 60° C., and 65° C. for 10 minutes. The molecules retaining comparable binding to hu11B6 after incubation at 55° C., 60° C., and 65° C. and improved thermostability were matrixed in both orientations (VH-linker-VL; VL-linker-VH) and converted to mammalian scFvs for further characterization as described below.

KL2B30 was generated as a result of the Ablexis® mice immunization campaign, and expressed in a Fab format, a mAb format, a scFv format in the VH-linker-VL orientation or a scFv format in VL-linker-VH orientation and were further analyzed as described below. The linker sequence of SEQ ID NO: 166 was used to conjugate the VH/VL regions.

Sequences of KL2B30 antibodies and their variants are summarized in the Table 17 below.

TABLE 17
Amino acid and nucleotide sequences of
KL2B30 antibodies and variants

| Antibody | Region/ | Complete Sequence |
|---|---|---|
| Name | Description | (SEQ ID NO) |
| KL2B30 | VH/Amino | QVQLQESGPGLVKPSETLSLTCTVSGGSISSYYWS |
| | Acid | WIRQPPGKGLEWIGYIYYSGSTNYNPSLKSRVTISV |
| | Sequence | DTSKNQFSLKLSSVTAADTAVYYCAGTTIFGVVTP |
| | | NFYYGMDVWGQGTTVTVSS (SEQ ID NO: 107) |
| KL2B30 | VL/Amino | DIQMTQSPSFLSASVGDRVTITCRASQGISSYLAWY |
| | Acid | QQKPGKAPKFLIYAASTLQSGVPSRFSGSGSGTEFT |
| | Sequence | LTISSLQPEDFATYYCQQLNSYPLTFGGGTKVEIK |
| | | (SEQ ID NO: 108) |
| KL2B30 | Heavy Chain/ | QVQLQESGPGLVKPSETLSLTCTVSGGSISSYYWS |
| | Amino Acid | WIRQPPGKGLEWIGYIYYSGSTNYNPSLKSRVTISV |
| | Sequence | DTSKNQFSLKLSSVTAADTAVYYCAGTTIFGVVTP |
| | | NFYYGMDVWGQGTTVTVSSASTKGPSVFPLAPCS |
| | | RSTSESTAALGCLVKDYFPEPVTVSWNSGALTSGV |
| | | HTFPAVLQSSGLYSLSSVVTVPSSSLGTKTYTCNVD |
| | | HKPSNTKVDKRVESKYGPPCPPCPAPEAAGGPSVF |
| | | LFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPSQEEMTKNQVSLTCLVKGFYPSDIAVE |
| | | WESNGQPENNYKTTPPVLDSDGSFFLYSRLTVDKS |
| | | RWQEGNVFSCSVMHEALHNHYTQKSLSLSLGK |
| | | (SEQ ID NO: 145) |
| KL2B30 | Light Chain/ | DIQMTQSPSFLSASVGDRVTITCRASQGISSYLAWY |
| | Amino Acid | QQKPGKAPKFLIYAASTLQSGVPSRFSGSGSGTEFT |
| | Sequence | LTISSLQPEDFATYYCQQLNSYPLTFGGGTKVEIKR |
| | | TVAAPSVFIFPPSDEQLKSGTASVVCLLNNFYPREA |
| | | KVQWKVDNALQSGNSQESVTEQDSKDSTYSLSST |
| | | LTLSKADYEKHKVYACEVTHQGLSSPVTKSFNRGE |
| | | C (SEQ ID NO: 146) |
| scFv41 | KL2B30_HL/ | QVQLQESGPGLVKPSETLSLTCTVSGGSISSYYWS |
| | Amino Acid | WIRQPPGKGLEWIGYIYYSGSTNYNPSLKSRVTISV |
| | Sequence | DTSKNQFSLKLSSVTAADTAVYYCAGTTIFGVVTP |
| | | NFYYGMDVWGQGTTVTVSSGGSEGKSSGSGSESK |
| | | STGGSDIQMTQSPSFLSASVGDRVTITCRASQGISSY |
| | | LAWYQQKPGKAPKFLIYAASTLQSGVPSRFSGSGS |
| | | GTEFTLTISSLQPEDFATYYCQQLNSYPLTFGGGTK |
| | | VEIK (SEQ ID NO: 147) |
| scFv42 | KL2B30_LH/ | DIQMTQSPSFLSASVGDRVTITCRASQGISSYLAWY |
| | Amino Acid | QQKPGKAPKFLIYAASTLQSGVPSRFSGSGSGTEFT |
| | Sequence | LTISSLQPEDFATYYCQQLNSYPLTFGGGTKVEIKG |
| | | GSEGKSSGSGSESKSTGGSQVQLQESGPGLVKPSET |
| | | LSLTCTVSGGSISSYYWSWIRQPPGKGLEWIGYIYY |
| | | SGSTNYNPSLKSRVTISVDTSKNQFSLKLSSVTAAD |
| | | TAVYYCAGTTIFGVVTPNFYYGMDVWGQGTTVTV |
| | | SS (SEQ ID NO: 148) |
| KLCB80 | Heavy Chain/ | QVQLQESGPGLVKPSETLSLTCTVSGGSISSYYWS |
| (KL2B30 | Amino Acid | WIRQPPGKGLEWIGYIYYSGSTNYNPSLKSRVTISV |
| Fab) | Sequence | DTSKNQFSLKLSSVTAADTAVYYCAGTTIFGVVTP |
| | | NFYYGMDVWGQGTTVTVSSASTKGPSVFPLAPSS |
| | | KSTSGGTAALGCLVKDYFPEPVTVSWNSGALTSGV |
| | | HTFPAVLQSSGLYSLSSVVTVPSSSLGTQTYICNVN |
| | | HKPSNTKVDKKVEPKSCDKTHTCPPCPAPEAAGGP |
| | | SVFLFPPKPKDTLMISRTPEVTCVVVSVSHEDPEVK |
| | | FNWYVDGVEVHNAKTKPREEQYNSTYRVVSVLTV |
| | | LHQDWLNGKEYKCKVSNKALPAPIEKTISKAKGQP |
| | | REPQVYVLPPSREEMTKNQVSLLCLVKGFYPSDIA |
| | | VEWESNGQPENNYLTWPPVLDSDGSFFLYSKLTVD |
| | | KSRWQQGNVFSCSVMHEALHNHYTQKSLSLSPG |
| | | (SEQ ID NO: 149) |
| KL2B30 | Heavy Chain/ | QVQLQESGPGLVKPSETLSLTCTVSGGSISSYYWS |
| with | Amino Acid | WIRQPPGKGLEWIGYIYYSGSTNYNPSLKSRVTISV |
| K477 Fab | Sequence | DTSKNQFSLKLSSVTAADTAVYYCAGTTIFGVVTP |
| | | NFYYGMDVWGQGTTVTVSSASTKGPSVFPLAPSS |
| | | KSTSGGTAALGCLVKDYFPEPVTVSWNSGALTSGV |
| | | HTFPAVLQSSGLYSLSSVVTVPSSSLGTQTYICNVN |
| | | HKPSNTKVDKKVEPKSCDKTHTCPPCPAPEAAGGP |
| | | SVFLFPPKPKDTLMISRTPEVTCVVVSVSHEDPEVK |
| | | FNWYVDGVEVHNAKTKPREEQYNSTYRVVSVLTV |
| | | LHQDWLNGKEYKCKVSNKALPAPIEKTISKAKGQP |
| | | REPQVYVLPPSREEMTKNQVSLLCLVKGFYPSDIA |
| | | VEWESNGQPENNYLTWPPVLDSDGSFFLYSKLTVD |
| | | KSRWQQGNVFSCSVMHEALHNHYTQKSLSLSPGK |
| | | (SEQ ID NO: 150) |
| KL2B30_HL | Amino Acid | MAWVWTLLFLMAAAQSIQAQVQLQESGPGLVKPS |
| | Sequence | ETLSLTCTVSGGSISSYYWSWIRQPPGKGLEWIGYI |
| | | YYSGSTNYNPSLKSRVTISVDTSKNQFSLKLSSVTA |
| | | ADTAVYYCAGTTIFGVVTPNFYYGMDVWGQGTT |
| | | VTVSSGGSEGKSSGSGSESKSTGGSDIQMTQSPSFL |
| | | SASVGDRVTITCRASQGISSYLAWYQQKPGKAPKF |
| | | LIYAASTLQSGVPSRFSGSGSGTEFTLTISSLQPEDF |
| | | ATYYCQQLNSYPLTFGGGTKVEIK (SEQ ID NO: |
| | | 151) |
| KL2B30_LH | Amino Acid | MAWVWTLLFLMAAAQSIQADIQMTQSPSFLSASV |
| | Sequence | GDRVTITCRASQGISSYLAWYQQKPGKAPKFLIYA |
| | | ASTLQSGVPSRFSGSGSGTEFTLTISSLQPEDFATYY |
| | | CQQLNSYPLTFGGGTKVEIKGGSEGKSSGSGSESKS |
| | | TGGSQVQLQESGPGLVKPSETLSLTCTVSGGSISSY |
| | | YWSWIRQPPGKGLEWIGYIYYSGSTNYNPSLKSRV |
| | | TISVDTSKNQFSLKLSSVTAADTAVYYCAGTTIFGV |
| | | VTPNFYYGMDVWGQGTTVTVSS (SEQ ID NO: 152) |
| | linker | GGSEGKSSGSGSESKSTGGS (SEQ ID No: 166) |
| KL2B30 | Heavy Chain/ | CAGGTGCAGCTGCAGGAGTCGGGCCCAGGACTG |
| | Nucleotide | GTGAAGCCTTCGGAGACCCTGTCCCTCACCTGCA |
| | Sequence | CTGTCTCTGGTGGCTCCATCAGTAGTTACTACTG |
| | | GAGCTGGATCCGGCAGCCCCCAGGGAAGGGACT |
| | | GGAGTGGATTGGATATATCTATTACAGTGGGAG |
| | | CACCAACTACAACCCCTCCCTCAAGAGTCGAGTC |
| | | ACCATATCAGTAGACACGTCCAAGAACCAGTTCT |
| | | CCCTGAAGCTGAGCTCTGTGACCGCTGCGGACAC |
| | | GGCCGTGTATTACTGTGCGGGGACTACGATTTTT |
| | | GGAGTGGTTACCCCCAACTTCTACTACGGTATGG |
| | | ACGTCTGGGGCCAAGGGACCACGGTCACCGTCT |
| | | CCTCAGCTTCCACCAAGGGCCCATCCGTCTTCCC |
| | | CCTGGCGCCCTGCTCCAGGAGCACCTCCGAGAG |
| | | CACAGCCGCCCTGGGCTGCCTGGTCAAGGACTA |
| | | CTTCCCCGAACCGGTGACGGTGTCGTGGAACTCA |
| | | GGCGCCCTGACCAGCGGCGTGCACACCTTCCCG |
| | | GCTGTCCTACAGTCCTCAGGACTCTACTCCCTCA |
| | | GCAGCGTGGTGACCGTGCCCTCCAGCAGCTTGG |
| | | GCACGAAAACCTACACTTGCAACGTAGATCACA |
| | | AGCCCAGCAACACCAAGGTGGACAAGAGAGTTG |
| | | AGTCCAAATATGGTCCCCCATGCCCACCATGCCC |
| | | AGCACCTGAGGCCGCCGGGGGACCATCAGTCTT |
| | | CCTGTTCCCCCCAAAACCCAAGGACACTCTCATG |
| | | ATCTCCCGGACCCCTGAGGTCACGTGCGTGGTGG |
| | | TGGACGTGAGCCAGGAAGACCCCGAGGTCCAGT |
| | | TCAACTGGTACGTGGATGGCGTGGAGGTGCATA |
| | | ATGCCAAGACAAAGCCGCGGGAGGAGCAGTTCA |
| | | ACAGCACGTACCGTGTGGTCAGCGTCCTCACCGT |
| | | CCTGCACCAGGACTGGCTGAACGGCAAGGAGTA |
| | | CAAGTGCAAGGTCTCCAACAAAGGCCTCCCGTC |
| | | CTCCATCGAGAAAACCATCTCCAAAGCCAAAGG |
| | | GCAGCCCCGAGAGCCACAGGTGTACACCCTGCC |
| | | CCCATCCCAGGAGGAGATGACCAAGAACCAGGT |
| | | CAGCCTGACCTGCCTGGTCAAAGGCTTCTACCCC |
| | | AGCGACATCGCCGTGGAGTGGGAGAGCAATGGG |
| | | CAGCCGGAGAACAACTACAAGACCACGCCTCCC |
| | | GTGCTGGACTCCGACGGCTCCTTCTTCCTCTACA |
| | | GCAGGCTAACCGTGGACAAGAGCAGATGGCAGG |
| | | AGGGGAATGTCTTCTCATGCTCCGTGATGCATGA |
| | | GGCTCTGCACAACCACTACACACAGAAGAGCCT |
| | | CTCCCTGTCTCTGGGTAAA (SEQ ID NO: 158) |
| KL2B30 | Light Chain/ | GACATCCAGATGACCCAGTCTCCTTCCTTCCTGT |
| | Nucleotide | CTGCATCTGTAGGAGACAGAGTCACCATCACTTG |
| | Sequence | CCGGGCCAGTCAGGGCATTAGCAGTTATTTAGCC |
| | | TGGTATCAGCAAAAACCAGGGAAAGCCCCTAAG |
| | | TTCCTGATCTATGCTGCATCCACTTTGCAAAGTG |
| | | GGGTCCCATCAAGGTTCAGCGGCAGTGGATCTG |
| | | GGACAGAATTCACTCTCACAATCAGCAGCCTGC |
| | | AGCCTGAAGATTTTGCAACTTATTACTGTCAACA |
| | | GCTTAATAGTTACCCTCTCACTTTCGGCGGAGGG |
| | | ACCAAGGTGGAAATCAAACGTACGGTGGCTGCA |
| | | CCATCTGTCTTCATCTTCCCGCCATCTGATGAGC |
| | | AGTTGAAATCTGGAACTGCCTCTGTTGTGTGCCT |
| | | GCTGAATAACTTCTATCCCAGAGAGGCCAAAGT |
| | | ACAGTGGAAGGTGGATAACGCCCTCCAATCGGG |
| | | TAACTCCCAGGAGAGTGTCACAGAGCAGGACAG |
| | | CAAGGACAGCACCTACAGCCTCAGCAGCACCCT |
| | | GACGCTGAGCAAAGCAGACTACGAGAAACACAA |
| | | AGTCTACGCCTGCGAAGTCACCCATCAGGGCCTG |
| | | AGCTCGCCCGTCACAAAGAGCTTCAACAGGGGA |
| | | GAGTGT (SEQ ID NO: 159) |

### Example 5—Biophysical Characterization of Anti-KLK2 Antibodies

### Affinity and Thermal Stability of Anti-KLK2 Antibodies

Affinity of selected hKLK2 antibodies for soluble hKLK2 was measured by surface plasmon resonance (SPR). SPR is a label-free technique to study the strength of an interaction between two binding partners by measuring the change in mass upon complex formation and dissociation. Antibodies were captured on a sensor chip coated with an anti-Fc antibody followed by injection of soluble hK2 at various concentrations and specified association and dissociation times. Post dissociation, the surface was regenerated with an appropriate solution to prepare for the next interaction. Kinetic information (on-rate and off-rate constants) were extracted by fitting sensorgrams to the 1:1 Langmuir model. Binding affinity (K_{D}) are reported as the ratio of rate constants (k_{off}/kₒₙ). The K_{D} value of the KL2B30 Fab (KLCB80) antibody was measured to be 0.460 nM.

Thermostability was determined by Differential Scanning Fluorimetry (NanoDSF) using an automated Prometheus instrument. NanoDSF was used to measure the Tm of molecules at a concentration of 0.5 mg/mL in Phosphate Buffered Saline, pH 7.4. Measurements were made by loading samples into 24 well capillary from a 384 well sample plate. Duplicate runs were performed for each sample. The thermal scans range from 20° C. to 95° C. at a rate of 1.0° C./minute. Intrinsic tryptophan and tyrosine fluorescence were monitored at the emission wavelengths of 330 nm and 350 nm, and the F350/F330 nm ratios were plotted against temperatures to generate unfolding curves. The measured Tm for the KL2B30 Fab (KLCB80) antibody was >70° C.

### Epitope Mapping

The epitope on the KL2B30 antibody was determined by hydrogen-deuterium exchange mass spectrometry (HDX-MS). Human KLK2 antigen was used for epitope mapping experiments.

Briefly, purified KLK2 antigen was incubated with and without anti-KLK2 antibodies in deuterium oxide labeling buffer. The hydrogen-deuterium exchange (HDX) mixture was quenched at different time point by the addition of 8 M urea, 1M TCEP, pH 3.0. The quenched sample was passed over an immobilized pepsin/FPXIII column at 600 μL/min equilibrated with buffer A (1% acetonitrile, 0.1% FA in H₂O) at room temperature. Peptic fragments were loaded onto a reverse phase trap column at 600 μL/min with buffer A and desalted for 1 min (600 μL buffer A). The desalted fragments were separated by a C18 column with a linear gradient of 8% to 35% buffer B (95% acetonitrile, 5% H₂O, 0.0025% TFA) at 100 μL/min over 20 min and analyzed by mass spectrometry. Mass spectrometric analyses were carried out using an LTQ™ Orbitrap Fusion Lumos mass spectrometer (Thermo Fisher Scientific) with the capillary temperature at 275° C., resolution 150,000, and mass range (m/z) 300-1,800. BioPharma Finder 3.0 (Thermo Fisher Scientific) was used for the peptide identification of non-deuterated samples prior to the HDX experiments. HDExaminer version 2.5 (Sierra Analytics, Modesto, CA) was used to extract centroid values from the MS raw data files for the HDX experiments.

Incubation of the hKLK2 antibody KL2B30 with soluble hKLK2 protein resulted in different patterns of hydrogen exchange and overall protection. The protected segments were mapped onto the sequence of hKLK2 antigen to visualize the binding epitopes. KL2B30 bound to common sequences of (i) residues 174-178 with a sequence of KVTEF (SEQ ID NO: 164) and (ii) residues 230-234 with a sequence of HYRKW (SEQ ID NO: 165).

### Effector Function Analysis of Anti-KLK2 Antibodies

Anti-KLK2 antibodies and their variants with modified constant regions (i.e., with L234A/L235A/D265S mutations, low fucosylated and/or with K248E and T437R (RE) mutations) were generated and tested for their ability to mediate tumor cell killing via ADCC in the Vertebral-Cancer of the Prostate (VcaP) cell line, a cell line established from prostate cancer tissue, and these antibodies are summarized in the Table 18 below.

TABLE 18
Anti-KLK2 antibodies and their variants

| Antibody | Description | EC50 (M) | 95% CI (M) |
|---|---|---|---|
| KL2B870 | IgG1, K248E, | 1.001 × 10⁻⁹ | 5.149 × 10⁻¹⁰ to |
| (KL2B30 in | T437R (RE) | | 2.221 × 10⁻⁹ |
| hIgG1-RE) | | | |
| KL2B871 | L234A, L235A, | | |
| (KL2B30 in | D265S | | |
| hIgG1-AAS) | | | |
| KL2B872 | Wildtype IgG1 | | |
| (KL2B30 in | | | |
| hIgG1) | | | |
| KL2B872.CLF | IgG1, low | 4.118 × 10⁻¹⁰ | 1.747 × 10⁻¹⁰ to |
| | fucosylation | | 8.869 × 10⁻¹⁰ |
| KL2B870.CLF | IgG1, K248E, | 3.542 × 10⁻¹⁰ | 1.789 × 10¹⁰ to |
| | T437R (RE), | | 6.006 × 10⁻¹⁰ |
| | low fucosylation | | |

The parental antibody KL2B30 was modified in its Fc region to introduce the L234A, L235A and D265S mutations (AAS mutations), which resulted in an Fc region that does not bind Fc receptors. The resulting KL2B871 was generated as a negative control. The KL2B30 antibody was modified in its Fc region to introduce the K248E and T437R mutations (RE mutations), and the resulting antibody is KL2B870. KL2B870 and KL2B872 antibodies were expressed in fucosylation-deficient cells to produce antibodies with low fucosylation (e.g., expressing these antibodies in fucosylation-deficient Chinese Hamster Ovary cells produces antibodies with less than 10% fucosylation), which were designated as KL2B870.CLF and KL2B872.CLF, respectively (see Table 18).

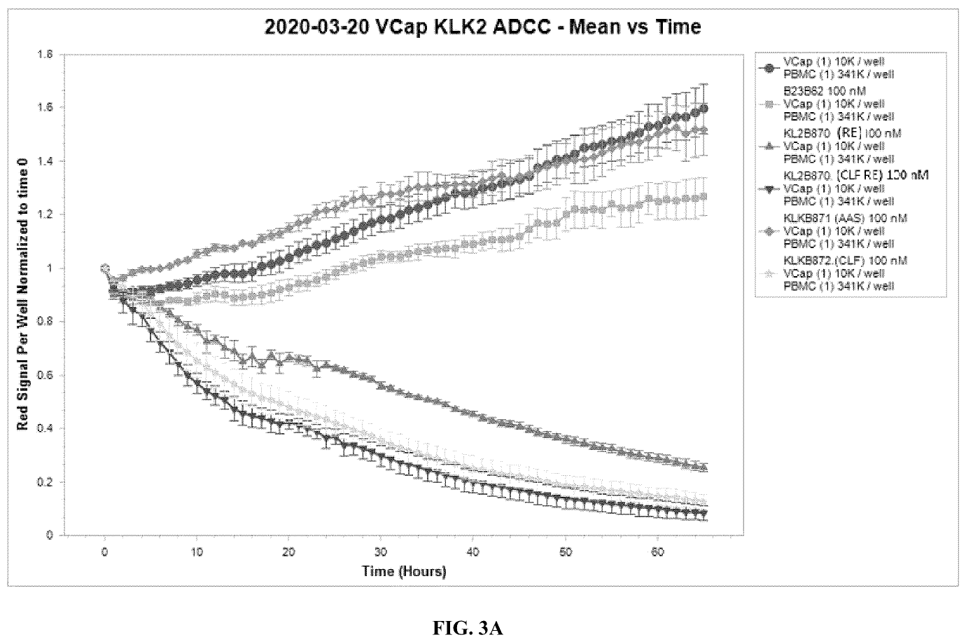

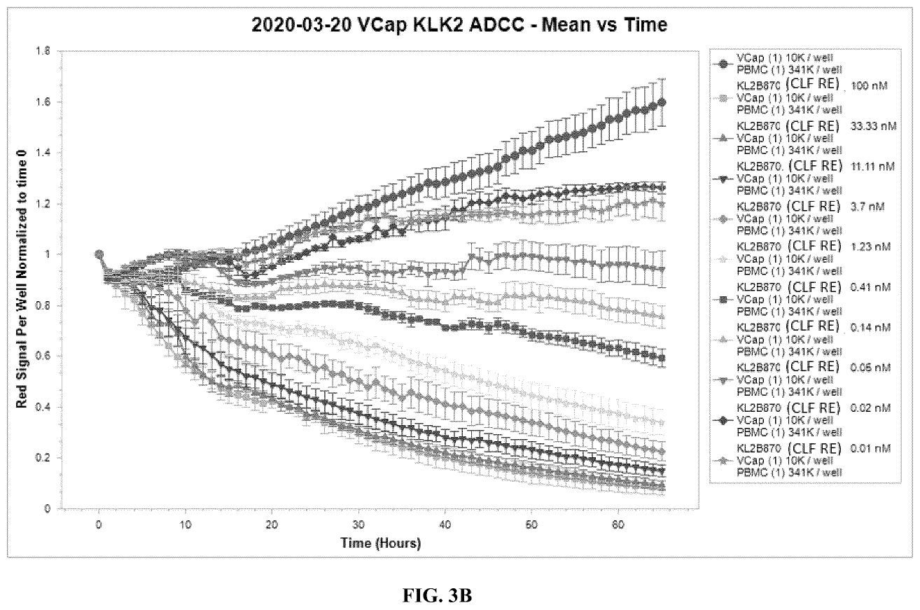

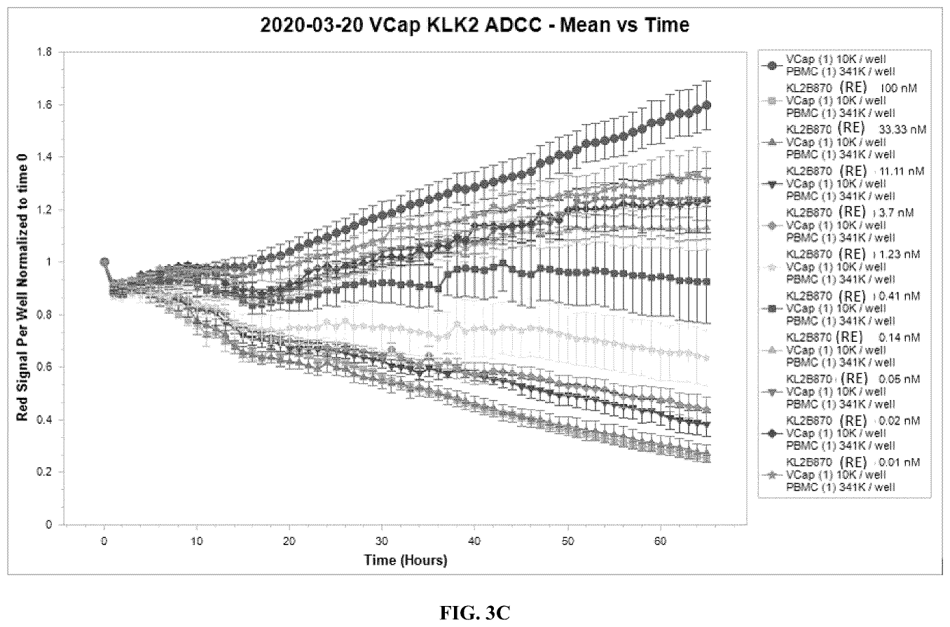

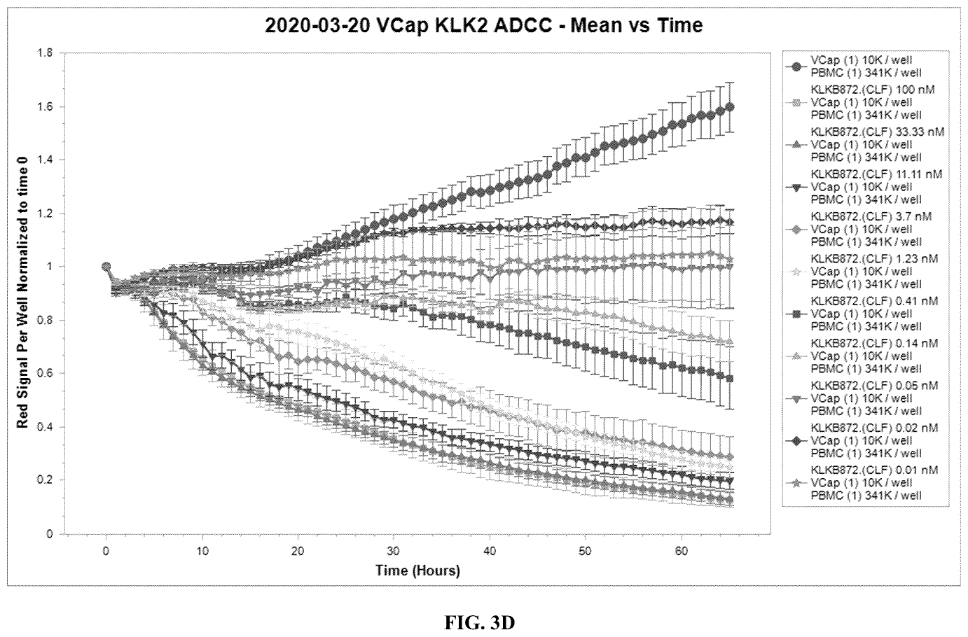

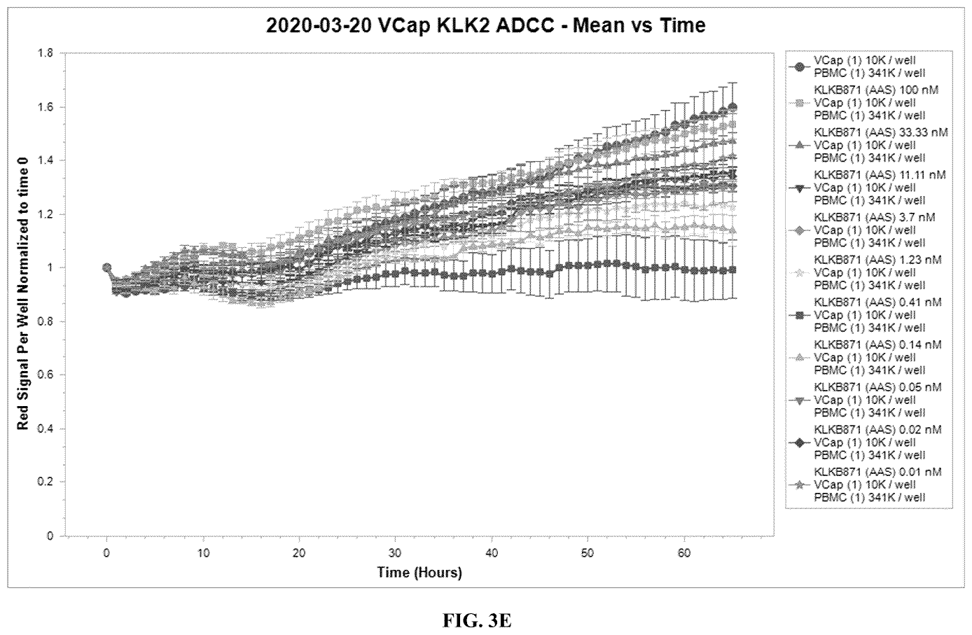

VcaP cells stably transfected with Nuclight Red (Incucyte®, Essen Bioscience) were plated at 10,000 cells per well in a 384-well plate (Perkin Elmer ViewPlate) in clear media (RPMI 1641+10% FBS, Thermo Fisher Scientific) to allow for cell adherence overnight. ADCC assay was performed with freshly thawed PBMC (Hemcare, PB009C-3). The ratio of effector to target cell per well was 34:1 for PBMCs as effector cells. KLK2 antibodies were tested with final concentrations ranging from 100 nM to 0.01 nM. After effector cells and antibodies were added to target cells, real time imaging was performed under Incucyte® S3 instrument (Essen BioScience) (FIGS. 3A-3E). Total red intergraded signal per well was quantified with Incucyte® software. Data analysis were performed by Incucyte® software and Prism (GraphPad Software) based on values of quadruplicates. The percentage of cell killing was calculated as: (1−KLK2 mAb/no mAb control)×100%.

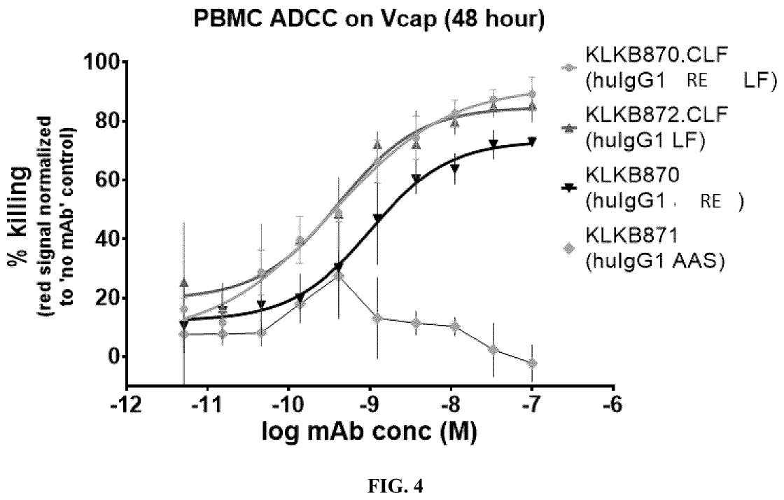

KLK2 antibodies demonstrated obvious dose dependent ADCC activities by PBMC on VcaP cells. The kinetic showed that ADCC activities initiated immediately after addition of effector cells and antibodies and continued with time (FIG. 4). KLKB870.CLF and KLKB872.CLF antibodies showed the highest ADCC activity with approximately 89% and 85% cell killing, followed by approximately 73% by KLKB870. KLKB87, the IgG1 antibody with AAS mutations, did not demonstrate any ADCC activity (FIG. 4). Dose-response curve generated at a certain time point (48 hours after effector cells and antibodies were added to target cells) showed the killing EC50 is approximately 354 pM for KLKB870.CLF, the antibody with low fucosylation. The EC50 for normal fucosylation antibody KLKB870 was approximately 1 nM (Table 18).

The VH and VL amino acid sequences of KL2B870, KL2B871 and KL2B872 antibodies are summarized in Table 19 below.

TABLE 19
VH and VL amino acid sequences of Anti-KLK2 antibodies

| Antibody | | Complete Sequence |
|---|---|---|
| Name | VH/VL | (SEQ ID NO) |
| KL2B870 | VH | QVQLQESGPGLVKPSETLSLTCTVSGGSISSYYWSWIR |
| | | QPPGKGLEWIGYIYYSGSTNYNPSLKSRVTISVDTSKN |
| | | QFSLKLSSVTAADTAVYYCAGTTIFGVVTPNFYYGMD |
| | | VWGQGTTVTVSS (SEQ ID NO: 107) |
| KL2B870 | VL | DIQMTQSPSFLSASVGDRVTITCRASQGISSYLAWYQQ |
| | | KPGKAPKFLIYAASTLQSGVPSRFSGSGSGTEFTLTISS |
| | | LQPEDFATYYCQQLNSYPLTFGGGTKVEIK (SEQ ID |
| | | NO: 108) |
| KL2B871 | VH | QVQLQESGPGLVKPSETLSLTCTVSGGSISSYYWSWIR |
| | | QPPGKGLEWIGYIYYSGSTNYNPSLKSRVTISVDTSKN |
| | | QFSLKLSSVTAADTAVYYCAGTTIFGVVTPNFYYGMD |
| | | VWGQGTTVTVSS (SEQ ID NO: 107) |
| KL2B871 | VL | DIQMTQSPSFLSASVGDRVTITCRASQGISSYLAWYQQ |
| | | KPGKAPKFLIYAASTLQSGVPSRFSGSGSGTEFTLTISS |
| | | LQPEDFATYYCQQLNSYPLTFGGGTKVEIK (SEQ ID |
| | | NO: 108) |
| KL2B872 | VH | QVQLQESGPGLVKPSETLSLTCTVSGGSISSYYWSWIR |
| | | QPPGKGLEWIGYIYYSGSTNYNPSLKSRVTISVDTSKN |
| | | QFSLKLSSVTAADTAVYYCAGTTIFGVVTPNFYYGMD |
| | | VWGQGTTVTVSS (SEQ ID NO: 107) |
| KL2B872 | VL | DIQMTQSPSFLSASVGDRVTITCRASQGISSYLAWYQQ |
| | | KPGKAPKFLIYAASTLQSGVPSRFSGSGSGTEFTLTISS |
| | | LQPEDFATYYCQQLNSYPLTFGGGTKVEIK (SEQ ID |
| | | NO: 108) |

The VH and VL nucleotide sequences of KL2B870, KL2B871 and KL2B872 antibodies are summarized in Table 20 below.

TABLE 20
VH and VL nucleotide sequences of
Anti-KLK2 antibodies

| Antibody | VH/ | Complete Sequence |
|---|---|---|
| Name | VL | (SEQ ID NO) |
| KL2B870 | VH | CAGGTGCAGCTGCAGGAGTCGGGCCCAGGACTGGT |
| | | GAAGCCTTCGGAGACCCTGTCCCTCACCTGCACTGT |
| | | CTCTGGTGGCTCCATCAGTAGTTACTACTGGAGCTG |
| | | GATCCGGCAGCCCCCAGGGAAGGGACTGGAGTGGA |
| | | TTGGATATATCTATTACAGTGGGAGCACCAACTACA |
| | | ACCCCTCCCTCAAGAGTCGAGTCACCATATCAGTAG |
| | | ACACGTCCAAGAACCAGTTCTCCCTGAAGCTGAGCT |
| | | CTGTGACCGCTGCGGACACGGCCGTGTATTACTGTG |
| | | CGGGGACTACGATTTTTGGAGTGGTTACCCCCAACT |
| | | TCTACTACGGTATGGACGTCTGGGGCCAAGGGACC |
| | | ACGGTCACCGTCTCCTCA (SEQ ID NO: 156) |
| KL2B870 | VL | GACATCCAGATGACCCAGTCTCCTTCCTTCCTGTCT |
| | | GCATCTGTAGGAGACAGAGTCACCATCACTTGCCG |
| | | GGCCAGTCAGGGCATTAGCAGTTATTTAGCCTGGTA |
| | | TCAGCAAAAACCAGGGAAAGCCCCTAAGTTCCTGA |
| | | TCTATGCTGCATCCACTTTGCAAAGTGGGGTCCCAT |
| | | CAAGGTTCAGCGGCAGTGGATCTGGGACAGAATTC |
| | | ACTCTCACAATCAGCAGCCTGCAGCCTGAAGATTTT |
| | | GCAACTTATTACTGTCAACAGCTTAATAGTTACCCT |
| | | CTCACTTTCGGCGGAGGGACCAAGGTGGAAATCAA |
| | | A (SEQ ID NO: 157) |
| KL2B871 | VH | CAGGTGCAGCTGCAGGAGTCGGGCCCAGGACTGGT |
| | | GAAGCCTTCGGAGACCCTGTCCCTCACCTGCACTGT |
| | | CTCTGGTGGCTCCATCAGTAGTTACTACTGGAGCTG |
| | | GATCCGGCAGCCCCCAGGGAAGGGACTGGAGTGGA |
| | | TTGGATATATCTATTACAGTGGGAGCACCAACTACA |
| | | ACCCCTCCCTCAAGAGTCGAGTCACCATATCAGTAG |
| | | ACACGTCCAAGAACCAGTTCTCCCTGAAGCTGAGCT |
| | | CTGTGACCGCTGCGGACACGGCCGTGTATTACTGTG |
| | | CGGGGACTACGATTTTTGGAGTGGTTACCCCCAACT |
| | | TCTACTACGGTATGGACGTCTGGGGCCAAGGGACC |
| | | ACGGTCACCGTCTCCTCA (SEQ ID NO: 156) |
| KL2B871 | VL | GACATCCAGATGACCCAGTCTCCTTCCTTCCTGTCT |
| | | GCATCTGTAGGAGACAGAGTCACCATCACTTGCCG |
| | | GGCCAGTCAGGGCATTAGCAGTTATTTAGCCTGGTA |
| | | TCAGCAAAAACCAGGGAAAGCCCCTAAGTTCCTGA |
| | | TCTATGCTGCATCCACTTTGCAAAGTGGGGTCCCAT |
| | | CAAGGTTCAGCGGCAGTGGATCTGGGACAGAATTC |
| | | ACTCTCACAATCAGCAGCCTGCAGCCTGAAGATTTT |
| | | GCAACTTATTACTGTCAACAGCTTAATAGTTACCCT |
| | | CTCACTTTCGGCGGAGGGACCAAGGTGGAAATCAA |
| | | A (SEQ ID NO: 157) |
| KL2B872 | VH | CAGGTGCAGCTGCAGGAGTCGGGCCCAGGACTGGT |
| | | GAAGCCTTCGGAGACCCTGTCCCTCACCTGCACTGT |
| | | CTCTGGTGGCTCCATCAGTAGTTACTACTGGAGCTG |
| | | GATCCGGCAGCCCCCAGGGAAGGGACTGGAGTGGA |
| | | TTGGATATATCTATTACAGTGGGAGCACCAACTACA |
| | | ACCCCTCCCTCAAGAGTCGAGTCACCATATCAGTAG |
| | | ACACGTCCAAGAACCAGTTCTCCCTGAAGCTGAGCT |
| | | CTGTGACCGCTGCGGACACGGCCGTGTATTACTGTG |
| | | CGGGGACTACGATTTTTGGAGTGGTTACCCCCAACT |
| | | TCTACTACGGTATGGACGTCTGGGGCCAAGGGACC |
| | | ACGGTCACCGTCTCCTCA (SEQ ID NO: 156) |
| KL2B872 | VL | GACATCCAGATGACCCAGTCTCCTTCCTTCCTGTCT |
| | | GCATCTGTAGGAGACAGAGTCACCATCACTTGCCG |
| | | GGCCAGTCAGGGCATTAGCAGTTATTTAGCCTGGTA |
| | | TCAGCAAAAACCAGGGAAAGCCCCTAAGTTCCTGA |
| | | TCTATGCTGCATCCACTTTGCAAAGTGGGGTCCCAT |
| | | CAAGGTTCAGCGGCAGTGGATCTGGGACAGAATTC |
| | | ACTCTCACAATCAGCAGCCTGCAGCCTGAAGATTTT |
| | | GCAACTTATTACTGTCAACAGCTTAATAGTTACCCT |
| | | CTCACTTTCGGCGGAGGGACCAAGGTGGAAATCAA |
| | | A (SEQ ID NO: 157) |

The complete amino acid sequences of the light chains and heavy chains of KL2B870, KL2B871 and KL2B872 antibodies are summarized in Table 21 below.

TABLE 21
Heavy chain and light chain amino acid sequences of
Anti-KLK2 antibodies

| Antibody | | Complete Sequence |
|---|---|---|
| Name | Region | (SEQ ID NO) |
| KL2B870 | Heavy | QVQLQESGPGLVKPSETLSLTCTVSGGSISSYYWSWIRQPP |
| | chain | GKGLEWIGYIYYSGSTNYNPSLKSRVTISVDTSKNQFSLKL |
| | | SSVTAADTAVYYCAGTTIFGVVTPNFYYGMDVWGQGTTV |
| | | TVSSASTKGPSVFPLAPSSKSTSGGTAALGCLVKDYFPEPV |
| | | TVSWNSGALTSGVHTFPAVLQSSGLYSLSSVVTVPSSSLGT |
| | | QTYICNVNHKPSNTKVDKKVEPKSCDKTHTCPPCPAPELL |
| | | GGPSVFLFPPKPEDTLMISRTPEVTCVVVDVSHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EEMTKNQVSLTCLVKGFYPSDIAVEWESNGQPENNYKTTP |
| | | PVLDSDGSFFLYSKLTVDKSRWQQGNVFSCSVMHEALHN |
| | | HYRQKSLSLSPG (SEQ ID NO: 153) |
| KL2B870 | Light | DIQMTQSPSFLSASVGDRVTITCRASQGISSYLAWYQQKPG |
| | chain | KAPKFLIYAASTLQSGVPSRFSGSGSGTEFTLTISSLQPEDF |
| | | ATYYCQQLNSYPLTFGGGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC (SEQ ID NO: 146) |
| KL2B871 | Heavy | QVQLQESGPGLVKPSETLSLTCTVSGGSISSYYWSWIRQPP |
| | chain | GKGLEWIGYIYYSGSTNYNPSLKSRVTISVDTSKNQFSLKL |
| | | SSVTAADTAVYYCAGTTIFGVVTPNFYYGMDVWGQGTTV |
| | | TVSSASTKGPSVFPLAPSSKSTSGGTAALGCLVKDYFPEPV |
| | | TVSWNSGALTSGVHTFPAVLQSSGLYSLSSVVTVPSSSLGT |
| | | QTYICNVNHKPSNTKVDKKVEPKSCDKTHTCPPCPAPEAA |
| | | GGPSVFLFPPKPKDTLMISRTPEVTCVVVSVSHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EEMTKNQVSLTCLVKGFYPSDIAVEWESNGQPENNYKTTP |
| | | PVLDSDGSFFLYSKLTVDKSRWQQGNVFSCSVMHEALHN |
| | | HYTQKSLSLSPG (SEQ ID NO: 154) |
| KL2B871 | Light | DIQMTQSPSFLSASVGDRVTITCRASQGISSYLAWYQQKPG |
| | chain | KAPKFLIYAASTLQSGVPSRFSGSGSGTEFTLTISSLQPEDF |
| | | ATYYCQQLNSYPLTFGGGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC (SEQ ID NO: 146) |
| KL2B872 | Heavy | QVQLQESGPGLVKPSETLSLTCTVSGGSISSYYWSWIRQPP |
| | chain | GKGLEWIGYIYYSGSTNYNPSLKSRVTISVDTSKNQFSLKL |
| | | SSVTAADTAVYYCAGTTIFGVVTPNFYYGMDVWGQGTTV |
| | | TVSSASTKGPSVFPLAPSSKSTSGGTAALGCLVKDYFPEPV |
| | | TVSWNSGALTSGVHTFPAVLQSSGLYSLSSVVTVPSSSLGT |
| | | QTYICNVNHKPSNTKVDKKVEPKSCDKTHTCPPCPAPELL |
| | | GGPSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EEMTKNQVSLTCLVKGFYPSDIAVEWESNGQPENNYKTTP |
| | | PVLDSDGSFFLYSKLTVDKSRWQQGNVFSCSVMHEALHN |
| | | HYTQKSLSLSPG (SEQ ID NO: 155) |
| KL2B872 | Light | DIQMTQSPSFLSASVGDRVTITCRASQGISSYLAWYQQKPG |
| | chain | KAPKFLIYAASTLQSGVPSRFSGSGSGTEFTLTISSLQPEDF |
| | | ATYYCQQLNSYPLTFGGGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC (SEQ ID NO: 146) |

The complete nucleotide sequences of the light chains and heavy chains of KL2B870, KL2B871 and KL2B872 antibodies are summarized in Table 22 below.

TABLE 22
Heavy chain and light chain nucleotide sequences of
Anti-KLK2 antibodies

| Antibody | | Complete Sequence |
|---|---|---|
| Name | Region | (SEQ ID NO) |
| KL2B870 | Heavy | CAAGTACAGCTTCAGGAGTCAGGCCCCGGCTTGGTG |
| | chain | AAACCAAGTGAGACCCTCTCCCTCACCTGCACAGTT |
| | | AGCGGCGGTTCAATATCATCATACTATTGGTCTTGGA |
| | | TTCGTCAGCCACCTGGCAAAGGTCTCGAGTGGATTG |
| | | GCTATATATATTATTCAGGGTCAACAAATTACAATCC |
| | | TTCACTCAAGTCTCGCGTCACCATTAGCGTAGACACC |
| | | AGCAAAAATCAGTTTTCCCTCAAGCTCTCATCAGTGA |
| | | CCGCCGCAGATACCGCTGTATATTACTGCGCTGGTAC |
| | | CACAATATTTGGAGTGGTTACCCCTAATTTCTATTAT |
| | | GGAATGGACGTATGGGGCCAGGGGACCACCGTGACA |
| | | GTTTCCTCTGCCTCCACCAAGGGCCCATCGGTCTTCC |
| | | CCCTGGCACCCTCCTCCAAGAGCACCTCTGGGGGCA |
| | | CAGCGGCCCTGGGCTGCCTGGTCAAGGACTACTTCC |
| | | CCGAACCGGTGACGGTGTCGTGGAACTCAGGCGCCC |
| | | TGACCAGCGGCGTGCACACCTTCCCGGCTGTCCTACA |
| | | GTCCTCAGGACTCTACTCCCTCAGCAGCGTGGTGACC |
| | | GTGCCCTCCAGCAGCTTGGGCACCCAGACCTACATCT |
| | | GCAACGTGAATCACAAGCCCAGCAACACCAAGGTGG |
| | | ACAAGAAAGTTGAGCCCAAATCTTGTGACAAAACTC |
| | | ACACATGTCCACCGTGCCCAGCACCTGAACTGCTGG |
| | | GGGGACCGTCAGTCTTCCTCTTCCCCCCAAAACCCGA |
| | | GGACACCCTCATGATCTCCCGGACCCCTGAGGTCAC |
| | | ATGCGTGGTGGTGGACGTGAGCCACGAAGACCCTGA |
| | | GGTCAAGTTCAACTGGTACGTGGACGGCGTGGAGGT |
| | | GCATAATGCCAAGACAAAGCCGCGGGAGGAGCAGT |
| | | ACAACAGCACGTACCGTGTGGTCAGCGTCCTCACCG |
| | | TCCTGCACCAGGACTGGCTGAATGGCAAGGAGTACA |
| | | AGTGCAAGGTCTCCAACAAAGCCCTCCCAGCCCCCA |
| | | TCGAGAAAACCATCTCCAAAGCCAAAGGGCAGCCCC |
| | | GAGAACCACAGGTGTACACCCTGCCCCCATCCCGGG |
| | | AGGAGATGACCAAGAACCAGGTCAGCCTGACCTGCC |
| | | TGGTCAAAGGCTTCTATCCCAGCGACATCGCCGTGG |
| | | AGTGGGAGAGCAATGGGCAGCCGGAGAACAACTAC |
| | | AAGACCACGCCTCCCGTGCTGGACTCCGACGGCTCC |
| | | TTCTTCCTCTACAGCAAGCTCACCGTGGACAAGTCTA |
| | | GATGGCAGCAGGGGAACGTCTTCTCATGCTCCGTGA |
| | | TGCATGAGGCTCTGCACAACCACTACCGCCAGAAGA |
| | | GCCTCTCCCTGTCTCCGGGT (SEQ ID NO: 160) |
| KL2B870 | Light | GATATACAAATGACCCAATCTCCTTCCTTTTTGTCAG |
| | chain | CAAGCGTTGGTGACCGCGTCACCATAACTTGTAGGG |
| | | CTAGCCAGGGTATTTCTAGTTATCTGGCTTGGTATCA |
| | | GCAAAAACCTGGCAAAGCCCCTAAATTTCTTATCTAC |
| | | GCCGCAAGCACTCTGCAATCAGGCGTGCCCAGCAGA |
| | | TTCTCAGGCTCTGGTTCAGGCACAGAGTTTACTCTGA |
| | | CTATATCCAGCCTGCAACCTGAAGACTTTGCTACATA |
| | | CTATTGTCAGCAACTCAACAGTTACCCCCTTACTTTC |
| | | GGGGGCGGCACTAAGGTGGAAATAAAGCGTACGGT |
| | | GGCTGCACCATCTGTCTTCATCTTCCCGCCATCTGAT |
| | | GAGCAGTTGAAATCTGGAACTGCCTCTGTTGTGTGCC |
| | | TGCTGAATAACTTCTATCCCAGAGAGGCCAAAGTAC |
| | | AGTGGAAGGTGGATAACGCCCTCCAATCGGGTAACT |
| | | CCCAGGAGAGTGTCACAGAGCAGGACAGCAAGGAC |
| | | AGCACCTACAGCCTCAGCAGCACCCTGACGCTGAGC |
| | | AAAGCAGACTACGAGAAACACAAAGTCTACGCCTGC |
| | | GAAGTCACCCATCAGGGCCTGAGCTCGCCCGTCACA |
| | | AAGAGCTTCAACAGGGGAGAGTGT (SEQ ID NO: 161) |
| KL2B871 | Heavy | CAAGTACAGCTTCAGGAGTCAGGCCCCGGCTTGGTG |
| | chain | AAACCAAGTGAGACCCTCTCCCTCACCTGCACAGTT |
| | | AGCGGCGGTTCAATATCATCATACTATTGGTCTTGGA |
| | | TTCGTCAGCCACCTGGCAAAGGTCTCGAGTGGATTG |
| | | GCTATATATATTATTCAGGGTCAACAAATTACAATCC |
| | | TTCACTCAAGTCTCGCGTCACCATTAGCGTAGACACC |
| | | AGCAAAAATCAGTTTTCCCTCAAGCTCTCATCAGTGA |
| | | CCGCCGCAGATACCGCTGTATATTACTGCGCTGGTAC |
| | | CACAATATTTGGAGTGGTTACCCCTAATTTCTATTAT |
| | | GGAATGGACGTATGGGGCCAGGGGACCACCGTGACA |
| | | GTTTCCTCTGCCTCCACCAAGGGCCCATCGGTCTTCC |
| | | CCCTGGCACCCTCCTCCAAGAGCACCTCTGGGGGCA |
| | | CAGCGGCCCTGGGCTGCCTGGTCAAGGACTACTTCC |
| | | CCGAACCGGTGACGGTGTCGTGGAACTCAGGCGCCC |
| | | TGACCAGCGGCGTGCACACCTTCCCGGCTGTCCTACA |
| | | GTCCTCAGGACTCTACTCCCTCAGCAGCGTGGTGACC |
| | | GTGCCCTCCAGCAGCTTGGGCACCCAGACCTACATCT |
| | | GCAACGTGAATCACAAGCCCAGCAACACCAAGGTGG |
| | | ACAAGAAAGTTGAGCCCAAATCTTGTGACAAAACTC |
| | | ACACATGTCCACCGTGCCCAGCACCTGAAGCAGCAG |
| | | GGGGACCGTCAGTCTTCCTCTTCCCCCCAAAACCCAA |
| | | GGACACCCTCATGATCTCCCGGACCCCTGAGGTCAC |
| | | ATGCGTGGTGGTGAGCGTGAGCCACGAAGACCCTGA |
| | | GGTCAAGTTCAACTGGTACGTGGACGGCGTGGAGGT |
| | | GCATAATGCCAAGACAAAGCCGCGGGAGGAGCAGT |
| | | ACAACAGCACGTACCGTGTGGTCAGCGTCCTCACCG |
| | | TCCTGCACCAGGACTGGCTGAATGGCAAGGAGTACA |
| | | AGTGCAAGGTCTCCAACAAAGCCCTCCCAGCCCCCA |
| | | TCGAGAAAACCATCTCCAAAGCCAAAGGGCAGCCCC |
| | | GAGAACCACAGGTGTACACCCTGCCCCCATCCCGGG |
| | | AGGAGATGACCAAGAACCAGGTCAGCCTGACCTGCC |
| | | TGGTCAAAGGCTTCTATCCCAGCGACATCGCCGTGG |
| | | AGTGGGAGAGCAATGGGCAGCCGGAGAACAACTAC |
| | | AAGACCACGCCTCCCGTGCTGGACTCCGACGGCTCC |
| | | TTCTTCCTCTACAGCAAGCTCACCGTGGACAAGTCTA |
| | | GATGGCAGCAGGGGAACGTCTTCTCATGCTCCGTGA |
| | | TGCATGAGGCTCTGCACAACCACTACACGCAGAAGA |
| | | GCCTCTCCCTGTCTCCGGGT (SEQ ID NO: 162) |
| KL2B871 | Light | GATATACAAATGACCCAATCTCCTTCCTTTTTGTCAG |
| | chain | CAAGCGTTGGTGACCGCGTCACCATAACTTGTAGGG |
| | | CTAGCCAGGGTATTTCTAGTTATCTGGCTTGGTATCA |
| | | GCAAAAACCTGGCAAAGCCCCTAAATTTCTTATCTAC |
| | | GCCGCAAGCACTCTGCAATCAGGCGTGCCCAGCAGA |
| | | TTCTCAGGCTCTGGTTCAGGCACAGAGTTTACTCTGA |
| | | CTATATCCAGCCTGCAACCTGAAGACTTTGCTACATA |
| | | CTATTGTCAGCAACTCAACAGTTACCCCCTTACTTTC |
| | | GGGGGCGGCACTAAGGTGGAAATAAAGCGTACGGT |
| | | GGCTGCACCATCTGTCTTCATCTTCCCGCCATCTGAT |
| | | GAGCAGTTGAAATCTGGAACTGCCTCTGTTGTGTGCC |
| | | TGCTGAATAACTTCTATCCCAGAGAGGCCAAAGTAC |
| | | AGTGGAAGGTGGATAACGCCCTCCAATCGGGTAACT |
| | | CCCAGGAGAGTGTCACAGAGCAGGACAGCAAGGAC |
| | | AGCACCTACAGCCTCAGCAGCACCCTGACGCTGAGC |
| | | AAAGCAGACTACGAGAAACACAAAGTCTACGCCTGC |
| | | GAAGTCACCCATCAGGGCCTGAGCTCGCCCGTCACA |
| | | AAGAGCTTCAACAGGGGAGAGTGT (SEQ ID NO: 161) |
| KL2B872 | Heavy | CAAGTACAGCTTCAGGAGTCAGGCCCCGGCTTGGTG |
| | chain | AAACCAAGTGAGACCCTCTCCCTCACCTGCACAGTT |
| | | AGCGGCGGTTCAATATCATCATACTATTGGTCTTGGA |
| | | TTCGTCAGCCACCTGGCAAAGGTCTCGAGTGGATTG |
| | | GCTATATATATTATTCAGGGTCAACAAATTACAATCC |
| | | TTCACTCAAGTCTCGCGTCACCATTAGCGTAGACACC |
| | | AGCAAAAATCAGTTTTCCCTCAAGCTCTCATCAGTGA |
| | | CCGCCGCAGATACCGCTGTATATTACTGCGCTGGTAC |
| | | CACAATATTTGGAGTGGTTACCCCTAATTTCTATTAT |
| | | GGAATGGACGTATGGGGCCAGGGGACCACCGTGACA |
| | | GTTTCCTCTGCCTCCACCAAGGGCCCATCGGTCTTCC |
| | | CCCTGGCACCCTCCTCCAAGAGCACCTCTGGGGGCA |
| | | CAGCGGCCCTGGGCTGCCTGGTCAAGGACTACTTCC |
| | | CCGAACCGGTGACGGTGTCGTGGAACTCAGGCGCCC |
| | | TGACCAGCGGCGTGCACACCTTCCCGGCTGTCCTACA |
| | | GTCCTCAGGACTCTACTCCCTCAGCAGCGTGGTGACC |
| | | GTGCCCTCCAGCAGCTTGGGCACCCAGACCTACATCT |
| | | GCAACGTGAATCACAAGCCCAGCAACACCAAGGTGG |
| | | ACAAGAAAGTTGAGCCCAAATCTTGTGACAAAACTC |
| | | ACACATGTCCACCGTGCCCAGCACCTGAACTGCTGG |
| | | GGGGACCGTCAGTCTTCCTCTTCCCCCCAAAACCCAA |
| | | GGACACCCTCATGATCTCCCGGACCCCTGAGGTCAC |
| | | ATGCGTGGTGGTGGACGTGAGCCACGAAGACCCTGA |
| | | GGTCAAGTTCAACTGGTACGTGGACGGCGTGGAGGT |
| | | GCATAATGCCAAGACAAAGCCGCGGGAGGAGCAGT |
| | | ACAACAGCACGTACCGTGTGGTCAGCGTCCTCACCG |
| | | TCCTGCACCAGGACTGGCTGAATGGCAAGGAGTACA |
| | | AGTGCAAGGTCTCCAACAAAGCCCTCCCAGCCCCCA |
| | | TCGAGAAAACCATCTCCAAAGCCAAAGGGCAGCCCC |
| | | GAGAACCACAGGTGTACACCCTGCCCCCATCCCGGG |
| | | AGGAGATGACCAAGAACCAGGTCAGCCTGACCTGCC |
| | | TGGTCAAAGGCTTCTATCCCAGCGACATCGCCGTGG |
| | | AGTGGGAGAGCAATGGGCAGCCGGAGAACAACTAC |
| | | AAGACCACGCCTCCCGTGCTGGACTCCGACGGCTCC |
| | | TTCTTCCTCTACAGCAAGCTCACCGTGGACAAGTCTA |
| | | GATGGCAGCAGGGGAACGTCTTCTCATGCTCCGTGA |
| | | TGCATGAGGCTCTGCACAACCACTACACGCAGAAGA |
| | | GCCTCTCCCTGTCTCCGGGT (SEQ ID NO: 163) |
| KL2B872 | Light | GATATACAAATGACCCAATCTCCTTCCTTTTTGTCAG |
| | chain | CAAGCGTTGGTGACCGCGTCACCATAACTTGTAGGG |
| | | CTAGCCAGGGTATTTCTAGTTATCTGGCTTGGTATCA |
| | | GCAAAAACCTGGCAAAGCCCCTAAATTTCTTATCTAC |
| | | GCCGCAAGCACTCTGCAATCAGGCGTGCCCAGCAGA |
| | | TTCTCAGGCTCTGGTTCAGGCACAGAGTTTACTCTGA |
| | | CTATATCCAGCCTGCAACCTGAAGACTTTGCTACATA |
| | | CTATTGTCAGCAACTCAACAGTTACCCCCTTACTTTC |
| | | GGGGGCGGCACTAAGGTGGAAATAAAGCGTACGGT |
| | | GGCTGCACCATCTGTCTTCATCTTCCCGCCATCTGAT |
| | | GAGCAGTTGAAATCTGGAACTGCCTCTGTTGTGTGCC |
| | | TGCTGAATAACTTCTATCCCAGAGAGGCCAAAGTAC |
| | | AGTGGAAGGTGGATAACGCCCTCCAATCGGGTAACT |
| | | CCCAGGAGAGTGTCACAGAGCAGGACAGCAAGGAC |
| | | AGCACCTACAGCCTCAGCAGCACCCTGACGCTGAGC |
| | | AAAGCAGACTACGAGAAACACAAAGTCTACGCCTGC |
| | | GAAGTCACCCATCAGGGCCTGAGCTCGCCCGTCACA |
| | | AAGAGCTTCAACAGGGGAGAGTGT (SEQ ID NO: 161) |

### Example 6—ADCC and CDC Functional Analysis of Specific Anti-HLA-G Antibodies

To test the ability of anti-HLA-G antibodies to mediate tumor cell killing via ADCC in the choriocarcinoma cell line JEG-3 (ATCC HTB-36), which endogenously expresses HLA-G, antibodies were added to JEG-3 cells labeled with BATDA dye (Perkin Elmer cat. #C136-100), which can unidirectionally penetrate into the cells. Upon cell lysis, the dye is released into the solution containing Europium which reacts with the dye to form a fluorescent chelate, whose fluorescence signal can be measured. PBMCs cultured overnight were added at an E:T ratio of 50:1 to JEG-3 cells at 5,000 cells/well and the mixture was incubated for 4 hours at 37° C. The cell mixture was added at 1:10 into Europium solution, incubated for 15 min at room temperature and fluorescence at 610 nm was monitored to determine signal. The fluorescence signal for 100% killing was determined using a well containing BATDA-labeled target cells mixed with Triton-X 100 detergent.

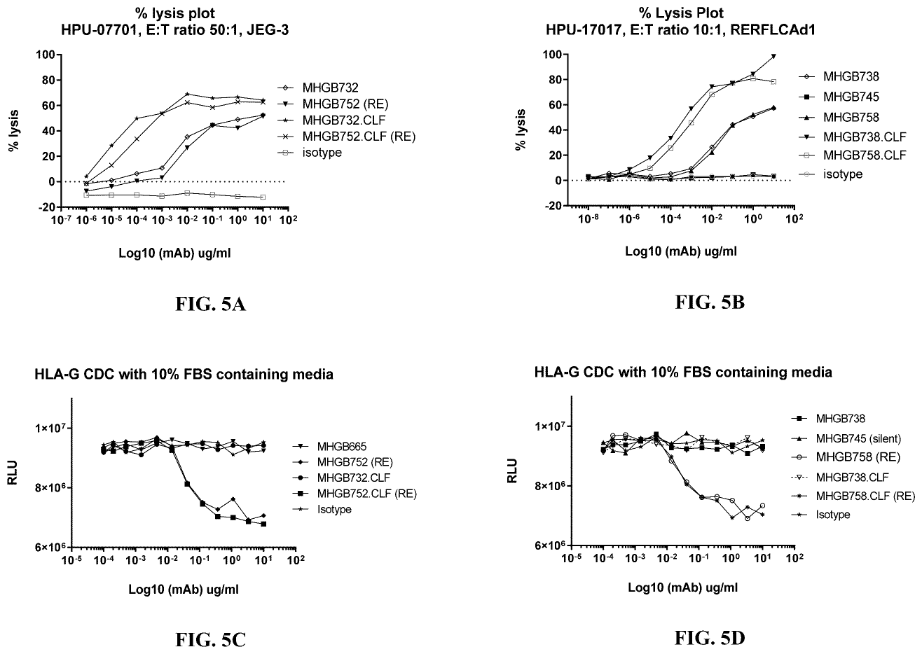

Since the anti-HLA-G Abs could display ADCC in vitro, whether this activity could be enhanced was explored. Several studies showed that antibodies having less than 10% terminal fucosylated Fc regions display enhanced effector function due to higher affinity binding to Fc receptors. Thus, anti-HLA-G antibodies MHGB732 and MHGB738 were generated in a low fucose Chinese Hamster Ovary (CHO) host to produce an antibody with less than 10% fucosylation (MHGB732.CLF and MHGB738.CLF) (Table 23, FIGS. 5A-5D). As a negative control, a version of MHGB738 with an Fc region that could not bind Fc receptors was generated, and this antibody was called MHGB745.

The normal fucose and low fucose antibodies were tested for their abilities to induce NK cell-based ADCC against either JEG-3 cells (FIG. 5A) or against RERF-LC-Ad-1 cells (human lung adenocarcinoma cell line, JCRB1020) (FIG. 5B). Antibodies with low fucosylation were generated by expression of the constructs encoding the heavy chain and light chain in CHO cells with low expression levels of the fucosyltransferase enzyme, leading to production of antibodies having less than 10% fucosylation.

The ADCC activity assay was performed as described above. The ratio of effector cells to target cells is shown in FIGS. 5A and 5B. Both the MHGB745 and the isotype control did not induce ADCC in the assay. The two IgG1 antibodies, MHGB732 and MHGB738 could induce ADCC while the same antibodies having Fc regions with low fucosylation displayed approximately 10-fold enhanced ADCC activity. This showed that ADCC enhancement could be obtained by use of an antibody with low fucosylation.

Next the abilities of these antibodies to mediate CDC were tested (FIGS. 5C and 5D). Briefly, assays were run in 10% FBS containing DMEM (JEG-3) or RPMI (RERF-LC-Ad-1). Antibodies were added to target cells and incubated for 30 minutes at 37° C. After incubation, 15-20% (stock concentration) of rabbit complement (Cedarlane cat. #CL3441-S) and heat inactivated complement was added to the wells respectively to a volume of 25 μl/well. The mixture was incubated for 4-12 hours at 37° C. Target cell lysis was detected by addition of 100 μl of CellTitre-Glo (Promega cat. #G9242) reagent followed by incubation for 10 minutes at room temperature. Luminescence was monitored using a Tecan Microplate reader SPARK®. The two IgG1 antibodies, MHGB732 and MHGB738 did not mediate CDC. Since the IgG1 antibodies could not mediate CDC, the variable regions thereof were cloned into an IgG1 Fc harboring the K248E and T437R mutations (RE mutations), which were shown to specifically enhance CDC activity. These antibodies, having the identical variable regions as their IgG1 counterparts, could mediate CDC activity. As a control, anti-HLA-G antibody MHGB665 was also tested in the CDC activity assay. Whether the RE Fc variants would impact the ADCC activity enhancement in antibodies with low fucosylation and whether Fc regions with low fucosylation would impact the CDC activity of the RE Fc variants were examined. Antibodies bearing the RE mutations produced in a low fucose host (with less than 10% fucosylation), MHGB752 and MHGB758 had identical ADCC activities to the IgG1 antibodies MHGB732 and MHGB738 with low fucosylation (FIGS. 5A and 5B). Analogously, antibodies bearing the RE mutations produced in a low fucose host had identical CDC activities to the same antibodies produced in a normal fucose host (FIGS. 5C and 5D).

Antibodies MHGB732 and MHGB738 and their variants with modified constant regions are summarized in the Table 23 below.

TABLE 23
Antibodies MHGB732 and MHGB738 and their variants

| Antibody Name | Description |
|---|---|
| MHGB732 | IgG1 |
| MHGB738 | IgG1 |
| MHGB745 | L234A, L235A, D265S |
| MHGB752 | IgG1, K248E, T437R (RE) |
| MHGB758 | IgG1, K248E, T437R (RE) |
| MHGB732.CLF | IgG1, low fucosylation |
| MHGB738.CLF | IgG1, low fucosylation |
| MHGB752.CLF | IgG1, K248E, T437R (RE), |
| | low fucosylation |
| MHGB758.CLF | IgG1, K248E, T437R (RE), |
| | low fucosylation |

The VL and VH amino acid sequences of exemplary anti-HLA-G antibodies are summarized in Table 24 below.

TABLE 24
VL and VH amino acid sequences of exemplary
anti-HLA-G antibodies

| Anti- | | |
|---|---|---|
| body | VL/ | Variable Sequence |
| Name | VH | (SEQ ID NO) |
| MHGB732 | VL | DIVMTQSPDSLAVSLGERATINCKSSQSVLHSSNNKNY |
| | | LTWFQQKPGQPPKLLIYWASTRESGVPDRFSGSGSGT |
| | | DFTLTISSLQAEDVAVYYCHQYYSTPPTFGQGTKVEIK |
| | | (SEQ ID NO: 172) |
| MHGB738 | VL | DIVMTQSPDSLAVSLGERATINCKSSQSVLFSSNNKNY |
| | | LAWYQQKPGQPPKLLIYWASTRESGVPDRFSGSVSGT |
| | | DFTLTISSLQAEDVAVYYCQQYHSTPWTFGQGTKVEI |
| | | K (SEQ ID NO: 173) |
| MHGB732 | VH | QVQLQQSGPGLVKPSQTLSLTCAISGDSVSSNSAAWN |
| | | WIRQSPSRGLEWLGRTYYRSKWYNDYAVSVKSRITIN |
| | | PDTSKNQISLQLNSVTPEDTAVYYCAGDRRYGIVGLPF |
| | | AYWGQGTLVTVSS (SEQ ID NO: 174) |
| MHGB738 | VH | QVQLQQSGPGLVKPSQTLSLTCAISGDSVSSNRAAWN |
| | | WIRQSPSRGLEWLGRTYYRSKWYNDYAVSVKSRITIN |
| | | PDTSKNQISLQLNSVTPEDTAVYYCARVRPGIPFDYWG |
| | | QGTPVTVSS (SEQ ID NO: 175) |

The complete amino acid sequences of the light chains and heavy chains of exemplary anti-HLA-G antibodies are summarized in Table 25 below.

TABLE 25
Complete amino acid sequences of the light chains
and heavy chains of exemplary anti-HLA-G
antibodies

| | Light | |
|---|---|---|
| Anti- | chain/ | |
| body | Heavy | Complete Sequence |
| Name | chain | (SEQ ID NO) |
| MHGB732 | Light | DIVMTQSPDSLAVSLGERATINCKSSQSVLHSSNN |
| | chain | KNYLTWFQQKPGQPPKLLIYWASTRESGVPDRFSG |
| | | SGSGTDFTLTISSLQAEDVAVYYCHQYYSTPPTFG |
| | | QGTKVEIKRTVAAPSVFIFPPSDEQLKSGTASVVC |
| | | LLNNFYPREAKVQWKVDNALQSGNSQESVTEQDSK |
| | | DSTYSLSSTLTLSKADYEKHKVYACEVTHQGLSSP |
| | | VTKSFNRGEC (SEQ ID NO: 176) |
| MHGB738 | Light | DIVMTQSPDSLAVSLGERATINCKSSQSVLFSSNN |
| | chain | KNYLAWYQQKPGQPPKLLIYWASTRESGVPDRFSG |
| | | SVSGTDFTLTISSLQAEDVAVYYCQQYHSTPWTFG |
| | | QGTKVEIKRTVAAPSVFIFPPSDEQLKSGTASVVC |
| | | LLNNFYPREAKVQWKVDNALQSGNSQESVTEQDSK |
| | | DSTYSLSSTLTLSKADYEKHKVYACEVTHQGLSSP |
| | | VTKSFNRGEC (SEQ ID NO: 177) |
| MHGB732 | Heavy | QVQLQQSGPGLVKPSQTLSLTCAISGDSVSSNSAA |
| | chain | WNWIRQSPSRGLEWLGRTYYRSKWYNDYAVSVKSR |
| | | ITINPDTSKNQISLQLNSVTPEDTAVYYCAGDRRY |
| | | GIVGLPFAYWGQGTLVTVSSASTKGPSVFPLAPSS |
| | | KSTSGGTAALGCLVKDYFPEPVTVSWNSGALTSGV |
| | | HTFPAVLQSSGLYSLSSVVTVPSSSLGTQTYICNV |
| | | NHKPSNTKVDKKVEPKSCDKTHTCPPCPAPELLGG |
| | | PSVFLFPPKPKDTLMISRTPEVTCVVVDVSHEDPE |
| | | VKFNWYVDGVEVHNAKTKPREEQYNSTYRVVSVLT |
| | | VLHQDWLNGKEYKCKVSNKALPAPIEKTISKAKGQ |
| | | PREPQVYTLPPSREEMTKNQVSLTCLVKGFYPSDI |
| | | AVEWESNGQPENNYKTTPPVLDSDGSFFLYSKLTV |
| | | DKSRWQQGNVFSCSVMHEALHNHYTQKSLSLSPG |
| | | (SEQ ID NO: 178) |
| MHGB738 | Heavy | QVQLQQSGPGLVKPSQTLSLTCAISGDSVSSNRAA |
| | chain | WNWIRQSPSRGLEWLGRTYYRSKWYNDYAVSVKSR |
| | | ITINPDTSKNQISLQLNSVTPEDTAVYYCARVRPG |
| | | IPFDYWGQGTPVTVSSASTKGPSVFPLAPSSKSTS |
| | | GGTAALGCLVKDYFPEPVTVSWNSGALTSGVHTFP |
| | | AVLQSSGLYSLSSVVTVPSSSLGTQTYICNVNHKP |
| | | SNTKVDKKVEPKSCDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVLHQ |
| | | DWLNGKEYKCKVSNKALPAPIEKTISKAKGQPREP |
| | | QVYTLPPSREEMTKNQVSLTCLVKGFYPSDIAVEW |
| | | ESNGQPENNYKTTPPVLDSDGSFFLYSKLTVDKSR |
| | | WQQGNVFSCSVMHEALHNHYTQKSLSLSPG |
| | | (SEQ ID NO: 179) |

The complete nucleotide sequences of the light chains and heavy chains of exemplary anti-HLA-G antibodies are summarized in Table 26 below.

TABLE 26
Complete nucleotide sequences of the light chains
and heavy chains of exemplary anti-HLA-G
antibodies

| | Light | |
|---|---|---|
| | chain/ | |
| Antibody | Heavy | Complete Sequence |
| Name | chain | (SEQ ID NO) |
| MHGB732 | Light | GACATCGTAATGACACAGTCACCAGATTCATTGG |
| | chain | CAGTTAGTCTGGGTGAAAGGGCAACAATCAACTG |
| | | CAAGTCTTCTCAGAGTGTACTGCATAGTTCTAAC |
| | | AATAAGAACTACCTTACCTGGTTTCAACAGAAAC |
| | | CAGGTCAGCCCCCCAAGTTGCTGATTTACTGGGC |
| | | AAGCACCCGCGAATCCGGCGTTCCCGATCGATTT |
| | | TCAGGTTCCGGGAGTGGGACCGACTTTACCTTGA |
| | | CCATCTCTTCCTTGCAGGCCGAAGATGTAGCCGT |
| | | CTATTACTGCCATCAGTATTACTCTACTCCCCCC |
| | | ACATTCGGTCAAGGTACAAAAGTTGAGATAAAAC |
| | | GGACAGTGGCCGCTCCTTCCGTGTTCATCTTCCC |
| | | ACCTTCCGACGAGCAGCTGAAGTCCGGCACAGCT |
| | | TCTGTCGTGTGCCTGCTGAACAACTTCTACCCTC |
| | | GGGAAGCCAAGGTGCAGTGGAAGGTGGACAATGC |
| | | CCTGCAGTCCGGCAACTCCCAAGAGTCTGTGACC |
| | | GAGCAGGACTCCAAGGACAGCACCTACAGCCTGT |
| | | CCTCCACACTGACCCTGTCCAAGGCCGACTACGA |
| | | GAAGCACAAGGTGTACGCCTGCGAAGTGACCCAT |
| | | CAGGGCCTGTCTAGCCCTGTGACCAAGTCTTTCA |
| | | ACCGGGGCGAGTGT (SEQ ID NO: 180) |
| MHGB738 | Light | GATATTGTTATGACACAGTCCCCAGATTCATTGG |
| | chain | CAGTAAGCCTCGGTGAACGGGCTACTATTAACTG |
| | | TAAGTCTTCCCAGAGTGTATTGTTCTCTTCAAAT |
| | | AACAAAAACTACCTGGCATGGTATCAGCAAAAGC |
| | | CTGGTCAACCCCCTAAACTTCTCATATACTGGGC |
| | | ATCCACTCGGGAGAGCGGTGTGCCAGACCGTTTC |
| | | TCAGGGAGTGTGTCAGGTACAGATTTTACACTCA |
| | | CAATTTCCAGCCTCCAAGCCGAAGACGTTGCAGT |
| | | ATATTATTGCCAACAATATCACTCTACACCTTGG |
| | | ACATTTGGTCAAGGTACTAAAGTCGAAATCAAAC |
| | | GGACAGTGGCCGCTCCTTCCGTGTTCATCTTCCC |
| | | ACCTTCCGACGAGCAGCTGAAGTCCGGCACAGCT |
| | | TCTGTCGTGTGCCTGCTGAACAACTTCTACCCTC |
| | | GGGAAGCCAAGGTGCAGTGGAAGGTGGACAATGC |
| | | CCTGCAGTCCGGCAACTCCCAAGAGTCTGTGACC |
| | | GAGCAGGACTCCAAGGACAGCACCTACAGCCTGT |
| | | CCTCCACACTGACCCTGTCCAAGGCCGACTACGA |
| | | GAAGCACAAGGTGTACGCCTGCGAAGTGACCCAT |
| | | CAGGGCCTGTCTAGCCCTGTGACCAAGTCTTTCA |
| | | ACCGGGGCGAGTGT (SEQ ID NO: 181) |
| MHGB732 | Heavy | CAAGTACAACTGCAACAAAGTGGTCCTGGGCTCG |
| | chain | TGAAGCCTTCCCAGACTCTCAGCCTCACATGCGC |
| | | TATAAGTGGGGATTCTGTTTCCTCAAATTCAGCA |
| | | GCCTGGAATTGGATACGACAGTCTCCATCCCGTG |
| | | GCCTTGAGTGGCTTGGTAGAACTTATTACCGATC |
| | | CAAGTGGTACAATGATTACGCCGTTTCAGTGAAG |
| | | TCCCGCATTACTATTAATCCCGACACATCTAAGA |
| | | ATCAAATTTCATTGCAACTGAATAGCGTAACACC |
| | | CGAAGATACAGCAGTTTATTATTGTGCAGGTGAT |
| | | CGACGCTACGGCATAGTGGGACTTCCTTTCGCCT |
| | | ATTGGGGCCAAGGGACACTGGTCACTGTGTCATC |
| | | CGCCTCCACCAAGGGCCCATCGGTCTTCCCCCTG |
| | | GCACCCTCCTCCAAGAGCACCTCTGGGGGCACAG |
| | | CGGCCCTGGGCTGCCTGGTCAAGGACTACTTCCC |
| | | CGAACCGGTGACGGTGTCGTGGAACTCAGGCGCC |
| | | CTGACCAGCGGCGTGCACACCTTCCCGGCTGTCC |
| | | TACAGTCCTCAGGACTCTACTCCCTCAGCAGCGT |
| | | GGTGACCGTGCCCTCCAGCAGCTTGGGCACCCAG |
| | | ACCTACATCTGCAACGTGAATCACAAGCCCAGCA |
| | | ACACCAAGGTGGACAAGAAAGTTGAGCCCAAATC |
| | | TTGTGACAAAACTCACACATGTCCACCGTGCCCA |
| | | GCACCTGAACTGCTGGGGGGACCGTCAGTCTTCC |
| | | TCTTCCCCCCAAAACCCAAGGACACCCTCATGAT |
| | | CTCCCGGACCCCTGAGGTCACATGCGTGGTGGTG |
| | | GACGTGAGCCACGAAGACCCTGAGGTCAAGTTCA |
| | | ACTGGTACGTGGACGGCGTGGAGGTGCATAATGC |
| | | CAAGACAAAGCCGCGGGAGGAGCAGTACAACAGC |
| | | ACGTACCGTGTGGTCAGCGTCCTCACCGTCCTGC |
| | | ACCAGGACTGGCTGAATGGCAAGGAGTACAAGTG |
| | | CAAGGTCTCCAACAAAGCCCTCCCAGCCCCCATC |
| | | GAGAAAACCATCTCCAAAGCCAAAGGGCAGCCCC |
| | | GAGAACCACAGGTGTACACCCTGCCCCCATCCCG |
| | | GGAGGAGATGACCAAGAACCAGGTCAGCCTGACC |
| | | TGCCTGGTCAAAGGCTTCTATCCCAGCGACATCG |
| | | CCGTGGAGTGGGAGAGCAATGGGCAGCCGGAGAA |
| | | CAACTACAAGACCACGCCTCCCGTGCTGGACTCC |
| | | GACGGCTCCTTCTTCCTCTACAGCAAGCTCACCG |
| | | TGGACAAGTCTAGATGGCAGCAGGGGAACGTCTT |
| | | CTCATGCTCCGTGATGCATGAGGCTCTGCACAAC |
| | | CACTACACGCAGAAGAGCCTCTCCCTGTCTCCGG |
| | | GT (SEQ ID NO: 182) |
| MHGB738 | Heavy | CAGGTGCAGCTTCAACAGAGCGGACCTGGTCTGG |
| | chain | TTAAGCCTTCCCAAACCCTGAGCCTGACTTGTGC |
| | | TATTTCCGGGGATAGTGTTAGCTCCAATAGGGCA |
| | | GCATGGAACTGGATCAGACAGTCCCCAAGCCGTG |
| | | GACTTGAGTGGCTTGGACGTACTTATTACAGGAG |
| | | TAAATGGTACAATGATTATGCCGTTTCTGTGAAG |
| | | AGCCGTATTACTATAAACCCAGATACTTCTAAAA |
| | | ATCAAATTTCCCTTCAGCTCAACTCAGTTACACC |
| | | AGAGGATACTGCAGTCTATTATTGCGCAAGAGTT |
| | | CGACCTGGCATTCCCTTCGATTATTGGGGGCAGG |
| | | GGACACCCGTTACTGTGTCCTCAGCCTCCACCAA |
| | | GGGCCCATCGGTCTTCCCCCTGGCACCCTCCTCC |
| | | AAGAGCACCTCTGGGGGCACAGCGGCCCTGGGCT |
| | | GCCTGGTCAAGGACTACTTCCCCGAACCGGTGAC |
| | | GGTGTCGTGGAACTCAGGCGCCCTGACCAGCGGC |
| | | GTGCACACCTTCCCGGCTGTCCTACAGTCCTCAG |
| | | GACTCTACTCCCTCAGCAGCGTGGTGACCGTGCC |
| | | CTCCAGCAGCTTGGGCACCCAGACCTACATCTGC |
| | | AACGTGAATCACAAGCCCAGCAACACCAAGGTGG |
| | | ACAAGAAAGTTGAGCCCAAATCTTGTGACAAAAC |
| | | TCACACATGTCCACCGTGCCCAGCACCTGAACTG |
| | | CTGGGGGGACCGTCAGTCTTCCTCTTCCCCCCAA |
| | | AACCCAAGGACACCCTCATGATCTCCCGGACCCC |
| | | TGAGGTCACATGCGTGGTGGTGGACGTGAGCCAC |
| | | GAAGACCCTGAGGTCAAGTTCAACTGGTACGTGG |
| | | ACGGCGTGGAGGTGCATAATGCCAAGACAAAGCC |
| | | GCGGGAGGAGCAGTACAACAGCACGTACCGTGTG |
| | | GTCAGCGTCCTCACCGTCCTGCACCAGGACTGGC |
| | | TGAATGGCAAGGAGTACAAGTGCAAGGTCTCCAA |
| | | CAAAGCCCTCCCAGCCCCCATCGAGAAAACCATC |
| | | TCCAAAGCCAAAGGGCAGCCCCGAGAACCACAGG |
| | | TGTACACCCTGCCCCCATCCCGGGAGGAGATGAC |
| | | CAAGAACCAGGTCAGCCTGACCTGCCTGGTCAAA |
| | | GGCTTCTATCCCAGCGACATCGCCGTGGAGTGGG |
| | | AGAGCAATGGGCAGCCGGAGAACAACTACAAGAC |
| | | CACGCCTCCCGTGCTGGACTCCGACGGCTCCTTC |
| | | TTCCTCTACAGCAAGCTCACCGTGGACAAGTCTA |
| | | GATGGCAGCAGGGGAACGTCTTCTCATGCTCCGT |
| | | GATGCATGAGGCTCTGCACAACCACTACACGCAG |
| | | AAGAGCCTCTCCCTGTCTCCGGGT |
| | | (SEQ ID NO: 183) |

### Example 7—ADCC Functional Analysis of Anti-PSMA Antibodies

Anti-PSMA antibodies PSMB896 and PSMB898 were modified in its Fc region to introduce the K248E and T437R mutations (RE mutations). The anti-PSMA antibodies with RE mutations were expressed in fucosylation-deficient cells to produce antibodies with low fucosylation. The resulting antibody for PSMB896 is PSMB952; the resulting antibody for PSMB898 is PSMB956. The binding and thermal stability of PSMB952 and PSMB956 were assayed. The results indicate that low fucosylation and RE mutations do not impact these biophysical characteristics of the PSMB896 and PSMB898 antibodies.

C42B and LNCap cells stably transfected with GFP were plated at 9,000 cells per well in a 384-well plate (Perkin Elmer ViewPlate) in clear media (RPMI 1641+10% FBS, Thermo Fisher Scientific) to allow for cell adherence overnight. ADCC assay was performed with freshly thawed PBMC (Hemcare, PB009C-3) or NK cells isolated from the frozen PBMC by RoboSep™ Cell Separation Instruments. Isolated NK cells were either used immediately or primed overnight with low dose IL-2 (1 ng/ml, Miltenyi Biotec). The ratio of effector to target cell per well was 34:1 for PBMC and 5:1 for isolated NK cells. The PSMB952 and PSMB956 antibodies were tested with final concentrations ranging from 100 nM to 0.01 nM. After effector cells and antibodies were added to target cells, real time imaging was performed under Incucyte® S3 instrument (Essen BioScience). Total GFP intergraded signal per well was quantified with Incucyte® software. Data analysis was performed by Incucyte® software and Prism (GraphPad Software) based on values of quadruplicates. The percentage of cell killing was calculated as: (1−PSMA mAb/no mAb control)×100%.

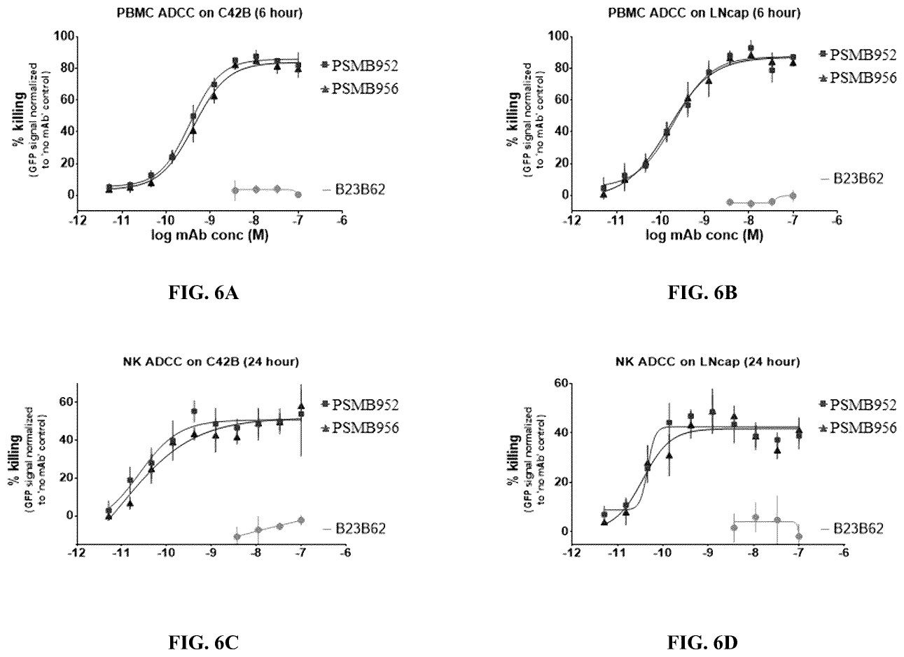

The PSMB952 and PSMB956 antibodies demonstrated obvious dose dependent ADCC activities by effector cells on C42B and LNCap cells (FIGS. 6A-6D). The kinetic showed that ADCC activities initiated immediately after addition of effector cells and antibodies and continued with time. The maximal ADCC activity are ~97% for PBMC and 40-50% for purified NK cells.

Dose-response curves generated at certain time points (6 hour for PBMC, 24 hour for NK cells) showed the killing EC50 is in the range of 10⁻¹⁰~10⁻¹¹M (Table 27).

TABLE 27
In vitro ADCC killing EC50 (M) by PBMC at 6 hour
and NK cells at 24 hour on C42B and LNCap cells

| Target cell | Effector cell | PSMB952 | PSMB956 |
|---|---|---|---|
| C42B | PBMC | 3.51e−010 | 4.231e−010 |
| LNCap | PBMC | 2.011e−010 | 1.519e−010 |
| C42B | NK | 2.453e−011 | 9.956e−012 |
| LNCap | NK | 4.585e−011 | 3.579e−011 |

The VH and VL amino acid sequences of exemplary anti-PSMA antibodies are summarized in Table 28 below.

TABLE 28
VH and VL amino acid sequences of exemplary anti-
PSMA antibodies

| Anti- | | |
|---|---|---|
| body | VH/ | Complete Sequence |
| Name | VL | (SEQ ID NO) |
| PSMB896 | VL | QSVLTQPPSVSAAPGQKVTISCSGSSSNIGINYV |
| | | SWYQQLPGTAPKLLIYDNNKRPSGIPDRFSGSKS |
| | | GTSATLGITGLQTGDEADYYCGTWDSSLSAVVFG |
| | | GGTKLTVL (SEQ ID NO: 232) |
| PSMB898 | VL | QSVLTQPPSVSAAPGQKVTISCSGSSSNIGINYV |
| | | SWYQQLPGTAPKLLIYDNNKRPSGIPDRFSGSKS |
| | | GTSATLGITGLQTGDEADYYCGTWDSSLSAVVFG |
| | | GGTKLTVL (SEQ ID NO: 232) |
| PSMB896 | VH | EVQLVESGGGLVQPGGSLRLSCAASGFTFSSYAM |
| | | SWVRQAPGKGLEWVSAISGGIGSTYYADSVKGRF |
| | | TISRDNSKNTLWLQMNSLRAEDTAVYYCAKDGVG |
| | | ATPYYFDYWGQGTLVTVSS |
| | | (SEQ ID NO: 233) |
| PSMB898 | VH | EVQLVESGGGLVQPGGSLRLSCAASGFTFSSYAM |
| | | SWVRQAPGKGLEWVSAISGGSGSTYYADSVKGRF |
| | | TISRDNSKNTLYLQMNSLRAEDSAVYYCAKDGVG |
| | | ATPYYFDYWGQGTLVTVSS |
| | | (SEQ ID NO: 234) |

The VH and VL nucleotide sequences of exemplary anti-PSMA antibodies are summarized in Table 29 below.

TABLE 29
VH and VL nucleotide sequences of exemplary anti-
PSMA antibodies

| Anti- | | |
|---|---|---|
| body | VH/ | Complete Sequence |
| Name | VL | (SEQ ID NO) |
| PSMB896 | VL | CAGAGTGTCCTTACTCAGCCTCCTAGCGTTAGCG |
| | | CCGCCCCTGGACAGAAGGTTACTATCTCCTGCTC |
| | | AGGGAGTTCCAGTAATATTGGAATCAATTATGTG |
| | | AGTTGGTATCAGCAGTTGCCCGGCACCGCTCCTA |
| | | AATTGCTTATCTATGACAACAATAAACGACCTAG |
| | | TGGTATCCCTGATCGTTTTTCTGGATCAAAATCT |
| | | GGTACTAGCGCAACCCTCGGTATCACCGGACTGC |
| | | AAACAGGTGATGAAGCAGACTATTATTGCGGCAC |
| | | CTGGGACTCATCACTCTCCGCCGTCGTTTTCGGG |
| | | GGCGGAACCAAACTTACAGTATTG |
| | | (SEQ ID NO: 235) |
| PSMB898 | VL | CAGAGTGTCCTTACTCAGCCTCCTAGCGTTAGCG |
| | | CCGCCCCTGGACAGAAGGTTACTATCTCCTGCTC |
| | | AGGGAGTTCCAGTAATATTGGAATCAATTATGTG |
| | | AGTTGGTATCAGCAGTTGCCCGGCACCGCTCCTA |
| | | AATTGCTTATCTATGACAACAATAAACGACCTAG |
| | | TGGTATCCCTGATCGTTTTTCTGGATCAAAATCT |
| | | GGTACTAGCGCAACCCTCGGTATCACCGGACTGC |
| | | AAACAGGTGATGAAGCAGACTATTATTGCGGCAC |
| | | CTGGGACTCATCACTCTCCGCCGTCGTTTTCGGG |
| | | GGCGGAACCAAACTTACAGTATTG |
| | | (SEQ ID NO: 235) |
| PSMB896 | VH | GAGGTACAACTTGTGGAAAGTGGAGGCGGTCTTG |
| | | TCCAACCTGGAGGATCTCTCCGATTGAGTTGCGC |
| | | AGCCAGCGGGTTTACTTTTTCTTCATACGCCATG |
| | | TCCTGGGTGCGGCAAGCACCAGGTAAAGGACTTG |
| | | AGTGGGTATCTGCTATTTCAGGGGGGATAGGCTC |
| | | AACATACTATGCTGATAGCGTGAAAGGTAGGTTC |
| | | ACCATTTCCCGTGACAATAGTAAAAACACATTGT |
| | | GGCTCCAAATGAACAGCCTTAGGGCTGAAGACAC |
| | | CGCTGTTTACTACTGCGCTAAAGACGGTGTAGGG |
| | | GCAACTCCCTATTACTTCGATTATTGGGGACAAG |
| | | GAACCTTGGTAACAGTTTCAAGC |
| | | (SEQ ID NO: 236) |
| PSMB898 | VH | GAAGTCCAGTTGGTAGAATCTGGAGGCGGGCTGG |
| | | TACAGCCTGGCGGTTCCTTGCGCCTCTCATGTGC |
| | | CGCAAGCGGGTTTACCTTCAGCTCTTACGCAATG |
| | | TCATGGGTGCGTCAGGCCCCTGGAAAAGGTCTCG |
| | | AGTGGGTCAGTGCCATTTCTGGGGGCTCCGGCTC |
| | | CACCTACTACGCAGATTCAGTTAAAGGGAGATTT |
| | | ACAATCTCAAGAGATAACAGTAAAAACACCCTCT |
| | | ACCTCCAGATGAACTCACTTCGAGCTGAGGATTC |
| | | AGCAGTATATTACTGTGCTAAAGACGGTGTAGGT |
| | | GCAACTCCCTACTATTTCGACTATTGGGGCCAAG |
| | | GGACTTTGGTGACAGTAAGTAGT |
| | | (SEQ ID NO: 237) |

The complete amino acid sequences of the light chains and heavy chains of exemplary anti-PSMA antibodies are summarized in Table 30 below.

TABLE 30
Heavy chain and light chain amino acid sequences
of exemplary anti-PSMA antibodies

| Anti- | | |
|---|---|---|
| body | | Complete Sequence |
| Name | Region | (SEQ ID NO) |
| PSMB896 | Light | QSVLTQPPSVSAAPGQKVTISCSGSSSNIGINYV |
| | chain | SWYQQLPGTAPKLLIYDNNKRPSGIPDRFSGSKS |
| | | GTSATLGITGLQTGDEADYYCGTWDSSLSAVVFG |
| | | GGTKLTVLGQPKAAPSVTLFPPSSEELQANKATL |
| | | VCLISDFYPGAVTVAWKADSSPVKAGVETTTPSK |
| | | QSNNKYAASSYLSLTPEQWKSHRSYSCQVTHEGS |
| | | TVEKTVAPTECS (SEQ ID NO: 238) |
| PSMB898 | Light | QSVLTQPPSVSAAPGQKVTISCSGSSSNIGINYV |
| | chain | SWYQQLPGTAPKLLIYDNNKRPSGIPDRFSGSKS |
| | | GTSATLGITGLQTGDEADYYCGTWDSSLSAVVFG |
| | | GGTKLTVLGQPKAAPSVTLFPPSSEELQANKATL |
| | | VCLISDFYPGAVTVAWKADSSPVKAGVETTTPSK |
| | | QSNNKYAASSYLSLTPEQWKSHRSYSCQVTHEGS |
| | | TVEKTVAPTECS (SEQ ID NO: 238) |
| PSMB896 | Heavy | EVQLVESGGGLVQPGGSLRLSCAASGFTFSSYAM |
| | chain | SWVRQAPGKGLEWVSAISGGIGSTYYADSVKGRF |
| | | TISRDNSKNTLWLQMNSLRAEDTAVYYCAKDGVG |
| | | ATPYYFDYWGQGTLVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYIC |
| | | NVNHKPSNTKVDKKVEPKSCDKTHTCPPCPAPEA |
| | | AGGPSVFLFPPKPKDTLMISRTPEVTCVVVSVSH |
| | | EDPEVKFNWYVDGVEVHNAKTKPREEQYNSTYRV |
| | | VSVLTVLHQDWLNGKEYKCKVSNKALPAPIEKTI |
| | | SKAKGQPREPQVYTLPPSREEMTKNQVSLTCLVK |
| | | GFYPSDIAVEWESNGQPENNYKTTPPVLDSDGSF |
| | | FLYSRLTVDKSRWQQGNVFSCSVMHEALHNHYTQ |
| | | KSLSLSPG (SEQ ID NO: 239) |
| PSMB898 | Heavy | EVQLVESGGGLVQPGGSLRLSCAASGFTFSSYAM |
| | chain | SWVRQAPGKGLEWVSAISGGSGSTYYADSVKGRF |
| | | TISRDNSKNTLYLQMNSLRAEDSAVYYCAKDGVG |
| | | ATPYYFDYWGQGTLVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYIC |
| | | NVNHKPSNTKVDKKVEPKSCDKTHTCPPCPAPEA |
| | | AGGPSVFLFPPKPKDTLMISRTPEVTCVVVSVSH |
| | | EDPEVKFNWYVDGVEVHNAKTKPREEQYNSTYRV |
| | | VSVLTVLHQDWLNGKEYKCKVSNKALPAPIEKTI |
| | | SKAKGQPREPQVYTLPPSREEMTKNQVSLTCLVK |
| | | GFYPSDIAVEWESNGQPENNYKTTPPVLDSDGSF |
| | | FLYSRLTVDKSRWQQGNVFSCSVMHEALHNHYTQ |
| | | KSLSLSPG (SEQ ID NO: 240) |

The complete nucleotide sequences of the light chains and heavy chains of exemplary anti-PSMA antibodies are summarized in Table 31 below.

TABLE 31
Heavy chain and light chain nucleotide sequences
of exemplary anti-PSMA antibodies

| Anti- | | |
|---|---|---|
| body | | Complete Sequence |
| Name | Region | (SEQ ID NO) |
| PSMB896 | Light | CAGAGTGTCCTTACTCAGCCTCCTAGCGTTAGCG |
| | chain | CCGCCCCTGGACAGAAGGTTACTATCTCCTGCTC |
| | | AGGGAGTTCCAGTAATATTGGAATCAATTATGTG |
| | | AGTTGGTATCAGCAGTTGCCCGGCACCGCTCCTA |
| | | AATTGCTTATCTATGACAACAATAAACGACCTAG |
| | | TGGTATCCCTGATCGTTTTTCTGGATCAAAATCT |
| | | GGTACTAGCGCAACCCTCGGTATCACCGGACTGC |
| | | AAACAGGTGATGAAGCAGACTATTATTGCGGCAC |
| | | CTGGGACTCATCACTCTCCGCCGTCGTTTTCGGG |
| | | GGCGGAACCAAACTTACAGTATTGGGTCAGCCCA |
| | | AGGCTGCACCCAGTGTCACTCTGTTCCCGCCCTC |
| | | CTCTGAGGAGCTTCAAGCCAACAAGGCCACACTG |
| | | GTGTGTCTCATAAGTGACTTCTACCCGGGAGCCG |
| | | TGACAGTGGCCTGGAAGGCCGATAGCAGCCCCGT |
| | | CAAGGCGGGAGTGGAGACCACCACACCCTCCAAA |
| | | CAAAGCAACAACAAGTACGCGGCCAGCAGCTATC |
| | | TGAGCCTGACGCCTGAGCAGTGGAAGTCCCACAG |
| | | AAGCTACAGCTGCCAGGTCACGCATGAAGGGAGC |
| | | ACCGTGGAGAAGACAGTGGCCCCTACAGAATGTT |
| | | CA (SEQ ID NO: 241) |
| PSMB898 | Light | CAGAGTGTCCTTACTCAGCCTCCTAGCGTTAGCG |
| | chain | CCGCCCCTGGACAGAAGGTTACTATCTCCTGCTC |
| | | AGGGAGTTCCAGTAATATTGGAATCAATTATGTG |
| | | AGTTGGTATCAGCAGTTGCCCGGCACCGCTCCTA |
| | | AATTGCTTATCTATGACAACAATAAACGACCTAG |
| | | TGGTATCCCTGATCGTTTTTCTGGATCAAAATCT |
| | | GGTACTAGCGCAACCCTCGGTATCACCGGACTGC |
| | | AAACAGGTGATGAAGCAGACTATTATTGCGGCAC |
| | | CTGGGACTCATCACTCTCCGCCGTCGTTTTCGGG |
| | | GGCGGAACCAAACTTACAGTATTGGGTCAGCCCA |
| | | AGGCTGCACCCAGTGTCACTCTGTTCCCGCCCTC |
| | | CTCTGAGGAGCTTCAAGCCAACAAGGCCACACTG |
| | | GTGTGTCTCATAAGTGACTTCTACCCGGGAGCCG |
| | | TGACAGTGGCCTGGAAGGCCGATAGCAGCCCCGT |
| | | CAAGGCGGGAGTGGAGACCACCACACCCTCCAAA |
| | | CAAAGCAACAACAAGTACGCGGCCAGCAGCTATC |
| | | TGAGCCTGACGCCTGAGCAGTGGAAGTCCCACAG |
| | | AAGCTACAGCTGCCAGGTCACGCATGAAGGGAGC |
| | | ACCGTGGAGAAGACAGTGGCCCCTACAGAATGTT |
| | | CA (SEQ ID NO: 241) |
| PSMB896 | Heavy | GAGGTACAACTTGTGGAAAGTGGAGGCGGTCTTG |
| | chain | TCCAACCTGGAGGATCTCTCCGATTGAGTTGCGC |
| | | AGCCAGCGGGTTTACTTTTTCTTCATACGCCATG |
| | | TCCTGGGTGCGGCAAGCACCAGGTAAAGGACTTG |
| | | AGTGGGTATCTGCTATTTCAGGGGGGATAGGCTC |
| | | AACATACTATGCTGATAGCGTGAAAGGTAGGTTC |
| | | ACCATTTCCCGTGACAATAGTAAAAACACATTGT |
| | | GGCTCCAAATGAACAGCCTTAGGGCTGAAGACAC |
| | | CGCTGTTTACTACTGCGCTAAAGACGGTGTAGGG |
| | | GCAACTCCCTATTACTTCGATTATTGGGGACAAG |
| | | GAACCTTGGTAACAGTTTCAAGCGCTTCCACCAA |
| | | GGGCCCATCCGTCTTCCCCCTGGCACCCTCCTCC |
| | | AAGAGCACCTCTGGGGGCACAGCGGCCCTGGGCT |
| | | GCCTGGTCAAGGACTACTTCCCCGAACCGGTGAC |
| | | GGTGTCGTGGAACTCAGGCGCCCTGACCAGCGGC |
| | | GTGCACACCTTCCCGGCTGTCCTACAGTCCTCAG |
| | | GACTCTACTCCCTCAGCAGCGTGGTGACCGTGCC |
| | | CTCCAGCAGCTTGGGCACCCAGACCTACATCTGC |
| | | AACGTGAATCACAAGCCCAGCAACACCAAGGTGG |
| | | ACAAGAAAGTTGAGCCCAAATCTTGTGACAAAAC |
| | | TCACACATGTCCACCGTGCCCAGCACCTGAAGCA |
| | | GCAGGGGGACCGTCAGTCTTCCTCTTCCCCCCAA |
| | | AACCCAAGGACACCCTCATGATCTCCCGGACCCC |
| | | TGAGGTCACATGCGTGGTGGTGAGCGTGAGCCAC |
| | | GAAGACCCTGAGGTCAAGTTCAACTGGTACGTGG |
| | | ACGGCGTGGAGGTGCATAATGCCAAGACAAAGCC |
| | | GCGGGAGGAGCAGTACAACAGCACGTACCGTGTG |
| | | GTCAGCGTCCTCACCGTCCTGCACCAGGACTGGC |
| | | TGAATGGCAAGGAGTACAAGTGCAAGGTCTCCAA |
| | | CAAAGCCCTCCCAGCCCCCATCGAGAAAACCATC |
| | | TCCAAAGCCAAAGGGCAGCCCCGAGAACCACAGG |
| | | TGTACACCCTGCCCCCATCCCGGGAGGAGATGAC |
| | | CAAGAACCAGGTCAGCCTGACCTGCCTGGTCAAA |
| | | GGCTTCTATCCCAGCGACATCGCCGTGGAGTGGG |
| | | AGAGCAATGGGCAGCCGGAGAACAACTACAAGAC |
| | | CACGCCTCCCGTGCTGGACTCCGACGGCTCCTTC |
| | | TTCCTCTACAGCCGGCTCACCGTGGACAAGTCTA |
| | | GATGGCAGCAGGGGAACGTCTTCTCATGCTCCGT |
| | | GATGCATGAGGCTCTGCACAACCACTACACGCAG |
| | | AAGAGCCTCTCCCTGTCTCCGGGT |
| | | (SEQ ID NO: 242) |
| PSMB898 | Heavy | GAAGTCCAGTTGGTAGAATCTGGAGGCGGGCTGG |
| | chain | TACAGCCTGGCGGTTCCTTGCGCCTCTCATGTGC |
| | | CGCAAGCGGGTTTACCTTCAGCTCTTACGCAATG |
| | | TCATGGGTGCGTCAGGCCCCTGGAAAAGGTCTCG |
| | | AGTGGGTCAGTGCCATTTCTGGGGGCTCCGGCTC |
| | | CACCTACTACGCAGATTCAGTTAAAGGGAGATTT |
| | | ACAATCTCAAGAGATAACAGTAAAAACACCCTCT |
| | | ACCTCCAGATGAACTCACTTCGAGCTGAGGATTC |
| | | AGCAGTATATTACTGTGCTAAAGACGGTGTAGGT |
| | | GCAACTCCCTACTATTTCGACTATTGGGGCCAAG |
| | | GGACTTTGGTGACAGTAAGTAGTGCTTCCACCAA |
| | | GGGCCCATCCGTCTTCCCCCTGGCACCCTCCTCC |
| | | AAGAGCACCTCTGGGGGCACAGCGGCCCTGGGCT |
| | | GCCTGGTCAAGGACTACTTCCCCGAACCGGTGAC |
| | | GGTGTCGTGGAACTCAGGCGCCCTGACCAGCGGC |
| | | GTGCACACCTTCCCGGCTGTCCTACAGTCCTCAG |
| | | GACTCTACTCCCTCAGCAGCGTGGTGACCGTGCC |
| | | CTCCAGCAGCTTGGGCACCCAGACCTACATCTGC |
| | | AACGTGAATCACAAGCCCAGCAACACCAAGGTGG |
| | | ACAAGAAAGTTGAGCCCAAATCTTGTGACAAAAC |
| | | TCACACATGTCCACCGTGCCCAGCACCTGAAGCA |
| | | GCAGGGGGACCGTCAGTCTTCCTCTTCCCCCCAA |
| | | AACCCAAGGACACCCTCATGATCTCCCGGACCCC |
| | | TGAGGTCACATGCGTGGTGGTGAGCGTGAGCCAC |
| | | GAAGACCCTGAGGTCAAGTTCAACTGGTACGTGG |
| | | ACGGCGTGGAGGTGCATAATGCCAAGACAAAGCC |
| | | GCGGGAGGAGCAGTACAACAGCACGTACCGTGTG |
| | | GTCAGCGTCCTCACCGTCCTGCACCAGGACTGGC |
| | | TGAATGGCAAGGAGTACAAGTGCAAGGTCTCCAA |
| | | CAAAGCCCTCCCAGCCCCCATCGAGAAAACCATC |
| | | TCCAAAGCCAAAGGGCAGCCCCGAGAACCACAGG |
| | | TGTACACCCTGCCCCCATCCCGGGAGGAGATGAC |
| | | CAAGAACCAGGTCAGCCTGACCTGCCTGGTCAAA |
| | | GGCTTCTATCCCAGCGACATCGCCGTGGAGTGGG |
| | | AGAGCAATGGGCAGCCGGAGAACAACTACAAGAC |
| | | CACGCCTCCCGTGCTGGACTCCGACGGCTCCTTC |
| | | TTCCTCTACAGCCGGCTCACCGTGGACAAGTCTA |
| | | GATGGCAGCAGGGGAACGTCTTCTCATGCTCCGT |
| | | GATGCATGAGGCTCTGCACAACCACTACACGCAG |
| | | AAGAGCCTCTCCCTGTCTCCGGGT |
| | | (SEQ ID NO: 243) |

### Example 8—ADCC and CDC Functional Analysis of Bispecific PSMA×CD3 Antibodies

Exemplary bispecific PSMA×CD3 antibodies are modified in its Fc region to introduce the RE mutations (K248E and T437R mutations). The bispecific PSMA×CD3 antibodies with RE mutations are expressed in fucosylation-deficient cells to produce antibodies with low fucosylation. The binding and thermal stability of the resulting antibodies are assayed.

C42B and LNCap cells stably transfected with GFP are plated at 9,000 cells per well in a 384-well plate (Perkin Elmer ViewPlate) in clear media (RPMI 1641+10% FBS, Thermo Fisher Scientific) to allow for cell adherence overnight. ADCC assay is performed with freshly thawed PBMC (Hemcare, PB009C-3) or NK cells isolated from the frozen PBMC by RoboSep™ Cell Separation Instruments. Isolated NK cells are either used immediately or primed overnight with low dose IL-2 (1 ng/ml, Miltenyi Biotec). The ratio of effector to target cell per well is 34:1 for PBMC and 5:1 for isolated NK cells. The bispecific PSMA×CD3 antibodies with low fucosylation and RE mutations are tested with final concentrations ranging from 100 nM to 0.01 nM. After effector cells and antibodies are added to target cells, real time imaging is performed under Incucyte® S3 instrument (Essen BioScience). Total GFP intergraded signal per well is quantified with Incucyte® software. Data analysis is performed by Incucyte® software and Prism (GraphPad Software) based on values of quadruplicates. The percentage of cell killing is calculated as: (1−PSMA mAb/no mAb control)×100%.

Next, the abilities of these antibodies to mediate CDC are tested. Briefly, assays are run in 10% FBS containing DMEM (JEG-3) or RPMI (RERF-LC-Ad-1). Antibodies are added to target cells and incubated for 30 minutes at 37° C. After incubation, 15-20% (stock concentration) of rabbit complement (Cedarlane cat. #CL3441-S) and heat inactivated complement are added to the wells respectively to a volume of 25 μl/well. The mixture is incubated for 4-12 hours at 37° C. Target cell lysis is detected by addition of 100 μl of CellTitre-Glo (Promega cat. #G9242) reagent followed by incubation for 10 minutes at room temperature. Luminescence is monitored using a Tecan Microplate reader SPARK®.

### Example 9—ADCC and CDC Functional Analysis of Anti-BCMA Antibodies

Exemplary anti-BCMA antibody BCMB519 is modified in its Fc region to introduce the RE mutations (K248E and T437R mutations). The anti-BCMA antibody with the RE mutations are expressed in fucosylation-deficient cells to produce antibodies with low fucosylation. The binding and thermal stability of the resulting antibodies are assayed.

C42B and LNCap cells stably transfected with GFP are plated at 9,000 cells per well in a 384-well plate (Perkin Elmer ViewPlate) in clear media (RPMI 1641+10% FBS, Thermo Fisher Scientific) to allow for cell adherence overnight. ADCC assay is performed with freshly thawed PBMC (Hemcare, PB009C-3) or NK cells isolated from the frozen PBMC by RoboSep™ Cell Separation Instruments. Isolated NK cells are either used immediately or primed overnight with low dose IL-2 (1 ng/ml, Miltenyi Biotec). The ratio of effector to target cell per well is 34:1 for PBMC and 5:1 for isolated NK cells. The anti-BCMA antibodies with low fucosylation and RE mutations are tested with final concentrations ranging from 100 nM to 0.01 nM. After effector cells and antibodies are added to target cells, real time imaging is performed under Incucyte® S3 instrument (Essen BioScience). Total GFP intergraded signal per well is quantified with Incucyte® software. Data analysis is performed by Incucyte® software and Prism (GraphPad Software) based on values of quadruplicates. The percentage of cell killing is calculated as: (1−BCMA mAb/no mAb control)×100%.

Next, the abilities of these antibodies to mediate CDC are tested. Briefly, assays are run in 10% FBS containing DMEM (JEG-3) or RPMI (RERF-LC-Ad-1). Antibodies are added to target cells and incubated for 30 minutes at 37° C. After incubation, 15-20% (stock concentration) of rabbit complement (Cedarlane cat. #CL3441-S) and heat inactivated complement are added to the wells respectively to a volume of 25 μl/well. The mixture is incubated for 4-12 hours at 37° C. Target cell lysis is detected by addition of 100 μl of CellTitre-Glo (Promega cat. #G9242) reagent followed by incubation for 10 minutes at room temperature. Luminescence is monitored using a Tecan Microplate reader SPARK®.

The VH CDR and VL CDR amino acid sequences of the exemplary anti-BCMA antibody (BCMB519) are shown in Table 32 below.

TABLE 32
CDR amino acid sequences of an exemplary anti-
BCMA antibody

| | | SEQ | | SEQ | | SEQ |
|---|---|---|---|---|---|---|
| | | ID | | ID | | ID |
| mAb ID | HCDR1 | NO: | HCDR2 | NO: | HCDR3 | NO: |
| BCMB519 | GFTFSSYA | 292 | ISGSG | 293 | AKDEGYSSGH | 294 |
| | | | GST | | YYGMDV | |
| BCMB519 | QSISSSF | 295 | GAS | 296 | QHYGSSPMYT | 297 |

The VH and VL amino acid sequences of the exemplary anti-BCMA antibody (BCMB519) are summarized in Table 33 below.

TABLE 33
VH and VL amino acid sequences of an exemplary anti-BCMA
antibody

| | | SEQ ID |
|---|---|---|
| mAb ID | | NO: |
| | VH Amino Acid Sequence | |
| BCMB519 | EVQLLESGGGLVQPGGSLRLSCAASGFTFSSYAMSWVRQAPGKGLE | 298 |
| | WVSAISGSGGSTYYADSVKGRFTISRDNSKNTLYLQMNSLRAEDTAV | |
| | YYCAKDEGYSSGHYYGMDVWGQGTTVTVSS | |
| | VL Amino Acid Sequence | |
| BCMB519 | EIVLTQSPGTLSLSPGERATLSCRASQSISSSFLTWYQQKPGQAPRLLIY | 299 |
| | GASSRATGIPDRFSGGGSGTDFTLTISRLEPEDFAVYYCQHYGSSPMYT | |
| | FGQGTKLEIK | |

The complete amino acid sequences of the heavy chain of the exemplary anti-BCMA antibody (BCMB519) is summarized in Table 34 below.

TABLE 34
Heavy chain amino acid sequences of the exemplary anti-BCMA
antibody

| | | SEQ ID |
|---|---|---|
| mAb ID | Heavy Chain Amino Acid Sequence | NO: |
| BCMB519 | MAWVWTLLFLMAAAQSIQAEIVLTQSPGTLSLSPGERATLSCRASQSI | 300 |
| | SSSFLTWYQQKPGQAPRLLIYGASSRATGIPDRFSGGGSGTDFTLTISR | |
| | LEPEDFAVYYCQHYGSSPMYTFGQGTKLEIKGGSEGKSSGSGSESKST | |
| | GGSEVQLLESGGGLVQPGGSLRLSCAASGFTFSSYAMSWVRQAPGKG | |
| | LEWVSAISGSGGSTYYADSVKGRFTISRDNSKNTLYLQMNSLRAEDT | |
| | AVYYCAKDEGYSSGHYYGMDVWGQGTTVTVSSEPKSSDKTHTCPPC | |
| | PAPEAAGGPSVFLFPPKPKDTLMISRTPEVTCVVVSVSHEDPEVKFNW | |
| | YVDGVEVHNAKTKPREEQYNSTYRVVSVLTVLHQDWLNGKEYKCK | |
| | VSNKALPAPIEKTISKAKGQPREPQVYVLPPSREEMTKNQVSLLCLVK | |
| | GFYPSDIAVEWESNGQPENNYLTWPPVLDSDGSFFLYSKLTVDKSRW | |
| | QQGNVFSCSVMHEALHNHYTQKSLSLSPG | |

1. A population of antibodies,
(i) wherein less than 80% of the oligosaccharides covalently attached to the population of the antibodies via N297 residues thereof comprise a core fucose residue; and
(ii) wherein the population of the antibodies comprises an antibody which Fc region comprises K338A and T437R mutations, or K248E and T437R mutations (RE mutations),
wherein amino acid residue numbering is according to the EU numbering system, and
wherein each antibody in the population of the antibodies comprises the RE mutations.

2. The population of the antibodies of claim 1, wherein:
(i) less than 70% of the oligosaccharides covalently attached to the population of the antibodies via N297 residues thereof comprise a core fucose residue;
(ii) less than 60% of the oligosaccharides covalently attached to the population of the antibodies via N297 residues thereof comprise a core fucose residue;
(iii) less than 50% of the oligosaccharides covalently attached to the population of the antibodies via N297 residues thereof comprise a core fucose residue;
(iv) less than 40% of the oligosaccharides covalently attached to the population of the antibodies via N297 residues thereof comprise a core fucose residue;
(v) less than 30% of the oligosaccharides covalently attached to the population of the antibodies via N297 residues thereof comprise a core fucose residue;
(vi) less than 20% of the oligosaccharides covalently attached to the population of the antibodies via N297 residues thereof comprise a core fucose residue; or
(vii) less than 10% of the oligosaccharides covalently attached to the population of the antibodies via N297 residues thereof comprise a core fucose residue.

3-8. (canceled)

9. A population of antibodies comprising an antibody, wherein the oligosaccharide covalently attached to the antibody via N297 residues thereof does not comprise a core fucose residue; and wherein the antibody comprises an Fc region comprising K338A and T437R mutations, or K248E and T437R mutations (RE mutations), wherein amino acid residue numbering is according to the EU numbering system, and wherein the antibody is an IgG antibody.

10. The population of the antibodies of claim 1, wherein the antibodies are produced by expressing a polynucleotide encoding the antibodies or a fragment thereof in a host cell that is deficient in adding a fucose to an oligosaccharide attached to an antibody.

11. (canceled)

12. The population of the antibodies of claim 1, wherein the population of the antibodies have both enhanced antibody-dependent cellular cytotoxicity (ADCC) and enhanced complement-dependent cytotoxicity (CDC).

13. The population of the antibodies of claim 1, wherein the antibodies are IgG1.

14. The population of the antibodies of claim 1, wherein the antibodies bind to HLA-G, CD37, GPRC5D, KLK2, PSMA or BCMA.

15-19. (canceled)

20. The population of the antibodies of claim 1, wherein the antibodies are monospecific antibodies, or multispecific antibodies.

21-22. (canceled)

23. A population of antibodies,
(i) wherein less than 80% of the oligosaccharides covalently attached to the population of the antibodies via N297 residues thereof comprise a core fucose residue;
(ii) wherein each antibody in the population of the antibodies comprises one or more mutations in the Fc region thereof for increasing CDC activity of the antibody.

24. (canceled)

25. A pharmaceutical composition comprising the population of the antibodies of claim 1, and a pharmaceutically acceptable excipient.

26. A method of making a population of antibodies of claim 1, comprising expressing a polynucleotide encoding the antibodies or a fragment thereof in a host cell that is deficient in adding a fucose residue to an oligosaccharide attached to an antibody via N297 residue.

27. The method of claim 26, wherein the host cell has:
(i) reduced α-1,6 fucosyltransferase activity; and/or
(ii) reduced GDP-mannose 4,6-dehydratase activity,
wherein optionally the gene encoding α-1,6 fucosyltransferase is mutated, expressed at a lower than normal level, or knocked out in the host cell, or the gene encoding GDP-mannose 4,6-dehydratase is mutated, expressed at a lower than normal level, or knocked out in the host cell.

28-30. (canceled)

31. A method of making a population of antibodies of claim 1, comprising
(i) a step for introducing K338A and T437R mutations, or K248E and T437R mutations (RE mutations) in the Fc regions of the population of the antibodies; and
(ii) a step for producing the population of antibodies with reduced amount of core fucoses in the oligosaccharides attached to the antibodies via N297 residues.

32. A method of treating a disease or disorder in a subject, comprising administering to the subject the population of the antibodies of claim 1.

33. The method of claim 32, wherein
(i) the disease or disorder is solid tumor cancer, or
(ii) the disease or disorder is selected from a group consisting of renal, pancreatic or lung adenocarcinoma, non-small cell lung cancer, and ovarian cancer.

34. (canceled)

35. A method of modulating an immunity in a host, comprising administering the population of the antibodies of claim 1.
